SEQUENCE LISTING (92 sequences)

SEQ ID NO: 1 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 2 - protein, 282 aa, Macaca fascicularis
MASLGQILFWSIISIIFILAGAIALIIGFGISGRHSITVTTVASAGNIGEDGILSCTFEPDIKLSDIVIQWLKEGVIGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFLAISWALLPLAPYLMLK

SEQ ID NO: 3 - protein, 283 aa, Mus musculus
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFVAGWALLSLSCCLMLR

SEQ ID NO: 4 - protein, 282 aa, Rattus norvegicus
MASLGQIIFWSIINVIIILAGAIVLIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIHTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLELLNSGPSPCVSSVSAAGWALLSLSCCLMLR

SEQ ID NO: 5 - protein, 11 aa, synthetic construct
GSIKSGSYYWG

SEQ ID NO: 6 - protein, 16 aa, synthetic construct
NIYYSGSTYYNPSLRS

SEQ ID NO: 7 - protein, 12 aa, synthetic construct
AREGSYPNQFDP

SEQ ID NO: 8 - protein, 11 aa, synthetic construct
RASQSVSSNLA

SEQ ID NO: 9 - protein, 7 aa, synthetic construct
GASTRAT

SEQ ID NO: 10 - protein, 9 aa, synthetic construct
QQYHSFPFT

SEQ ID NO: 11 - protein, 120 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 12 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 13 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 9 aa, synthetic construct
GTFSSYAIS

SEQ ID NO: 16 - protein, 9 aa, synthetic construct
FTFSSYAMS

SEQ ID NO: 17 - protein, 11 aa, synthetic construct
GSISSGGYYWS

SEQ ID NO: 18 - protein, 11 aa, synthetic construct
GSISHGGYYWS

SEQ ID NO: 19 - protein, 11 aa, synthetic construct
GSISDGSYYWS

SEQ ID NO: 20 - protein, 9 aa, synthetic construct
GSIISYYWG

SEQ ID NO: 21 - protein, 11 aa, synthetic construct
GSISSSVYYWS

SEQ ID NO: 22 - protein, 17 aa, synthetic construct
GIIPIFGTASYAQKFQG

SEQ ID NO: 23 - protein, 17 aa, synthetic construct
AISGSGGSTYYADSVKG

SEQ ID NO: 24 - protein, 16 aa, synthetic construct
NIYYSGSTYYNPSLKS

SEQ ID NO: 25 - protein, 16 aa, synthetic construct
YIYSSGSTSYNPSLKS

SEQ ID NO: 26 - protein, 17 aa, synthetic construct
TISGSGGSTYYADSVKG

SEQ ID NO: 27 - protein, 17 aa, synthetic construct
GIIPIFGTANYAQKFQG

SEQ ID NO: 28 - protein, 16 aa, synthetic construct
SILVSGSTYYNPSLKS

SEQ ID NO: 29 - protein, 13 aa, synthetic construct
ARQQYDGRRYFGL

SEQ ID NO: 30 - protein, 10 aa, synthetic construct
ARVGFRALNY

SEQ ID NO: 31 - protein, 13 aa, synthetic construct
ARESSTISADFDL

SEQ ID NO: 32 - protein, 13 aa, synthetic construct
ARGLSTIDEAFDP

SEQ ID NO: 33 - protein, 16 aa, synthetic construct
ARGSGLYAAPDYGLDV

SEQ ID NO: 34 - protein, 13 aa, synthetic construct
ARGAGHYDLVGRY

SEQ ID NO: 35 - protein, 9 aa, synthetic construct
ARGGPWFDP

SEQ ID NO: 36 - protein, 9 aa, synthetic construct
ARAVSFLDV

SEQ ID NO: 37 - protein, 11 aa, synthetic construct
RASQDISSWLA

SEQ ID NO: 38 - protein, 11 aa, synthetic construct
RASQGISRWLA

SEQ ID NO: 39 - protein, 11 aa, synthetic construct
RASQSISSWLA

SEQ ID NO: 40 - protein, 11 aa, synthetic construct
RASQSISSYLN

SEQ ID NO: 41 - protein, 7 aa, synthetic construct
SASTRAT

SEQ ID NO: 42 - protein, 7 aa, synthetic construct
AASSLQS

SEQ ID NO: 43 - protein, 7 aa, synthetic construct
KASSLES

SEQ ID NO: 44 - protein, 7 aa, synthetic construct
GASSLQS

SEQ ID NO: 45 - protein, 9 aa, synthetic construct
QQVNVWPPT

SEQ ID NO: 46 - protein, 10 aa, synthetic construct
QQATSYPPWT

SEQ ID NO: 47 - protein, 9 aa, synthetic construct
QQAHTFPYT

SEQ ID NO: 48 - protein, 9 aa, synthetic construct
QQDNSYPYT

SEQ ID NO: 49 - protein, 9 aa, synthetic construct
QQDNSFPFT

SEQ ID NO: 50 - protein, 9 aa, synthetic construct
QQLYSLPPT

SEQ ID NO: 51 - protein, 10 aa, synthetic construct
QQYNSYPPFT

SEQ ID NO: 52 - protein, 9 aa, synthetic construct
QQSYDPPWT

SEQ ID NO: 53 - protein, 120 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSS

SEQ ID NO: 54 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSS

SEQ ID NO: 55 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSS

SEQ ID NO: 56 - protein, 121 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSS

SEQ ID NO: 57 - protein, 121 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 58 - protein, 122 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSS

SEQ ID NO: 59 - protein, 120 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSS

SEQ ID NO: 60 - protein, 116 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSS

SEQ ID NO: 61 - protein, 117 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSS

SEQ ID NO: 62 - protein, 107 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIK

SEQ ID NO: 63 - protein, 108 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIK

SEQ ID NO: 64 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIK

SEQ ID NO: 65 - protein, 107 aa, synthetic construct; identical to SEQ ID NO: 64

SEQ ID NO: 66 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIK

SEQ ID NO: 67 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIK

SEQ ID NO: 68 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIK

SEQ ID NO: 69 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIK

SEQ ID NO: 70 - protein, 107 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIK

SEQ ID NO: 71 - protein, 450 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 451 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 451 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 452 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 450 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 446 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 447 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 214 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 215 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 214 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 214 aa, synthetic construct; identical to SEQ ID NO: 82

SEQ ID NO: 84 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 215 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 214 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 107 aa, synthetic construct [sequence omitted]

SEQ ID NO: 90 - DNA, 321 bp, synthetic construct
  source 1..321: mol_type = other DNA
cggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 91 - protein, 328 aa, synthetic construct
ASTKGPSVFPLAPSSKSTSGGTAALGCLVKDYFPEPVTVSWNSGALTSGVHTFPAVLQSSGYSSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - DNA, 990 bp, synthetic construct
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2023/0117205 A1
(43) Pub. Date: Apr. 20, 2023

(54) B7-H4 ANTIBODY-DRUG CONJUGATES FOR THE TREATMENT OF CANCER

(71) Applicant: SEAGEN INC., BOTHELL, WA (US)

(72) Inventors: Shyra GARDAI, Bothell, WA (US); Elizabeth E. GRAY, Seattle, WA (US)

(21) Appl. No.: 17/936,814

(22) Filed: Sep. 29, 2022

Related U.S. Application Data
(60) Provisional application No. 63/261,949, filed on Sep. 30, 2021.
(60) Provisional application No. 63/293,625, filed on Dec. 23, 2021.
(60) Provisional application No. 63/317,536, filed on Mar. 7, 2022.

(51) Int. Cl. A61K 47/68; A61K 47/50; A61P 35/00

(52) U.S. Cl. CPC A61K 47/6851; A61K 47/50; A61K 47/6803; A61P 35/00; A61K 2039/505

(57) ABSTRACT

Methods for using anti-B7-H4 antibodies and antibody-drug conjugates, including anti-B7-H4 antibody-drug conjugates, to inhibit proliferation of a cell, such as a B7-H4-expressing cell, as well as for the treatment of cancers, such as, e.g., B7-H4-associated solid tumors and breast cancer (e.g., locally advanced or metastatic breast cancer), are provided.

### CROSS REFERENCE TO RELATED APPLICATIONS

This application claims priority to U.S. Provisional Application No. 63/261,949, filed Sep. 30, 2021, U.S. Provisional Application No. 63/293,625, filed Dec. 23, 2021, and U.S. Provisional Application No. 63/317,536, filed Mar. 7, 2022, the contents of each of which are hereby incorporated by references in its entirety.

### REFERENCE TO AN ELECTRONIC SEQUENCE LISTING

The contents of the electronic sequence listing (761682007500seqlist.xml; Size: 92,098 bytes; and Date of Creation: Sep. 29, 2022) is herein incorporated by reference in its entirety.

### FIELD OF THE INVENTION

The present invention relates to the field of antibody-based cancer therapeutics. In particular, the present invention relates to B7-H4 antibody-drug conjugates (B7-H4-ADCs), and the use thereof for the treatment of cancer, such as solid tumors, such as, e.g., locally advanced or metastatic solid tumors (e.g., ovarian cancer, lung cancer, adenoid cystic carcinoma, cholangiocarcinoma and endometrial cancer), and breast cancer (e.g., locally advanced or metastatic breast cancer).

### BACKGROUND

B7-H4 is a member of the B7 family of immune checkpoint ligands whose expression is elevated on a variety of solid tumors, in particular breast and ovarian tumors (Leong et al., 2015, Mol Pharm 12, 1717-1729). Similar to B7-H1/PD-L1, B7-H4 has been shown to negatively regulate T cell function and targeted killing of B7-H4-expressing tumor cells may relieve this inhibitory signal (Dangaj et al., 2013, Cancer Res 73, 4820-4829; Prasad et al., 2003, Immunity 18, 863-873; Sica et al., 2003, Immunity 18, 849-861; Zang et al., 2003, Proc Natl Acad Sci USA 100, 10388-10392).

B7-H4 (also known as B7X; B7H4; B7S1; B7h.5; VCTN1; PRO1291; GenBank Accession No Q7Z7D3) is an immune regulatory molecule that shares homology with other B7 family members, including PD-L1. Human B7-H4 is encoded by VTCN1. It is a type I transmembrane protein comprised of both IgV and IgC ectodomains. While B7-H4 expression in healthy tissues is relatively limited at the protein level, B7-H4 is expressed in several solid tumors such as gynecological carcinomas of the breast, ovary, and endometrium. Expression of B7-H4 in tumors tends to correlate with poor prognosis. The receptor for B7-H4 is unknown, but it is believed to be expressed on T cells. B7-H4 is believed to directly inhibit T cell activity.

Cancer remains to be one of the deadliest threats to human health. In the U.S., cancer affects nearly 1.3 million new patients each year, and is the second leading cause of death after heart disease, accounting for approximately 1 in 4 deaths. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

Lung cancer remains the leading cause of death from cancer in the United States, with over 155,000 deaths estimated in 2017. Treatments with curative intent for patients with early stage disease include surgery, chemotherapy, radiation therapy, or a combined modality approach. However, a majority of patients are diagnosed with advanced stage disease, which is usually incurable. Non-small cell lung cancer (NSCLC) represents up to 80% of all lung cancers. Within the subtypes of NSCLC, squamous cell carcinoma (SCC/NSCLC) represents approximately 30% of NSCLC. Systemic therapies used in the metastatic setting for SCC/NSCLC have shown limited benefit and are primarily aimed at prolonging survival and maintaining the quality of life for as long as possible, while minimizing side effects due to treatment. First line treatment for patients with SCC/NSCLC whose tumors do not express high levels of PD-L1 include a platinum-based chemotherapy doublet that does not contain pemetrexed, anti-VEGF antibody, or an anti-EGFR antibody necitumumab in combination with gemcitabine and cisplatin. Patients with at least 50% tumor cell staining for PD-L1 are offered first-line treatment with the anti-PD-1 inhibitor pembrolizumab. Patients who progress on an initial combination chemotherapy regimen may receive an anti-PD-1 or PD-L1 antibody, and combination chemotherapy is considered for patients whose disease has progressed after receiving PD-1/L1 inhibitors. New classes of therapy are urgently needed that can provide meaningful benefit to SCC/NSCLC patients.

Breast cancers are classified on the basis of three protein expression markers: estrogen receptor (ER), progesterone receptor (PgR), and the overexpression of the growth factor receptor HER2/neu. Hormonal therapies, including tamoxifen and aromatase inhibitors, can be effective in treating tumors that express the hormone receptors ER and PgR. HER2-directed therapies are useful for tumors that express HER2/neu; these tumors are the only class of breast cancer that is currently eligible for monoclonal antibody therapy. For these patients, unconjugated antibodies, such as Herceptin or Perjeta, are generally used in combination with chemotherapy.

Ovarian cancers are classified on the basis of the origin cell types. Ovarian epithelial carcinoma, is the most common type of ovarian cancer, representing approximately 90% of ovarian cancers. It includes serous, endometrioid, and clear cell tumors. Less common ovarian epithelial tumors are mucinous and malignant Brenner tumors. Epithelial ovarian cancers develop from the epithelium, a layer of cells that covers the ovary. Poorly differentiated epithelial ovarian cancer is defined as high grade serous ovarian carcinoma (HGSOC) and it includes fallopian tube and primary peritoneal epithelial serous tumors. HGSOC is treated by cytotoxic therapy, including platinum chemotherapy regimens and taxanes. Targeted agents, such as PARP inhibitors, are used in the treatment and maintenance setting. Immunotherapy is a topic of current research in ovarian cancer. In some cases, the antibody bevacizumab, though still a topic of active research, is used to treat advanced cancer along with chemotherapy. Relapsed platinum resistant and refractory HGSOC is an area of high unmet medical need.

Cholangiocarcinoma, also known as bile duct cancer, is a disease in which malignant (cancer) cells form in the bile ducts. Bile duct cancer can be intrahepatic or extrahepatic. Risk factors for cholangiocarcinoma include primary sclerosing cholangitis, ulcerative colitis, cirrhosis, hepatitis C, hepatitis B, infection with certain liver flukes, and some congenital liver malformations. Cholangiocarcinoma is typically incurable at diagnosis.

Endometrial cancer is a cancer that arises from the endometrium. It is the result of abnormal growth of cells that have the ability to invade or spread to other parts of the body. Endometrial cancer is associated with obesity, excessive estrogen exposure, high blood pressure and diabetes. It is the third most common cause of death in cancers which only affect women, behind ovarian and cervical cancer.

Fallopian tube cancer, also known as tubal cancer, develops in the fallopian tubes that connect the ovaries and the uterus. It is more common for cancer to spread, or metastasize, from other parts of the body, such as the ovaries or endometrium, than for cancer to actually originate in the fallopian tubes. To date, little is known regarding what causes fallopian tube cancer, but genetics are suspected to play a role.

Peritoneal cancer is also known as serous surface papillary carcinoma, primary peritoneal carcinoma, extra-ovarian serous carcinoma, primary serous papillary carcinoma, and psammomacarcinoma. It develops in the peritoneum, a thin layer of tissue lining the abdomen that is made of epithelial cells. The causes of peritoneal cancer are unclear. Peritoneal cancer can be hard to detect in the early stages. The median survival of primary peritoneal carcinomas is usually shorter by 2-6 months time when compared with serous ovarian cancer.

Gallbladder cancer is an abnormal growth of cells that begins in the gallbladder. If diagnosed early enough, it can be treated by removing the gallbladder, part of the liver and associated lymph nodes. Most often it is found after symptoms such as abdominal pain, jaundice and vomiting occur, by which time it has spread to other organs such as the liver. The outlook is poor for recovery if the cancer is found after symptoms have started to occur, with a 5-year survival rate of close to 3%.

There is clearly a significant need for effective treatments for solid tumors, particularly locally advanced or metastatic solid tumors, and breast cancer, particularly late-stage breast cancer. The present invention meets the need for improved treatment of solid tumors, such as, e.g., locally advanced or metastatic solid tumors (e.g., ovarian cancer, lung cancer, cholangiocarcinoma and endometrial cancer), and breast cancer by providing a highly specific and effective anti-B7-H4-antibody-drug conjugate. The present invention also meets the need for improved treatment of solid tumors, such as, e.g., locally advanced or metastatic solid tumors (e.g. peritoneal cancer, fallopian tube cancer, gallbladder cancer) by providing a highly specific and effective anti-B7-H4-antibody-drug conjugate.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating a subject having or at risk of having a B7-H4-associated cancer, comprising administering to the subject a therapeutically effective dose of a B7-H4 antibody-drug conjugate (B7-H4-ADC), wherein the B7-H4-ADC comprises a human anti-B7-H4 antibody conjugated to a vcMMAE (valine-citruline-monomethyl auristatin E), wherein the anti-B7-H4 antibody comprises a heavy chain variable region comprising the sequence of SEQ ID NO: 11 and a light chain variable region comprising the sequence of SEQ ID NO: 12, wherein the vcMMAE has the structure:

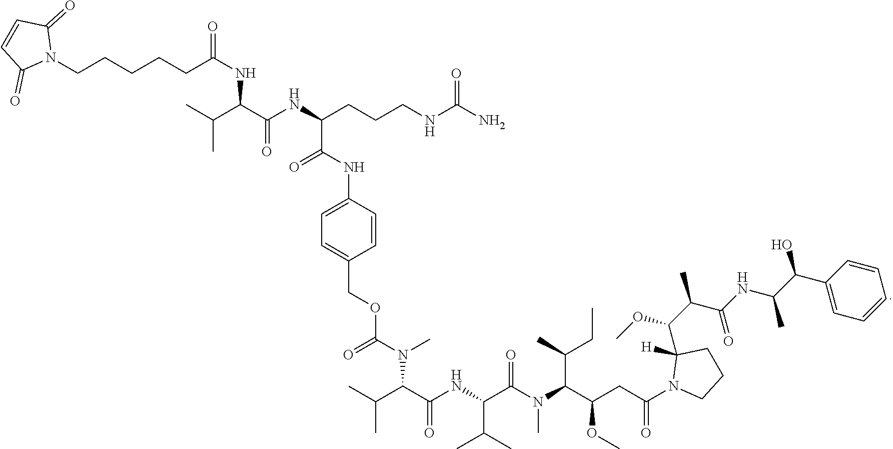 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

Also provided herein are B7-H4 antibody-drug conjugates (B7-H4-ADC), wherein the B7-H4-ADC comprises a human anti-B7-H4 antibody conjugated to a vcMMAE (valine-citruline-monomethyl auristatin E), wherein the anti-B7-H4 antibody comprises a heavy chain variable region comprising the sequence of SEQ ID NO:11 and a light chain variable region comprising the sequence of SEQ ID NO:12, wherein the vcMMAE has the structure:

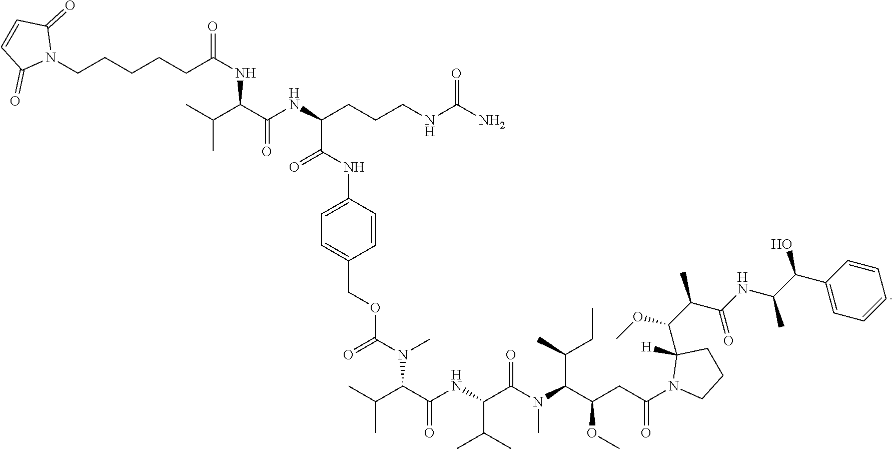 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, a vcMMAE to anti-B7-H4 ratio is from 1 to 8. In some embodiments, the average value of the vcMMAE to anti-B7-H4 ratio in a population of the B7-H4-ADC is about 4. In some embodiments, the B7-H4-associated cancer is a breast cancer. In some embodiments, the breast cancer is estrogen receptor positive (ER+) breast cancer. In some embodiments, the breast cancer is progesterone receptor positive/human epidermal growth factor receptor 2 negative breast (PR+/HER2−) cancer. In some embodiments, the breast cancer is a triple negative breast cancer. In some embodiments, the breast cancer is hormone receptor positive (HR+) breast cancer. In some embodiments, the breast cancer is HER2 positive breast cancer. In some embodiments, the breast cancer is HR+/HER2 negative breast cancer. In some embodiments, the cancer is an adenoid cystic carcinoma. In some embodiments, the adenoid cystic carcinoma is an adenoid cystic carcinoma of the head and neck. In some embodiments, the adenoid cystic carcinoma of the head and neck is an adenoid cystic carcinoma of the salivary glands. In some embodiments, the adenoid cystic carcinoma is an adenoid cystic carcinoma of the ovary. In some embodiments, the adenoid cystic carcinoma is an adenoid cystic carcinoma of the prostate. In some embodiments, the adenoid cystic carcinoma is an adenoid cystic carcinoma of the breast. In some embodiments, the adenoid cystic carcinoma is an adenoid cystic carcinoma of the skin. In some embodiments, the adenoid cystic carcinoma is an adenoid cystic carcinoma of the cervix. In some embodiments, the cancer is an advanced stage cancer. In some embodiments, the advanced stage cancer is a stage 3 or stage 4 cancer. In some embodiments, the cancer is metastatic cancer. In some embodiments, the cancer is unresectable. In some embodiments, the cancer is locally advanced. In some embodiments, the cancer is recurrent cancer. In some embodiments, the subject received prior treatment with standard of care therapy for the cancer and failed the prior treatment. In some embodiments, the subject has been previously treated with one or more therapeutic agents and did not respond to the treatment, wherein the one or more therapeutic agents is not the B7-H4-ADC. In some embodiments, the subject has been previously treated with one or more therapeutic agents and relapsed after the treatment, wherein the one or more therapeutic agents is not the B7-H4-ADC. In some embodiments, the subject has been previously treated with one or more therapeutic agents and has experienced disease progression during treatment, wherein the one or more therapeutic agents is not the B7-H4-ADC. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express B7-H4. In some embodiments, one or more therapeutic effects in the subject is improved after administration of the B7-H4-ADC relative to a baseline. In some embodiments, the one or more therapeutic effects is selected from the group comprises size of a tumor derived from the cancer. In some embodiments, the route of administration for the B7-H4-ADC is intravenous infusion. In some embodiments, the B7-H4-ADC is administered as a monotherapy. In some embodiments, the B7-H4-ADC is in a pharmaceutical composition comprising the B7-H4-ADC and a pharmaceutically acceptable carrier. In some embodiments, the subject is a human.

Also provided here are kits comprising (a) a dosage ranging from about 0.5 mg/kg to about 3.0 mg/kg of a B7-H4-ADC; and (b) instructions for using the B7-H4-ADC according to any of the methods provided herein.

Also provided herein are methods of treating a subject having or at risk of having a B7-H4-associated cancer, comprising administering to the subject a therapeutically effective dose of an antibody or an antigen-binding fragment thereof that specifically binds human B7-H4, wherein the antibody or antigen-binding fragment thereof comprises a heavy chain variable region (HCVR) having at least 95% identity to SEQ ID NO: 11, and a light chain variable region (LCVR) having at least 95% identity to SEQ ID NO: 12, wherein the cancer is a solid tumor. In some embodiments, the heavy chain variable region of the antibody or antigen-binding fragment thereof comprises the three complementarity determining regions (CDRs) of SEQ ID NO: 11 and the light chain variable region of the antibody or antigen-binding fragment thereof comprises the three CDRs of SEQ ID NO: 12. In some of the embodiments herein, the heavy chain variable region comprises the sequence of SEQ ID NO: 11 and the light chain variable region comprises the sequence of SEQ ID NO: 12. In some of the embodiments herein, the antibody or antigen-binding fragment thereof is conjugated to monomethyl auristatin E (MMAE):

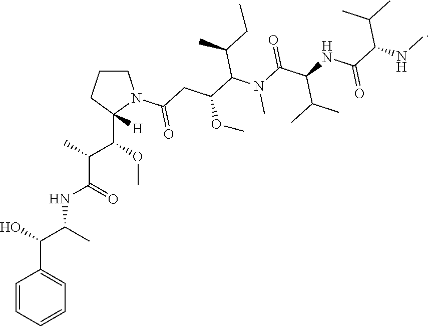 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In some of the embodiments herein, the antibody or antigen-binding fragment thereof is conjugated to valine-citrulline-monomethyl auristatin E (vcMMAE):

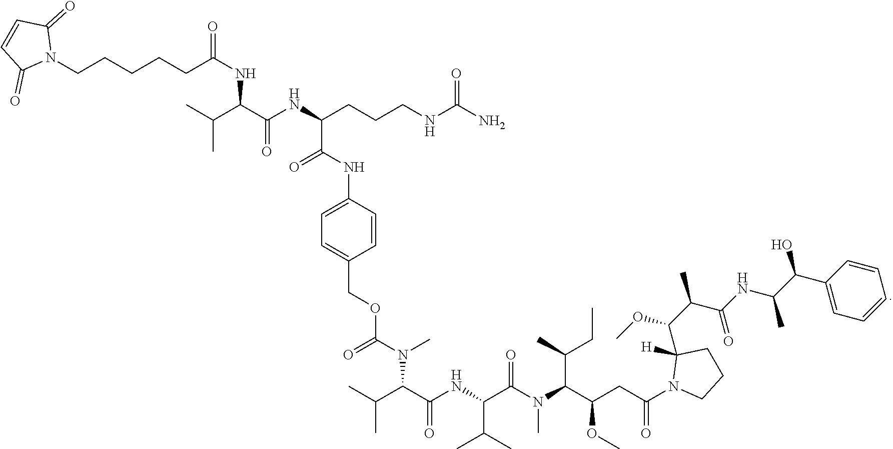 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some of the embodiments herein, the subject has been previously treated with one or more therapeutic agents and did not respond to the treatment, wherein the one or more therapeutic agents is not the antibody or antigen-binding fragment thereof. In some of the embodiments herein, the subject has been previously treated with one or more therapeutic agents and relapsed after the treatment, wherein the one or more therapeutic agents is not the antibody or antigen-binding fragment thereof. In some of the embodiments herein, the subject has been previously treated with one or more therapeutic agents and has experienced disease progression during treatment, wherein the one or more therapeutic agents is not the antibody or antigen-binding fragment thereof. In some of the embodiments herein, the cancer is selected from breast cancer, ovarian cancer, lung cancer, cholangiocarcinoma and endometrial cancer. In some of the embodiments herein, the cancer is selected from peritoneal cancer, fallopian tube cancer, and gallbladder cancer. In one preferred embodiment, the cancer is selected from the group consisting of ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma and gallbladder carcinoma. In some of the embodiments herein, the solid tumor is lung cancer. In some of the embodiments herein, the lung cancer is small cell lung cancer. In some of the embodiments herein, the lung cancer is non-small cell lung cancer. In some embodiments, the cancer is an adenoid cystic carcinoma. In some embodiments, the adenoid cystic carcinoma is an adenoid cystic carcinoma of the head and neck. In some embodiments, the adenoid cystic carcinoma of the head and neck is an adenoid cystic carcinoma of the salivary glands. In some embodiments, the adenoid cystic carcinoma is an adenoid cystic carcinoma of the ovary. In some embodiments, the adenoid cystic carcinoma is an adenoid cystic carcinoma of the prostate. In some embodiments, the adenoid cystic carcinoma is an adenoid cystic carcinoma of the breast. In some embodiments, the adenoid cystic carcinoma is an adenoid cystic carcinoma of the skin. In some embodiments, the adenoid cystic carcinoma is an adenoid cystic carcinoma of the cervix. In some of the embodiments herein, the non-small cell lung cancer is non-squamous cell carcinoma. In some of the embodiments herein, the non-small cell lung cancer is squamous cell carcinoma. In some of the embodiments herein, the cancer is an advanced stage cancer. In some of the embodiments herein, the advanced stage cancer is a stage 3 or stage 4 cancer. In some of the embodiments herein, the advanced stage cancer is metastatic cancer. In some of the embodiments herein, the cancer is recurrent cancer. In some of the embodiments herein, the cancer is unresectable. In some of the embodiments herein, the subject received prior treatment with standard of care therapy for the cancer and failed the prior treatment. In some of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express B7-H4. In some of the embodiments herein, one or more therapeutic effects in the subject is improved after administration of the antibody or antigen-binding fragment thereof relative to a baseline. In some of the embodiments herein, the one or more therapeutic effects is selected from the group comprises size of a tumor derived from the cancer. In some of the embodiments herein, the route of administration for the antibody or antigen-binding fragment thereof is intravenous infusion. In some of the embodiments herein, the antibody or antigen-binding fragment thereof is administered as a monotherapy. In some of the embodiments herein, the antibody or antigen-binding fragment thereof is in a pharmaceutical composition comprising the antibody or antigen-binding fragment thereof and a pharmaceutically acceptable carrier. In some of the embodiments herein, the subject is a human.

In some embodiments, administration of the B7-H4 ADC induces an anti-tumor immune response in the subject. In some embodiments, administration of the B7-H4 ADC induces upregulation of expression of one or more chemokines and/or one or more type I interferon response genes.

In some embodiments, administration of the B7-H4-ADC induces upregulation of expression of CXCL10, CXCL9, CXCL1, IFTIT2, and/or MX1. In some embodiments, administration of the B7-H4-ADC promotes recruitment of innate immune cells and/or adaptive immune cells to a tumor site. In some embodiments, the immune cells are tumor infiltrating cells. In some embodiments, administration of the B7-H4-ADC promotes recruitment of CD11c+ dendritic cells, F4/80+ macrophages, and/or cells expressing CD86 to a tumor site. In some embodiments administration of the B7-H4-ADC causes an increase in Baft3, Cd68, H2Aa, H2-eb1, CD80, CD86, CD3e, CD4, Cd8a, Pdcd1, Cd27, Cxcr6, Lag3, Nkg7, Cc15, Cd274, Cmklr1, Cxcl9, Psmb10, Stat1, and/or Icos1 transcript level at a tumor site. In some embodiments, administration of the B7-H4-ADC promotes recruitment of CD3+ cells, CD4+ cells, CD8+ cells, PD1+ cells to a tumor site. In some embodiments, administration of the B7-H4-ADC causes an increase in the level of gene expression of a gene associated with responsiveness to PD-1 therapy. In some embodiments, administration of the B7-H4-ADC causes an increase in the level of Ki67, CD163, CD206, ChiL3, and/or Granzyme B positive cells in the tumor

Also provided herein are kits comprising: (a) a dosage ranging from about 0.5 mg/kg to about 3.0 mg/kg of an antibody or antigen-binding fragment thereof that binds B7-H4; and (b) instructions for using the B7-H4-ADC according to some of the methods provided herein.

### BRIEF DESCRIPTION OF THE DRAWINGS

FIG. 1A shows the structure of B7H41001 IgG1 monoclonal antibody (mAb). FIG. 1B shows the amino acid sequence of B7H41001 mAb Heavy Chain. FIG. 1C shows the amino acid sequence of B7H41001 mAb Light Chain.

FIG. 2 is a plot showing the expression of VTCN1 RNA, which encodes B7-H4, in The Cancer Genome Atlas (retrieved October 2020). Gene-level expression values, subsequent analysis and visualization steps were performed in the R computing environment.

FIG. 3 shows the B7-H4 IHC staining of non-transfected HEK293T cells (parental) or HEK293T cells transfected with expression plasmids encoding mouse B7-H4 (mB7-H4) or human B7-H4 (hB7-H4).

FIG. 4 shows the B7-H4 IHC staining of cancer cell lines that endogenously express a range of B7-H4 copy number as measured by quantitative flow cytometry.

FIG. 5 shows the B7-H4 IHC staining of formalin-fixed paraffin-embedded breast (left) and ovarian (right) tumors.

FIG. 6 is a bar chart showing the score of B7-H4 staining of formalin-fixed paraffin-embedded tumors using mAb clone D1M8I (CST). Slides were scored as follows: Intensity: 0=none, 1=weak, 2=moderate, 3=strong; Frequency: 1=1-25%, 2=26-50%, 3=51-75%, 4=>75%. For prevalence calculations, tumors were considered positive if membrane (M) and/or apical membrane (A) staining (any intensity) was observed on greater than 25% of tumor cells. Tumors scored with intensities “1-2” or “2-3” were plotted as the lower intensity score number.

FIG. 7 is a series of sensorgrams depicting monovalent and bivalent binding of SGN-B7H4V and B7H41001 mAb binding to human B7-H4 protein measured by BLI on an Octet HTX system (ForteBio). Lines indicate a range of concentration of antigen (monovalent) or mAb/ADC (bivalent) in nM; binding affinities (KD) are indicated in text.

FIG. 8 is a series of graphs depicting the binding of SGN-B7H4V, B7H41001 mAb, non-binding control ADC, and non-binding mAb to B7-H4-expressing SKBR3 cells. Mean and range of the % of maximum binding are plotted for two replicate experiments.

FIG. 9 is a series of plots showing internalization of the B7H41001 mAb in a cell-based assay. MX-1 and SKBR3 cells, which endogenously express B7-H4, were incubated with quenched fluorophore (vcQF01) conjugates, which use the same vcPAB linker as in SGN-B7H4V, for up to 24 hours. Normalized mean red fluorescence intensity per cell is shown (normalized to t=0 hours) with the mean and standard deviation plotted for triplicate cells.

FIG. 10 is a series of plots showing in vitro cytotoxicity of B7-H4-expressing cell lines when treated with SGN-B7H4V compared to a non-binding control ADC.

FIG. 11A and FIG. 11B are a series of sensorgrams depicting the binding of SGN-B7H4V, B7H41001 mAb, and positive control mAb (varied by row) to human Fc-receptors (varied by column). Equilibrium dissociation constants are listed in the top right corner of each sensorgram.

FIG. 12 is a series of plots showing the cellular FcγR signaling by SGN-B7H4V and B7H41001 mAb, as assayed by measuring FcγR-mediated luciferase reporter signal on an Envision plate reader (PerkinElmer). Data shown is the average and standard deviation of each condition performed in duplicate or triplicate.

FIG. 13 is a series of plots showing the ADCC mediated by SGN-B7H4V and B7H41001 mAb, where the percent lysis was determined using the CytoTox 96 Non-Radioactive Cytotoxicity Assay kit. Data shown is the mean and standard deviation of the % of maximum cell lysis for each condition performed in triplicate or duplicate; outlier values were excluded.

FIG. 14 is a series of plots showing the ADCP mediated by SGN-B7H4V and B7H41001 mAb, where the phagocytic activity was determined by calculating the PKF26 geometric mean fluorescence intensity (gMFI) on CD14+/CD45+ monocytes/macrophages. Data shown is the average and standard deviation of the gMFI for each condition performed in duplicate or triplicate.

FIG. 15 is a series of plots showing the lack of CDC mediated by SGN-B7H4V and B7H41001 mAb, where SYTOX® Green reagent was used as a measure of cell death by fluorescence readout on an Envision plate read. Data shown is the mean and standard deviation of the % of maximum cell lysis for each condition performed in duplicate.

FIG. 16 is a plot showing the anti-tumor activity in MX-1 xenograft model in mice treated with SGN-B7H4V compared to a non-binding control ADC.

FIG. 17 is a plot showing the anti-tumor activity in MDA-MB-468 xenograft model in mice treated with SGN-B7H4V compared to a non-binding control ADC.

FIG. 18 is a plot showing the anti-tumor activity in MDA-MB-468 xenograft model in mice treated with SGN-B7H4V compared to a non-binding control ADC, or B7H41001 mAb

FIG. 19 is a plot showing the anti-tumor activity in HCC1569 xenograft model in mice treated with SGN-B7H4V compared to a non-binding control ADC.

FIG. 20 is a plot showing the anti-tumor activity in OVCAR3 xenograft model in mice treated with SGN-B7H4V compared to a non-binding control ADC.

FIG. 21 shows representative images of formalin-fixed paraffin-embedded untreated MX-1 (top) and HCC1569 (bottom) tumors stained for B7-H4.

FIG. 22 shows representative images of formalin-fixed paraffin-embedded untreated (top left), non-binding control ADC-treated (top right), SGN-B7H4V-treated (bottom left), OVCAR3 tumors as well as untreated MDA-MB-468 (bottom right) tumors stained for B7-H4.

FIG. 23 is a series of plots showing the quantification of B7-H4 staining on OVCAR3 and MDA-MB-468 tumors. Halo image analysis software was used to quantify the percentage of B7-H4+ tumor tissue (left panel) and B7-H4 H-score (right panel) for OVCAR3 and MDA-MB-468 tumors treated as indicated. Values for each individual tumor as well as the mean for each group are plotted.

FIG. 24 is a series of plots showing the anti-tumor activity in TNBC PDX models in mice treated with SGN-B7H4V compared to a non-binding control ADC. Mean tumor volumes for untreated animals (n=1 or 2) and tumors volumes for individual animals in non-binding control ADC (n=1) and SGN-B7H4V (n=3) treatment groups. Animals were treated with 3 mg/kg of ADC on days 0, 7, and 14.

FIG. 25 is a series of plots showing the anti-tumor activity in HR⁺ BC PDX models in mice treated with SGN-B7H4V compared to a non-binding control ADC. Mean tumor volumes (untreated and non-binding control ADC-treated animals) and tumors volumes for individual animals (SGN-B7H4V treatment group, n=3 per group). Animals were treated with 3 mg/kg of ADC on days 0, 7, and 14.

FIG. 26 is a series of plots showing the anti-tumor activity in Ovarian PDX models in mice treated with SGN-B7H4V compared to a non-binding control ADC. Mean tumor volumes (untreated and non-binding control ADC-treated animals) and tumors volumes for individual animals (SGN-B7H4V treatment group, n=3 per group). Animals were treated with 3 mg/kg of ADC on days 0, 7, and 14.

FIG. 27A-D are a series of plots and corresponding data showing the antitumor activity in the TNBC_1 PDX model of TNBC (FIG. 27A, top panel) with heterogeneous B7-H4 staining (FIG. 27A, bottom panel), the antitumor activity in the Ovarian_1 model of ovarian cancer (FIG. 27B, top panel) with uniformly high B7-H4 staining (FIG. 27B, bottom panel), and the antitumor activity in the heavily-pretreated Ovarian_2 model of ovarian cancer (FIG. 27C, top panel) with heterogeneous B7-H4 staining (FIG. 27C, bottom panel). FIG. 27D is a table showing metadata in PDX model analysis.

FIG. 28A and FIG. 28B show plots depicting the expression of VTCN1 RNA, which encodes B7-H4 protein, in BLUEPRINT (retrieved May 2019). Gene-level expression values, subsequent analysis and visualization steps were performed in the R computing environment.

FIG. 29 is a bar chart showing the B7-H4 expression on human peripheral blood monocytes and differentiated macrophage subsets, as assayed by flow analysis (anti-B7-H4 mAbs clones B7H41001 and MIH43; the anti-B7-H3 mAb clone 7-517). The geometric mean fluorescent intensity of cells stained with the test articles relative to cells stained with an isotype control mAb (“isotype FMO”) is plotted. Bar plots indicate the mean fold change.

FIG. 30 is a bar chart showing the B7-H4 expression on human monocyte-derived immature and mature dendritic cells, as assayed by flow analysis (anti-B7-H4 mAb clone B7H41001; anti-41BBL mAb clone 5F4). The geometric mean fluorescent intensity of cells stained with the test articles relative to cells stained with an isotype control mAb (“isotype FMO”) is plotted. Bar plots indicate the mean fold change.

FIG. 31 is a series of immunofluorescence images showing the detection of B7-H4 and CD163. Two TNBC tumor sections co-stained for B7-H4 (left) and CD163 (right). No co-staining of B7-H4 on CD163+ macrophages was observed.

FIG. 32A shows the effect of SGN-B7H4V and MMAE on ATP release (left panel), HMGB1 release (middle panel), as well as cell surface exposure of calreticulin (right panel) by SKBR3 cells 48 hours following treatment with 1 μg/mL SGN-B7H4V, or non-binding control ADC, or 100 nM MMAE free drug. For cell surface exposure of calreticulin, the percentage of cells that are propidium iodide (PI) negative and calreticulin (CAR) positive is plotted. FIG. 32B demonstrates that SGN-B7H4V, but not B7H41001 mAb, elicits upregulation of CD86 on CD14+ monocytes and release of MIP-1β following treatment of MDA-MB-468 tumor and peripheral blood mononuclear cell (PBMC) co-cultures.

FIG. 33 shows the mean tumor volume of MDA-MB-468 xenograft tumors in NSG mice that were treated with a single 3 mg/kg dose of vehicle control, unconjugated B7H41001 mAb, or SGN-B7H4V or a single 6 mg/kg dose of B7H41001 mAb-DM1 or B7H41001 mAb-DM4 conjugates.

FIG. 34A shows representative images of IHC staining for F4/80+ macrophages in MDA-MB-468 xenograft tumors that were treated with a single 3 mg/kg dose of vehicle control, or SGN-B7H4V. FIG. 34B shows the percentage of F4/80+ macrophages in MDA-MB-468 xenograft tumors following treatment with a single 3 mg/kg dose of vehicle control, unconjugated B7H41001 mAb, or SGN-B7H4V or a single 6 mg/kg dose of B7H41001 mAb-DM1 or B7H41001 mAb-DM4 conjugates.

FIG. 35 is a series of RNAseq analyses showing the relative amount of human transcripts encoding cytokines (CXCL10 and CXCL1) and type I IFN response genes (IFIT2 and MX1) in MDA-MB-468 xenograft tumors in NSG mice that were treated with a single 3 mg/kg dose of vehicle control, unconjugated B7H41001 mAb, or SGN-B7H4V or a single 6 mg/kg dose of B7H41001 mAb-DM1 or B7H41001 mAb-DM4 conjugates.

FIG. 36A is a FACs plot showing the B7-H4 expression of Renca tumor cells engineered to express full length murine B7-H4 (mB7-H4) versus isotype control. FIG. 36B shows the mean tumor volume of B7-H4-Renca tumors in tumor-bearing mice treated with either SGN-B7H4V mIgG2a, non-binding control ADC mIgG2a, unconjugated mAb B7H41001 mIgG2a, or the afucosylated mAb SEA-B7H41001 mIgG2a. ADCs and mAbs with a murine IgG2a (mIgG2a) Fc backbone were used to avoid anti-drug antibody responses that can occur upon repeat treatment of human IgG1 (hIgG1) antibodies in immunocompetent mice.

FIG. 37 is RNAseq analysis of mB7H4-expressing Renca tumors treated for 6-7 days with a single 3 mg/kg dose of vehicle control, non-binding control ADC mIgG2a, unconjugated B7H41001 mIgG2a mAb, or SGN-B7H4V mIgG2a. RNAseq analysis revealed a significant increase in transcripts encoding cytokines and type I IFN response genes cells following treatment with SGN-B7H4V compared to the unconjugated mAb B7H41001.

FIG. 38A shows representative images of IHC staining for CD11c+ antigen-presenting cells, F4/80+ macrophages, and CD86+ cells in B7-H4-Renca tumors 6-7 days following treatment with a single 3 mg/kg dose of naked B7H41001 mIgG2a mAb, or SGN-B7H4V mIgG2a. FIG. 38B shows the percentage of CD11c+ antigen-presenting cells, F4/80+ macrophages, or CD86+ cells, respectively, in B7-H4-Renca tumors following treatment with a single 3 mg/kg dose of vehicle, naked B7H41001 mIgG2a mAb, SGN-B7H4V mIgG2a, or non-binding ADC mIgG2a. FIG. 38C is an RNAseq analysis showing the relative amount of murine transcripts Itgax (encodes dendritic cell marker CD11c), Batf3 (encodes BatF3, a transcription factor associated with antigen cross-presentation), Cd68 (encodes the macrophage marker CD68), H2-Aa & H2-eb1 (encode MHC class II molecules), and Cd80, Cd86, & Icos1 (encode costimulatory molecules) in B7-H4-Renca tumors following treatment with a single 3 mg/kg dose of vehicle, naked B7H41001 mIgG2a mAb, SGN-B7H4V mIgG2a, or non-binding ADC mIgG2a.

FIG. 39A shows representative images of IHC staining for CD3+ T cells, CD4+ cells, CD8+ cells, and PD1+ cells in mB7-H4-Renca tumors 6-7 days following treatment with a single 3 mg/kg dose of naked B7H41001 mIgG2a mAb or SGN-B7H4V mIgG2a. FIG. 39B shows the percentage of CD3+ T cells, CD4+ cells, CD8+ cells, and PD1+ cells in mB7-H4-Renca tumors following treatment with a single 3 mg/kg dose of vehicle, naked B7H41001 mIgG2a mAb, SGN-B7H4V mIgG2a, or non-binding mIgG2a ADC. FIG. 39C is an RNAseq analysis showing the relative amount of murine transcripts Cd3e, Cd4, and Cd8a as well as markers associated with early T cell activation including Pdcd1 (encodes PD-1), Cd27, and Icos in mB7-H4-Renca tumors following treatment with a single 3 mg/kg dose of vehicle, naked B7H41001 mIgG2a mAb, SGN-B7H4V mIgG2a, or non-binding mIgG2a ADC.

FIG. 40A shows representative images of IHC staining for PD-L1 in mB7-H4-Renca tumors 6-7 days following treatment with a single 3 mg/kg dose of naked B7H41001 mIgG2a mAb or SGN-B7H4V mIgG2a in the left panel. Quantification of the percentage of PD-L1+ cells is plotted in the right panel. FIG. 40B shows RNAseq analysis showing the relative amount of multiple “T cell-inflamed” gene transcripts that have been associated clinically with response to PD-1 blockade in mB7-H4-Renca tumors 6-7 days following treatment with a single 3 mg/kg dose of vehicle, naked B7H41001 mIgG2a mAb, SGN-B7H4V mIgG2a, or non-binding mIgG2a ADC.

FIG. 41A show quantification of the percentage of Ki67+ cells in mB7-H4-Renca tumors 6-7 days following treatment with a single 3 mg/kg dose of vehicle, naked B7H41001 mIgG2a mAb, SGN-B7H4V mIgG2a, or non-binding mIgG2a ADC. FIG. 41B show quantification of the percentage of CD163+, CD206+, Chi3L3+, and Granzyme B+ cells in mB7-H4-Renca tumors 6-7 days following treatment with a single 3 mg/kg dose of vehicle, naked B7H41001 mIgG2a mAb, SGN-B7H4V mIgG2a, or non-binding mIgG2a ADC.

FIG. 42 shows percent survival of B7-H4-Renca tumor-bearing mice treated with non-binding control mIgG2a ADC, SGN-B7H4V mIgG2a, anti-PD-1 mAb, or either the combination of SGN-B7H4V mIgG2a and anti-PD-1 mAb, or the combination of SGN-B7H4V mIgG2a and control mAb, at the indicated doses.

FIG. 43 shows the tumor volumes of B7-H4-Renca tumors in tumor-bearing mice treated with non-binding control mIgG2a ADC, SGN-B7H4V mIgG2a, anti-PD-1 mAb, or either the combination of SGN-B7H4V mIgG2a and anti-PD-1 mAb, or the combination of SGN-B7H4V mIgG2a and control mAb, at the indicated doses.

### DETAILED DESCRIPTION

Provided herein are B7-H4 antibody drug conjugates (ADC) comprising an antibody that binds to B7-H4 conjugated to vcMMAE that are effective for treating cancer (such as solid tumors). In some embodiments, the present ADC induce an immunological response at the tumor site that causes recruitment of immune cells that kill tumor cells. The immunological response triggered by the ADCs disclosed herein can be measured in a number of ways including the presence/absence of particular immune cells (e.g. CD4+, CD3+, CD8+ cells), the release of pro inflammatory cytokines and interferons, the expression of certain transcripts associated with an inflammatory response, and the detection of markers of certain cell types such as macrophages that are able to phagocytose tumor cells. In some embodiments, the ADCs provided herein trigger an immune signature associated with responsiveness to an immune therapy, for example a PD-1 antibody. Accordingly, in some embodiments, the ADCs provided herein can be used as a combination therapy with a PD-1 antibody.

The ADCs provided herein that comprise anti-B7-H4 antibodies conjugated to vcMMAE also show benefits as compared to B7-H4 ADC conjugates with other microtubule inhibitors. For example, in some embodiments, the ADCs provided herein cause a more potent immunological response compared to B7-H4 antibodies conjugated to DM1 or DM4. In some embodiments, the vcMMAE conjugates provided herein cause an increase in the presence of particular immune cells (e.g. CD4+, CD3+, CD8+ cells) associated with inflammation, release of pro inflammatory cytokines and interferons, expression of certain transcripts associated with an inflammatory response, and the presence markers of certain cell types such as macrophages that are able to phagocytose tumor cells as compared to ADCs comprising an antibody that binds to B7-H4 conjugated to DM1 or DM4.

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### I. DEFINITIONS

As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a cytotoxic agent or cytostatic agent. Typically, antibody-drug conjugates bind to a target antigen (e.g., B7-H4) on a cell surface, followed by internalization of the antibody-drug conjugate into the cell and subsequent release of the drug into the cell. In certain exemplary embodiments, an antibody-drug conjugate is a B7-H4-ADC.

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures. Substituents such as carbohydrate groups are generally not specified, but may be present nonetheless.

The terms “amino-terminal” and “carboxy-terminal” denote positions within polypeptides. Where the context allows, these terms are used with reference to a particular sequence or portion of a polypeptide to denote proximity or relative position. For example, a certain sequence positioned carboxy-terminal to a reference sequence within a polypeptide is located proximal to the carboxy terminus of the reference sequence, but is not necessarily at the carboxy terminus of the complete polypeptide.

For purposes of classifying amino acids substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine. Conservative substitutions can also mean substitutions between amino acids in the same class. Classes are as follows: Group I (hydrophobic side chains): Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range.

In antibodies or other proteins described herein, reference to amino acid residues corresponding to those specified by SEQ ID NO includes post-translational modifications of such residues.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and antigen-binding antibody fragments, such as a F(ab′)₂, a Fv fragment, a diabody, a single-chain antibody, an scFv fragment, or an scFv-Fc. Genetically, engineered intact antibodies and fragments such as chimeric antibodies, humanized antibodies, fully human antibodies, single-chain Fv fragments, single-chain antibodies, diabodies, minibodies, linear antibodies, multivalent or multi-specific (e.g., bispecific) hybrid antibodies, and the like, are also included. Thus, the term “antibody” is used expansively to include any protein that comprises an antigen-binding site of an antibody and is capable of specifically binding to its antigen.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a cytostatic or cytotoxic drug.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “human” antibody or antigen-binding fragment thereof means an antibody or antigen-binding fragment thereof having an amino acid sequence derived from a human immunoglobulin gene locus, where such antibody or antigen-binding fragment is made using techniques known in the art. This definition of a human antibody or antigen-binding fragment thereof includes intact or full-length antibodies and fragments thereof.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994. The term “immunoglobulin” is used herein for its common meaning, denoting an intact antibody, its component chains, or fragments of chains, depending on the context.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, Md., 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, Md., 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is substantially from a corresponding CDR in a non-human antibody when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Antibodies are typically provided in isolated form. This means that an antibody is typically at least about 50% w/w pure of interfering proteins and other contaminants arising from its production or purification but does not exclude the possibility that the antibody is combined with an excess of pharmaceutical acceptable carrier(s) or other vehicle intended to facilitate its use. Sometimes antibodies are at least about 60%, about 70%, about 80%, about 90%, about 95% or about 99% w/w pure of interfering proteins and contaminants from production or purification. Antibodies, including isolated antibodies, can be conjugated to cytotoxic agents and provided as antibody drug conjugates.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc-binding cells or complement components result in inhibition and/or depletion of the B7-H4-targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity. In certain exemplary embodiments, an anti-B7-H4 IgG1 antibody of the invention mediates equal or increased ADCC relative to a parental antibody and/or relative to an anti-B7-H4 IgG3 antibody.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig. In certain exemplary embodiments, an anti-B7-H4 IgG1 antibody of the invention mediates equal or increased ADCP relative to a parental antibody and/or relative to an anti-B7-H4 IgG3 antibody.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component C1q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

As used herein, the terms “subject” and “patient” refer to organisms to be treated by the methods of the present invention. Such organisms preferably include, but are not limited to, mammals (e.g., murines, simians, equines, bovines, porcines, canines, felines, and the like), and more preferably includes humans. As used herein, the terms “treat,” “treatment” and “treating” include any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

“Tumor” as it applies to a subject diagnosed with, or suspected of having, cancer (e.g., solid cancer or breast cancer), refers to a malignant or potentially malignant neoplasm or tissue mass of any size.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

As used herein, the term “effective amount” refers to the amount of a compound (e.g., an anti-B7-H4 antibody or antigen-binding fragment thereof or antibody-drug conjugate) sufficient to effect beneficial or desired results. An effective amount of an antibody or antigen-binding fragment thereof or antibody-drug conjugate (e.g., a B7-H4-ADC) can be administered in one or more administrations, applications or dosages and is not intended to be limited to a particular formulation or administration route.

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an anti-B7-H4 antibody (e.g., a B7-H4-ADC) is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

A “platinum-based therapy” refers to treatment with a platinum-based agent. A “platinum-based agent” refers to a molecule or a composition comprising a molecule containing a coordination complex comprising the chemical element platinum and useful as a chemotherapy drug. Platinum-based agents generally act by inhibiting DNA synthesis and some have alkylating activity. Platinum-based agents encompass those that are currently being used as part of a chemotherapy regimen, those that are currently in development, and those that may be developed in the future.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

### I. Anti-B7-H4 Antibodies, Antigen-Binding Fragments and Antibody-Drug Conjugates

In some aspects, provided herein is an antibody or antigen-binding fragment thereof which specifically binds to B7-H4. In some embodiments, the antibody is an anti-B7-H4 antibody.

In some aspects, provided herein is an antibody drug conjugate (ADC) comprising an antibody or antigen-binding fragment thereof which specifically binds to B7-H4. In some embodiments, the ADC is a B7-H4-ADC.

In some aspects, provided herein are methods of treating a patient having or at risk of cancer, comprising administering to the patient an effective amount of an antibody drug conjugate (ADC) comprising an antibody or antigen-binding fragment thereof which specifically binds to B7-H4. In some embodiments, the ADC is B7-H4-ADC.

In some embodiments, the antibody or antigen-binding fragment thereof is an anti-B7-H4 antibody. In some embodiments, the antibody or antigen-binding fragment thereof is an anti-B7-H4 monoclonal antibody (mAb). In some embodiments, the antibody or antigen-binding fragment thereof is a fully human antibody. In some embodiments, the antibody or antigen-binding fragment thereof is a humanized antibody. In some embodiments, the antibody or antigen-binding fragment thereof is conjugated to a moiety such as a cytotoxic agent (for example, but not limited to, an anti-tubulin agent).

SGN-B7H4V is an antibody drug conjugate (ADC) composed of a fully human IgG1 anti-B7-H4 monoclonal antibody (mAb) conjugated to the microtubule disrupting agent monomethyl auristatin E (MMAE) via a protease-cleavable peptide linker (Doronina et al., 2003. Nat Biotechnol 21, 778-784). This “vedotin” drug linker system has been clinically validated by multiple ADC programs, including brentuximab vedotin (Adcetris™), enfortumab vedotin (PADCEV™), and polatuzumab vedotin (POLIVY™) (Rosenberg et al., 2019, J Clin Oncol 37, 2592-2600; Senter and Sievers, 2012, Nat Biotechnol 30, 631-637; Tilly et al., 2019, Lancet Oncol 20, 998-1010). The antibody component of SGN-B7H4V is a fucosylated mAb that should have a similar profile to B7H41001, an afucosylated mAb targeting B7-H4 that exhibited a favorable safety profile in a Phase 1 clinical trial (Wainberg, 2019, “Phase 1 Update in Advanced Solid Tumors: Monotherapy and in Combination with Pembrolizumab,” presented at: ESMO 2019 Congress (Annals of Oncology)).

The present invention provides isolated, recombinant and/or synthetic human, primate, rodent, mammalian, chimeric, humanized and/or CDR-grafted antibodies and antigen-binding fragments and antibody-drug conjugates (e.g., a B7-H4-ADC) thereof, as well as compositions and nucleic acid molecules comprising at least one polynucleotide encoding at least a portion of one antibody molecule. The present invention further includes, but is not limited to, methods of making and using such nucleic acids and antibodies including diagnostic and therapeutic compositions, methods and devices. In certain exemplary embodiments, humanized anti-B7-H4 IgG1 antibodies are provided. In other exemplary embodiments, humanized anti-B7-H4 IgG1 antibody-drug conjugates are provided. In certain exemplary embodiments, fully human anti-B7-H4 IgG1 antibodies are provided. In other exemplary embodiments, fully human anti-B7-H4 IgG1 antibody-drug conjugates are provided.

In some embodiments, the invention provides an antibody-drug conjugate for the treatment of cancer. In some embodiments, the antibody-drug conjugate comprises an antibody conjugated to an auristatin. In some embodiments, the auristatin is a monomethyl auristatin. In some embodiments, the monomethyl auristatin is monomethyl auristatin E.

Unless otherwise indicated, an anti-B7-H4-antibody drug conjugate (i.e., a B7-H4-ADC) includes an antibody specific for the human B7-H4 protein conjugated to a cytotoxic agent.

SGN-B7H4V comprises a fully human anti-B7-H4 monoclonal IgG1 antibody (mAb), which is conjugated to monomethyl auristatin E (MMAE) via a protease-cleavable linker (i.e., a valine-citrulline linker). Upon binding to a B7-H4 expressing cell, SGN-B7H4V is internalized and releases MMAE, which disrupts microtubulin and induces apoptosis.

B7-H4 ADCs (such as but not limited to SGN-B7H4V) comprises a fully human anti-B7-H4 antibody, where examples of such antibodies were described in US Patent Publication US20190085080. Methods of making certain anti-B7-H4 antibodies are also disclosed in US Patent Publication US20190085080, which is incorporated herein by reference in its entirety for all purposes.

In some embodiments, the antibodies (e.g., monoclonal antibodies, such as chimeric, humanized, or human antibodies) or antigen-binding fragments thereof which specifically bind to B7-H4 (e.g., human B7-H4). The amino acid sequences for human, cynomolgus monkey, murine, and rat B7-H4 are known in the art and also provided herein as represented by SEQ ID NOs: 1-4, respectively.

TABLE 1A
Amino acid sequences for human, cynomolgus monkey, murine, and rat B7-H4

| PROTEIN | AMINO ACD SEQUENCE |
|---|---|
| Human | MASLGQILFWSIISIIIILAGAIALIIGFGISGRHSITVTTVASAGNIGEDGILSCTFEP |
| B7-H4 | DIKLSDIVIQWLKEGVLGLVHEFKEGKDELSEQDEMFRGRTAVFADQVIV |
| | GNASLRLKNVQLTDAGTYKCYIITSKGKGNANLEYKTGAFSMPEVNVDYN |
| | ASSETLRCEA PRWFPQPTVV WASQVDQGAN FSEVSNTSFELNSENVTMKV |
| | VSVLYNVTINNTYSCMIENDIAKATGDIKVTESEIKRRSHLQLLNSKASL |
| | CVSSFFAISWALLPLSPYLMLK (SEQ ID NO: 1) |
| Cynomolgus | MASLGQILFW SIISIIFILA GAIALIIGFG ISGRHSITVT TVASAGNIGE |
| monkey B7-H4 | DGILSCTFEP DIKLSDIVIQ WLKEGVIGLV HEFKEGKDEL SEQDEMFRGR |
| | TAVFADQVIV GNASLRLKNV QLTDAGTYKC YIITSKGKGN ANLEYKTGAF |
| | SMPEVNVDYN ASSETLRCEA PRWFPQPTVV WASQVDQGAN FSEVSNTSFE |
| | LNSENVTMKV VSVLYNVTIN NTYSCMIEND IAKATGDIKV TESEIKRRSH |
| | LQLLNSKASL CVSSFLAISW ALLPLAPYLM LK (SEQ ID NO: 2) |
| Murine B7-H4 | MASLGQIIFW SIINIIIILA GAIALIIGFG ISGKHFITVT TFTSAGNIGE |
| | DGTLSCTFEP DIKLNGIVIQ WLKEGIKGLV HEFKEGKDDL SQQHEMFRGR |
| | TAVFADQVVV GNASLRLKNV QLTDAGTYTC YIRTSKGKGN ANLEYKTGAF |
| | SMPEINVDYN ASSESLRCEA PRWFPQPTVA WASQVDQGAN FSEVSNTSFE |
| | LNSENVTMKV VSVLYNVTIN NTYSCMIEND IAKATGDIKV TDSEVKRRSQ |
| | LQLLNSGPSP CVFSSAFVAG WALLSLSCCL MLR (SEQ ID NO: 3) |
| Rat B7-H4 | MASLGQIIFW SIINVIIILA GAIVLIIGFG ISGKHFITVT TFTSAGNIGE |
| | DGTLSCTFEP DIKLNGIVIQ WLKEGIKGLV HEFKEGKDDL SQQHEMFRGR |
| | TAVFADQVVV GNASLRLKNV QLTDAGTYTC YIHTSKGKGN ANLEYKTGAF |
| | SMPEINVDYN ASSESLRCEA PRWFPQPTVA WASQVDQGAN FSEVSNTSFE |
| | LNSENVTMKV VSVLYNVTIN NTYSCMIEND IAKATGDIKV TDSEVKRRSQ |
| | LELLNSGPSP CVSSVSAAGW ALLSLSCCLM LR (SEQ ID NO: 4) |

In certain embodiments according to any one of the methods, antibodies, or antibody-conjugates described herein, the antibody or antigen-binding fragment thereof described herein binds to human B7-H4. In certain embodiments, an antibody or antigen-binding fragment thereof binds to human and cynomolgus monkey B7-H4. In certain embodiments, an antibody or antigen-binding fragment thereof binds to human, murine, and rat B7-H4. In certain embodiments, an antibody or antigen-binding fragment thereof specifically binds to one or more of: human, cynomolgus monkey, murine, and rat B7-H4.

B7-H4 contains an IgC ectodomain (amino acids 153-241 of SEQ ID NO: 1) and an IgV domain (amino acids 35-146 of SEQ ID NO:1).

In certain embodiments, an antibody or antigen-binding fragment thereof described herein binds to the IgV domain of human B7-H4. In some embodiments, the antibodies and antigen-binding fragments thereof bind to a polypeptide consisting of amino acids 35-146 of SEQ ID NO: 1.

In certain embodiments, the antibody or antigen-binding fragment thereof described herein binds to human B7-H4 and comprises the six CDRs of an antibody listed in Table 1B (i.e., the three VH CDRs of the antibody listed in Table 1B and the three VL CDRs of the same antibody listed in Table 1B). In certain embodiments, the antibody or antigen-binding fragment thereof described herein binds to human B7-H4 and comprises the six CDRs comprising the SEQ ID NOs: 5, 6, 7, 8, 9, and 10 (i.e., the three VH CDRs of the antibody comprising the SEQ ID NOs: 5, 6, and 7 and the three VL CDRs comprising the SEQ ID NOs: 8, 9 and 10.

In some embodiments, the antibody or antigen-binding fragment thereof described herein binds to human B7-H4, wherein the antibody or antigen-binding fragment thereof comprises a VH comprising a VH-CDR1 comprising the amino acid sequence of SEQ ID NO: 5, an VH-CDR2 comprising the amino acid sequence of SEQ ID NO: 6, and a VH-CDR3 comprising the amino acid sequence of SEQ ID NO: 7; and a VL comprising a VL-CDR1 comprising the amino acid sequence of SEQ ID NO: 8, a VL-CDR2 comprising the amino acid sequence of SEQ ID NO: 9, and a VL-CDR3 comprising the amino acid sequence of SEQ ID NO: 10.

In some embodiments, the antibody or antigen-binding fragment thereof comprises a VH comprising a sequence with at least 80% sequence identity to the amino acid sequence of SEQ ID NO: 11 and a VL comprising a sequence with at least 80% sequence identity to the amino acid sequence of SEQ ID NO: 12. In some embodiments, the antibody or antigen-binding fragment thereof comprises a VH comprising a sequence with at least 90% sequence identity to the amino acid sequence of SEQ ID NO: 11 and a VL comprising a sequence with at least 90% sequence identity to the amino acid sequence of SEQ ID NO: 12. In some embodiments, the antibody or antigen-binding fragment thereof comprises a VH comprising a sequence with at least 95% sequence identity to the amino acid sequence of SEQ ID NO: 11 and a VL comprising a sequence with at least 95% sequence identity to the amino acid sequence of SEQ ID NO: 12. In some embodiments, the antibody or antigen-binding fragment thereof comprises a VH comprising a sequence with at least 98% sequence identity to the amino acid sequence of SEQ ID NO: 11 and a VL comprising a sequence with at least 98% sequence identity to the amino acid sequence of SEQ ID NO: 12. In some embodiments, the antibody or antigen-binding fragment thereof comprises a VH comprising a sequence with at least 99% sequence identity to the amino acid sequence of SEQ ID NO: 11 and a VL comprising a sequence with at least 99% sequence identity to the amino acid sequence of SEQ ID NO: 12. In some embodiments, the antibody or antigen-binding fragment thereof comprises a VH comprising the amino acid sequence of SEQ ID NO: 11 and a VL comprising the amino acid sequence of SEQ ID NO: 12. In certain embodiments, an antibody or antigen-binding fragment thereof described herein binds to human B7-H4 and comprises the heavy chain sequence of SEQ ID NO: 13. In certain embodiments, an antibody or antigen-binding fragment thereof described herein binds to human B7-H4 and comprises the light chain sequence of SEQ ID NO: 14.

TABLE IB
CDRs, variable regions, and full-length antibody
amino acid sequences for B7- H41001 mAb.

| Sequence | SEQ ID NO: | SEQUENCE |
|---|---|---|
| VH CDR1 | 5 | GSIKSGSYYWG |
| VH CDR2 | 6 | NIYYSGSTYYNPSLRS |
| VH CDR3 | 7 | AREGSYPNQFDP |
| VL CDR1 | 8 | RASQSVSSNLA |
| VL CDR2 | 9 | GASTRAT |
| VL CDR3 | 10 | QQYHSFPFT |
| VH | 11 | QLQLQESGPGLVKPSETLSLTCTVSGGSIKSGSYYWGWIRQPPGKGLEW |
| | | IGNIYYSGSTYYNPSLRSRVTISVDTSKNQFSLKLSSVTAADTAVYYCA |
| | | REGSYPNQFDPWGQGTLVTVSS |
| VL | 12 | EIVMTQSPATLSVSPGERATLSCRASQSVSSNLAWYQQKPGQAPRLLIY |
| | | GASTRATGIPARFSGSGSGTEFTLTISSLQSEDFAVYYCQQYHSFPFTF |
| | | GGGTKVEIK |
| Full-length | 13 | QLQLQESGPGLVKPSETLSLTCTVSGGSIKSGSYYWGWIRQPPGKGLEW |
| Heavy chain | | IGNIYYSGSTYYNPSLRSRVTISVDTSKNQFSLKLSSVTAADTAVYYCA |
| | | REGSYPNQFDPWGQGTLVTVSSASTKGPSVFPLAPSSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQ |
| | | KSLSLSPGK |
| Full-length | 14 | EIVMTQSPATLSVSPGERATLSCRASQSVSSNLAWYQQKPGQAPRLLIY |
| Light chain | | GASTRATGIPARFSGSGSGTEFTLTISSLQSEDFAVYYCQQYHSFPFTF |
| | | GGGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |

In certain embodiments, the antibody or antigen-binding fragment thereof described herein binds to human B7-H4 and comprises the six CDRs of an antibody listed in Tables 2 and 3 (i.e., the three VH CDRs of the antibody listed in Table 2 and the three VL CDRs of the same antibody listed in Table 3).

TABLE 2
VH CDR amino acid sequences (Kabat)

| ANTIBODY | VH CDR1 | VH CDR2 | VH CDR 3 |
|---|---|---|---|
| 15495 | GTFSSYAIS | GIIPIFGTASYAQKFQG | ARQQYDGRRY |
| | (SEQ ID | (SEQ ID NO: 22) | FGL |
| | NO: 15) | | (SEQ ID |
| | | | NO: 29) |
| 15503 | FTFSSYAMS | AISGSGGSTYYADSVKG | ARVGFRALNY |
| | (SEQ ID | (SEQ ID NO: 23) | (SEQ ID |
| | NO: 16) | | NO: 30) |
| 15465 | GSISSGGYYWS | NIYYSGSTYYNPSLKS | ARESSTISAD |
| | (SEQ ID | (SEQ ID NO: 24) | FDL |
| | NO: 17) | | (SEQ ID |
| | | | NO: 31) |
| 20506 | GSISHGGYYWS | NIYYSGSTYYNPSLKS | ARESSTISAD |
| | (SEQ ID | (SEQ ID NO: 24) | FDL |
| | NO: 18) | | (SEQ ID |
| | | | NO: 31) |
| 20513 | GSISDGSYYWS | NIYYSGSTYYNPSLRS | ARGLSTIDEA |
| | (SEQ ID | (SEQ ID NO: 6) | FDP |
| | NO: 19) | | (SEQ ID |
| | | | NO: 32) |
| 20516 | GSIISYYWG | YIYSSGSTSYNPSLKS | ARGSGLYAAP |
| | (SEQ ID | (SEQ ID NO: 25) | DYGLDV |
| | NO: 20) | | (SEQ ID |
| | | | NO: 33) |
| 15472 | FTFSSYAMS | TISGSGGSTYYADSVKG | ARGAGHYDLV |
| | (SEQ ID | (SEQ ID NO: 26) | GRY |
| | NO: 16) | | (SEQ ID |
| | | | NO: 34) |
| 15478 | GTFSSYAIS | GIIPIFGTANYAQKFQG | ARGGPWFDP |
| | (SEQ ID | (SEQ ID NO: 27) | (SEQ ID |
| | NO: 15) | | NO: 35) |
| 20496 | GSISSSVYYWS | SILVSGSTYYNPSLKS | ARAVSFLDV |
| | (SEQ ID | (SEQ ID NO: 28) | (SEQ ID |
| | NO: 21) | | NO: 36) |

TABLE 3
VL CDR amino acid sequences (Kabat)

| ANTIBODY | VL CDR1 | VL CDR2 | VL CDR 3 |
|---|---|---|---|
| 15495 | RASQSVSSNLA | SASTRAT | QQVNVWPPT |
| | (SEQ ID NO: 8) | (SEQ ID | (SEQ ID NO: 45) |
| | | NO: 41) | |
| 15503 | RASQDISSWLA | AASSLQS | QQATSYPPWT |
| | (SEQ ID NO: 37) | (SEQ ID | (SEQ ID NO: 46) |
| | | NO: 42) | |
| 15465 | RASQGISRWLA | AASSLQS | QQAHTFPYT |
| | (SEQ ID NO: 38) | (SEQ ID | (SEQ ID NO: 47) |
| | | NO: 42) | |
| 20506 | RASQGISRWLA | AASSLQS | QQAHTFPYT |
| | (SEQ ID NO: 38) | (SEQ ID | (SEQ ID NO: 47) |
| | | NO: 42) | |
| 20513 | RASQSISSWLA | KASSLES | QQDNSYPYT |
| | (SEQ ID NO: 39) | (SEQ ID | (SEQ ID NO: 48) |
| | | NO: 43) | |
| 20516 | RASQSISSWLA | KASSLES | QQDNSFPFT |
| | (SEQ ID NO: 39) | (SEQ ID | (SEQ ID NO: 49) |
| | | NO: 43) | |
| 15472 | RASQSISSYLN | AASSLQS | QQLYSLPPT |
| | (SEQ ID NO: 40) | (SEQ ID | (SEQ ID NO: 50) |
| | | NO: 42) | |
| 15478 | RASQSISSWLA | KASSLES | QQYNSYPPFT |
| | (SEQ ID NO: 39) | (SEQ ID | (SEQ ID NO: 51) |
| | | NO: 43) | |
| 20496 | RASQSISSYLN | GASSLQS | QQSYDPPWT |
| | (SEQ ID NO: 40) | (SEQ ID | (SEQ ID NO: 52) |
| | | NO: 44) | |

In certain embodiments, the antibody or antigen-binding fragment thereof described herein binds to human B7-H4 and comprises the VH and the VL of an antibody listed in Tables 4 and 5 (i.e., the VH of the antibody listed in Table 4 and the VL of the same antibody listed in Table 5)

TABLE 4
VH amino acid sequences

| ANTIBODY | VH AMINO ACID SEQUENCE |
|---|---|
| 15495 | QVQLVQSGAEVKKPGSSVKVSCKASGGTFSSYAISWVRQAP |
| | GQGLEWMGGIIPIFGTASYAQKFQGRVTITADESTSTAYME |
| | LSSLRSEDTAVYYCARQQYDGRRYFGLWGRGTLVTVSS |
| | (SEQ ID NO: 53) |
| 15503 | EVQLLESGGGLVQPGGSLRLSCAASGFTFSSYAMSWVRQAP |
| | GKGLEWVSAISGSGGSTYYADSVKGRFTISRDNSKNTLYLQ |
| | MNSLRAEDTAVYYCARVGFRALNYWGQGTTVTVSS |
| | (SEQ ID NO: 54) |
| 15465 | QVQLQESGPGLVKPSQTLSLTCTVSGGSISSGGYYWSWIRQ |
| | HPGKGLEWIGNIYYSGSTYYNPSLKSRVTISVDTSKNQFSL |
| | KLSSVTAADTAVYYCARESSTISADFDLWGRGTLVTVSS |
| | (SEQ ID NO: 55) |
| 20506 | QLQLQESGPGLVKPSETLSLTCTASGGSISHGGYYWSWIRQ |
| | HPGKGLEWIGNIYYSGSTYYNPSLKSRVTMSVDTSKNQFSL |
| | KLSSVTAADTAVYYCARESSTISADFDLWGRGTLVTVSS |
| | (SEQ ID NO: 56) |
| 20513 | QLQLQESGPGLVKPSETLSLTCTVSGGSISDGSYYWSWIRQ |
| | HPGKGLEWIGNIYYSGSTYYNPSLRSRVTMSVDTSKNQFSL |
| | KLSSVTAADTAVYYCARGLSTIDEAFDPWGQGTLVTVSS |
| | (SEQ ID NO: 57) |
| 20516 | QVQLQESGPGLVKPSETLSLTCTVSGGSIISYYWGWIRQPP |
| | GKGLEWIGYIYSSGSTSYNPSLKSRVTISVDTSKNQFSLKL |
| | SSVTAADTAVYYCARGSGLYAAPDYGLDVWGQGTTVTVSS |
| | (SEQ ID NO: 58) |
| 15472 | EVQLLESGGGLVQPGGSLRLSCAASGFTFSSYAMSWVRQAP |
| | GKGLEWVSTISGSGGSTYYADSVKGRFTISRDNSKNTLYLQ |
| | MNSLRAEDTAVYYCARGAGHYDLVGRYWGQGTLVTVSS |
| | (SEQ ID NO: 59) |
| 15478 | QVQLVQSGAEVKKPGSSVKVSCKASGGTFSSYAISWVRQAP |
| | GQGLEWMGGIIPIFGTANYAQKFQGRVTITADESTSTAYME |
| | LSSLRSEDTAVYYCARGGPWFDPWGQGTLVTVSS |
| | (SEQ ID NO: 60) |
| 20496 | QLQLQESGPGLVKPSETLSLTCTVSGGSISSSVYYWSWIRQ |
| | PPGKGLEWIGSILVSGSTYYNPSLKSRVTISVDTSKNQFSL |
| | KLSSVTAADTAVYYCARAVSFLDVWGQGTMVIVSS |
| | (SEQ ID NO: 61) |

TABLE 5
VL amino acid sequences

| ANTIBODY | VL AMINO ACID SEQUENCE |
|---|---|
| 15495 | EIVMTQSPATLSVSPGERATLSCRASQSVSSNLAWYQQKPG |
| | QAPRLLIYSASTRATGIPARFSGSGSGTEFTLTISSLQSED |
| | FAVYYCQQVNVWPPTFGGGTKVEIK (SEQ ID NO: 62) |
| 15503 | DIQLTQSPSSVSASVGDRVTITCRASQDISSWLAWYQQKPG |
| | KAPKLLIYAASSLQSGVPSRFSGSGSGTDFTLTISSLQPED |
| | FATYYCQQATSYPPWTFGGGTKVEIK (SEQ ID NO: 63) |
| 15465 | DIQMTQSPSSVSASVGDRVTITCRASQGISRWLAWYQQKPG |
| | KAPKLLIYAASSLQSGVPSRFSGSGSGTDFTLTISSLQPED |
| | FATYYCQQAHTFPYTFGGGTKVEIK (SEQ ID NO: 64) |
| 20506 | DIQMTQSPSSVSASVGDRVTITCRASQGISRWLAWYQQKPG |
| | KAPKLLIYAASSLQSGVPSRFSGSGSGTDFTLTISSLQPED |
| | FATYYCQQAHTFPYTFGGGTKVEIK (SEQ ID NO: 65) |
| 20513 | DIQMTQSPSTLSASVGDRVTITCRASQSISSWLAWYQQKPG |
| | KAPKLLIYKASSLESGVPSRFSGSGSGTEFTLTISSLQPDD |
| | FATYYCQQDNSYPYTFGGGTKVEIK (SEQ ID NO:66) |
| 20516 | DIQMTQSPSTLSASVGDRVTITCRASQSISSWLAWYQQKPG |
| | KAPKLLIYKASSLESGVPSRFSGSGSGTEFTLTISSLQPDD |
| | FATYYCQQDNSFPFTFGGGTKVEIK (SEQ ID NO: 67) |
| 15472 | DIQMTQSPSSLSASVGDRVTITCRASQSISSYLNWYQQKPG |
| | KAPKLLIYAASSLQSGVPSRFSGSGSGTDFTLTISSLQPED |
| | FATYYCQQLYSLPPTFGGGTKVEIK (SEQ ID NO: 68) |
| 15478 | DIQMTQSPSTLSASVGDRVTITCRASQSISSWLAWYQQKPG |
| | KAPKLLIYKASSLESGVPSRFSGSGSGTEFTLTISSLQPDD |
| | FATYYCQQYNSYPPFTFGGGTKVEIK (SEQ ID NO: 69) |
| 20496 | DIQMTQSPSSLSASVGDRVTITCRASQSISSYLNWYQQKPG |
| | KAPKLLIYGASSLQSGVPSRFSGSGSGTDFTLTISSLQPED |
| | FATYYCQQSYDPPWTFGGGTKVEIK (SEQ ID NO: 70) |

In certain embodiments, an antibody or antigen-binding fragment thereof described herein binds to human B7-H4 and comprises the heavy chain sequence of an antibody listed in Table 6.

TABLE 6
Full-length heavy chain amino acid sequences

| ANTI- | |
|---|---|
| BODY | Full-Length Heavy Chain Amino Acid Sequence |
| 15495 | QVQLVQSGAEVKKPGSSVKVSCKASGGTFSSYAISWVRQAPGQ |
| | GLEWMGGIIPIFGTASYAQKFQGRVTITADESTSTAYMELSSL |
| | RSEDTAVYYCARQQYDGRRYFGLWGRGTLVTVSSASTKGPSVF |
| | PLAPSSKSTSGGTAALGCLVKDYFPEPVTVSWNSGALTSGVHT |
| | FPAVLQSSGLYSLSSVVTVPSSSLGTQTYICNVNHKPSNTKVD |
| | KKVEPKSCDKTHTCPPCPAPELLGGPSVFLFPPKPKDTLMISR |
| | TPEVTCVVVDVSHEDPEVKFNWYVDGVEVHNAKTKPREEQYNS |
| | TYRVVSVLTVLHQDWLNGKEYKCKVSNKALPAPIEKTISKAKG |
| | QPREPQVYTLPPSRDELTKNQVSLTCLVKGFYPSDIAVEWESN |
| | GQPENNYKTTPPVLDSDGSFFLYSKLTVDKSRWQQGNVFSCSV |
| | MHEALHNHYTQKSLSLSPGK (SEQ ID NO: 71) |
| 15503 | EVQLLESGGGLVQPGGSLRLSCAASGFTFSSYAMSWVRQAPGK |
| | GLEWVSAISGSGGSTYYADSVKGRFTISRDNSKNTLYLQMNSL |
| | RAEDTAVYYCARVGFRALNYWGQGTTVTVSSASTKGPSVFPLA |
| | PSSKSTSGGTAALGCLVKDYFPEPVTVSWNSGALTSGVHTFPA |
| | VLQSSGLYSLSSVVTVPSSSLGTQTYICNVNHKPSNTKVDKKV |
| | EPKSCDKTHTCPPCPAPELLGGPSVFLFPPKPKDTLMISRTPE |
| | VTCVVVDVSHEDPEVKFNWYVDGVEVHNAKTKPREEQYNSTYR |
| | VVSVLTVLHQDWLNGKEYKCKVSNKALPAPIEKTISKAKGQPR |
| | EPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGK (SEQ ID NO: 72) |
| 15465 | QVQLQESGPGLVKPSQTLSLTCTVSGGSISSGGYYWSWIRQHP |
| | GKGLEWIGNIYYSGSTYYNPSLKSRVTISVDTSKNQFSLKLSS |
| | VTAADTAVYYCARESSTISADFDLWGRGTLVTVSSASTKGPSV |
| | FPLAPSSKSTSGGTAALGCLVKDYFPEPVTVSWNSGALTSGVH |
| | TFPAVLQSSGLYSLSSVVTVPSSSLGTQTYICNVNHKPSNTKV |
| | DKKVEPKSCDKTHTCPPCPAPELLGGPSVFLFPPKPKDTLMIS |
| | RTPEVTCVVVDVSHEDPEVKFNWYVDGVEVHNAKTKPREEQYN |
| | STYRVVSVLTVLHQDWLNGKEYKCKVSNKALPAPIEKTISKAK |
| | GQPREPQVYTLPPSRDELTKNQVSLTCLVKGFYPSDIAVEWES |
| | NGQPENNYKTTPPVLDSDGSFFLYSKLTVDKSRWQQGNVFSCS |
| | VMHEALHNHYTQKSLSLSPGK (SEQ ID NO: 73) |
| 20506 | QLQLQESGPGLVKPSETLSLTCTASGGSISHGGYYWSWIRQHP |
| | GKGLEWIGNIYYSGSTYYNPSLKSRVTMSVDTSKNQFSLKLSS |
| | VTAADTAVYYCARESSTISADFDLWGRGTLVTVSSASTKGPSV |
| | FPLAPSSKSTSGGTAALGCLVKDYFPEPVTVSWNSGALTSGVH |
| | TFPAVLQSSGLYSLSSVVTVPSSSLGTQTYICNVNHKPSNTKV |
| | DKKVEPKSCDKTHTCPPCPAPELLGGPSVFLFPPKPKDTLMIS |
| | RTPEVTCVVVDVSHEDPEVKFNWYVDGVEVHNAKTKPREEQYN |
| | STYRVVSVLTVLHQDWLNGKEYKCKVSNKALPAPIEKTISKAK |
| | GQPREPQVYTLPPSRDELTKNQVSLTCLVKGFYPSDIAVEWES |
| | NGQPENNYKTTPPVLDSDGSFFLYSKLTVDKSRWQQGNVFSCS |
| | VMHEALHNHYTQKSLSLSPGK (SEQ ID NO: 74) |
| 20513 | QLQLQESGPGLVKPSETLSLTCTVSGGSISDGSYYWSWIRQHP |
| | GKGLEWIGNIYYSGSTYYNPSLRSRVTMSVDTSKNQFSLKLSS |
| | VTAADTAVYYCARGLSTIDEAFDPWGQGTLVTVSSASTKGPSV |
| | FPLAPSSKSTSGGTAALGCLVKDYFPEPVTVSWNSGALTSGVH |
| | TFPAVLQSSGLYSLSSVVTVPSSSLGTQTYICNVNHKPSNTKV |
| | DKKVEPKSCDKTHTCPPCPAPELLGGPSVFLFPPKPKDTLMIS |
| | RTPEVTCVVVDVSHEDPEVKFNWYVDGVEVHNAKTKPREEQYN |
| | STYRVVSVLTVLHQDWLNGKEYKCKVSNKALPAPIEKTISKAK |
| | GQPREPQVYTLPPSRDELTKNQVSLTCLVKGFYPSDIAVEWES |
| | NGQPENNYKTTPPVLDSDGSFFLYSKLTVDKSRWQQGNVFSCS |
| | VMHEALHNHYTQKSLSLSPGK (SEQ ID NO: 75) |
| 20516 | QVQLQESGPGLVKPSETLSLTCTVSGGSIISYYWGWIRQPPGK |
| | GLEWIGYIYSSGSTSYNPSLKSRVTISVDTSKNQFSLKLSSVT |
| | AADTAVYYCARGSGLYAAPDYGLDVWGQGTTVTVSSASTKGPS |
| | VFPLAPSSKSTSGGTAALGCLVKDYFPEPVTVSWNSGALTSGV |
| | HTFPAVLQSSGLYSLSSVVTVPSSSLGTQTYICNVNHKPSNTK |
| | VDKKVEPKSCDKTHTCPPCPAPELLGGPSVFLFPPKPKDTLMI |
| | SRTPEVTCVVVDVSHEDPEVKFNWYVDGVEVHNAKTKPREEQY |
| | NSTYRVVSVLTVLHQDWLNGKEYKCKVSNKALPAPIEKTISKA |
| | KGQPREPQVYTLPPSRDELTKNQVSLTCLVKGFYPSDIAVEWE |
| | SNGQPENNYKTTPPVLDSDGSFFLYSKLTVDKSRWQQGNVFSC |
| | SVMHEALHNHYTQKSLSLSPGK (SEQ ID NO: 76) |
| 15472 | EVQLLESGGGLVQPGGSLRLSCAASGFTFSSYAMSWVRQAPGK |
| | GLEWVSTISGSGGSTYYADSVKGRFTISRDNSKNTLYLQMNSL |
| | RAEDTAVYYCARGAGHYDLVGRYWGQGTLVTVSSASTKGPSVF |
| | PLAPSSKSTSGGTAALGCLVKDYFPEPVTVSWNSGALTSGVHT |
| | FPAVLQSSGLYSLSSVVTVPSSSLGTQTYICNVNHKPSNTKVD |
| | KKVEPKSCDKTHTCPPCPAPELLGGPSVFLFPPKPKDTLMISR |
| | TPEVTCVVVDVSHEDPEVKFNWYVDGVEVHNAKTKPREEQYNS |
| | TYRVVSVLTVLHQDWLNGKEYKCKVSNKALPAPIEKTISKAKG |
| | QPREPQVYTLPPSRDELTKNQVSLTCLVKGFYPSDIAVEWESN |
| | GQPENNYKTTPPVLDSDGSFFLYSKLTVDKSRWQQGNVFSCSV |
| | MHEALHNHYTQKSLSLSPGK (SEQ ID NO: 77) |
| 15478 | QVQLVQSGAEVKKPGSSVKVSCKASGGTFSSYAISWVRQAPGQ |
| | GLEWMGGIIPIFGTANYAQKFQGRVTITADESTSTAYMELSSL |
| | RSEDTAVYYCARGGPWFDPWGQGTLVTVSSASTKGPSVFPLAP |
| | SSKSTSGGTAALGCLVKDYFPEPVTVSWNSGALTSGVHTFPAV |
| | LQSSGLYSLSSVVTVPSSSLGTQTYICNVNHKPSNTKVDKKVE |
| | PKSCDKTHTCPPCPAPELLGGPSVFLFPPKPKDTLMISRTPEV |
| | TCVVVDVSHEDPEVKFNWYVDGVEVHNAKTKPREEQYNSTYRV |
| | VSVLTVLHQDWLNGKEYKCKVSNKALPAPIEKTISKAKGQPRE |
| | PQVYTLPPSRDELTKNQVSLTCLVKGFYPSDIAVEWESNGQPE |
| | NNYKTTPPVLDSDGSFFLYSKLTVDKSRWQQGNVFSCSVMHEA |
| | LHNHYTQKSLSLSPGK (SEQ ID NO: 78) |
| 20496 | QLQLQESGPGLVKPSETLSLTCTVSGGSISSSVYYWSWIRQPP |
| | GKGLEWIGSILVSGSTYYNPSLKSRVTISVDTSKNQFSLKLSS |
| | VTAADTAVYYCARAVSFLDVWGQGTMVIVSSASTKGPSVFPLA |
| | PSSKSTSGGTAALGCLVKDYFPEPVTVSWNSGALTSGVHTFPA |
| | VLQSSGLYSLSSVVTVPSSSLGTQTYICNVNHKPSNTKVDKKV |
| | EPKSCDKTHTCPPCPAPELLGGPSVFLFPPKPKDTLMISRTPE |
| | VTCVVVDVSHEDPEVKFNWYVDGVEVHNAKTKPREEQYNSTYR |
| | VVSVLTVLHQDWLNGKEYKCKVSNKALPAPIEKTISKAKGQPR |
| | EPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGK (SEQ ID NO: 79) |

In certain embodiments, the antibody or antigen-binding fragment thereof described herein binds to human B7-H4 and comprises the light chain sequence of an antibody listed in Table 7.

TABLE 7
Full-length light chain amino acid sequences

| ANTI- | |
|---|---|
| BODY | Full-Length Heavy Chain Amino Acid Sequence |
| 15495 | EIVMTQSPATLSVSPGERATLSCRASQSVSSNLAWYQQKPGQA |
| | PRLLIYSASTRATGIPARFSGSGSGTEFTLTISSLQSEDFAVY |
| | YCQQVNVWPPTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 80) |
| 15503 | DIQLTQSPSSVSASVGDRVTITCRASQDISSWLAWYQQKPGKA |
| | PKLLIYAASSLQSGVPSRFSGSGSGTDFTLTISSLQPEDFATY |
| | YCQQATSYPPWTFGGGTKVEIKRTVAAPSVFIFPPSDEQLKSG |
| | TASVVCLLNNFYPREAKVQWKVDNALQSGNSQESVTEQDSKDS |
| | TYSLSSTLTLSKADYEKHKVYACEVTHQGLSSPVTKSFNRGEC |
| | (SEQ ID NO: 81) |
| 15465 | DIQMTQSPSSVSASVGDRVTITCRASQGISRWLAWYQQKPGKA |
| | PKLLIYAASSLQSGVPSRFSGSGSGTDFTLTISSLQPEDFATY |
| | YCQQAHTFPYTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 82) |
| 20506 | DIQMTQSPSSVSASVGDRVTITCRASQGISRWLAWYQQKPGKA |
| | PKLLIYAASSLQSGVPSRFSGSGSGTDFTLTISSLQPEDFATY |
| | YCQQAHTFPYTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 83) |
| 20513 | DIQMTQSPSTLSASVGDRVTITCRASQSISSWLAWYQQKPGKA |
| | PKLLIYKASSLESGVPSRFSGSGSGTEFTLTISSLQPDDFATY |
| | YCQQDNSYPYTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 84) |
| 20516 | DIQMTQSPSTLSASVGDRVTITCRASQSISSWLAWYQQKPGKA |
| | PKLLIYKASSLESGVPSRFSGSGSGTEFTLTISSLQPDDFATY |
| | YCQQDNSFPFTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 85) |
| 15472 | DIQMTQSPSSLSASVGDRVTITCRASQSISSYLNWYQQKPGKA |
| | PKLLIYAASSLQSGVPSRFSGSGSGTDFTLTISSLQPEDFATY |
| | YCQQLYSLPPTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 86) |
| 15478 | DIQMTQSPSTLSASVGDRVTITCRASQSISSWLAWYQQKPGKA |
| | PKLLIYKASSLESGVPSRFSGSGSGTEFTLTISSLQPDDFATY |
| | YCQQYNSYPPFTFGGGTKVEIKRTVAAPSVFIFPPSDEQLKSG |
| | TASVVCLLNNFYPREAKVQWKVDNALQSGNSQESVTEQDSKDS |
| | TYSLSSTLTLSKADYEKHKVYACEVTHQGLSSPVTKSFNRGEC |
| | (SEQ ID NO: 87) |
| 20496 | DIQMTQSPSSLSASVGDRVTITCRASQSISSYLNWYQQKPGKA |
| | PKLLIYGASSLQSGVPSRFSGSGSGTDFTLTISSLQPEDFATY |
| | YCQQSYDPPWTFGGGTKVEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 88) |

In certain embodiments, the antibody or antigen-binding fragment thereof described herein binds to human B7-H4 and comprises the heavy chain sequence and the light chain sequence of an antibody listed in Tables 6 and 7 (i.e., the heavy chain sequence of the antibody listed in Table 6 and the light chain sequence of the same antibody listed in Table 7).

In certain embodiments, the antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 80% identical to the VH sequence of the same antibody in Table 4 and a VL comprising a sequence at least 80% identical to the VL sequence of the same antibody in Table 5. In certain embodiments, an antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 85% identical to the VH sequence of the same antibody in Table 4 and a VL comprising a sequence at least 85% identical to the VL sequence of the same antibody in Table 5.

In certain embodiments, the antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 90% identical to the VH sequence of the same antibody in Table 4 and a VL comprising a sequence at least 90% identical to the VL sequence of the same antibody in Table 5. In certain embodiments, an antibody or antigen-binding fragment thereof described herein binds to human B7-H4, comprises the six CDRs of an antibody listed in Tables 2 and 3 (i.e., the three VH CDRs of the antibody listed in Table 1B and the three VL CDRs of the same antibody listed in Table 2), and comprises a VH comprising a sequence at least 95% identical to the VH sequence of the same antibody in Table 4 and a VL comprising a sequence at least 95% identical to the VL sequence of the same antibody in Table 5.

In certain embodiments, an antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 96% identical to the VH sequence of the same antibody in Table 4 and a VL comprising a sequence at least 96% identical to the VL sequence of the same antibody in Table 5. In certain embodiments, an antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 97% identical to the VH sequence of the same antibody in Table 4 and a VL comprising a sequence at least 97% identical to the VL sequence of the same antibody in Table 5. In certain embodiments, an antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 98% identical to the VH sequence of the same antibody in Table 4 and a VL comprising a sequence at least 98%) identical to the VL sequence of the same antibody in Table 5. In certain embodiments, an antibody or antigen-binding fragment thereof described herein binds to human B7-H4, comprises the six CDRs of an antibody listed in Tables 2 and 3 (i.e., the three VH CDRs of the antibody listed in Table 2 and the three VL CDRs of the same antibody listed in Table 3), and comprises a VH comprising a sequence at least 99% identical to the VH sequence of the same antibody in Table 4 and a VL comprising a sequence at least 99% identical to the VL sequence of the same antibody in Table 5. In some embodiments, the antibody or antigen-binding fragment thereof binds to human, cynomolgus monkey, rat, and/or mouse B7-H4.

In some embodiments, the antibody or antigen-binding fragment thereof increases T cell proliferation. In some embodiments, the antibody or antigen-binding fragment thereof increases IFN-gamma production. In some embodiments, the antibody or antigen-binding fragment thereof mediates ADCC activity against B7-H4-expressing cells. In some embodiments, the antibody or antigen-binding fragment thereof mediates ADCC activity against B7-H4-expressing cells. In some embodiments, the antibody or antigen-binding fragment thereof does not mediate CDC activity against B7-H4-expressing cells.

In certain aspects, an antibody or antigen-binding fragment thereof described herein may be described by its VL domain alone, or its VH domain alone, or by its 3 VL CDRs alone, or its 3 VH CDRs alone. See, for example, Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, provided herein are antibodies and antigen-binding fragments thereof that specifically bind to B7-H4 (e.g., human B7-H4) and comprise the Chothia VH and VL CDRs of an antibody listed in Tables 4 and 5. In certain embodiments, antibodies or antigen-binding fragments thereof that specifically bind to B7-H4 (e.g., human B7-H4) comprise one or more CDRs, in which the Chothia and Kabat CDRs have the same amino acid sequence. In certain embodiments, provided herein are antibodies and antigen-binding fragments thereof that specifically bind to B7-H4 (e.g., human B7-H4) and comprise combinations of Kabat CDRs and Chothia CDRs.

In certain aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In a particular embodiment, provided herein are antibodies and antigen-binding fragments thereof that specifically bind to B7-H4 (e.g., human B7-H4) and comprise the IMGT VH and VL CDRs of an antibody listed in Tables 4 and 5, for example, as described in Lefranc M-P (1999) supra and Lefranc M-P et al., (1999) supra).

In certain aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Diibel, eds., Chapter 31, pp. 422-439, Springer-Verlag, Berlin (2001). In a particular embodiment, provided herein are antibodies or antigen-binding fragments thereof that specifically bind to B7-H4 (e.g., human B7-H4) and comprise VH and VL CDRs of an antibody listed in Tables 4 and 5 as determined by the method in MacCallum R M et al.

In certain aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In a particular embodiment, provided herein are antibodies or antigen-binding fragments thereof that specifically bind to B7-H4 (e.g., human B7-H4) and comprise VH and VL CDRs of an antibody listed in Tables 4 and 5 as determined by the AbM numbering scheme.

In specific aspects, provided herein are antibodies that comprise a heavy chain and a light chain. With respect to the heavy chain, in a specific embodiment, the heavy chain of an antibody described herein can be an alpha (a), delta (δ), epsilon (ε), gamma (γ) or mu (μ) heavy chain. In another specific embodiment, the heavy chain of an antibody described can comprise a human alpha (a), delta (δ), epsilon (ε), gamma (γ) or mu (μ) heavy chain. In a particular embodiment, an antibody described herein, which immunospecifically binds to B7-H4 (e.g., human B7-H4), comprises a heavy chain wherein the amino acid sequence of the VH domain comprises an amino acid sequence set forth in Table 4 and wherein the constant region of the heavy chain comprises the amino acid sequence of a human gamma (γ) heavy chain constant region. In a specific embodiment, an antibody described herein, which specifically binds to B7-H4 (e.g., human B7-H4), comprises a heavy chain wherein the amino acid sequence of the VH domain comprises a sequence set forth in Table 4, and wherein the constant region of the heavy chain comprises the amino acid of a human heavy chain described herein or known in the art. Non-limiting examples of human constant region sequences have been described in the art, e.g., see U.S. Pat. No. 5,693,780 and Kabat E A et al., (1991) supra.

With respect to the light chain, in a specific embodiment, the light chain of an antibody described herein is a kappa light chain. The constant region of a human kappa light chain can comprise the following amino acid sequence:

(SEQ ID NO: 89)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

The constant region of a human kappa light chain can be encoded by the following nucleotide sequence:

(SEQ ID NO: 90)
CGGACCGTGGCTGCACCATCTGTCTTCATCTTCCCGCCATCTGATGAGC
AGTTGAAATCTGGAACTGCCTCTGTTGTGTGCCTGCTGAATAACTTCTA
TCCCAGAGAGGCCAAAGTACAGTGGAAGGTGGATAACGCCCTCCAATCG
GGTAACTCCCAGGAGAGTGTCACAGAGCAGGACAGCAAGGACAGCACCT
ACAGCCTCAGCAGCACCCTGACGCTGAGCAAAGCAGACTACGAGAAACA
CAAAGTCTACGCCTGCGAAGTCACCCATCAGGGCCTGAGCTCGCCCGTC
ACAAAGAGCTTCAACAGGGGAGAGTGT.

In another specific embodiment, the light chain of an antibody described herein is a lambda light chain. In yet another specific embodiment, the light chain of an antibody described herein is a human kappa light chain or a human lambda light chain. In a particular embodiment, an antibody described herein, which immunospecifically binds to a B7-H4 polypeptide (e.g., human B7-H4) comprises a light chain wherein the amino acid sequence of the VL domain comprises a sequence set forth in Table 5, and wherein the constant region of the light chain comprises the amino acid sequence of a human kappa light chain constant region. In another particular embodiment, an antibody described herein, which immunospecifically binds to B7-H4 (e.g., human B7-H4) comprises a light chain wherein the amino acid sequence of the VL domain comprises a sequence set forth in Table 5 and wherein the constant region of the light chain comprises the amino acid sequence of a human lambda light chain constant region. In a specific embodiment, an antibody described herein, which immunospecifically binds to B7-H4 (e.g., human B7-H4) comprises a light chain wherein the amino acid sequence of the VL domain comprises a sequence set forth in Table 5 and wherein the constant region of the light chain comprises the amino acid sequence of a human kappa or lambda light chain constant region. Non-limiting examples of human constant region sequences have been described in the art, e.g., see U.S. Pat. No. 5,693,780 and Kabat E A et al., (1991) supra.

In a specific embodiment, an antibody described herein, which immunospecifically binds to B7-H4 (e.g., human B7-H4) comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In another specific embodiment, an antibody described herein, which immunospecifically binds to B7-H4 (e.g., human B7-H4) comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In a particular embodiment, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

The constant region of a human IgG1 heavy chain can comprise the following amino acid sequence:

(SEQ ID NO: 91)
ASTKGPSVFPLAPSSKSTSGGTAALGCLVKDYFPEPVTVSWNSGALTSG
VHTFPAVLQSSGYSSSVVTVPSSSLGTQTYICNVNHKPSNTKVDKKVEP
KSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVD
KSRWQQGNVFSCSVMHEALHNHYTQKSLSLSPGK.

The constant region of a human IgG1 heavy chain can be encoded by the following nucleotide sequence:

(SEQ ID NO: 92)
GCCTCCACCAAGGGCCCATCGGTCTTCCCCCTGGCACCCTCCTCCAAGA
GCACCTCTGGGGGCACAGCGGCCCTGGGCTGCCTGGTCAAGGACTACTT
CCCCGAACCGGTGACGGTGTCGTGGAACTCAGGCGCCCTGACCAGCGGC
GTGCACACCTTCCCGGCTGTCCTACAGTCCTCAGGACTCTACTCCCTCA
GCAGCGTGGTGACCGTGCCCTCCAGCAGCTTGGGCACCCAGACCTACAT
CTGCAACGTGAATCACAAGCCCAGCAACACCAAGGTGGACAAGAAAGTT
GAGCCCAAATCTTGTGACAAAACTCACACATGCCCACCGTGCCCAGCAC
CTGAACTCCTGGGGGGACCGTCAGTCTTCCTCTTCCCCCCAAAACCCAA
GGACACCCTCATGATCTCCCGGACCCCTGAGGTCACATGCGTGGTGGTG
GACGTGAGCCACGAAGACCCTGAGGTCAAGTTCAACTGGTACGTGGACG
GCGTGGAGGTGCATAATGCCAAGACAAAGCCGCGGGAGGAGCAGTACAA
CAGCACGTACCGGGTGGTCAGCGTCCTCACCGTCCTGCACCAGGACTGG
CTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACC
ACAGGTGTACACCCTGCCCCCATCCCGGGATGAGCTGACCAAGAACCAG
GTCAGCCTGACCTGCCTGGTCAAAGGCTTCTATCCCAGCGACATCGCCG
TGGAGTGGGAGAGCAATGGGCAGCCGGAGAACAACTACAAGACCACGCC
TCCCGTGCTGGACTCCGACGGCTCCTTCTTCCTCTACAGCAAGCTCACC
GTGGACAAGAGCAGGTGGCAGCAGGGGAACGTCTTCTCATGCTCCGTGA
TGCATGAGGCTCTGCACAACCACTACACGCAGAAGAGCCTCTCCCTGTC
TCCGGGTAAA.

Non-limiting examples of human constant regions are described in the art, e.g., see Kabat E A et al, (1991) supra.

In certain embodiments, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity.

In certain embodiments, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some embodiments, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody or antigen-binding fragment thereof for an Fc receptor (e.g., an activated Fc receptor) on the surface of an effector cell. Mutations in the Fc region that decrease or increase affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In a specific embodiment, one, two, or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody or antigen-binding fragment thereof in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody or antigen-binding fragment thereof in vivo. In some embodiments, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the antibody or antigen-binding fragment thereof in vivo. In other embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody or antigen-binding fragment thereof in vivo. In a specific embodiment, the antibodies or antigen-binding fragments thereof may have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In a specific embodiment, the constant region of the IgG1 comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In certain embodiments, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In a further embodiment, one, two, or more amino acid substitutions are introduced into an IgG constant domain Fc region to alter the effector function(s) of the antibody or antigen-binding fragment thereof. For example, one or more amino acids selected from amino acid residues 234, 235, 236, 237, 297, 318, 320 and 322, numbered according to the EU index as in Kabat, can be replaced with a different amino acid residue such that the antibody or antigen-binding fragment thereof has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain may reduce Fc receptor binding of the circulating antibody or antigen-binding fragment thereof thereby increasing tumor localization. See, e.g., U.S. Pat. Nos. 5,585,097 and 8,591,886 for a description of mutations that delete or inactivate the constant domain and thereby increase tumor localization. In certain embodiments, one or more amino acid substitutions can be introduced into the Fc region to remove potential glycosylation sites on Fc region, which may reduce Fc receptor binding (see, e.g., Shields R L et al., (2001) J Biol Chem 276: 6591-604).

In certain embodiments, one or more amino acids selected from amino acid residues 329, 331, and 322 in the constant region, numbered according to the EU index as in Kabat, can be replaced with a different amino acid residue such that the antibody or antigen-binding fragment thereof has altered C1q binding and/or reduced or abolished complement dependent cytotoxicity (CDC). This approach is described in further detail in U.S. Pat. No. 6,194,551 (Idusogie et al). In some embodiments, one or more amino acid residues within amino acid positions 231 to 238 in the N-terminal region of the CH2 domain are altered to thereby alter the ability of the antibody to fix complement. This approach is described further in International Publication No. WO 94/29351. In certain embodiments, the Fc region is modified to increase the ability of the antibody or antigen-binding fragment thereof to mediate antibody dependent cellular cytotoxicity (ADCC) and/or to increase the affinity of the antibody or antigen-binding fragment thereof for an Fey receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU index as in Kabat. This approach is described further in International Publication No. WO 00/42072.

In certain embodiments, an antibody or antigen-binding fragment thereof described herein comprises the constant domain of an IgG1 with a mutation (e.g., substitution) at position 267, 328, or a combination thereof, numbered according to the EU index as in Kabat. In certain embodiments, an antibody or antigen-binding fragment thereof described herein comprises the constant domain of an IgG1 with a mutation (e.g., substitution) selected from the group consisting of S267E, L328F, and a combination thereof. In certain embodiments, an antibody or antigen-binding fragment thereof described herein comprises the constant domain of an IgG1 with a S267E/L328F mutation (e.g., substitution). In certain embodiments, an antibody or antigen-binding fragment thereof described herein comprising the constant domain of an IgG1 with a S267E/L328F mutation (e.g., substitution) has an increased binding affinity for FcγRIIA, FcγRIIB, or FcγRIIA and FcγRIIB

In specific embodiments, an antibody or antigen-binding fragment thereof (i) comprises the CDR sequences of B7H41001 mAb (e.g., the amino acid sequences of SEQ ID NOs:5-10), the VH and VL sequences of 20502 (the amino acid sequences of SEQ ID NOs:11 and 12, respectively), or the heavy and light chain sequences of 20502 (the amino acid sequences of SEQ ID NOs:13 and 14, respectively) and (ii) is fucosylated.

The amino acid sequence of the heavy chain variable region of SGN-B7H4V is provided herein as SEQ ID NO: 11. The amino acid sequence of the light chain variable region of SGN-B7H4V is provided herein as SEQ ID NO: 12.

### II. ANTIBODY-DRUG CONJUGATES

In certain embodiments, the antibodies of the invention (e.g., anti-B7-H4 antibodies) can be conjugated to a drug to form antibody-drug conjugates (ADCs). An exemplary anti-B7-H4-ADC is SGN-B7H4V. An exemplary antibody comprised within the anti-B7-H4-ADC is B7H41001 mAb.

In certain embodiments, an antibody or antigen-binding fragment thereof can be conjugated to a drug to form an antibody-drug conjugate (ADC) and may have a ratio of drug moieties per antibody of about 1 to about 8. In certain embodiments, an antibody or antigen-binding fragment thereof (e.g., anti-B7-H4 antibody) can be conjugated to a drug to form an ADC and may have a ratio of drug moieties per antibody of about 2 to about 5. In some embodiments, the ratio of drug moieties per antibody is 1, 2, 3, 4, 5, 6, 7, 8, 9, or 10. In certain exemplary embodiments, an anti-B7-H4 antibody or antigen-binding fragment thereof can be conjugated to a drug to form an ADC and have a ratio of drug moieties per antibody of about 4. In some embodiments, the average number of drug moieties per antibody in a population of antibody-drug conjugates is about 1 to about 8. In some embodiments, the average number of drug moieties per antibody in a population of antibody-drug conjugates is about 4. Methods of determining the ratio of drug moieties per antibody or antigen-binding fragment thereof of an ADC are readily known to those skilled in the art.

According to certain exemplary embodiments, a B7-H4-ADC comprises monomethyl auristatin E (MMAE) (PubChem CID: 53297465):

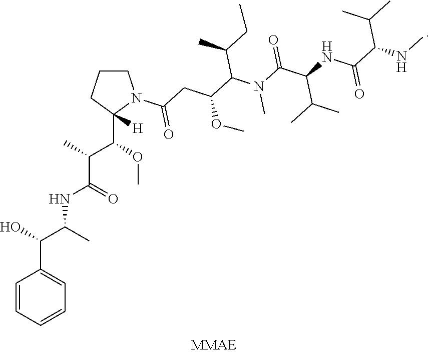 [SMILES: CC[C@H](C)C([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

According to certain exemplary embodiments, a B7-H4-ADC comprises vcMMAE conjugated thereto. vcMMAE is a drug-linker conjugate for ADC with potent anti-tumor activity comprising the anti-mitotic agent, MMAE, linked via the lysosomally cleavable dipeptide valine-citrulline (vc):

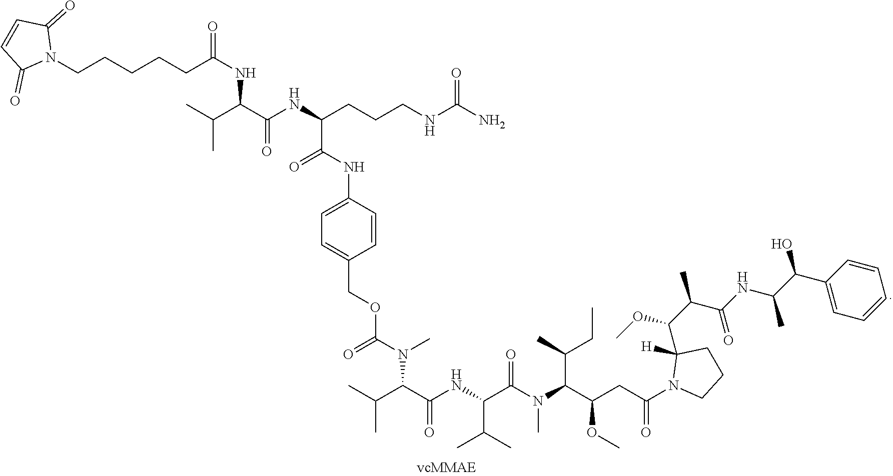 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

vcMMAE may also be referred to as MC-Val-Cit-PABC-MMAE, where MC refers to a maleimidocaproyl group, Val-Cit refers to the dipeptide valine-citrulline, PABC refers to a para-aminobenzylcarbamate group, and MMAE refers to the drug monomethyl auristatin E.

The structure of a vcMMAE-antibody conjugate (e.g., a B7-H4-ADC) according to certain exemplary embodiments is set forth below. In this structure, the drug-linker portion shown within the parentheses may be referred to in some instances as vedotin. The drug-linker may be attached to the antibody via a sulfur atom of a cysteine residue of the antibody. In some embodiments, the ADC shown below is formed by reaction of the maleimide group of the vc-MMAE drug-linker precursor with a thiol of a cysteine residue of the antibody to form a succinamide bonded to the sulfur atom of the cysteine residue.

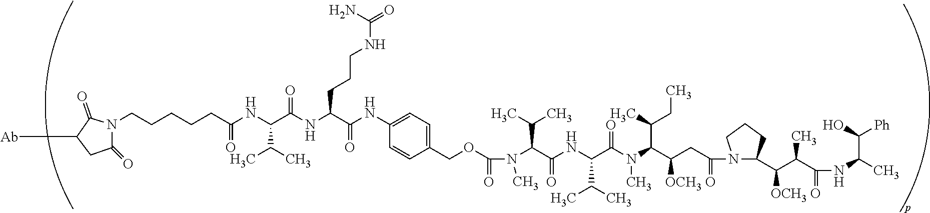

In some embodiments, the succinamide moiety of the ADC may undergo a ring-opening hydrolysis to form one of the ring-opened structures shown below.

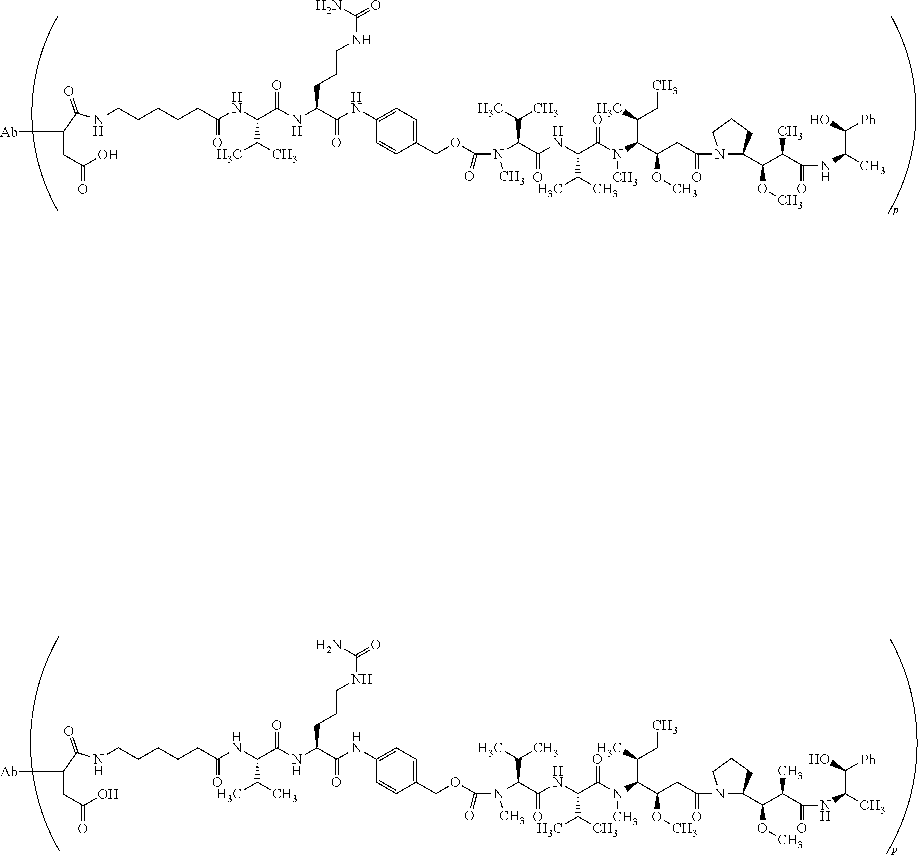

According to certain exemplary embodiments, a vcMMAE-antibody conjugate (e.g., a B7-H4-ADC) is provided as set forth above, wherein Ab may include an anti-B7-H4 antibody or antigen-binding fragment thereof (e.g., B7H41001 mAb), and wherein p may be any integer from about 1 to about 8. In some embodiments, a vcMMAE-antibody conjugate (e.g., a B7-H4-ADC) is provided as set forth above, wherein Ab may include an anti-B7-H4 antibody or antigen-binding fragment thereof (e.g., B7H41001 mAb), and wherein p is 1, representing a vcMMAE to antibody or antigen-binding fragment thereof ratio of 1. In some embodiments, a vcMMAE-antibody conjugate (e.g., a B7-H4-ADC) is provided as set forth above, wherein Ab may include an anti-B7-H4 antibody or antigen-binding fragment thereof (e.g., B7H41001 mAb), and wherein p is 2, 3, 4, 5, 6, 7, 8, 9, or 10, representing a vcMMAE to antibody or antigen-binding fragment thereof ratio (also known as a “Drug-to-Antibody Ratio” or “DAR”) of 2, 3, 4, 5, 6, 7, 8, 9, or 10, respectively. Accordingly, in some embodiments, a vcMMAE-antibody conjugate (e.g., a B7-H4-ADC) is provided as set forth above, wherein a vcMMAE to antibody or antigen-binding fragment thereof ratio is 1, 2, 3, 4, 5, 6, 7, 8, 9, or 10. In certain exemplary embodiments, a vcMMAE-antibody conjugate (e.g., a B7-H4-ADC) is provided as set forth above, wherein Ab may include an anti-B7-H4 antibody or antigen-binding fragment thereof (e.g., B7H41001 mAb), and wherein p is 4, representing a vcMMAE to antibody or antigen-binding fragment thereof ratio of 4. Accordingly, in certain exemplary embodiments, a vcMMAE-antibody conjugate (e.g., a B7-H4-ADC) is provided as set forth above, wherein a vcMMAE to antibody or antigen-binding fragment thereof ratio is 4.

SGN-B7H4V can be administered to subjects at a level that inhibits cancer cell growth, while at the same time is tolerated by the subject.

In certain exemplary embodiments, an anti-B7-H4 antibody or antigen-binding fragment thereof comprises CDRs from an HCVR set forth as SEQ ID NO: 11 and/or CDRs from an LCVR set forth as SEQ ID NO: 12. In certain exemplary embodiments, an anti-B7-H4 antibody or antigen-binding fragment thereof comprises an HCVR set forth as SEQ ID NO: 11 and/or an LCVR set forth as SEQ ID NO: 12. In other embodiments, an anti-B7-H4 antibody or antigen-binding fragment thereof comprises an HCVR/LCVR pair SEQ ID NO: 11/SEQ ID NO: 12. In other embodiments, an anti-B7-H4 antibody or antigen-binding fragment thereof comprises an HCVR that has at least about 80% homology or identity (e.g., 80%, 85%, 90%, 91%, 92%, 93%, 94%, 95%, 96%, 97%, 98% or 99%) to SEQ ID NO: 11 and/or comprises an LCVR that has at least about 80% homology or identity (e.g., 80%, 85%, 90%, 91%, 92%, 93%, 94%, 95%, 96%, 97%, 98% or 99%) to SEQ ID NO: 12.

Antibodies and antigen-binding fragments thereof and antibody-drug conjugates described herein (e.g., anti-B7-H4 antibodies or B7-H4-ADCs) can be expressed in a modified form. For instance, a region of additional amino acids, particularly charged amino acids, can be added to the N-terminus of an antibody or an antigen-binding fragment thereof or antibody-drug conjugates (e.g., a B7-H4-ADC) to improve stability and persistence in the host cell, during purification, or during subsequent handling and storage. Also, peptide moieties can be added to an antibody or an antigen-binding fragment thereof or antibody-drug conjugates (e.g., a B7-H4-ADC) of the present invention to facilitate purification. Such regions can be removed prior to final preparation of an antibody molecule or at least one fragment thereof. Such methods are described in many standard laboratory manuals, such as Sambrook, supra; Ausubel, et al., ed., Current Protocols In Molecular Biology, John Wiley & Sons, Inc., NY, N.Y. (1987-2001).

The antibodies or antigen-binding fragments thereof or antibody-drug conjugates (e.g., anti-B7-H4 antibodies or B7-H4-ADCs) described herein typically bind the target antigen (e.g., B7-H4) with an equilibrium binding constant of about ≤1 μM, e.g., about ≤100 nM, about ≤10 nM, or about ≤1 nM, as measured using standard binding assays, for example, a Biacore-based binding assay.

In some embodiments, the antibody-drug conjugate (such as B7-H4-ADC) increases T cell proliferation. In some embodiments, the antibody-drug conjugate (such as B7-H4-ADC) increases IFNy production. In some embodiments, the antibody-drug conjugate (such as B7-H4-ADC) mediates ADCC activity against B7-H4-expressing cells. In some embodiments, the antibody-drug conjugate (such as B7-H4-ADC) mediates ADCC activity against B7-H4-expressing cells. In some embodiments, the antibody-drug conjugate (such as B7-H4-ADC) thereof does not mediate CDC activity against B7-H4-expressing cells

In some embodiments, the increase in T cell proliferation induced by the antibody-drug conjugate comprising an anti-B7-H4 antibody differ from that induced by the antibody B7-H4 antibody by no more than 1%, 5%, 10%, 15%, 20%, 25%, or 30%. In some embodiments, the increase in IFNy production induced by the antibody-drug conjugate comprising an anti-B7-H4 antibody differ from that induced by the antibody B7-H4 antibody by no more than any one of 1%, 5%, 10%, 15%, 20%, 25%, 30%, or 50%. In some embodiments, the increase in ADCC activity mediated by the antibody-drug conjugate comprising an anti-B7-H4 antibody differ from that mediated by the antibody B7-H4 antibody by no more than any one of 1%, 5%, 10%, 15%, 20%, 25%, 30%, or 50%. In some embodiments, the increase in ADCP activity mediated by the antibody-drug conjugate comprising an anti-B7-H4 antibody differ from that mediated by the antibody B7-H4 antibody by no more than any one of 1%, 5%, 10%, 15%, 20%, 25%, 30%, or 50%.

### III. THERAPEUTIC APPLICATIONS

The invention provides methods of treating disorders associated with cells that express B7-H4, e.g., cancers. In one aspect, the invention provides the use of human anti-B7-H4 antibodies and antigen-binding fragments or antibody-drug conjugates thereof (e.g., B7-H4-antibody-drug conjugates (B7-H4-ADCs)) for the treatment of cancers, such as breast cancer, ovarian cancer, lung cancer, cholangiocarcinoma and endometrial cancer. In one aspect, the invention provides the use of human anti-B7-H4 antibodies and antigen-binding fragments or conjugates thereof (e.g., B7-H4-antibody-drug conjugates (B7-H4-ADCs)) for the treatment of cancers, such as breast cancer. In one aspect, the invention provides the use of human anti-B7-H4 antibodies and antigen-binding fragments or antibody-drug conjugates thereof (e.g., B7-H4-ADCs) for the treatment of cancers, such as peritoneal cancer, fallopian tube cancer, or gallbladder cancer. In some embodiments, the cancer is an adenoid cystic carcinoma. In some embodiments, the adenoid cystic carcinoma is an adenoid cystic carcinoma of the head and neck. In some embodiments, the adenoid cystic carcinoma of the head and neck is an adenoid cystic carcinoma of the salivary glands. In some embodiments, the adenoid cystic carcinoma is an adenoid cystic carcinoma of the ovary. In some embodiments, the adenoid cystic carcinoma is an adenoid cystic carcinoma of the prostate. In some embodiments, the adenoid cystic carcinoma is an adenoid cystic carcinoma of the breast. In some embodiments, the adenoid cystic carcinoma is an adenoid cystic carcinoma of the skin. In some embodiments, the adenoid cystic carcinoma is an adenoid cystic carcinoma of the cervix. In one preferred embodiment, the invention provides the use of human anti-B7-H4 antibodies and antigen-binding fragments or antibody-drug conjugates thereof (e.g., B7-H4-ADCs) for the treatment of cancers, such as ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma or gallbladder carcinoma. In some embodiments, provided is a composition comprising any one of the human anti-B7-H4 antibodies, or antigen-binding fragments or antibody-drug conjugates thereof (e.g., B7-H4-ADCs) described herein. In some embodiments, provided is a composition comprising any one of the human anti-B7-H4 antibodies, or antigen-binding fragments or antibody-drug conjugates thereof (e.g., B7-H4-ADCs) described herein for use in treatment of a cancer. In some embodiments, provided is a composition comprising any one of the human anti-B7-H4 antibodies, or antigen-binding fragments or antibody-drug conjugates thereof (e.g., B7-H4-ADCs) described herein for use in treatment of a cancer. In some embodiments, provided are uses of a composition comprising any one of the human anti-B7-H4 antibodies, or antigen-binding fragments or antibody-drug conjugates thereof (e.g., B7-H4-ADCs) described herein in the manufacture of a medicament for treatment of a cancer.

In one aspect, the invention provides the use of human anti-B7-H4 antibodies or antigen-binding fragments or antibody-drug conjugates thereof (e.g., B7-H4-ADCs) in combination with an immune checkpoint inhibitor for the treatment of cancers, such as breast cancer, ovarian cancer, lung cancer, cholangiocarcinoma and endometrial cancer. In some embodiments, the cancer is an adenoid cystic carcinoma. In some embodiments, the adenoid cystic carcinoma is an adenoid cystic carcinoma of the head and neck. In some embodiments, the adenoid cystic carcinoma of the head and neck is an adenoid cystic carcinoma of the salivary glands. In some embodiments, the adenoid cystic carcinoma is an adenoid cystic carcinoma of the ovary. In some embodiments, the adenoid cystic carcinoma is an adenoid cystic carcinoma of the prostate. In some embodiments, the adenoid cystic carcinoma is an adenoid cystic carcinoma of the breast. In some embodiments, the adenoid cystic carcinoma is an adenoid cystic carcinoma of the skin. In some embodiments, the adenoid cystic carcinoma is an adenoid cystic carcinoma of the cervix. In some embodiments, provided is a composition comprising any one of the human anti-B7-H4 antibodies, or antigen-binding fragments or antibody-drug conjugates thereof (e.g., B7-H4-ADCs) described herein, and an immune checkpoint inhibitor. In some embodiments, provided is a composition comprising any one of the human anti-B7-H4 antibodies, or antigen-binding fragments or antibody-drug conjugates thereof (e.g., B7-H4-ADCs) described herein for use in treatment of a cancer, wherein the B7-H4 antibody, antigen-binding fragment or antibody-drug conjugate thereof is for use in combination with an immune checkpoint inhibitor. In some embodiments, provided are uses of a composition comprising any one of the human anti-B7-H4 antibodies, or antigen-binding fragments or antibody-drug conjugates thereof (e.g., B7-H4-ADCs) described herein in the manufacture of a medicament for treatment of a cancer, wherein the medicament is for use in combination with an immune checkpoint inhibitor. In some embodiments, provided are uses of a composition comprising any one of the human anti-B7-H4 antibodies, or antigen-binding fragments or antibody-drug conjugates thereof (e.g., B7-H4-ADCs) described herein and an immune checkpoint inhibitor in the manufacture of a medicament for treatment of a cancer.

Exemplary immune checkpoint inhibitor is targeted to, without limitation, PD-1, PD-L1, CTLA-4, LAG3, TIM-3, TIGIT, VISTA, TIM1, or BTLA. In some embodiments, the immune checkpoint inhibitor is targeted to one or more of PD-1, PD-L1, CTLA-4, LAG3, TIM-3, TIGIT, VISTA, TIM1, or BTLA. In some embodiments, the immune checkpoint inhibitor is one or more of: an antibody that binds to PD-1, an antibody that binds PD-L1, an antibody that binds CTLA-4, an antibody that binds LAG3, an antibody that binds TIM-3, an antibody that binds TIGIT, an antibody that binds VISTA, an antibody that binds TIM-1, or an antibody that binds BTLA. In some embodiments, the immune checkpoint inhibitor is targeted to one or more of PD-1, PD-L1, CTLA-4, or TIGIT. In some embodiments, the immune checkpoint inhibitor is target to PD-1. In some embodiments, the immune checkpoint inhibitor is one or more of: an antibody that binds to PD-1, an antibody that binds PD-L1, an antibody that binds CTLA-4, or an antibody that binds TIGIT. In some embodiments, the immune checkpoint inhibitor an antibody that binds to PD-1. In some embodiments, the immune checkpoint inhibitor is an anti-PD-1 antibody, such as one or more of: Nivolumab, Pembrolizumab, Cemiplimab, Dostarlimab, and Retifanlimab.

In one aspect, the invention provides the use of human anti-B7-H4 antibodies or antigen-binding fragments or antibody-drug conjugates thereof (e.g., B7-H4-ADCs) in combination with a PD-1 inhibitor (e.g. an anti-PD-1 antibody) for the treatment of cancers, such as breast cancer, ovarian cancer, lung cancer, cholangiocarcinoma and endometrial cancer. In some embodiments, the cancer is an adenoid cystic carcinoma. In some embodiments, the adenoid cystic carcinoma is an adenoid cystic carcinoma of the head and neck. In some embodiments, the adenoid cystic carcinoma of the head and neck is an adenoid cystic carcinoma of the salivary glands. In some embodiments, the adenoid cystic carcinoma is an adenoid cystic carcinoma of the ovary. In some embodiments, the adenoid cystic carcinoma is an adenoid cystic carcinoma of the prostate. In some embodiments, the adenoid cystic carcinoma is an adenoid cystic carcinoma of the breast. In some embodiments, the adenoid cystic carcinoma is an adenoid cystic carcinoma of the skin. In some embodiments, the adenoid cystic carcinoma is an adenoid cystic carcinoma of the cervix. In one aspect, the invention provides the use of human anti-B7-H4 antibodies or antigen-binding fragments or conjugates thereof (e.g., B7-H4-antibody-drug conjugates (B7-H4-ADCs)) in combination with a PD-1 inhibitor (e.g. anti-PD-1 antibody) for the treatment of cancers, such as breast cancer. In one aspect, the invention provides the use of human anti-B7-H4 antibodies or antigen-binding fragments or antibody-drug conjugates thereof (e.g., B7-H4-ADCs) in combination with a PD-1 inhibitor (e.g. anti-PD-1 antibody) for the treatment of cancers, such as peritoneal cancer, fallopian tube cancer, or gallbladder cancer. In one embodiment, the invention provides the use of human anti-B7-H4 antibodies or antigen-binding fragments or antibody-drug conjugates thereof (e.g., B7-H4-ADCs) in combination with a PD-1 inhibitor (e.g. anti-PD-1 antibody) for the treatment of cancers, such as ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma or gallbladder carcinoma.

In certain exemplary embodiments, the present invention provides a method for treating cancer in a cell, tissue, organ, animal or patient. In certain exemplary embodiments, the present invention provides a method for treating solid tumors, such as, e.g., breast cancer, ovarian cancer, lung cancer, cholangiocarcinoma and endometrial cancer in a human. In a particular exemplary embodiment, the breast cancer, ovarian cancer, lung cancer, cholangiocarcinoma and endometrial cancer is locally advance or metastatic. In certain exemplary embodiments, the present invention provides a method for treating solid tumors, such as, e.g., peritoneal cancer, fallopian tube cancer, or gallbladder cancer. In some embodiments, the tumor is an adenoid cystic carcinoma. In some embodiments, the adenoid cystic carcinoma is an adenoid cystic carcinoma of the head and neck. In some embodiments, the adenoid cystic carcinoma of the head and neck is an adenoid cystic carcinoma of the salivary glands. In some embodiments, the adenoid cystic carcinoma is an adenoid cystic carcinoma of the ovary. In some embodiments, the adenoid cystic carcinoma is an adenoid cystic carcinoma of the prostate. In some embodiments, the adenoid cystic carcinoma is an adenoid cystic carcinoma of the breast. In some embodiments, the adenoid cystic carcinoma is an adenoid cystic carcinoma of the skin. In some embodiments, the adenoid cystic carcinoma is an adenoid cystic carcinoma of the cervix. In certain exemplary embodiments, the present invention provides a method for treating solid tumors, such as, e.g., ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma or gallbladder carcinoma.

In some embodiments, the subject has been previously treated for breast cancer, or ovarian cancer. In some embodiments, the subject did not respond to the treatment (e.g., the subject experienced disease progression during treatment). In some embodiments, the subject relapsed after the treatment. In some embodiments, the subject experienced disease progression after the treatment. In some embodiments, the treatment previously administered to the subject was not an anti-B7-H4 antibody or antigen-binding fragment thereof as described herein.

Certain breast cancer, ovarian cancer, lung cancer, cholangiocarcinoma and endometrial cancer show detectable levels of B7-H4 measured at either the protein (e.g., by immunoassay using one of the exemplified antibodies) or the mRNA level. In certain embodiments, a breast cancer, ovarian cancer, lung cancer, cholangiocarcinoma or endometrial cancer shows elevated levels of B7-H4 relative to non-cancerous tissue or cells of the same type, e.g., breast, ovarian, lung, bile duct and endometrium cells from the same patient. In other embodiments, a breast cancer, ovarian cancer, lung cancer, cholangiocarcinoma or endometrial cancer shows similar levels of B7-H4 relative to non-cancerous breast, ovarian, lung, bile duct and endometrium cells of the same type, e.g., from the same patient.

Certain peritoneal cancer, fallopian tube cancer, and gallbladder cancer show detectable levels of B7-H4 measured at either the protein (e.g., by immunoassay using one of the exemplified antibodies) or the mRNA level. In certain embodiments, a peritoneal cancer, fallopian tube cancer, or gallbladder cancer shows elevated levels of B7-H4 relative to non-cancerous tissue or cells of the same type, e.g., peritoneum, fallopian tube, or gall bladder cells, respectively, from the same patient. In other embodiments, a peritoneal cancer, fallopian tube cancer, or gallbladder cancer shows similar levels of B7-H4 relative to e.g., non-cancerous peritoneum, fallopian tube, or gall bladder cells of the same type, from the same patient.

In some embodiments, B7-H4 protein is highly expressed on breast cancer, ovarian cancer, lung cancer, cholangiocarcinoma and endometrial cancer that are amenable to treatment, although cancers associated with higher or lower levels of B7-H4 expression can also be treated. Optionally, B7-H4 levels (e.g., B7-H4 protein levels) in a breast cancer, ovarian cancer, lung cancer, cholangiocarcinoma and endometrial cancer from a subject are measured before performing treatment. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express B7-H4. In some embodiments, expression of B7-H4 is low or absent on myeloid immune cell subsets, including monocytes, macrophages, and dendritic cells. In some embodiments, expression of B7-H4 is low or absent in CD163+ macrophages.

In some embodiments, B7-H4 protein is highly expressed on adenoid cystic carcinoma. (Panaccione et al. Clinical Breast Cancer 2017). In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express B7-H4.

In some embodiments, the cancer cell expresses B7-H4. In some embodiments, the cancer cell does not express B7-H4. In some embodiments, the cancer cell expresses a higher level of B7-H4 than a non-diseased cell of the same cell type. In some embodiments, the cancer cell expresses a comparable or lower level of B7-H4 than a non-diseased cell of the same cell type.

A. Lung Cancer

Lung cancer remains the leading cause of death from cancer in the United States, with over 155,000 deaths estimated in 2017. Treatments with curative intent for patients with early stage disease include surgery, chemotherapy, radiation therapy, or a combined modality approach. However, a majority of patients are diagnosed with advanced stage disease, which is usually incurable. Non-small cell lung cancer (NSCLC) represents up to 80% of all lung cancers. Within the subtypes of NSCLC, squamous cell carcinoma (SCC/NSCLC) represents approximately 30% of NSCLC. Systemic therapies used in the metastatic setting for SCC/NSCLC have shown limited benefit and are primarily aimed at prolonging survival and maintaining the quality of life for as long as possible, while minimizing side effects due to treatment. First line treatment for patients with SCC/NSCLC whose tumors do not express high levels of PD-L1 include a platinum-based chemotherapy doublet that does not contain pemetrexed, anti-VEGF antibody, or an anti-EGFR antibody necitumumab in combination with gemcitabine and cisplatin. Patients with at least 50% tumor cell staining for PD-L1 are offered first-line treatment with the anti-PD-1 inhibitor pembrolizumab. Patients who progress on an initial combination chemotherapy regimen may receive an anti-PD-1 or PD-L1 antibody, and combination chemotherapy is considered for patients whose disease has progressed after receiving PD-1/L1 inhibitors. New classes of therapy are urgently needed that can provide meaningful benefit to SCC/NSCLC patients.

The invention provides method for treating lung cancer with an anti-B7-H4 antibody or antigen-binding fragment thereof or antibody-drug conjugate (e.g., a B7-H4-ADC) described herein. In one aspect, the anti-B7-H4 antibody or antigen-binding fragment thereof or antibody-drug conjugate (e.g., a B7-H4-ADC) described herein are for use in a method of treating lung cancer in a subject. The invention also provides methods for treating lung cancer with an anti-B7-H4 antibody or antigen-binding fragment thereof or antibody-drug conjugate (e.g., a B7-H4-ADC) described herein in combination with an immune checkpoint inhibitor. In one aspect, the anti-B7-H4 antibody or antigen-binding fragment thereof or antibody-drug conjugate (e.g., a B7-H4-ADC) described herein is for use in combination with an immune checkpoint inhibitor in a method of treating lung cancer in a subject. In some embodiments, provided are methods for treating lung cancer with an anti-B7-H4 antibody or antigen-binding fragment thereof or antibody-drug conjugate (e.g., a B7-H4-ADC) described herein in combination with a PD-1 inhibitor (e.g. an anti-PD-1 antibody). In one aspect, the anti-B7-H4 antibody or antigen-binding fragment thereof or antibody-drug conjugate (e.g., a B7-H4-ADC) described herein is for use in combination with a PD-1 inhibitor (e.g. an anti-PD-1 antibody) in a method of treating lung cancer in a subject. In some embodiments, the lung cancer is small cell lung cancer. In some of the embodiments herein, the lung cancer is non-squamous cell carcinoma. In some of the embodiments herein, the lung cancer is squamous cell carcinoma. In some of the embodiments herein, the lung cancer is lung adenocarcinoma. In some embodiments, the lung cancer cell expresses B7-H4. In some embodiments, the lung cancer cell does not express B7-H4. In some embodiments, the lung cancer cell expresses a higher level of B7-H4 than a non-diseased cell of the same cell type. In some embodiments, the lung cancer cell expresses a comparable or lower level of B7-H4 than a non-diseased cell of the same cell type.

In some embodiments, the subject has received prior systemic therapy for the small cell lung cancer. In some embodiments, the subject experienced disease progression on or after the prior systemic therapy for the small cell lung cancer. In some embodiments, the subject received prior therapy with a cytotoxic chemotherapy. In some embodiments, the subject received prior therapy with an inhibitor of PD-1 or PD-L1. In some embodiments, the subject received prior therapy comprising an inhibitor of PD-1 and/or an inhibitor of PD-L1. In some embodiments, the subject received 1 line of systemic therapy for the small cell lung cancer. In some embodiments, the lung cancer is non-small cell lung cancer. In some embodiments, the non-small cell lung cancer is squamous cell carcinoma. In some embodiments, the non-small cell lung cancer is an adenocarcinoma. In some embodiments, the non-small cell lung cancer has predominant squamous histology. In some embodiments, greater than 85% of the non-small cell lung cancer cells have squamous histology. In some embodiments, the non-small cell lung cancer is non-squamous cell carcinoma. In some embodiments, the subject received prior systemic therapy for the non-small cell lung cancer. In some embodiments, the subject experienced disease progression on or after the prior systemic therapy for the non-small cell lung cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject received prior therapy with an inhibitor of PD-1 or PD-L1. In some embodiments, the subject received prior therapy comprising an inhibitor of PD-1 and/or an inhibitor of PD-L1. In some embodiments, the inhibitor of PD-1 is selected from the group consisting of nivolumab (OPDIVO®, BMS-936558, MDX-1106), pembrolizumab (KEYTRUDA®, MK-3475), pidilizumab (CT-011) and cemiplimab (REGN2810). In some embodiments, the inhibitor of PD-L1 is selected from the group consisting of atezolizumab (TECENTRIQ®, MPDL3280A), avelumab (BAVENCIO®), durvalumab and BMS-936559. In some embodiments, the subject received 1 line of prior systemic therapy for the non-small cell lung cancer. In some embodiments, the lung cancer is an advanced stage cancer. In some embodiments, the advanced stage cancer is a stage 3 or 4 cancer. In some embodiments, the lung cancer is a recurrent cancer. In some embodiments, the subject received prior treatment with standard of care therapy for the cancer and failed the prior treatment. In a particular embodiment, the subject is a human.

B. Breast Cancer

Breast cancers are classified on the basis of three protein expression markers: estrogen receptor (ER), progesterone receptor (PgR), and the overexpression of the growth factor receptor HER2/neu. Hormonal therapies, including tamoxifen and aromatase inhibitors, can be effective in treating tumors that express the hormone receptors ER and PgR. HER2-directed therapies are useful for tumors that express HER2/neu; these tumors are the only class of breast cancer that is currently eligible for immunotherapy. For these patients, unconjugated antibodies, such as Herceptin or Perjeta, are generally used in combination with chemotherapy.

The invention provides methods of treating cancers, such as breast cancer, with antibodies and antigen-binding fragments thereof or antibody-drug conjugates. In some embodiments, the invention provides methods of treating cancers, such as breast cancer, with antibody-drug conjugates. In some embodiments, the antibody-drug conjugate comprises an antibody conjugated to an auristatin. In some embodiments, the auristatin is a monomethyl auristatin. In some embodiments, the monomethyl auristatin is monomethyl auristatin E. In one aspect, the invention provides methods of treating disorders associated with cells that express B7-H4, e.g., cancers (e.g., breast cancers such as locally advanced breast cancer or metastatic breast cancer). As a result, the invention provides a method of treating a subject, for example, a subject with breast cancer, using the anti-B7-H4 antibodies and antigen-binding fragments thereof and antibody-drug conjugates described herein. The method comprises administering an effective amount of an anti-B7-H4 antibody or a composition comprising an anti-B7-H4 antibody or an antigen-binding fragment thereof or an antibody-drug conjugate (e.g., a B7-H4-ADC) to a subject in need thereof. In some embodiments, the cancer is an advanced stage cancer. In some embodiments, the advanced stage cancer is metastatic cancer. In some embodiments, the cancer is unresectable. In some embodiments, the cancer is locally advanced. In some embodiments, the cancer is recurrent cancer. In some embodiments, the subject received prior treatment with standard of care therapy for the cancer and failed the prior treatment. In some embodiments, the subject has been previously treated with one or more therapeutic agents and did not respond to the treatment, wherein the one or more therapeutic agents is not an antibody-drug conjugate (e.g., B7-H4-ADC). In some embodiments, the subject has been previously treated with one or more therapeutic agents and relapsed after the treatment, wherein the one or more therapeutic agents is not an antibody-drug conjugate (e.g., B7-H4-ADC). In some embodiments, the subject has been previously treated with one or more therapeutic agents and has experienced disease progression during treatment, wherein the one or more therapeutic agents is an antibody-drug conjugate (e.g., B7-H4-ADC). In some embodiments, the subject is a human.

The invention provides methods for treating breast cancer with an anti-B7-H4 antibody or antigen-binding fragment thereof or antibody-drug conjugate (e.g., a B7-H4-ADC) described herein. In one aspect, the anti-B7-H4 antibody or antigen-binding fragment thereof or antibody-drug conjugate (e.g., a B7-H4-ADC) described herein is for use in a method of treating breast cancer in a subject. The invention also provides methods for treating breast cancer with an anti-B7-H4 antibody or antigen-binding fragment thereof or antibody-drug conjugate (e.g., a B7-H4-ADC) described herein in combination with an immune checkpoint inhibitor. In one aspect, the anti-B7-H4 antibody or antigen-binding fragment thereof or antibody-drug conjugate (e.g., a B7-H4-ADC) described herein is for use in combination with an immune checkpoint inhibitor in a method of treating breast cancer in a subject. In some embodiments, provided are methods for treating breast cancer with an anti-B7-H4 antibody or antigen-binding fragment thereof or antibody-drug conjugate (e.g., a B7-H4-ADC) described herein in combination with a PD-1 inhibitor (e.g. an anti-PD-1 antibody). In one aspect, the anti-B7-H4 antibody or antigen-binding fragment thereof or antibody-drug conjugate (e.g., a B7-H4-ADC) described herein is for use in combination with a PD-1 inhibitor (e.g. an anti-PD-1 antibody) in a method of treating breast cancer in a subject.

Exemplary breast cancers are those that express B7-H4 in a cell expressing the cancer (i.e., B7-H4-expressing cancers). In certain exemplary embodiments, a breast cancer is selected from the group consisting of carcinomas, sarcomas, phyllodes, Paget disease, and angiosarcomas. The breast cancer may be in situ (e.g., ductal carcinoma in situ (DCIS), lobular carcinoma in situ (LCIS) and the like) or invasive/infiltrating (e.g., invasive ductal carcinoma (IDC), invasive lobular carcinoma (ILC), inflammatory breast cancer (IBC) and the like).

Breast cancer may have the following characteristics: estrogen receptor positive (ER+); estrogen receptor positive (ER−); progesterone receptor positive (PR+); progesterone receptor negative (PR−); hormone receptor positive (HR+); hormone receptor negative (HR−); HER2 gene overexpressing (HER2+); HER2 gene wild-type or under-expressing (HER2−); group 1 (luminal A), i.e., ER+/PR+/HER2−; group 2 (luminal B), i.e., ER+/PR−/HER2+; group 3 (HER2+), i.e., ER−/PR−/HER2+; and group 4 (basal-like or triple negative (TN)), i.e., ER−/PR−/HER2−.

A breast cancer can further be categorized as grade 1, 2 or 3. Grade 1 or well-differentiated (score 3, 4, or 5) breast cancer comprises cells that are slower-growing, and look more like normal breast tissue than the higher grades of breast cancer. Grade 2 or moderately differentiated (score 6, 7) breast cancer has cells that grow at a speed of and look like cells somewhere between grades 1 and 3. Grade 3 or poorly differentiated (score 8, 9) breast cancer has cells that look very different from normal cells and typically grow and spread faster than grades 1 or 2.

In certain exemplary embodiments, a breast cancer is an incurable, unresectable, locally advanced or metastatic breast cancer (LA/MBC). In certain embodiments, a breast cancer is either a triple negative (TN) (ER−/PR−/HER2−) breast cancer, an ER- and/or PR+/HER2− breast cancer, and an LA/MBC breast cancer. In certain exemplary embodiments, the breast cancer is HER2+ and LA/MBC. In certain exemplary embodiments, a breast cancer is TN and LA/MBC. In certain exemplary embodiments, a breast cancer is selected from the group consisting of a TN breast cancer, a metastatic breast cancer, and a metastatic, TN breast cancer. In some embodiments, the breast cancer is a HER2 negative breast neoplasm. In some embodiments, the breast cancer is a HER2 positive breast neoplasm. In some embodiments, the breast cancer is a triple negative breast neoplasm.

In some embodiments, the breast cancer cell expresses B7-H4. In some embodiments, the breast cancer cell does not express B7-H4. In some embodiments, the breast cancer cell expresses a higher level of B7-H4 than a non-diseased cell of the same cell type. In some embodiments, the breast cancer cell expresses a comparable or lower level of B7-H4 than a non-diseased cell of the same cell type.

In certain exemplary embodiments, the present invention provides a method for treating breast cancer in a human. In some embodiments, the present invention provides a method for treating ER+ breast cancer in a subject. In some embodiments, the subject with ER+ breast cancer is not a candidate for hormonal therapy. In some embodiments, the subject with ER+ breast cancer received one prior cytotoxic regimen. In some embodiments, the subject with ER+ breast cancer received two or more prior cytotoxic regimens. In some embodiments, the present invention provides a method for treating ER+/HER2− breast cancer in a subject. In some embodiments, the subject with ER+/HER2− breast cancer is not a candidate for hormonal therapy. In some embodiments, the subject with ER+/HER2− breast cancer has not received a prior cytotoxic regimen. In some embodiments, the subject with ER+/HER2− breast cancer received one prior cytotoxic regimen. In some embodiments, the subject with ER+/HER2− breast cancer received two or more prior cytotoxic regimens. In some embodiments, the present invention provides a method for treating PR+/HER2− breast cancer in a subject. In some embodiments, the subject with PR+/HER2− breast cancer is not a candidate for hormonal therapy. In some embodiments, the subject with PR+/HER2− breast cancer received one prior cytotoxic regimen. In some embodiments, the subject with PR+/HER2− breast cancer received two or more prior cytotoxic regimens. In some embodiments, the present invention provides a method of treating ER+/PR+HER2− breast cancer in a subject. In some embodiments, the subject with ER+/PR+/HER2− breast cancer is not a candidate for hormonal therapy. In some embodiments, the subject with ER+/PR+HER2− breast cancer received one prior cytotoxic regimen. In some embodiments, the subject with ER+/PR+HER2− breast cancer received two or more prior cytotoxic regimens. In some embodiments, the present invention provides a method of treating triple negative breast cancer in a subject. In some embodiments, the subject with triple negative breast cancer received one non-hormonally directed prior therapy. In some embodiments, the subject with triple negative breast cancer received one prior cytotoxic regimen. In some embodiments, the subject with triple negative breast cancer received two or more prior cytotoxic regimens. In some embodiments, the present invention provides a method of treating HR+ breast cancer in a subject. In some embodiments, the subject with HR+ breast cancer received one prior cytotoxic regimen. In some embodiments, the subject with HR+ breast cancer received two or more prior cytotoxic regimens. In some embodiments, the present invention provides a method of treating HR+/ER+/HER2− breast cancer in a subject. In some embodiments, the subject with HR+/ER+/HER2− breast cancer is not a candidate for hormonal therapy. In some embodiments, the subject with HR+/ER+/HER2− breast cancer is eligible for chemotherapy. In some embodiments, the subject with HR+/ER+/HER2− breast cancer received one prior cytotoxic regimen. In some embodiments, the subject with HR+/ER+/HER2− breast cancer received one prior non-hormonally-directed therapy regimen. In some embodiments, the present invention provides a method of treating HR+/PR+/HER2− breast cancer in a subject. In some embodiments, the subject with HR+/PR+/HER2− breast cancer is not a candidate for hormonal therapy. In some embodiments, the subject with HR+/PR+/HER2− breast cancer is eligible for chemotherapy. In some embodiments, the subject with HR+/PR+/HER2− breast cancer received one prior cytotoxic regimen. In some embodiments, the subject with HR+/PR+/HER2− breast cancer received one prior non-hormonally-directed therapy regimen. In some embodiments, the present invention provides a method of treating HR+/ER+/PR+/HER2− breast cancer in a subject. In some embodiments, the subject with HR+/ER+/PR+/HER2− breast cancer is not a candidate for hormonal therapy. In some embodiments, the subject with HR+/ER+/PR+/HER2− breast cancer is eligible for chemotherapy. In some embodiments, the subject with HR+/ER+/PR+/HER2− breast cancer received one prior cytotoxic regimen. In some embodiments, the subject with HR+/ER+/PR+HER2− breast cancer received one prior non-hormonally-directed therapy regimen. In some embodiments, the present invention provides a method of treating HER2+ breast cancer in a subject. In some embodiments, the subject with HER2+ breast cancer received one prior cytotoxic regimen. In some embodiments, the subject with HER2+ breast cancer received two or more prior cytotoxic regimens. In some embodiments, the present invention provides a method of treating HR+/HER2+ breast cancer in a subject. In some embodiments, the subject with HR+/HER2+ breast cancer is eligible for chemotherapy. In some embodiments, the subject with HR+/HER2+ breast cancer is not eligible for chemotherapy. In some embodiments, the subject with HR+/HER2+ breast cancer is not a candidate for hormonal therapy. In some embodiments, the breast cancer is an advanced breast stage cancer. In some embodiments, the advanced stage breast cancer is metastatic breast cancer. In some embodiments, the breast cancer is unresectable. In some embodiments, the breast cancer is locally advanced. In some embodiments, the breast cancer is recurrent breast cancer. In some embodiments, the subject received prior treatment with standard of care therapy for the breast cancer and failed the prior treatment. In some embodiments, the subject has been previously treated with one or more therapeutic agents and did not respond to the treatment, wherein the one or more therapeutic agents is not an antibody-drug conjugate (e.g., B7-H4-ADC). In some embodiments, the subject has been previously treated with one or more therapeutic agents and relapsed after the treatment, wherein the one or more therapeutic agents is not an antibody-drug conjugate (e.g., B7-H4-ADC). In some embodiments, the subject has been previously treated with one or more therapeutic agents and has experienced disease progression during treatment, wherein the one or more therapeutic agents is an antibody-drug conjugate (e.g., B7-H4-ADC). In some embodiments, the subject is a human

In some embodiments, the subject has received prior systemic therapy for the breast cancer. In some embodiments, the subject experienced disease progression on or after the prior systemic therapy for the breast cancer. In some embodiments, the subject received prior therapy with a cytotoxic chemotherapy. In some embodiments, the subject received prior therapy with an inhibitor of PD-1 or PD-L1. In some embodiments, the subject received prior therapy comprising an inhibitor of PD-1 and/or an inhibitor of PD-L1. In some embodiments, the subject received 1 line of systemic therapy for the breast cancer. In some embodiments, the subject experienced disease progression on or after the prior systemic therapy for the breast cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject received prior therapy with an inhibitor of PD-1 or PD-L1. In some embodiments, the subject received prior therapy comprising an inhibitor of PD-1 and/or an inhibitor of PD-L1. In some embodiments, the inhibitor of PD-1 is selected from the group consisting of nivolumab (OPDIVO®, BMS-936558, MDX-1106), pembrolizumab (KEYTRUDA®, MK-3475), pidilizumab (CT-011) and cemiplimab (REGN2810). In some embodiments, the inhibitor of PD-L1 is selected from the group consisting of atezolizumab (TECENTRIQ®, MPDL3280A), avelumab (BAVENCIO®), durvalumab and BMS-936559. In some embodiments, the subject received 1 line of prior systemic therapy for the breast cancer. In some embodiments, the breast cancer is an advanced stage cancer. In some embodiments, the advanced stage cancer is a stage 3 or 4 cancer. In some embodiments, the breast cancer is a recurrent cancer. In some embodiments, the subject received prior treatment with standard of care therapy for the cancer and failed the prior treatment. In a particular embodiment, the subject is a human.

C. Ovarian Cancer

The invention provides methods of treating cancers, such as ovarian cancer, with antibodies and antigen-binding fragments thereof and antibody-drug conjugates. In some embodiments, the invention provides methods of treating cancers, such as ovarian cancer, with antibody-drug conjugates. In some embodiments, the antibody-drug conjugate comprises an antibody conjugated to an auristatin. In some embodiments, the auristatin is a monomethyl auristatin. In some embodiments, the monomethyl auristatin is monomethyl auristatin E. In one aspect, the invention provides methods of treating disorders associated with cells that express B7-H4, e.g., cancers (e.g., ovarian cancers such as locally advanced ovarian cancer or metastatic ovarian cancer). As a result, the invention provides a method of treating a subject, for example, a subject with ovarian cancer, using the anti-B7-H4 antibodies and antigen-binding fragments thereof and antibody-drug conjugates described herein. The method comprises administering an effective amount of an anti-B7-H4 antibody or a composition comprising an anti-B7-H4 antibody or an antigen-binding fragment thereof or an antibody-drug conjugate (e.g., a B7-H4-ADC) to a subject in need thereof. In some embodiments, the cancer is an advanced stage cancer. In some embodiments, the advanced stage cancer is metastatic cancer. In some embodiments, the cancer is unresectable. In some embodiments, the cancer is locally advanced. In some embodiments, the cancer is recurrent cancer. In some embodiments, the subject received prior treatment with standard of care therapy for the cancer and failed the prior treatment. In some embodiments, the subject has been previously treated with one or more therapeutic agents and did not respond to the treatment, wherein the one or more therapeutic agents is not an antibody-drug conjugate (e.g., B7-H4-ADC). In some embodiments, the subject has been previously treated with one or more therapeutic agents and relapsed after the treatment, wherein the one or more therapeutic agents is not an antibody-drug conjugate (e.g., B7-H4-ADC). In some embodiments, the subject has been previously treated with one or more therapeutic agents and has experienced disease progression during treatment, wherein the one or more therapeutic agents is an antibody-drug conjugate (e.g., B7-H4-ADC). In some embodiments, the subject is a human.

Exemplary ovarian cancers are those that express B7-H4 in a cell expressing the cancer (i.e., B7-H4-expressing cancers). In certain exemplary embodiments, an ovarian cancer is selected from the group consisting of carcinomas, sarcomas, phyllodes, Paget disease, and angiosarcomas. In some embodiments, the ovarian cancer is an ovarian neoplasm. The ovarian cancer may be in situ or invasive/infiltrating.

The invention provides methods for treating ovarian cancer with an anti-B7-H4 antibody or antigen-binding fragment thereof or antibody-drug conjugate (e.g., a B7-H4-ADC) described herein. In one aspect, the anti-B7-H4 antibody or antigen-binding fragment thereof or antibody-drug conjugate (e.g., a B7-H4-ADC) described herein is for use in a method of treating ovarian cancer in a subject. The invention also provides methods for treating ovarian cancer with an anti-B7-H4 antibody or antigen-binding fragment thereof or antibody-drug conjugate (e.g., a B7-H4-ADC) described herein in combination with an immune checkpoint inhibitor. In one aspect, the anti-B7-H4 antibody or antigen-binding fragment thereof or antibody-drug conjugate (e.g., a B7-H4-ADC) described herein is for use in combination with an immune checkpoint inhibitor in a method of treating ovarian cancer in a subject. The invention also provides methods for treating ovarian cancer with an anti-B7-H4 antibody or antigen-binding fragment thereof or antibody-drug conjugate (e.g., a B7-H4-ADC) described herein in combination with a PD-1 inhibitor (e.g. an anti-PD-1 antibody). In one aspect, the anti-B7-H4 antibody or antigen-binding fragment thereof or antibody-drug conjugate (e.g., a B7-H4-ADC) described herein is for use in combination with a PD-1 inhibitor (e.g. an anti-PD-1 antibody) in a method of treating ovarian cancer in a subject.

A ovarian cancer can further be categorized as grade 1, 2 or 3. Grade 1 or well-differentiated (score 3, 4, or 5) ovarian cancer comprises cells that are slower-growing, and look more like normal ovarian tissue than the higher grades of ovarian cancer. Grade 2 or moderately differentiated (score 6, 7) ovarian cancer has cells that grow at a speed of and look like cells somewhere between grades 1 and 3. Grade 3 or poorly differentiated (score 8, 9) ovarian cancer has cells that look very different from normal cells and typically grow and spread faster than grades 1 or 2.

In certain exemplary embodiments, a ovarian cancer is an incurable, unresectable, locally advanced or metastatic ovarian cancer. In some embodiments, the ovarian cancer is Ovarian Serous Cystadenocarcinoma (OV).

In some embodiments, the ovarian cell expresses B7-H4. In some embodiments, the ovarian cancer cell does not express B7-H4. In some embodiments, the ovarian cancer cell expresses a higher level of B7-H4 than a non-diseased cell of the same cell type. In some embodiments, the ovarian cancer cell expresses a comparable or lower level of B7-H4 than a non-diseased cell.

In certain exemplary embodiments, the present invention provides a method for treating ovarian cancer in a human. In some embodiments, the subject with ovarian cancer received one prior cytotoxic regimen. In some embodiments, the subject with ovarian cancer received two or more prior cytotoxic regimens. In some embodiments, the subject with ovarian cancer received two or more prior cytotoxic regimens. In some embodiments, the ovarian cancer is an advanced with stage cancer. In some embodiments, the advanced stage with cancer is metastatic ovarian cancer. In some embodiments, the ovarian cancer is unresectable. In some embodiments, the ovarian cancer is locally advanced. In some embodiments, the ovarian cancer is recurrent ovarian cancer. In some embodiments, the subject received prior treatment with standard of care therapy for the ovarian cancer and failed the prior treatment. In some embodiments, the subject has been previously treated with one or more therapeutic agents and did not respond to the treatment, wherein the one or more therapeutic agents is not an antibody-drug conjugate (e.g., B7-H4-ADC). In some embodiments, the subject has been previously treated with one or more therapeutic agents and relapsed after the treatment, wherein the one or more therapeutic agents is not an antibody-drug conjugate (e.g., B7-H4-ADC). In some embodiments, the subject has been previously treated with one or more therapeutic agents and has experienced disease progression during treatment, wherein the one or more therapeutic agents is an antibody-drug conjugate (e.g., B7-H4-ADC). In some embodiments, the subject is a human

In some embodiments, the subject has received prior systemic therapy for the ovarian cancer. In some embodiments, the subject experienced disease progression on or after the prior systemic therapy for the ovarian cancer. In some embodiments, the subject received prior therapy with a cytotoxic chemotherapy. In some embodiments, the subject received prior therapy with an inhibitor of PD-1 or PD-L1. In some embodiments, the subject received prior therapy comprising an inhibitor of PD-1 and/or an inhibitor of PD-L1. In some embodiments, the subject received 1 line of systemic therapy for the ovarian cancer. In some embodiments, the subject experienced disease progression on or after the prior systemic therapy for the ovarian cancer. In some embodiments, the subject received prior therapy with a cytotoxic chemotherapy. In some embodiments, the subject received prior therapy with a platinum-based therapy or platinum-based combination therapy. In some embodiments, the platinum-based therapy is selected from the group consisting of carboplatin, cisplatin, oxaliplatin, nedaplatin, triplatin tetranitrate, phenanthriplatin, picoplatin and satraplatin. In some embodiments, the platinum-based therapy is carboplatin. In some embodiments, the platinum-based therapy is cisplatin. In some embodiments, the platinum-based therapy is oxaliplatin. In some embodiments, the platinum-based therapy is nedaplatin. In some embodiments, the platinum-based therapy is triplatin tetranitrate. In some embodiments, the platinum-based therapy is phenanthriplatin. In some embodiments, the platinum-based therapy is picoplatin. In some embodiments, the platinum-based therapy is satraplatin. In some embodiments, the subject received prior therapy with an inhibitor of PD-1 or PD-L1. In some embodiments, the subject received prior therapy comprising an inhibitor of PD-1 and/or an inhibitor of PD-L1. In some embodiments, the inhibitor of PD-1 is selected from the group consisting of nivolumab (OPDIVO®, BMS-936558, MDX-1106), pembrolizumab (KEYTRUDA®, MK-3475), pidilizumab (CT-011) and cemiplimab (REGN2810). In some embodiments, the inhibitor of PD-L1 is selected from the group consisting of atezolizumab (TECENTRIQ®, MPDL3280A), avelumab (BAVENCIO®), durvalumab and BMS-936559. In some embodiments, the subject received 1 line of prior systemic therapy for the ovarian cancer. In some embodiments, the ovarian cancer is an advanced stage cancer. In some embodiments, the advanced stage cancer is a stage 3 or 4 cancer. In some embodiments, the lung cancer is a recurrent cancer. In some embodiments, the subject received prior treatment with standard of care therapy for the cancer and failed the prior treatment. In a particular embodiment, the subject is a human.

In some embodiments, provided is an anti-B7-H4 antibody or antigen-binding fragment thereof or antibody-drug conjugate described herein (e.g., a B7-H4-ADC) for use in the treatment of a cancer. In some embodiments, provided is an anti-B7-H4 antibody or antigen-binding fragment thereof or antibody-drug conjugate (e.g., a B7-H4-ADC) for use in the treatment of a cancer, wherein the anti-B7-H4 antibody or antigen-binding fragment thereof comprises an HCVR that has at least about 95% (such as 95%, 97%, 98%, 99%, or 100%) homology or identity to SEQ ID NO: 11 and/or comprises an LCVR that has at least about 95% (such as 95%, 97%, 98%, 99%, or 100%) homology or identity to SEQ ID NO: 12. In some embodiments, provided is B7-H4 antibody-drug conjugate (e.g., a B7-H4-ADC) for use in the treatment of a cancer, wherein the anti-B7-H4 antibody or antigen-binding fragment thereof comprises an HCVR that has at least about 95% (such as 95%, 97%, 98%, 99%, or 100%) homology or identity to SEQ ID NO: 11 and/or comprises an LCVR that has at least about 95% (such as 95%, 97%, 98%, 99%, or 100%) homology or identity to SEQ ID NO: 12, and wherein the antibody is conjugated to vcMMAE, wherein the vcMMAE has the structure:

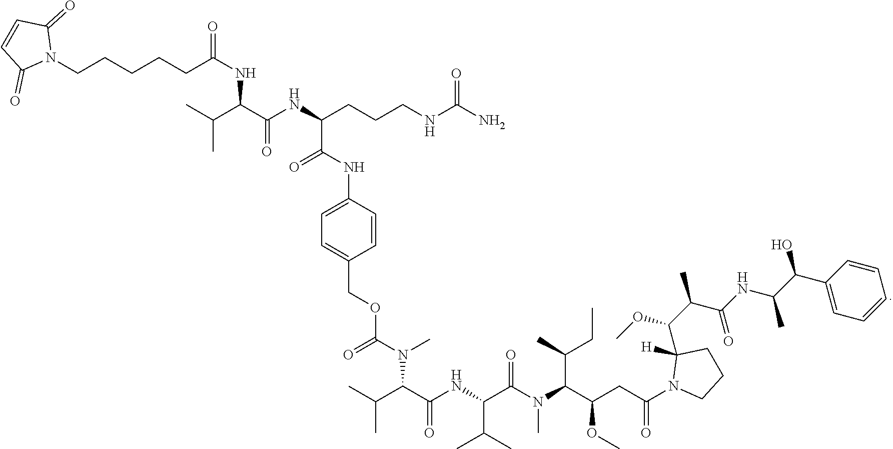 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, provided is the use of an anti-B7-H4 antibody or antigen-binding fragment thereof or antibody-drug conjugate described herein (e.g., a B7-H4-ADC) for the treatment of a cancer. In some embodiments, provided is the use of an anti-B7-H4 antibody or antigen-binding fragment thereof or antibody-drug conjugate (e.g., a B7-H4-ADC) for the treatment of a cancer, wherein the anti-B7-H4 antibody or antigen-binding fragment thereof comprises an HCVR that has at least about 95% (such as 95%, 97%, 98%, 99%, or 100%) homology or identity to SEQ ID NO: 11 and/or comprises an LCVR that has at least about 95% (such as 95%, 97%, 98%, 99%, or 100%) homology or identity to SEQ ID NO: 12. In some embodiments, provided is the use of a B7-H4 antibody-drug conjugate (e.g., a B7-H4-ADC) for the treatment of a cancer, wherein the anti-B7-H4 antibody or antigen-binding fragment thereof comprises an HCVR that has at least about 95% (such as 95%, 97%, 98%, 99%, or 100%) homology or identity to SEQ ID NO: 11 and/or comprises an LCVR that has at least about 95% (such as 95%, 97%, 98%, 99%, or 100%) homology or identity to SEQ ID NO: 12, and wherein the antibody is conjugated to vcMMAE, wherein the vcMMAE has the structure:

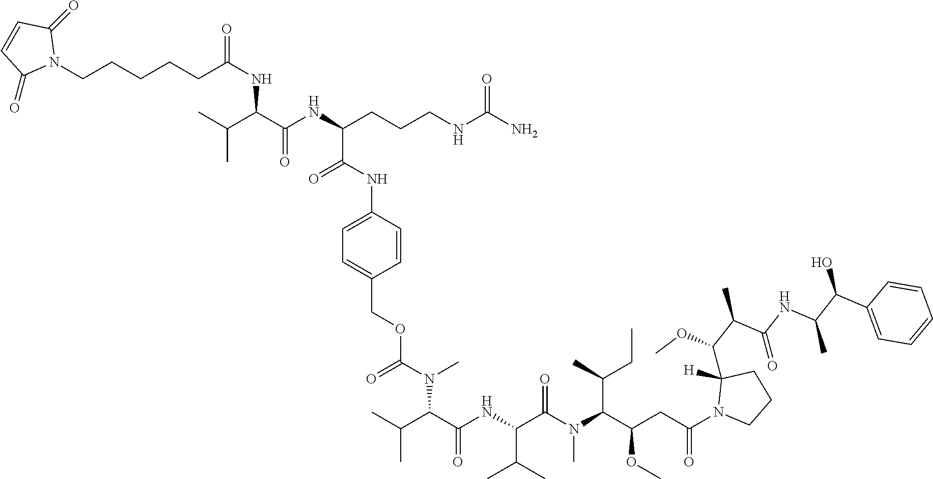 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, provided is the use of an anti-B7-H4 antibody or antigen-binding fragment thereof or antibody-drug conjugate described herein (e.g., a B7-H4-ADC) in the manufacture of a medicament for the treatment of a cancer. In some embodiments, provided is the use of an anti-B7-H4 antibody or antigen-binding fragment thereof or antibody-drug conjugate (e.g., a B7-H4-ADC) in the manufacture of a medicament for the treatment of a cancer, wherein the anti-B7-H4 antibody or antigen-binding fragment thereof comprises an HCVR that has at least about 95% (such as 95%, 97%, 98%, 99%, or 100%) homology or identity to SEQ ID NO: 11 and/or comprises an LCVR that has at least about 95% (such as 95%, 97%, 98%, 99%, or 100%) homology or identity to SEQ ID NO: 12. In some embodiments, provided is the use of a B7-H4 antibody-drug conjugate (e.g., a B7-H4-ADC) in the manufacture of a medicament the treatment of a cancer, wherein the anti-B7-H4 antibody or antigen-binding fragment thereof comprises an HCVR that has at least about 95% (such as 95%, 97%, 98%, 99%, or 100%) homology or identity to SEQ ID NO: 11 and/or comprises an LCVR that has at least about 95% (such as 95%, 97%, 98%, 99%, or 100%) homology or identity to SEQ ID NO: 12, and wherein the antibody is conjugated to vcMMAE, wherein the vcMMAE has the structure:

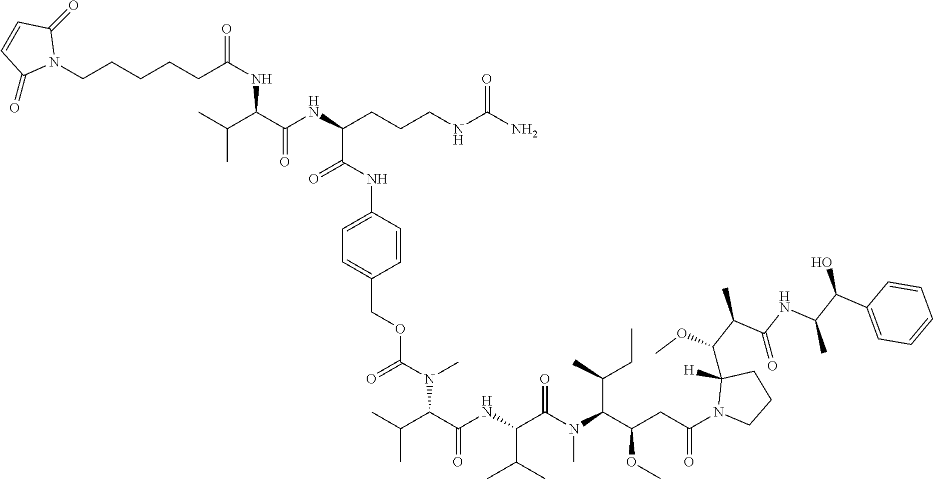 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

In some embodiments, the immune checkpoint inhibitor is targeted to PD-1 (i.e. a PD-1 inhibitor). In some embodiments, the PD-1 inhibitor is an anti-PD-1 antibody. In some embodiments, the anti-PD-1 antibody is an intact monoclonal antibody. In some embodiments, the immune checkpoint inhibitor is an anti-PD-1 antibody, such as one or more of: Nivolumab, Pembrolizumab, Cemiplimab, Dostarlimab, and Retifanlimab.

In some embodiments, the cancer is breast cancer, ovarian cancer, lung cancer, cholangiocarcinoma or endometrial cancer. In some embodiments, the cancer is peritoneal cancer, fallopian tube cancer, or gallbladder cancer. In one preferred embodiment, the cancer is selected from the group consisting of ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma and gallbladder carcinoma.

### Treatment Outcomes

In one embodiment of the methods or uses or product for uses provided herein, response to treatment with an antibody or antigen-binding fragment thereof or antibody-drug conjugate as described herein, such as e.g., a B7-H4-ADC, is assessed by measuring the size of a tumor derived from the cancer (e.g., breast cancer, ovarian cancer, lung cancer, cholangiocarcinoma or endometrial cancer). In one embodiment of the methods or uses or product for uses provided herein, response to treatment with B7-H4-ADC in combination with a PD-1 inhibitor (e.g. an anti-PD1 antibody) is assessed by measuring the size of a tumor derived from the cancer (e.g., breast cancer, ovarian cancer, lung cancer, cholangiocarcinoma or endometrial cancer).

In some embodiments, the cancer is selected from peritoneal cancer, fallopian tube cancer, and gallbladder cancer. In one preferred embodiment, the cancer is selected from the group consisting of ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma and gallbladder carcinoma. In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof or antibody-drug conjugate described herein (e.g., a B7-H4-ADC). In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antigen-binding fragment thereof or antibody-drug conjugate described herein (e.g., a B7-H4-ADC). In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In one embodiment, the size of a tumor derived from the cancer is measured by positron emission tomography (PET). In one embodiment, the size of a tumor derived from the cancer is measured by ultrasound. In some embodiments, the tumor cell expresses B7-H4. In some embodiments, the tumor cell does not express B7-H4. In some embodiments, the tumor cell expresses a higher level of B7-H4 than a non-diseased cell of the same cell type. In some embodiments, the tumor cell expresses a comparable or lower level of B7-H4 than a non-diseased cell of the same cell type.

In some embodiments, the reduction of tumor size induced by administration of a B7-H4-ADC comprising an anti-B7-H4 antibody of antigen-binding fragment thereof is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 50-fold, 100-fold, 200-fold, 500-fold, 1000-fold greater than that induced by the anti-B7-H4 antibody of antigen-binding fragment thereof. In some embodiments, the reduction of tumor size induced by administration of a B7-H4-ADC comprising an anti-B7-H4 antibody of antigen-binding fragment thereof is at least about 2-fold, 5-fold, 10-fold, or 50-fold greater than that induced by the anti-B7-H4 antibody of antigen-binding fragment thereof.

In some embodiments, the reduction of tumor size induced by administration of a B7-H4-ADC in combination with a PD-1 inhibitor (e.g. an anti-PD1 antibody) is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 50-fold, 100-fold, 200-fold, 500-fold, 1000-fold greater than that induced by administration of the B7-H4-ADC or administration of the PD-1 inhibitor. In some embodiments, the reduction of tumor size induced by administration of a B7-H4-ADC in combination with a PD-1 inhibitor (e.g. an anti-PD1 antibody) is at least about 2-fold, 5-fold, 10-fold, or 50-fold greater than that induced by administration of the B7-H4-ADC or administration of the PD-1 inhibitor.

In some embodiments, a similar reduction of tumor size can be induced by administration of a B7-H4-ADC comprising an anti-B7-H4 antibody of antigen-binding fragment thereof at a concentration that is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 100-fold, 200-fold, 500-fold, 1000-fold lower than concentration of administration of the anti-B7-H4 antibody of antigen-binding fragment thereof. In some embodiments, a similar reduction of tumor size can be induced by administration of a B7-H4-ADC comprising an anti-B7-H4 antibody of antigen-binding fragment thereof at a concentration that is at least about any one 10-fold lower than the concentration of the anti-B7-H4 antibody of antigen-binding fragment.

In some embodiments, a similar reduction of tumor size can be induced by administration of a B7-H4-ADC in combination with a PD-1 inhibitor (e.g. an anti-PD1 antibody) at a concentration of B7-H4-ADC that is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 100-fold, 200-fold, 500-fold, 1000-fold lower than the concentration of B7-H4-ADC when administered as monotherapy. In some embodiments, a similar reduction of tumor size can be induced by administration of a B7-H4-ADC in combination with a PD-1 inhibitor (e.g. an anti-PD1 antibody) at a concentration of B7-H4-ADC that is at least about any one 10-fold lower than the concentration of B7-H4-ADC when administered as monotherapy.

In some embodiments, the regression of tumor induced by administration of a B7-H4-ADC comprising an anti-B7-H4 antibody of antigen-binding fragment thereof is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 50-fold, 100-fold, 200-fold, 500-fold, 1000-fold greater than that induced by the anti-B7-H4 antibody of antigen-binding fragment thereof. In some embodiments, the regression of tumor induced by administration of a B7-H4-ADC comprising an anti-B7-H4 antibody of antigen-binding fragment thereof is at least about 50-fold or about 100-fold greater than that induced by the anti-B7-H4 antibody of antigen-binding fragment thereof.

In some embodiments, the regression of tumor induced by administration of a B7-H4-ADC in combination with a PD-1 inhibitor (e.g. an anti-PD1 antibody) is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 50-fold, 100-fold, 200-fold, 500-fold, 1000-fold greater than that induced by administration of the B7-H4-ADC or administration of the PD-1 inhibitor. In some embodiments, the regression of tumor induced by administration of a B7-H4-ADC in combination with a PD-1 inhibitor (e.g. an anti-PD1 antibody) is at least about 50-fold or about 100-fold greater than that induced by administration of the B7-H4-ADC or administration of the PD-1 inhibitor.

In some embodiments, a similar regression of tumor can be induced by administration of a B7-H4-ADC comprising an anti-B7-H4 antibody of antigen-binding fragment thereof at a concentration that is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 100-fold, 200-fold, 500-fold, 1000-fold lower than the concentration of the anti-B7-H4 antibody of antigen-binding fragment thereof. In some embodiments, a similar regression of tumor can be induced by administration of a B7-H4-ADC comprising an anti-B7-H4 antibody of antigen-binding fragment thereof at a concentration that is at least about any one 10-fold lower than the concentration of the anti-B7-H4 antibody of antigen-binding fragment thereof.

In some embodiments, a similar regression of tumor can be induced by administration of a B7-H4-ADC in combination with a PD-1 inhibitor (e.g. an anti-PD1 antibody) at a concentration of B7-H4-ADC that is at least about any one of: 10%, 20%, 30%, 40%, 50%, 60%, 70%, 80%, 90%, 100%, 2-fold, 3-fold, 5-fold, 10-fold, 15-fold, 20-fold, 30-fold, 100-fold, 200-fold, 500-fold, 1000-fold lower than the concentration of B7-H4-ADC when administered as monotherapy In some embodiments, a similar regression of tumor can be induced by administration of a B7-H4-ADC in combination with a PD-1 inhibitor (e.g. an anti-PD1 antibody) at a concentration of B7-H4-ADC that is at least about any one 10-fold lower than the concentration of B7-H4-ADC when administered as monotherapy.

In one embodiment of the methods or uses or product for uses provided described herein, response to treatment with an antibody or antigen-binding fragment thereof or antibody-drug conjugate described herein (e.g., a B7-H4-ADC), promotes regression of a tumor derived from the cancer (e.g., small cell lung cancer, non-small cell lung cancer, head and neck squamous cell carcinoma, esophageal squamous cell carcinoma, gastric and gastroesophageal junction adenocarcinoma, or breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the antibody or antigen-binding fragment thereof or antibody-drug conjugate described herein (e.g., a B7-H4-ADC). In one embodiment of the methods or uses or product for uses provided described herein, response to treatment with a B7-H4-ADC in combination with a PD-1 inhibitor (e.g. an anti-PD1 antibody) promotes regression of a tumor derived from the cancer (e.g., small cell lung cancer, non-small cell lung cancer, head and neck squamous cell carcinoma, esophageal squamous cell carcinoma, gastric and gastroesophageal junction adenocarcinoma, or breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the B7-H4-ADC and PD-1 inhibitor (e.g. an anti-PD1 antibody).

In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the antibody or antigen-binding fragment thereof or antibody-drug conjugate described herein (e.g., a B7-H4-ADC). In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the B7-H4-ADC and PD-1 inhibitor (e.g. an anti-PD1 antibody). In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In one embodiment, regression of a tumor is determined by measuring the size of the tumor by positron emission tomography (PET). In one embodiment, regression of a tumor is determined by measuring the size of the tumor by ultrasound.

In one embodiment, response to treatment with B7H4-ADC in combination with PD-1 inhibitor is assessed by measuring the duration of response to the B7H4-ADC in combination with PD-1 inhibitor after administration of the B7H4-ADC and PD-1 inhibitor. In some embodiments, the duration of response after administration of the B7-H4-ADC in combination with anti-PD-1-antibody is increased by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to administration of monotherapy of the B7-H4-ADC or monotherapy of the anti-PD-1 antibody. In some embodiments, the duration of response after administration of the B7-H4-ADC in combination with anti-PD-1-antibody is improved by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to before administration of the B7-H4-ADC and the anti-PD-1 antibody. In some embodiments, the duration of response is duration of immune response. In some embodiments, the duration of immune response comprises durable tumor regression of tumor cells. In some embodiments, the tumor cell expresses B7-H4. In some embodiments, the tumor cell does not express B7-H4. In some embodiments, the tumor cell expresses a higher level of B7-H4 than a non-diseased cell of the same cell type. In some embodiments, the tumor cell expresses a comparable or lower level of B7-H4 than a non-diseased cell of the same cell type.

In one embodiment, response to treatment with B7-H4-ADC in combination with PD-1 inhibitor is assessed by measuring the time of overall survival after administration of the B7H4-ADC in combination with PD-1 inhibitor. In some embodiments, the overall survival after administration of the B7-H4-ADC in combination with anti-PD-1-antibody is improved by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to administration of monotherapy of the B7-H4-ADC or monotherapy of the anti-PD-1 antibody. In some embodiments, the overall survival after administration of the B7-H4-ADC in combination with anti-PD-1-antibody is improved by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to before administration of the B7-H4-ADC and the anti-PD-1 antibody.

In some embodiments, administration of the B7-H4-ADC induces upregulation of expression of one or more cytokines and/or one or more type I interferon response genes. In some embodiments, the cytokine is CXCL10 and/or CXCL1. In some embodiments, the type I interferon response gene is IFIT2 and/or MX1. In some embodiments, administration of the B7-H4-ADC induces upregulation of expression of CXCL10 and/or CXCL1. In some embodiments, administration of the B7-H4-ADC induces upregulation of expression of IFIT2 and/or MX1. In some embodiments, administration of the B7-H4-ADC induces activation of immune cells. In some embodiments, administration of the B7-H4-ADC induces recruitment of immune cells to tumors.

In some embodiments, administration of the B7-H4-ADC induces immunogenic cell death (ICD). In some embodiments, administration of the B7-H4-ADC induces release of ATP by cancer cells. In some embodiments, administration of the B7-H4-ADC induces exposure of calreticulin on the cancer cell surface.

In some embodiments, administration of the B7-H4-ADC promotes recruitment of innate immune cells and/or adaptive immune cells to the tumor. In some embodiments, administration of the B7-H4-ADC promotes recruitment of innate immune cells and/or adaptive immune cells to the tumor, and wherein the recruited immune cells are tumor infiltrating. In some embodiments, the innate immune cells comprise antigen-presenting cells including macrophages (such as F4/80+ macrophages) or dendritic cells (such as CD11c+ dendritic cells). In some embodiments, the adaptive immune cells comprise T cells (such as CD8+ T cells, CD3+ T cells, and/or CD3+CD8+ T cells). In some embodiments, the tumor cell expresses B7-H4. In some embodiments, the tumor cell does not express B7-H4. In some embodiments, the tumor cell expresses a higher level of B7-H4 than a non-diseased cell of the same cell type. In some embodiments, the tumor cell expresses a comparable or lower level of B7-H4 than a non-diseased cell of the same cell type.

In some embodiments, administration of the B7-H4 ADC induces an anti-tumor immune response in the subject. In some embodiments, an anti-tumor immune response is determined by a change in a marker of local inflammation at the tumor site. In some embodiments, an anti-tumor response is measured by expression of a chemokine, expression of an interferon, recruitment of a pro-inflammatory immune cell, change in cell cycle marker expression level, or change in transcript level associated with inflammation.

In some embodiments, administration of the B7-H4 ADC induces upregulation of expression of one or more chemokines and/or one or more type I interferon response genes. In some embodiments, administration of the B7-H4-ADC induces upregulation of expression of CXCL10, CXCL9, CXCL1, IFTIT2, and/or MX1. In some embodiments, expression is determined by qPCR.

In some embodiments, wherein administration of the B7-H4-ADC promotes recruitment of innate immune cells and/or adaptive immune cells to a tumor site. In some embodiments, the innate immune cells and/or adaptive immune cells are tumor infiltrating cells. Ins some embodiments, administration of the ADC causes recruitment of dendritic cells to the tumor cite. In some embodiments, dendritic cells express CD11c. In some embodiments, administration of the ADC causes recruitment of macrophages to the tumor site. In some embodiments, macrophages express F4/80. In some embodiments, administration of the ADC causes recruitment of cells expressing CD86 to the tumor cite. In some embodiments, the presence or absence of cells is determined by immunohistochemistry. In some embodiments, administration of the B7-H4-ADC promotes recruitment of CD11c+ dendritic cells, F4/80+ macrophages, and/or cells expressing CD86 to a tumor site.

In some embodiments, administration of the ADC causes an increase in gene expression of one or more genes associated with inflammation at the tumor site. In some embodiments, administration of the B7-H4-ADC causes an increase in expression of a gene associated with responsiveness to PD-1 agents. In some embodiments, administration of the ADC causes an increase expression of Cxcl9. In some embodiments, administration of the ADC causes an increase expression of Cxcl9, Cxcl10, Ifit2, Ifit3, and/or Mx1.

In some embodiments, administration of the ADC causes an increase in expression of a dendritic cell and macrophage marker. In some embodiments, administration of the ADC causes an increase in embodiments of Itgax, Batf3, and/or Cd68.

In some embodiments, administration of the ADC causes an increase in expression of an MHC class II molecule. In some embodiments, administration of the ADC causes an increase in expression of H2Aa and/or H2-eb1.

In some embodiments, administration of the ADC causes an increase in expression of a costimulatory molecule. In some embodiments, administration of the ADC causes an increase in expression of Cd80, Cd86, and/or Icos1.

In some embodiments, administration of the ADC causes an increase in expression of Itgax, Batf3, Cd68, H2-Aa, H2-eb1, Cd80, Cd86, and/or Icos1.

In some embodiments, administration of the ADC causes an increase in the presence of inflammatory cells at the tumor site. In some embodiments, the presence of CD3+ cells is increased. In some embodiments, the presence of C4+ cells is increased. In some embodiments, the presence of C8+ cells is increased. In some embodiments, the presence of PD1+ cells is increased. In some embodiments, the presence of inflammatory cells is determined using immunhisotochemistry.

In some embodiments, administration of the ADC causes an inflammatory gene expression signature. In some embodiments, the level of expression of Cd27, Cxcr6, Lag3, Nkg7, Pdcd1Ig2, Cc15, Cd274, Cmk131, Cxcl9, Psmb10, and/or Stat1 is increased.

In some embodiments, the level of expression of one of more of the genes provided in Table 23 is increased upon administration of the ADC. In some embodiments, the level of a gene associated with a gene ontology term description provided in Table 24 is increased.

In some embodiments, administration of the B7-H4-ADC causes a change in expression of a marker of cell division and/or cell cycle progression. In some embodiment, the level of Ki67, CD163, CD206, ChiL3, and/or Granzyme B positive cells at a tumor site.

In some embodiments, the vcMMAE B7-H4 ADC provided herein trigger a more potent response compared to ADC with other microtubule inhibitor drugs. In some embodiments, the vcMMAE B7-H4 ADC provided herein trigger a more potent immune response compared to an ADC comprising the same antibody conjugated to DM1 or DM4. In some embodiments, a lower amount of the vcMMAE B7-H4 ADC is needed to trigger an immune response compared to an ADC comprising the same antibody conjugated to DM1 or DM4.

### IV. PHARMACEUTICAL COMPOSITIONS AND FORMULATIONS

For therapeutic use, an antibody or antigen-binding fragment thereof or antibody-drug conjugate (e.g., a B7-H4-ADC) is combined with a pharmaceutically acceptable carrier. In some embodiments according to any of the B7-H4-ADC compositions described herein (e.g. a composition comprising a B7-H4-ADC), the composition comprises a pharmaceutically acceptable carrier. In some embodiments according to any of the B7-H4-ADC compositions described herein (e.g. a composition comprising a B7-H4-ADC and an immune checkpoint inhibitor), the composition comprises a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, antibody or antigen-binding fragment thereof or antibody-drug conjugate (e.g., a B7-H4-ADC) compositions of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

Suitable pharmaceutical excipients and/or additives for use in the antibody molecule compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998).

Pharmaceutical compositions containing an antibody or antigen-binding fragment thereof or antibody-drug conjugate (e.g., a B7-H4-ADC) as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

The compositions of this invention may be in a variety of forms. These include, for example, liquid, semi-solid and solid dosage forms, such as liquid solutions (e.g., injectable and infusible solutions), dispersions or suspensions, and liposomes. The particular form depends on the intended mode of administration and therapeutic application. In exemplary embodiments, compositions provided are in the form of injectable or infusible solutions. Exemplary administration is parenteral (e.g., intravenous, subcutaneous, intraocular, intraperitoneal, intramuscular). In an exemplary embodiment, the preparation is administered by intravenous infusion or injection. In another preferred embodiment, the preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

In some embodiments, the therapeutically effective dose of an antibody or antigen binding fragment or antibody-drug conjugate (e.g. a B7-H4 ADC) is about 0.5 mg/kg to about 3.0 mg/kg of the subject's body weight. In some embodiments according to any one of the methods described above, the antibody or antigen binding fragment or antibody-drug conjugate (e.g. a B7-H4 ADC) is administered one or more times.

The present invention provides a kit, comprising packaging material and at least one vial comprising a solution of at least an antibody or antigen-binding fragment thereof or antibody-drug conjugate (e.g., a B7-H4-ADC) with the prescribed buffers and/or preservatives, optionally in an aqueous diluent. The concentration of preservative used in the formulation is a concentration sufficient to yield an anti-microbial effect. Such concentrations are dependent on the preservative selected and are readily determined by the skilled artisan.

Various delivery systems can be used to administer antibodies or antigen-binding fragments thereof or antibody-drug conjugate to a subject. In certain exemplary embodiments, administration of an antibody or antigen-binding fragment thereof or antibody-drug conjugate (e.g., a B7-H4-ADC) is by intravenous infusion.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized, it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present invention can be used with the methods described herein or with other methods for treating disease. The antibody or antigen-binding fragment thereof or antibody-drug conjugate (e.g., B7-H4-ADC) formulations may be further diluted before administration to a subject. In some embodiments, the formulations will be diluted with saline and held in IV bags or syringes before administration to a subject. Accordingly, in some embodiments, the methods for treating a cancer, such as a B7-H4-expressing cancer, in a subject will comprise administering to a subject in need thereof a weekly dose of a pharmaceutical composition comprising an antibody or antigen-binding fragment thereof or antibody-drug conjugate (e.g., a B7-H4-ADC).

### V. ARTICLES OF MANUFACTURE AND KITS

In another aspect, an article of manufacture or kit is provided which comprises an antibody or antigen-binding fragment thereof or antibody-drug conjugate described herein (e.g., a B7-H4-ADC). The article of manufacture or kit may further comprise instructions for use of the antibody or antigen-binding fragment thereof or antibody-drug conjugate described herein (e.g., a B7-H4-ADC) in the methods of the invention. Thus, in certain embodiments, the article of manufacture or kit comprises instructions for the use of an anti-B7-H4 antibody or antigen-binding fragment thereof or antibody-drug conjugate described herein (e.g., a B7-H4-ADC) in methods for treating cancer (e.g., breast cancer) in a subject comprising administering to the subject an effective amount of an anti-B7-H4 antibody or antigen-binding fragment thereof or antibody-drug conjugate described herein (e.g., a B7-H4-ADC). In one aspect, an article of manufacture or kit is provided which comprises an antibody or antigen-binding fragment thereof or antibody-drug conjugate described herein (e.g., a B7-H4-ADC), and an immune checkpoint inhibitor (e.g. an anti-PD1 antibody). The article of manufacture or kit may further comprise instructions for use of the antibody or antigen-binding fragment thereof or antibody-drug conjugate described herein (e.g., a B7-H4-ADC), and the immune checkpoint inhibitor (e.g. an anti-PD1 antibody) in the methods of the invention. In certain embodiments, the article of manufacture or kit comprises instructions for the use of an anti-B7-H4 antibody or antigen-binding fragment thereof or antibody-drug conjugate described herein (e.g., a B7-H4-ADC) and an immune checkpoint inhibitor (e.g. an anti-PD1 antibody) in methods for treating cancer (e.g., breast cancer) in a subject comprising administering to the subject an effective amount of an anti-B7-H4 antibody or antigen-binding fragment thereof or antibody-drug conjugate described herein (e.g., a B7-H4-ADC), and an effective amount of the immune checkpoint inhibitor (e.g. an anti-PD1 antibody). In some embodiments the cancer is a locally advanced cancer. In some embodiments, the cancer is a metastatic cancer. In some embodiments, the cancer is breast cancer as described herein. In certain embodiments, the article of manufacture or kit comprises instructions for the use of an anti-B7-H4 antibody or antigen-binding fragment thereof, or antibody-drug conjugate described herein (e.g., a B7-H4-ADC) in methods for treating cancer (e.g., locally advanced or metastatic solid tumors (e.g., small cell lung cancer, non-small cell lung cancer, head and neck squamous cell carcinoma, esophageal squamous cell carcinoma, and gastric and gastroesophageal junction adenocarcinoma)) in a subject comprising administering to the subject an effective amount of an anti-B7-H4 antibody or antigen-binding fragment thereof or antibody-drug conjugate described herein (e.g., a B7-H4-ADC). In some embodiments the cancer is a locally advanced solid tumor. In some embodiments, the cancer is a metastatic solid tumor. In some embodiments, the cancer is small cell lung cancer as described herein. In some embodiments, the cancer is non-small cell lung cancer as described herein. In some embodiments, the cancer is head and neck cancer as described herein. In some embodiments, the cancer is esophageal carcinoma as described herein. In some embodiments, the cancer is gastric cancer as described herein. In some embodiments, the cancer is gastroesophageal junction cancer as described herein. In some embodiments, the subject is a human. In some embodiments, the cancer is selected from breast cancer, ovarian cancer, lung cancer, cholangiocarcinoma and endometrial cancer. In some embodiments herein, the cancer is selected from peritoneal cancer, fallopian tube cancer, and gallbladder cancer. In one preferred embodiment, the cancer is selected from the group consisting of ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma and gallbladder carcinoma.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer, e.g., breast cancer, as described herein in a subject. The label or package insert may further indicate that the formulation is useful or intended for subcutaneous, intravenous (e.g., intravenous infusion), or other modes of administration for treating lung cancer, head and neck cancer, esophageal cancer, gastric cancer, or gastroesophageal junction cancer as described herein in a subject. The label or package insert may indicate that the formulation is useful or intended for subcutaneous, intravenous (e.g., intravenous infusion), or other modes of administration for treating breast cancer, ovarian cancer, lung cancer, cholangiocarcinoma, endometrial cancer, peritoneal cancer, fallopian tube cancer, or gallbladder cancer as described herein in a subject. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use. In some embodiments, the second medicament comprises an immune checkpoint inhibitor (e.g. an anti-PD1 antibody).

The article of manufacture or kit herein optionally further comprises a container comprising a second medicament, wherein an anti-B7-H4 antibody or antigen-binding fragment thereof or antibody-drug conjugate described herein (e.g., a B7-H4-ADC) is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

The article of manufacture or kit herein optionally further comprises a container comprising a second medicament, wherein the second medicament is for eliminating or reducing the severity of one or more adverse events, wherein an antibody or antigen-binding fragment thereof or antibody-drug conjugate described herein (e.g., a B7-H4-ADC) is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein. In some embodiments, the label or package insert indicates that the first medicament is to be administered prior to the administration of the second medicament. In some embodiments, the label or package insert indicates that second medicament is to be administered prior to the first medicament.

In some embodiments, an antibody or antigen-binding fragment thereof or antibody-drug conjugate described herein (e.g., a B7-H4-ADC) is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EMBODIMENTS

1. A B7-H4 antibody-drug conjugate (B7-H4-ADC), wherein the B7-H4-ADC comprises an anti-B7-H4 antibody conjugated to a vcMMAE (valine-citruline-monomethyl auristatin E), wherein the anti-B7-H4 antibody comprises heavy chain variable region (VH)-complementarity determining region (CDR) 1, VH-CDR2, VH-CDR3 and light chain variable region (VL)-CDR1, VL-CDR2, and VL-CDR3 sequences of SEQ ID NOs: 5-10, respectively; wherein the vcMMAE comprises the structure:

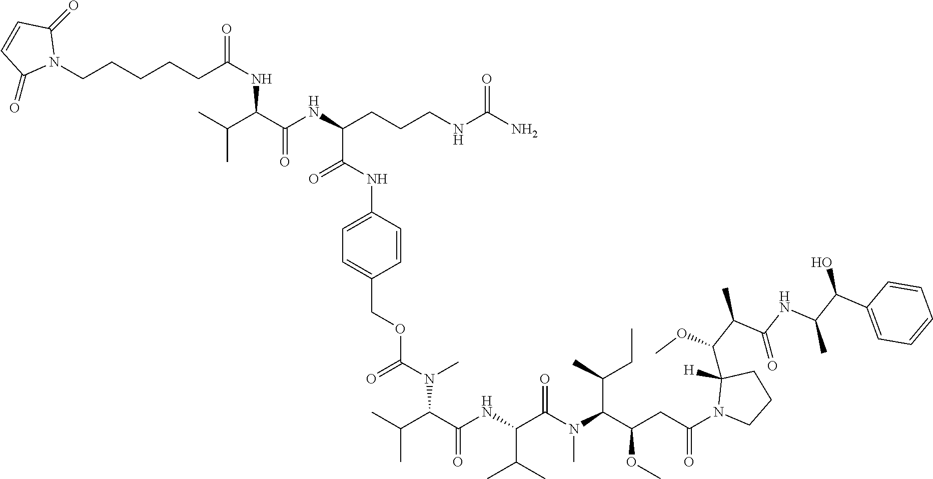 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
2. The B7-H4-ADC of embodiment 1, wherein the anti-B7-H4 antibody comprises a heavy chain variable region (HCVR) having at least 95% identity to SEQ ID NO: 11, and a light chain variable region (LCVR) having at least 95% identity to SEQ ID NO: 12.
3. A B7-H4 antibody-drug conjugate (B7-H4-ADC), wherein the B7-H4-ADC comprises an anti-B7-H4 antibody conjugated to a vcMMAE (valine-citruline-monomethyl auristatin E), wherein the anti-B7-H4 antibody comprises a heavy chain variable region (HCVR) having at least 95% identity to SEQ ID NO: 11, and a light chain variable region (LCVR) having at least 95% identity to SEQ ID NO: 12, wherein the vcMMAE comprises the structure:

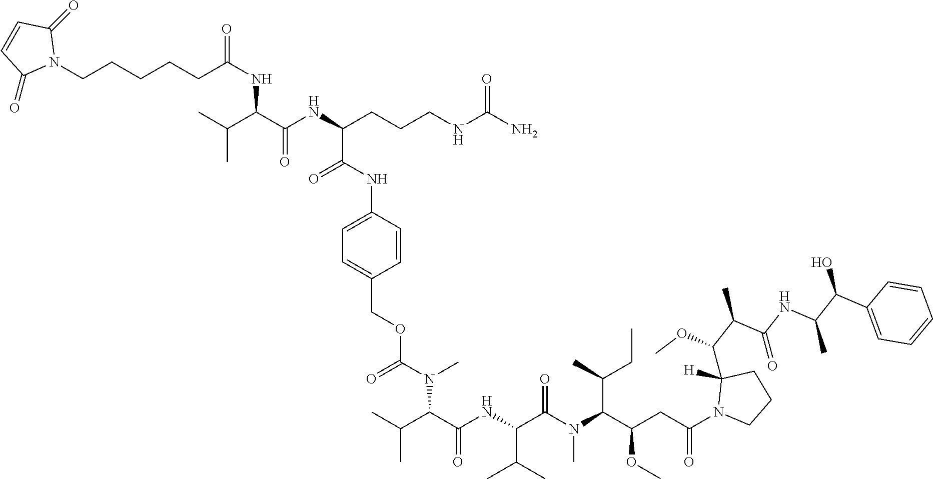 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
4. The B7-H4-ADC of embodiment 3, wherein the heavy chain variable region of the anti-B7-H4 antibody comprises the three complementarity determining regions (CDRs) of any one of SEQ ID NO: 11, and the light chain variable region of the antibody or antigen-binding fragment thereof comprises the three CDRs of SEQ ID NO: 12.
5. The B7-H4-ADC of any one of embodiments 1-4, wherein the heavy chain variable region has at least 98% identity to SEQ ID NO:11 and the light chain variable region has at least 98% identity to SEQ ID NO:12.
6. The B7-H4-ADC of any one of embodiments 1-5, wherein the heavy chain variable region has at least 99% identity to SEQ ID NO:11 and the light chain variable region has at least 99% identity to SEQ ID NO:12.
7. The B7-H4-ADC of any one of embodiments 1-6, wherein the heavy chain variable region comprises the sequence of SEQ ID NO: 11 and the light chain variable region comprises the sequence of SEQ ID NO: 12.
8. The B7-H4-ADC of any one of embodiments 1-7, wherein the B7-H4-ADC comprises the structure:

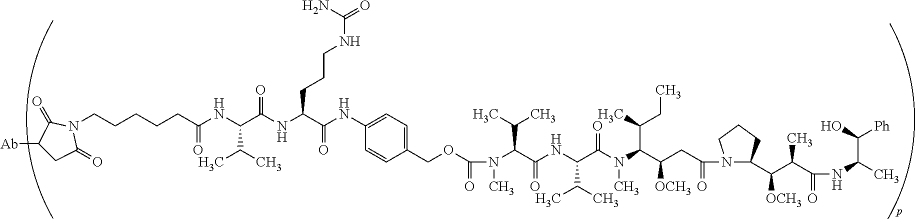

9. The B7-H4-ADC of any one of embodiments 1-7, wherein the B7-H4-ADC comprises the structure:

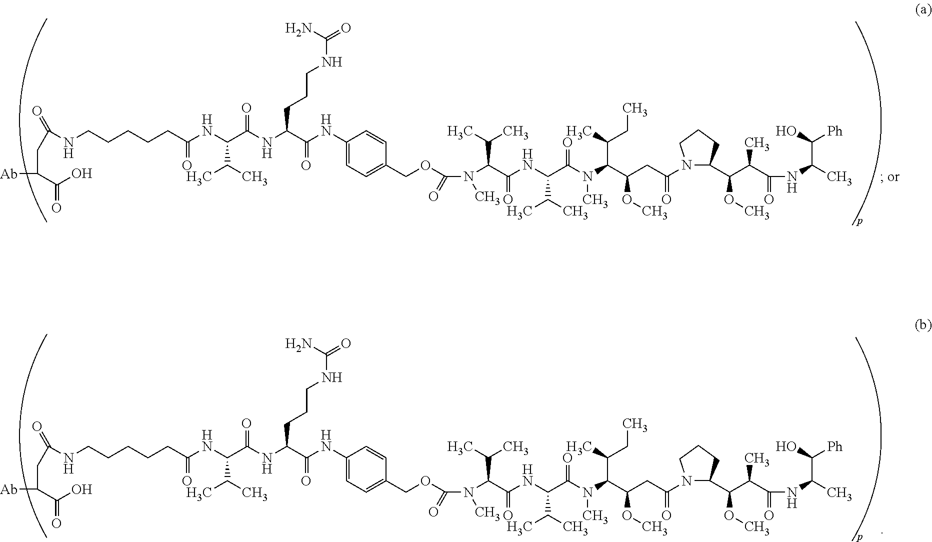

10. The B7-H4-ADC of any one of embodiments 1-9, wherein a vcMMAE to antibody ratio is from about 1 to about 8.
11. The B7-H4-ADC of any one of embodiments 1-10, wherein the vcMMAE to antibody ratio is about 4.
12. The B7-H4-ADC of any one of embodiments 1-11, wherein the anti-B7-H4 antibody is a fully human antibody.
12A. The B7-H4-ADC of any one of embodiments 1-11, wherein the anti-B7-H4 antibody is a humanized antibody.
13. The B7-H4-ADC of any one of embodiments 1-12A, wherein the anti-B7-H4 antibody is a IgG1 monoclonal antibody.
14. The B7-H4-ADC of any one of embodiments 1-13, wherein the B7-H4-ADC is within a heterogeneous population of B7-H4-ADCs, wherein the anti-B7-H4 antibodies comprised within the heterogeneous population of B7-H4-ADCs exhibit variable post-translational modifications.
15. The B7-H4-ADC of embodiment 14, wherein within at least 50%, 60%, 70%, 80%, 90%, or 95% of the anti-B7-H4 antibodies comprised within the heterogeneous population of B7-H4-ADCs:
(i) the C-terminal lysine residues are removed from both heavy chains; and/or
(ii) the N-terminal glutamine of each heavy chain cyclized to pyroglutamic acid; and/or
(iii) the consensus glycosylation site at Asn300 of each heavy chain occupied predominantly with biantennary, core fucosylated glycans without terminal galactose residues.
16. A method of treating a subject having or at risk of having a B7-H4-associated cancer, comprising:

administering to the subject a therapeutically effective dose of a B7-H4 antibody-drug conjugate (B7-H4-ADC),

wherein the B7-H4-ADC comprises an anti-B7-H4 antibody conjugated to a vcMMAE (valine-citruline-monomethyl auristatin E), wherein the anti-B7-H4 antibody comprises heavy chain variable region (VH)-complementarity determining region (CDR) 1, VH-CDR2, VH-CDR3 and light chain variable region (VL)-CDR1, VL-CDR2, and VL-CDR3 sequences of SEQ ID NOs: 5-10, respectively;

wherein the vcMMAE comprises the structure:

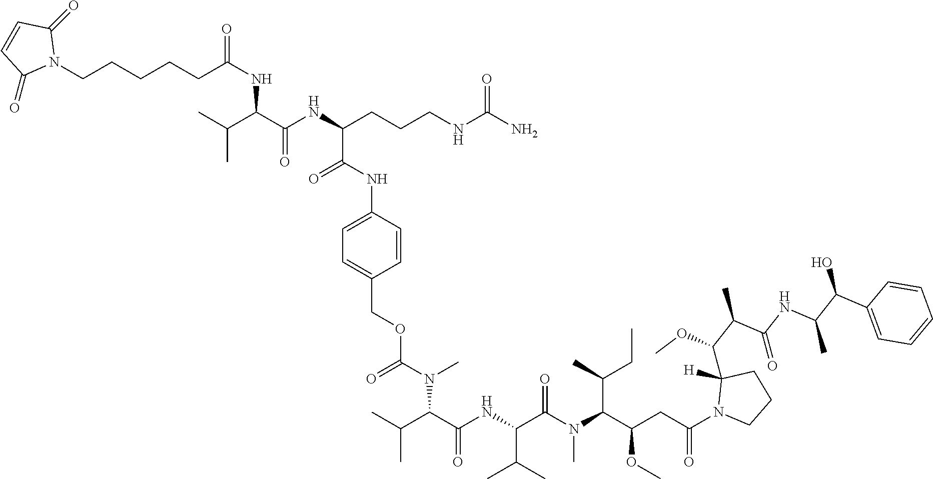 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
17. The method of embodiment 16, wherein the anti-B7-H4 antibody comprises a heavy chain variable region (HCVR) having at least 95% identity to SEQ ID NO: 11, and a light chain variable region (LCVR) having at least 95% identity to SEQ ID NO: 12.
18. A method of treating a subject having or at risk of having a B7-H4-associated cancer, comprising:

administering to the subject a therapeutically effective dose of a B7-H4 antibody-drug conjugate (B7-H4-ADC),

wherein the B7-H4-ADC comprises an anti-B7-H4 antibody conjugated to a vcMMAE (valine-citruline-monomethyl auristatin E), wherein the anti-B7-H4 antibody comprises a heavy chain variable region (HCVR) having at least 95% identity to SEQ ID NOs: 11, and a light chain variable region (LCVR) having at least 95% identity to SEQ ID NO: 12, wherein the vcMMAE has the structure:

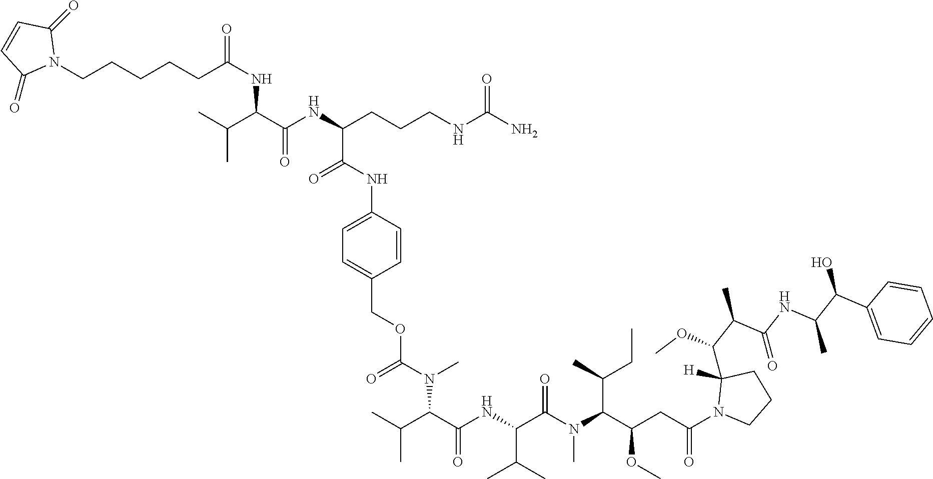 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]

or a pharmaceutically acceptable salt thereof.
19. The method of embodiment 18, wherein the heavy chain variable region of the anti-B7-H4 antibody comprises the three complementarity determining regions (CDRs) of SEQ ID NO: 11, and the light chain variable region of the antibody or antigen-binding fragment thereof comprises the three CDRs of SEQ ID NO: 12.
20. The method of any one of embodiments 16-19, wherein the heavy chain variable region has at least 98% identity to SEQ ID NO:11 and the light chain variable region has at least 98% identity to SEQ ID NO:12.
21. The method of any one of embodiments 16-20, wherein the heavy chain variable region has at least 99% identity to SEQ ID NO:11 and the light chain variable region has at least 99% identity to SEQ ID NO:12.
22. The method of any one of embodiments 16-21, wherein the heavy chain variable region comprises the sequence of SEQ ID NO:11 and the light chain variable region comprises the sequence of SEQ ID NO:12.
23. The method of any one of embodiments 16-22, wherein the B7-H4-ADC comprises the structure:

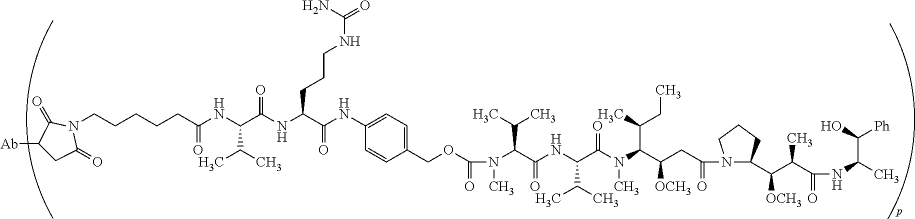

24. The method of any one of embodiments 16-23, wherein the B7-H4-ADC comprises the structure:

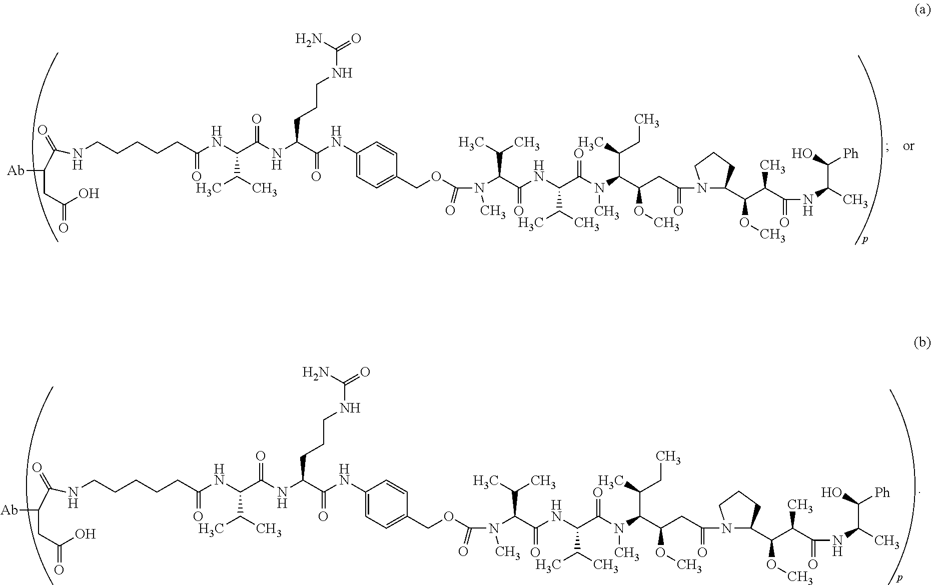

25. The method of any one of embodiments 16-24, wherein a vcMMAE to antibody ratio is from about 1 to about 8.
26. The method of any one of embodiments 16-25, wherein the vcMMAE to antibody ratio is about 4.
27. The method of any one of embodiments 16-26, wherein the anti-B7-H4 antibody is a IgG1 monoclonal antibody.
28. The method of any one of embodiments 16-27, wherein the anti-B7-H4 antibody is a fully human antibody.
28A. The method of any one of embodiments 1-28, wherein the anti-B7-H4 antibody is a humanized antibody.
29. The method of any one of embodiments 16-28A, wherein the B7-H4-ADC is within a heterogeneous population of B7-H4-ADCs, wherein the anti-B7-H4 antibodies comprised within the heterogeneous population of B7-H4-ADCs exhibit variable post-translational modifications.
30. The method of embodiment 29, wherein within at least 50%, 60%, 70%, 80%, 90%, or 95% of the anti-B7-H4 antibodies comprised within the heterogeneous population of B7-H4-ADCs:
(i) the C-terminal lysine residues are removed from both heavy chains; and/or
(ii) the N-terminal glutamine of each heavy chain cyclized to pyroglutamic acid; and/or
(iii) the consensus glycosylation site at Asn300 of each heavy chain occupied predominantly with biantennary, core fucosylated glycans without terminal galactose residues.
31. The method of any one of embodiments 16-30, wherein the subject has been previously treated with one or more therapeutic agents and did not respond to the treatment, wherein the one or more therapeutic agents is not the anti-B7-H4 antibody or antigen-binding fragment thereof.
32. The method of any one of embodiments 16-30, wherein the subject has been previously treated with one or more therapeutic agents and relapsed after the treatment, wherein the one or more therapeutic agents is not the B7-H4-ADC, the anti-B7-H4 antibody or antigen-binding fragment thereof.
33. The method of any one of embodiments 16-32 wherein the subject has been previously treated with one or more therapeutic agents and has experienced disease progression during treatment, wherein the one or more therapeutic agents is not the B7-H4-ADC, the anti-B7-H4 antibody or antigen-binding fragment thereof.
34. The method of any one of embodiments 16-33, wherein the cancer is selected from the group consisting of breast cancer, ovarian cancer, lung cancer, cholangiocarcinoma and endometrial cancer.
34A. The method of any one of embodiments 16-33, wherein the cancer is selected from the group consisting of peritoneal cancer, fallopian tube cancer, and gallbladder cancer.
34B. The method of any one of embodiments 16-33, wherein the cancer is selected from the group consisting of ovarian neoplasms, peritoneal neoplasms, fallopian tube neoplasms, HER2 negative breast neoplasms, HER2 positive breast neoplasms, triple negative breast neoplasms, endometrial neoplasms, non-small-cell lung carcinoma, cholangiocarcinoma and gallbladder carcinoma.
35. The method of embodiment 34, wherein the cancer is lung cancer, optionally wherein the lung cancer is lung squamous cell carcinoma (LUSC) or lung adenocarcinoma.
36. The method of embodiment 35, wherein the lung cancer is non-small cell lung cancer (NSCLC).
37. The method of embodiment 34, wherein the cancer is endometrial cancer, optionally wherein the endometrial cancer is uterine endometrial carcinoma (UCEC).
38. The method of embodiment 34, wherein the cancer is ovarian cancer.
39. The method of embodiment 38, wherein the ovarian cancer is ovarian serous adenocarcinoma (OV).
40. The method of embodiment 34, wherein the cancer is a breast cancer.
41. The method of embodiment 40, wherein the breast cancer is progesterone receptor positive/human epidermal growth factor receptor 2 negative breast (PR+/HER2−) cancer.
42. The method of embodiment 40, wherein the breast cancer is a triple negative breast cancer.
43. The method of embodiment 40, wherein the breast cancer is HR+/HER2 negative breast cancer.
44. The method of embodiment 40, wherein the breast cancer is HER2 positive breast cancer.
45. The method of embodiment 40, wherein the breast cancer is breast invasive carcinoma (BRCA).
46. The method of any one of embodiments 31-45, wherein the subject received one or more prior cytotoxic regimen.
47. The method of any one of embodiments 31-46, wherein the subject received two or more prior cytotoxic regimens.
48. The method of embodiment 46 or 47, wherein the subject received prior therapy with a cytotoxic chemotherapy.
49. The method of any one of embodiments 46-48, wherein the subject received prior therapy with a platinum-based therapy or platinum-based combination therapy.
50. The method of any one of embodiments 31-49, wherein the cancer is an advanced stage cancer.
51. The method of embodiment 50, wherein the advanced stage cancer is a stage 3 or stage 4 cancer.
52. The method of embodiment 50 or 51, wherein the advanced stage cancer is metastatic cancer.
53. The method of any one of embodiments 31-52, wherein the cancer is recurrent cancer. 54. The method of any one of embodiments 31-53, wherein the cancer is unresectable.
55. The method of any one of embodiments 31-54, wherein the subject received prior treatment with standard of care therapy for the cancer and failed the prior treatment.
56. The method of any one of embodiments 31-55, wherein the B7-H4-ADC is in a pharmaceutical composition comprising the B7-H4-ADC and a pharmaceutically acceptable carrier.
57. The method of any one of embodiments 31-56, wherein the subject is a human.
58. The method of any one of embodiments 31-57, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells express B7-H4.
59. The method of any one of embodiments 31-58, wherein one or more therapeutic effects in the subject is improved after administration of the B7-H4-ADC relative to a baseline.
60. The method of embodiment 59, wherein the one or more therapeutic effects comprises size of a tumor derived from the cancer.
61. The method of any one of embodiments 31-60, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the B7-H4-ADC.
62. The method of any one of embodiments 31-61, wherein the B7-H4-ADC is administered as a monotherapy.
63. A kit comprising:

(a) a dosage ranging from about 0.5 mg/kg to about 3 mg/kg of B7-H4-ADC, or an antibody or antigen-binding fragment thereof that binds B7-H4; and

(b) instructions for using the B7-H4-ADC according to the method of any one of embodiments 31-62.

64. The method of any one of embodiments 31-58, wherein one or more therapeutic effects in the subject is improved after administration of the B7-H4-ADC as compared to administration of a corresponding B7-H4 antibody not conjugated to an vcMMAE.
65. The method of embodiment 64, wherein the one or more therapeutic effects comprises reduction in size of a tumor derived from the cancer.
66. The method of embodiment 65, wherein the reduction in size of a tumor derived from the cancer after administration of the B7-H4-ADC is at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold more than the reduction in size of a tumor derived from the cancer after administration of a corresponding B7-H4 antibody not conjugated to an vcMMAE.
67. The method of any one of embodiments 31-61 and 64-66, wherein administration of the B7-H4-ADC induces upregulation of expression of:
(a) one or more chemokines; optionally wherein the chemokine is CXCL10 and/or CXCL1; and/or
(b) one or more type I interferon response genes; optionally wherein the type I interferon response gene is IFIT2 and/or MX1.
68. The method of any one of embodiments 31-61 and 64-67, wherein administration of the B7-H4-ADC promotes recruitment of innate immune cells and/or adaptive immune cells to the tumor site, optionally wherein the innate immune cells and/or adaptive immune cells are tumor infiltrating.
69. The method of embodiment 68, wherein:
(a) the innate immune cells comprise macrophages, dendritic cells, and/or antigen-presenting cells, optionally wherein the macrophages are F4/80+ macrophages and/or wherein the dendritic cells are CD11c+;
(b) the adaptive immune cells comprise T cells, optionally wherein the T cells are CD3+ and/or CD8+.
   70. The method of any one of embodiments 31-61 and 64-69, wherein administration of the B7-H4-ADC induces:
   (a) release of ATP by cancer cells; and/or
   (b) exposure of calreticulin in cancer cell surface.
   71. The method of any one of embodiments 31-61 and 64-70, wherein the B7-H4-ADC is administered as a monotherapy.
   72. The method of any one of embodiments 31-61 and 64-70, wherein the B7-H4-ADC is administered in combination with an anti-PD-1 antibody.
   73. The method of embodiment 72, wherein one or more therapeutic effects in the subject is improved after administration of the B7-H4-ADC in combination with the anti-PD1-antibody as compared to administration of monotherapy of the B7-H4-ADC or monotherapy of the anti-PD-1 antibody.
   74. The method of embodiment 73, wherein the one or more therapeutic effects comprises improved overall survival, increased duration of response and/or reduction in size of a tumor derived from the cancer.
   75. The method of embodiment 74, wherein the reduction in size of a tumor derived from the cancer after administration of the B7-H4-ADC in combination with the anti-PD-1-antibody is at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold more than the reduction in size of the tumor derived from the cancer after administration of monotherapy of the B7-H4-ADC or monotherapy of the anti-PD-1 antibody.
   76. The method of embodiment 74 or 75, wherein the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the B7-H4-ADC and the anti-PD-1 antibody.
   77. The method of embodiment 74, wherein the overall survival after administration of the B7-H4-ADC in combination with the anti-PD-1-antibody is improved by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to administration of monotherapy of the B7-H4-ADC or monotherapy of the anti-PD-1 antibody.
   78. The method of embodiment 74 or 77, wherein the overall survival after administration of the B7-H4-ADC in combination with the anti-PD-1-antibody is improved by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to before administration of the B7-H4-ADC and the anti-PD-1 antibody
   79. The method of embodiment 74, wherein the duration of response after administration of the B7-H4-ADC in combination with the anti-PD-1-antibody is increased by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to administration of monotherapy of the B7-H4-ADC or monotherapy of the anti-PD-1 antibody.
   80. The method of embodiment 74 or 79, wherein the duration of response after administration of the B7-H4-ADC in combination with the anti-PD-1-antibody is improved by at least about any one of: 10%, 15%, 20%, 25%, 30%, 35%, 40%, 45%, 50%, 60%, 70%, 80%, 90%, 100%, 2-fold, 5-fold, 10-fold, 50-fold, 100-fold, 500-fold or 1000-fold as compared to before administration of the B7-H4-ADC and the anti-PD-1 antibody.
   81. A kit comprising:

(a) a B7-H4-ADC, or an antibody or antigen-binding fragment thereof that binds B7-H4; and

(b) instructions for using the B7-H4-ADC according to the method of any one of embodiments 31-62 and 64-71.

82. A kit comprising:

(a) a B7-H4-ADC and an anti-PD-1 antibody; and

(b) instructions for using the B7-H4-ADC and the anti-PD-1 antibody according to the method of any one of embodiments 72-80.

### EXAMPLES

### Example 1: Structure of Fully Human Anti-B7-H4 IgG1 Monoclonal Antibody

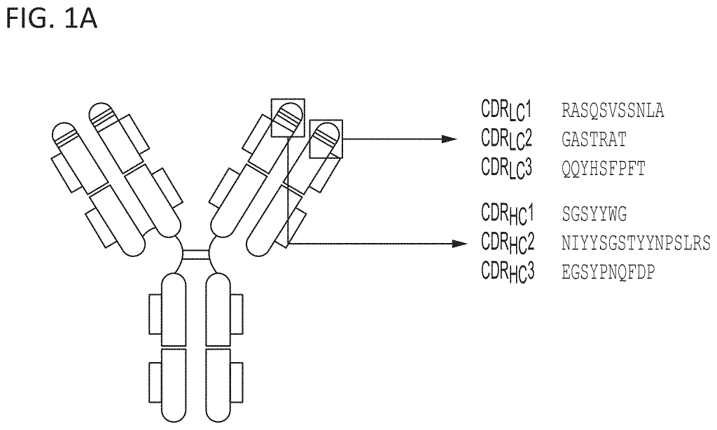

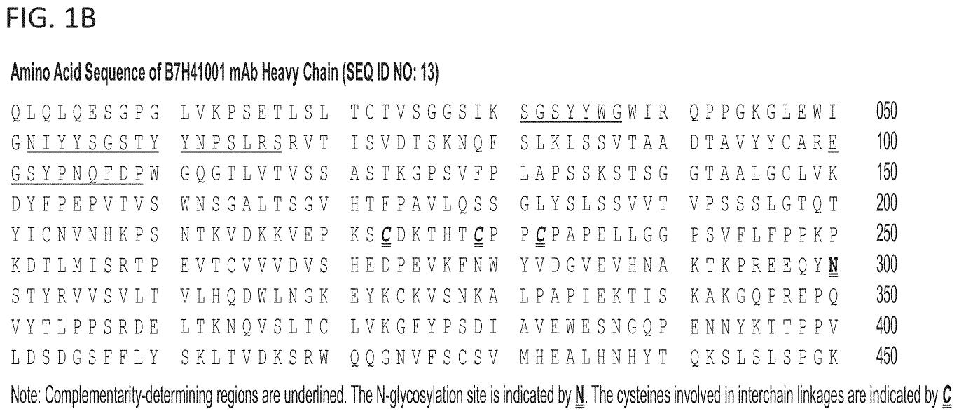

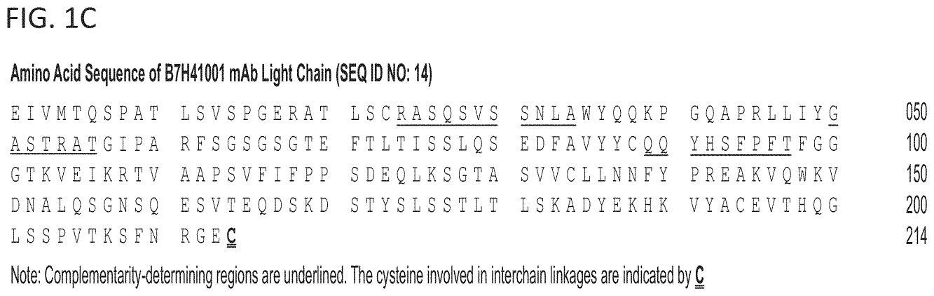

B7H41001 mAb is a fully human anti-B7-H4 immunoglobulin G1 (IgG1) monoclonal antibody (FIG. 1A) composed of two heavy chains (450 residues per chain) and two light chains (214 residues per chain) covalently linked by inter-chain disulfide bonds. The heavy chain is of the gamma 1 (G1) class and the primary sequence is shown in FIG. 1B. The light chain of B7H41001 is of the kappa class, and the primary sequence is shown in FIG. 1C.

B7H41001 mAb is a heterogeneous mixture of related species with variable post-translational modifications. The most abundant form exists with the C-terminal lysine residues removed from both heavy chains, the N-terminal glutamine of each heavy chain cyclized to pyroglutamic acid, and the consensus glycosylation site at Asn300 of each heavy chain occupied predominantly with biantennary, core fucosylated glycans without terminal galactose residues. The molecular formula and calculated molecular weight of this nominal form are presented in Table 8.

TABLE 8
Chemical Properties of B7H41001 mAb

| Property | Value |
|---|---|
| Molecular formula | C_{6,542}H_{10,110}N_{1,710}O_{2,090}S₃₈ |
| Molecular weight | 147,375 g mol⁻¹ |

### Example 2: RNA Expression of VTCN1 on Human Tumor Samples

TCGA RNA-seq data were quantified using the Toil quantification pipeline (Vivian et al., 2017) to produce gene-level normalized counts (Transcripts Per Kilobase Million, TPM) and downloaded from the UCSC Xena browser on Oct. 19, 2020 (https://xenabrowser.net/datapagesncohort=TCGA %20TARGET %20GTEx). Gene-level expression values, subsequent analysis and visualization steps were performed in the R computing environment. Gene expression was analyzed in ACC (Adrenocortical Carcinoma), DLBC (Lymphoid Neoplasm Diffuse Large B-cell Lymphoma), LAML (Acute Myeloid Leukemia), PCPG (Pheochromocytoma and Paraganglioma), THYM (Thymoma), UVM (Uveal Melanoma), MESO (Mesothelioma), READ (Rectum Adenocarcinoma), SKCM (Skin Cutaneous Melanoma), COAD (Colon Adenocarcinoma), LIHC (Liver Hepatocellular Carcinoma), SARC (Sarcoma), GBM (Glioblastoma Multiforme), KICH (Kidney Chromophobe), LGG (Brain Lower Grade Glioma), TGCT (Testicular Germ Cell Tumor), THCA (Thyroid Carcinoma), KIRC (Kidney Renal Clear Cell Carcinoma), STAD (Stomach Adenocarcinoma), HNSC (Head and Neck Squamous Carcinoma), PRAD (Prostate Adenocarcinoma), LUAD (Lung Adenocarcinoma), ESCA (Esophageal Carcinoma), CESC (Cervical Squamous Cell Carcinoma and Endocervical Adenocarcinoma), BLCA (Bladder Urothelial Carcinoma), KIRP (Kidney Renal Papillary Cell Carcinoma), UCS (Uterine Carcinosarcoma), LUSC (Lung Squamous Cell Carcinoma), PAAD (Pancreatic Adenocarcinoma), UCEC (Uterine Corpus Endometrial Carcinoma), CHOL (Cholangiocarcinoma), OV (Ovarian Serous Cystadenocarcinoma), and BRCA (Breast Invasive Carcinoma).

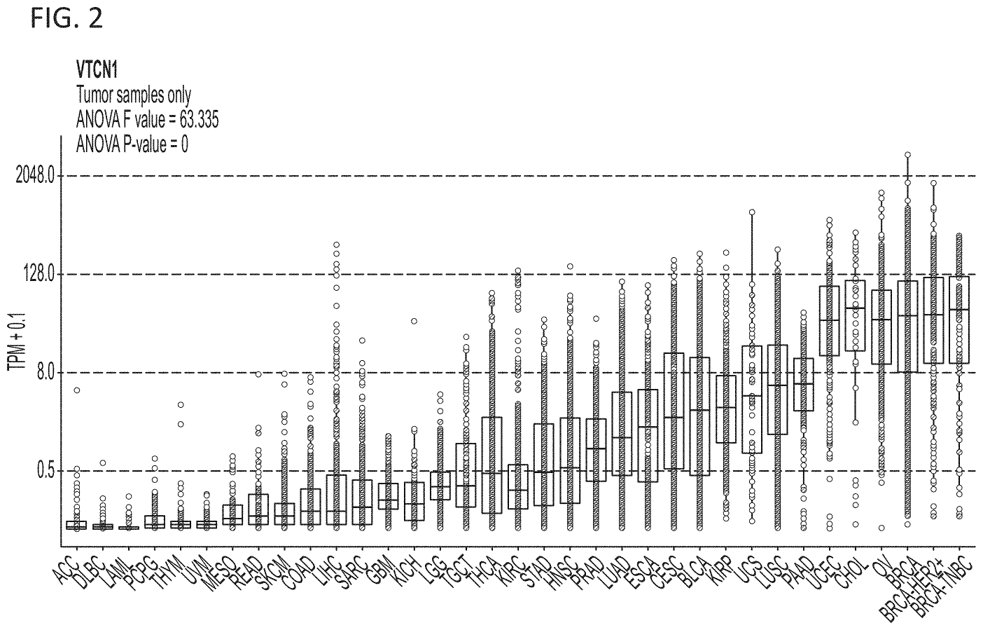

Expression of VTCN1, the gene that encodes B7-H4 protein, was detected in multiple solid tumor types based on publicly available gene expression data from The Cancer Genome Atlas (FIG. 2). Expression of VTCN1 was highest in breast invasive carcinoma (BRCA), ovarian serous adenocarcinoma (OV), cholangiocarcinoma (CHOL), and uterine endometrial carcinoma (UCEC). VTCN1 was also expressed in lung squamous cell carcinoma (LUSC) and, to a lesser extent, lung adenocarcinoma (LUAD).

### Example 3: Validation of Anti-B7-H4 Antibody Clone D1M8I for IHC Detection of Human B7-H4

IHC staining of B7-H4-negative and B7-H4-positive cell pellets was performed to confirm that the anti-B7-H4 antibody clone D1M8I was sensitive and specific for B7-H4.

### Preparation of Formalin-Fixed Paraffin-Embedded Cell Pellets

HEK293T cells were transfected with human VTCN1 (gene encoding B7-H4; RefSeq: NM_024626.4) and mouse Vtcn1 (RefSeq: NM_178594.3). Formalin-fixed paraffin embedded cell pellets were prepared by expanding HEK293T, HEK293T_hB7-H4, and HEK293T_mB7-H4 cell lines to grow 50 million cells for each cell pellet under the standard conditions. Adherent cells were lifted using Non-Enzymatic Dissociation solution (ATCC cat #30-2130) or Versene (Gibco #15040-066). Cells were then pelleted and washed twice with cold PBS and fixed with 10% buffered formalin. The cells were mixed gently by pipetting to make sure they were well mixed in the fixative. Cells were then transferred to 15 ml conical tubes and date and time of fixation was recorded. After 24 hours of fixation, cells were washed twice with PBS. Histogel (Fisher Scientific #22-045381) was warmed in a hot water bath until it reached liquid consistency. Next, 300 μL of histogel was mixed with the cell pellet and the suspension was transferred to an eppendorf tube and placed in ice for 30 minutes. After the incubation on ice, the pellet was removed and transferred to a vial with 70% ethanol. These pellets were then fixed in paraffin blocks and sectioned for IHC staining.

### Quantitative Flow Cytometry on Human Tumor Cell Lines

Cells were harvested with Versene, aliquoted at 200,000 cells/well into a 96-well round bottom plate and washed with BD stain buffer (BD #554657). Cells were then blocked with 50 μL of a 1:10 dilution of human IgG Fc fragment (Millipore 401104-5MG, lot #2951524) for 10 minutes on ice prior to adding 50 μL of either anti-B7-H4 mAb (clone MIH43, Biolegend #358102, lot #B245309) or mIgG1 isotype control mAb (clone MOPC21, BioXCell #BE0083, lot #701618J2) at a final concentration of 10 μg/mL. Cells were incubated for 30 minutes on ice and washed twice with BD stain buffer. Finally, cells, set-up, and calibration beads from the DAKO QIFIKIT kit (Dako #K0078, lot #20061434) were stained with anti-mouse IgG mAb-FITC per manufacturer's instructions and cells were analyzed on an Attune flow cytometer.

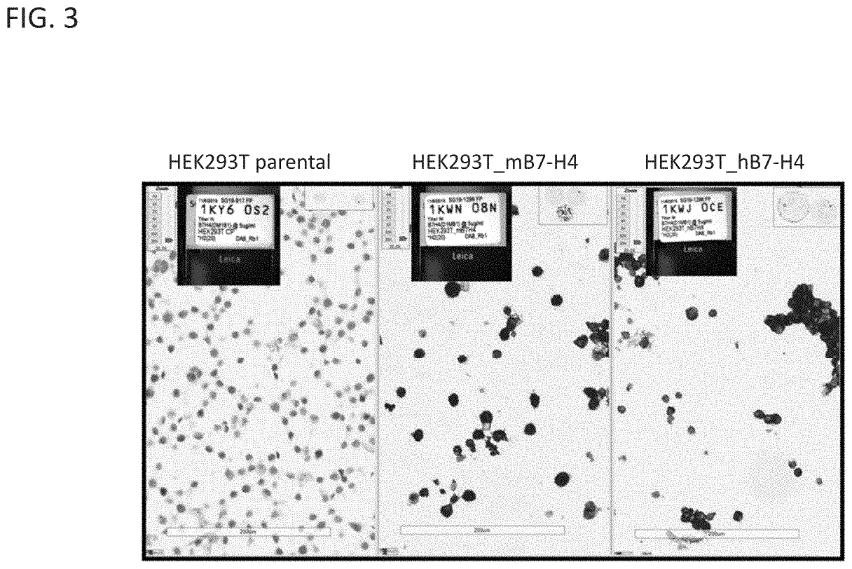

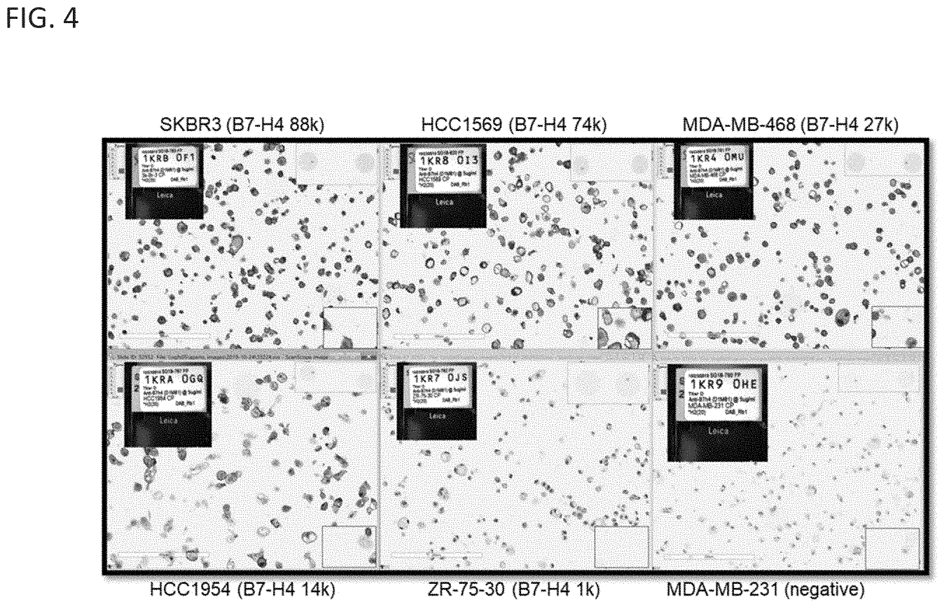

Staining was observed on B7-H4-expressing HEK293T cells, but not on B7-H4-negative parental HEK293T cells (FIG. 3). Moreover, differential staining was observed on tumor cells that endogenously expressed a range of B7-H4 on their cell surface (Table 9) with strong staining observed on SKBR3, HCC1569, and MDA-MB-468 cells (intensity score=3), moderate staining observed on HCC1954 cells (intensity score=2), and weak staining observed on ZR-75-30 cells (intensity score=1, FIG. 4). No staining was observed on the B7-H4-negative cell line MDA-MB-231 (FIG. 4). Altogether, this data indicates that the anti-B7-H4 antibody clone D1M8I selectively stains B7-H4-expressing cells.

TABLE 9
B7-H4 copy number on breast tumor cell lines
determined by quantitative flow cytometry

| Tumor cell line | B7-H4 copy number |
|---|---|
| MX-1 | 212,000 |
| SKBR3 | 88,000 |
| HCC1569 | 74,000 |
| MDA-MB-468 | 27,000 |
| HCC1954 | 14,000 |
| ZR-75-30 | 1,000 |
| MDA-MB-231 | negative |

Table 9 shows the B7-H4 copy number on breast tumor cell lines that endogenously expressed a range of B7-H4 levels as measured by quantitative flow cytometry.

### Example 4: IHC Staining for B7-H4 on Human Tumor Samples

Expression of B7-H4 in the following five indications was confirmed by immunohistochemical (IHC) staining of formalin-fixed paraffin-embedded (FFPE) tumor samples obtained from US Biomax or BioChain: Breast cancer, Ovarian cancer, Endometrial/uterine cancer, Cholangiocarcinoma, Lung cancer (adeno and squamous NSCLC).

IHC staining for B7-H4 on breast cancer, ovarian cancer, cholangiocarcinoma, non-small cell lung cancer (NSCLC), and endometrial cancer tissue microarrays (TMAs) was performed with rabbit IgG mAb clone D1M8I (Cell Signaling #14572). Freshly cut and unbaked formalin-fixed, paraffin-embedded (FFPE) TMAs were purchased from US Biomax Inc (BC11115c, BR1921c, BC09012b, EMC1501, EMC1502, LV1004a, LC706b, LC1923, LC808b, LC704) or BioChain (Z7020063). Slides were baked (Boekel Scientific, model 107800) for 1 hour at 58° C. immediately prior to the IHC run.

All samples were processed on a Bond-III™ autostainer (Leica Microsystems Inc., Buffalo Grove Ill.) at ambient temperature following the manufacturer's instructions. FFPE sections on glass slides were de-paraffinized using Bond™ Dewax solution (Leica, cat #AR9222) at 58-60° C. Antigen retrieval was performed using EDTA-based pH 9 Bond™ Epitope Retrieval Solution 2 (Leica, cat #AR9640) for 20 min at 98-100° C. The Peroxide Block was applied for 10 minutes followed by blocking of nonspecific background with Protein Block (Dako, cat #X090930) for 20 minutes. All antibodies were diluted to working concentration in BOND Primary Antibody Diluent (Leica, cat #AR9352). Isotype-matched rabbit IgG (Abcam, clone EPR25a cat #ab172730) was used as a negative control for background staining. For automated IHC staining we used the Bond™ Polymer Refine Detection (DAB) kit (Leica, cat #DS9800). Slides were incubated with rabbit monoclonal primary antibody against B7-H4 for 45 minutes at 5m/mL (primary antibody was dispensed twice for a total of 300 uL per TMA). Detection of HRP was done with DAB refine chromogen incubated for 10 minutes. Sections were counterstained with hematoxylin for 7 minutes.

Upon protocol completion on the autostainer, the slides were immediately removed and placed in deionized (DI) water before going through a series of dehydration steps (70% EtOH, 70% EtOH, 95% EtOH, 95% EtOH, 100% EtOH, 100% EtOH, 100% EtOH, Xylenes×3) to allow for cover slipping (Leica, CV5030) using Surgipath mounting medium (Leica, Surgipath Micromount, cat #3801731). Included in each IHC run was a B7-H4 positive control (HEK293T_B7-H4 over-expressing cell pellet) and a B7-H4 negative control (HEK293T parental cell pellet). Images were captured using a slide scanner (Leica, Aperio AT2) and slides and/or images were evaluated and scored by a pathologist.

Immunohistochemical staining for B7-H4 was also performed on untreated PDX tumors as follows:

Tumors were deparaffinized by incubation in: Xylene for 3 minutes (twice); 100% EtOH for 2 minutes (twice); 90% EtOH for 2 minutes; 100% EtOH for 2 minutes

Antigen retrieval was performed using a Decloaking Chamber NxGen Biocare and the 110° C. program with Diva Retrieval solution. Slides were stained on the IntelliPath Automated stainer per the following protocol: Removed hot containers from the decloaking chamber and rinse in deionized water; Placed slides in TB ST prior to adding to IntelliPath; Hydrated slides on stainer prior to automation; Added 300 uL/slide Peroxidaze and incubate 10 minutes; Washed slides in TBST; Added 300 uL/slide Sniper block and incubate 10 minutes; Blotted off blocking solution; Added 300 uL/slide primary antibody and incubate 1 hour; Washed slides twice in TBS; Added 300 uL/slide HRP polymer and incubate 30 minutes; Washed slides twice in TBST; Added 300 uL/slide DAB and incubate 5 minutes; Washed in TBST; Added 300 uL/slide hematoxylin and incubate 2 minutes; Washed in deionized water; Dehydrated slides by placing them in the oven for 30 minutes; Placed coverslip on slides

Images were analyzed using Halo image analysis software (Indica Labs). The H-score was calculated according to the following equation: (3×% Strong Signal)+(2×% Moderate Signal)+(% Weak Signal).

### Results

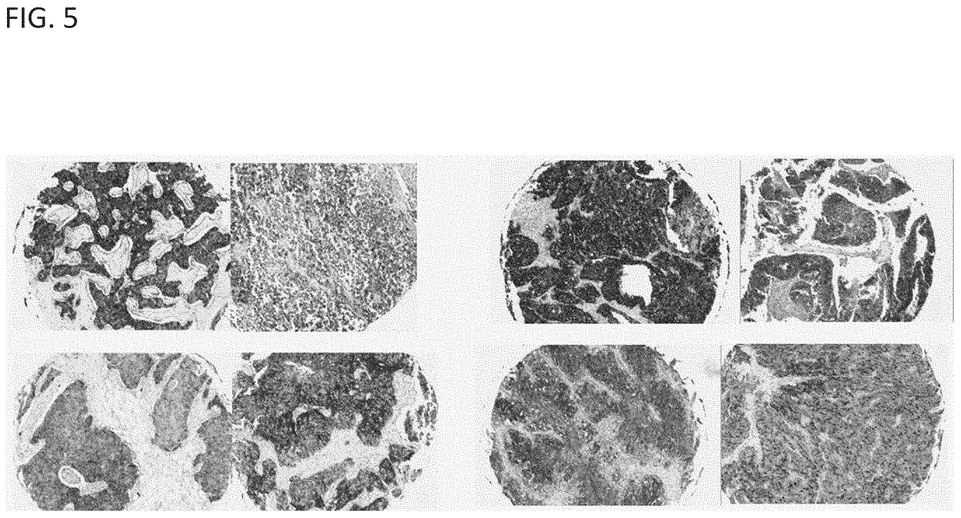

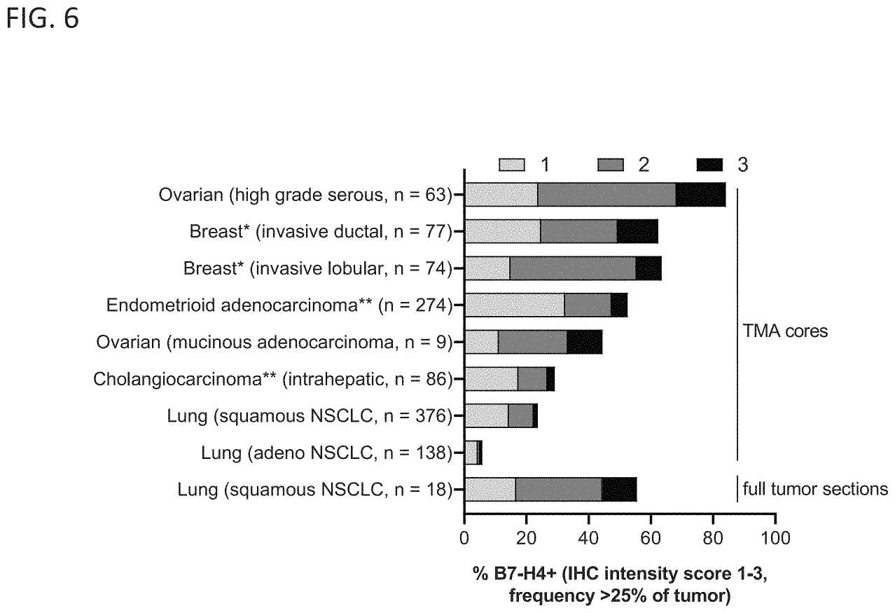

AS shown in FIG. 5, B7-H4 expression was observed on breast and ovarian tumor cores. FIG. 6 showed the summary of B7-H4 IHC scores on various tumor tissues. B7-H4 expression was observed across all three breast subtypes (Her2+, HR+, and TNBC), consistent with published data (Leong et al., 2015, Mol Pharm 12, 1717-1729; Sachdev, 2019, “Phase 1a/1b study of first-in-class B7-H4 antibody, B7H41001, as monotherapy in patients with advanced solid tumors,” presented at: ASCO (Journal of Clinical Oncology). In addition, all indications had uniform membrane staining except endometrioid adenocarcinoma and cholangiocarcinoma. For endometrioid adenocarcinoma and cholangiocarcinoma, both uniform and apical membrane staining patterns were observed. Tables 10 and 11 showed the summary of B7-H4 staining on TMAs and squamous NSCLC tumors, respectively.

TABLE 10
Summary of B7-H4 staining on TMAs

| | | Prevalence | | |
|---|---|---|---|---|
| | | (# of cores | Localization | |
| Indication | Tumor type | positive/total) | quantified | TMA ID# |
| Ovarian | High grade | 84% | M | BC11115c |
| | serous | (53/63) | | |
| Ovarian | Mucinous | 44% | M | BC11115c |
| | adenocarcinoma | (4/9) | | |
| Breast | Invasive ductal | 62% | M | BR1921c |
| | | (48/77) | | |
| Breast | Invasive lobular | 64% | M | BR1921c |
| | | (47/74) | | |
| Uterine | Endometrioid | 58% | M and/or A | BC09012b |
| | adenocarcinoma | (31/53) | | |
| Uterine | Endometrioid | 47% | M and/or A | EMC1501 |
| | adenocarcinoma | (53/112) | | |
| Uterine | Endometrioid | 55% | M and/or A | EMC1502 |
| | adenocarcinoma | (60/109) | | |
| Cholangiocarcinoma | Intrahepatic | 29% | M and/or A | LV1004a |
| | | (25/86) | | |
| Lung | Adeno NSCLC | 7% | M | LC706b |
| | | (5/71) | | |
| Lung | Adeno NSCLC | 4% | M | LC1923 |
| | | (3/67) | | |
| Lung | Squamous NSCLC | 32% | M | LC808b |
| | | (24/75) | | |
| Lung | Squamous NSCLC | 22% | M | LC808b |
| | | (17/78) | | |
| Lung | Squamous NSCLC | 23% | M | LC704 |
| | | (17/75) | | |
| Lung | Squamous NSCLC | 15% | M | Z7020063 |
| | | (11/71) | | |
| Lung | Squamous NSCLC | 26% | M | LC1923 |
| | | (20/77) | | |

For TMAs, formalin-fixed paraffin-embedded tumors were stained for B7-H4 using mAb clone D1M8I (CST). Slides were scored as follows: Intensity: 0=none, 1=weak, 2=moderate, 3=strong; Frequency: 1=1-25%, 2=26-50%, 3=51-75%, 4=>75%. For prevalence calculation, tumors were considered positive if membrane (M) and/or apical membrane (A) staining (any intensity) was observed on greater than 25% of tumor cells.

TABLE 11
Summary of B7-H4 staining on squamous NSCLC tumors

| | | | | | | | B7-H4 | B7-H4 | |
|---|---|---|---|---|---|---|---|---|---|
| Tumor | Patient Metadata | | | | | | Intensity | Frequency | |
| ID# | Sex | Age | Grade | TNM | Stage | Metastases | (0-3) | (0-4) | Localization |
| 1 | F | 68 | G2 | T1aN0M0 | IA | 0/8 lymph | 2 | 3 | M/C |
| | | | | | | nodes | | | |
| 2 | M | 58 | G2 | T3N1M0 | IIIA | 1/12 lymph | 2 | 1 | M/C |
| | | | | | | nodes | | | |
| 3 | M | 66 | G2 | T2bN2M0 | IIIA | 7/19 lymph | 3 | 4 | M/C |
| | | | | | | nodes | | | |
| 4 | M | 53 | G2-3 | T3N1M0 | IIIA | 4/14 lymph | 2 | 2 | M/C |
| | | | | | | nodes | | | |
| 5 | M | 68 | G3 | T2aN1M0 | IIA | 1/16 lymph | 1-2 | 1 | M/C |
| | | | | | | nodes | | | |
| 6 | M | 66 | G2 | T2bN0M0 | IIA | 0/24 lymph | 3 | 4 | M/C |
| | | | | | | nodes | | | |
| 7 | M | 66 | G2 | T2aN1M0 | IIA | 2/51 lymph | 2 | 3 | M/C |
| | | | | | | nodes | | | |
| 8 | F | 66 | G3 | T3N0M0 | IIB | 0/16 lymph | 1 | 3 | M/C |
| | | | | | | nodes | | | |
| 9 | M | 67 | G2 | T3N1M0 | IIIA | 5/24 lymph | 0 | 0 | not applicable |
| | | | | | | nodes | | | |
| 10 | M | 60 | G2 | T3N1M0 | IIIA | 3/12 lymph | 1-2 | 3 | M/C |
| | | | | | | nodes | | | |
| 11 | M | 65 | G3 | T3N1M1a | IV | 1/7 lymph | 2 | 3 | M/C |
| | | | | | | nodes | | | |
| 12 | M | 63 | G3 | T2bN0M0 | IIA | 0/15 lymph | 1-2 | 1 | M/C |
| | | | | | | nodes | | | |
| 13 | M | 56 | G3 | T4N1M0 | IIIA | 6/12 lymph | 2 | 1 | M/C |
| | | | | | | nodes | | | |
| 14 | F | 81 | G4 | T3N1M0 | IIIA | 3/12 lymph | 1 | 1 | C |
| | | | | | | nodes | | | |
| 15 | M | 56 | G2 | T2aN2M0 | IIIA | 4/16 lymph | 2 | 2 | M/C |
| | | | | | | nodes | | | |
| 16 | M | 72 | G2 | T3N1M0 | IIIA | 1/22 lymph | 1-2 | 3 | M/C |
| | | | | | | nodes | | | |
| 17 | M | 54 | G3 | T3N1M0 | IIIA | 3/26 lymph | 2 | 1 | M/C |
| | | | | | | nodes | | | |
| 18 | F | 65 | G3 | T2aN2M0 | IIIA | 11/22 lymph | 1 | 1 | M/C |
| | | | | | | nodes | | | |

For NSCLC, formalin-fixed paraffin-embedded tumors were stained for B7-H4 using mAb clone D1M8I (CST). Slides were scored as follows: Intensity: 0=none, 1=weak, 2=moderate, 3=strong; Frequency: 1=1-25%, 2=26-50%, 3=51-75%, 4=>75%; Localization: M=membrane, C=cytoplasm. Staining was not observed on serial tumor sections stained with a rabbit IgG isotype control mAb (clone EPR25A, Abcam).

B7-H4 is a member of the B7 family of immune checkpoint ligands whose expression is elevated on a variety of solid tumors. The presence of B7-H4 expression had been confirmed in this example in a variety of carcinoma-derived patient samples, including breast, ovarian, endometrial, cholangiocarcinoma, and NSCLC tumors.

### Example 5: SGN-B7H4V and B7H41001 mAb Binding to B7-H4

The fully human, fucosylated anti-B7-H4 antibody B7H41001 mAb was identified using an in vitro yeast display platform (Kaplan, 2017) and conjugated to the protease cleavable MMAE/SGD-1006 (vedotin) drug linker to form SGN-B7H4V. SGN-B7H4V and the unconjugated antibody, B7H41001 mAb, were evaluated for binding to recombinant human B7-H4 protein (hB7-H4; B7-H4 extracellular domain Phe29-Ala258 with a C-terminal 10-His tag) by biolayer interferometry (BLI).

### BLI Evaluation of SGN-B7H4V and B7H41001 mAb Binding to Recombinant B7-H4

For monovalent binding studies, SGN-B7H4V and B7H41001 mAb were diluted in kinetic buffer (0.1% BSA, 0.02% Tween20, 1×PBS pH 7.4) and loaded at 4 μg/mL for 300 seconds onto AHC (anti-human Fc) biosensors (from ForteBio). After a baseline in kinetic buffer, the hB7-H4 His antigen was serially diluted to 2.14, 5.96, 16.61, 46.3, 128.9, and 359 nM with kinetic buffer and associated for 450 seconds, followed by a 1000 second dissociation step in kinetic buffer. Sensorgrams were generated on an Octet HTX system (ForteBio) at 30° C. and globally fitted with the 1:1 Langmuir isotherm model (Rmax unlinked) after a reference subtraction of the antigen-loaded, 0 nM analyte sensor. A negative control with nothing immobilized and a high concentration of the hB7-H4 His antigen (1000 nM) was performed to verify the absence of nonspecific binding of the analyte to the AHC biosensors themselves. The fitting windows of the dissociation times for each interaction are noted in the affinity table within the results section.

For bivalent binding studies, NHS-biotinylated hB7-H4 His antigen was diluted in kinetic buffer and loaded at 0.25 μg/mL for 300 seconds onto SAX (streptavidin) biosensors (from ForteBio). After a baseline in kinetic buffer, SGN-B7H4V and B7H41001 mAb were serially diluted to 0.2, 0.51, 1.28, 3.2, and 8.0 nM with kinetic buffer and associated for 600 seconds, followed by a 2000 second dissociation step in kinetic buffer. Sensorgrams were generated on an Octet HTX system (ForteBio) at 37° C. and globally fitted with the 1:1 Langmuir isotherm model (Rmax unlinked) after a reference subtraction of the antigen-loaded, 0 nM analyte sensor.

### Results.

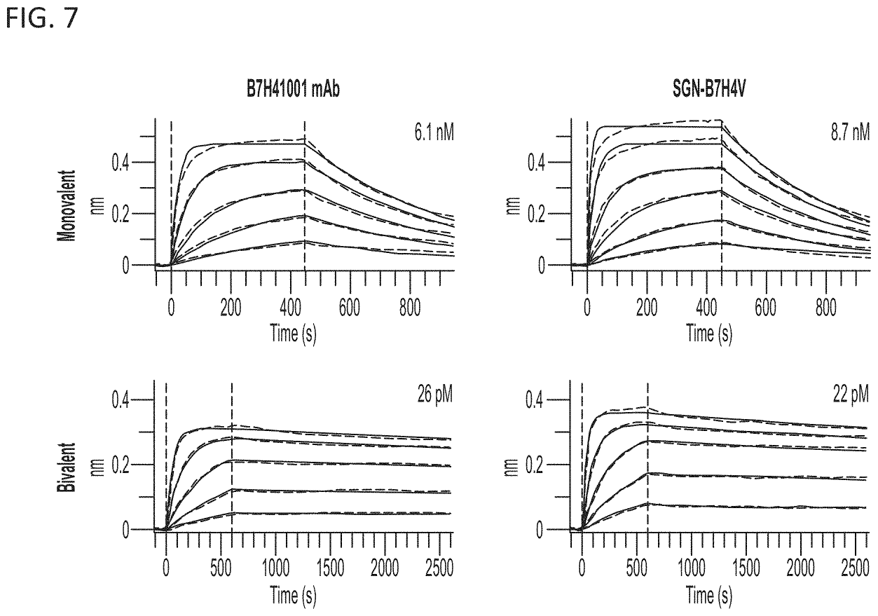

The binding affinities for hB7-H4 were similar between B7H41001 mAb and SGN-B7H4V (FIG. 7), suggesting the conjugation process did not alter the binding affinity of the B7H41001 mAb. The bivalent format enhanced affinity by 231 and 395-fold for mAb and ADC, respectively, compared to the monovalent format.

### Example 6: SGN-B7H4V and B7H41001 mAb Binding to B7-H4

SGN-B7H4V and the unconjugated antibody, B7H41001 mAb, were evaluated for binding to SKBR3 cells, which endogenously express human B7-H4.

### Saturation Binding Studies of SGN-B7H4V and B7H41001 mAb

After lifting with TrypLE Express and counting the cells, B7-H4-expressing SKBR3 cells were resuspended in 1 mL of FACS Wash Buffer with 5% mouse serum (Sigma #M5905) and incubated at room temperature for 5 minutes. SKBR3 cells were then diluted to 2 million cells/mL with FACS Wash Buffer and 100,000 cells/well were plated into a U-bottom plate (50 μL/well). Then, antibodies were titrated at a 1:3 dilution in FACS Wash Buffer with a starting concentration of 700 nM and diluted down to 0.004 nM (actual starting concentration is 1400 nM to account for 2x dilution in the well). Antibody titrations were plated over the SKBR3 cells at 50 μL/well in duplicate. The test plate was incubated for 20 minutes at room temperature and then another 35-45 minutes at 4° C. After the ˜1 hour incubation, the cells were washed 2 times with FACS Wash Buffer and secondary antibody (goat anti-human IgG (H+L)-PE, Jackson ImmunoResearch, #109-116-170) was added to the cells at a 1:200 dilution in FACS Wash Buffer (100 μL/well). The cells were incubated at room temperature for 20 minutes and then washed twice with FACS Wash Buffer. Prior to analyzing the cells on the Cytoflex Flow cytometer, the cells were resuspended in 100 μL of FACS Wash Buffer. The data was exported to FlowJo software and median fluorescent intensity (MFI) statistic was used to graph the results in GraphPad Prism software.

### Results

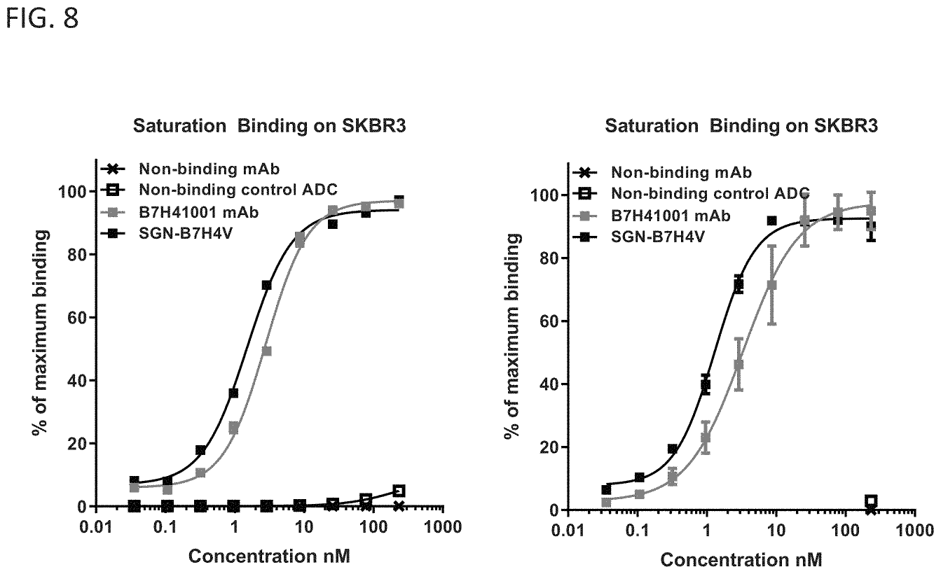

Saturation binding studies demonstrated that B7H41001 mAb and SGN-B7H4V bound to B7-H4 with comparable Kd values of ˜3 nM and ˜1.5 nM, respectively (FIG. 8). Binding of B7H41001 mAb and SGN-B7H4V was selective for B7-H4 as non-binding mAb and control ADC showed minimal binding to SKBR3 cells.

Collectively, the results in Example 4 and Example 5 suggest that SGN-B7H4V binds selectively to B7-H4 with high affinity and support its further evaluation as a therapeutic in solid tumors that express the antigen.

### Example 7: Cellular Internalization of the B7H41001 mAb In Vitro

Automated immunofluorescence was used to visualize internalization properties of the SGN-B7H4V antibody backbone B7H41001 mAb in a cell-based assay.

### B7H41001 mAb Internalization by Automated Fluorescence Microscopy.

Intracellular trafficking was performed on the B7-H4-expressing breast cancer cell lines SKBR3 and MX-1 by automated fluorescence microscopy (IncuCyte S3, Essen Bioscience). To evaluate internalization, the B7H41001 antibody was conjugated to a Cy5 dye and quencher pair linked using the same vc linker as in SGN-B7H4V. The quencher prevented the dye from emitting fluorescence until the antibody was internalized and the dye was cleaved away from the antibody and quencher. In the absence of expression of the specific antigen, no internalization occurred, and the fluorescence intensity of the labeled antibodies remained low. Specifically, The B7H41001 mAb was conjugated with a quenched fluorophore with a linker identical to the cleavable linker in the vc-PAB-MMAE drug linker that is used in SGN-B7H4V. The quencher prevented the dye from emitting fluorescence until the antibody was internalized and the dye was cleaved away from the antibody and quencher.

Cells were seeded at ˜2500 cells per well in 96-well flat clear bottom black-walled tissue culture-treated microplates (Corning #3603) and left to adhere overnight at 37° C. Quenched fluorophore antibody conjugates were diluted in culture medium and added to cells at 1 μg/mL (final concentration). Plates were immediately loaded onto microplate trays in the IncuCyte S3 in a 37° C. incubator. Scans were acquired using the Adherent Cell-by-Cell protocol. Phase data and red channel data (acquisition time set to 400 ms) were collected, with 4 images per well, every 2 to 6 hours for up to 24 hours with the objective set at 10×. Quantification of quenched fluor signal intensity was performed using the IncuCyte software analysis tool. The analysis was refined and tuned per cell line utilizing a label-free cell count and manual image selection for preview and training of the algorithm. Upon completion of analysis, data was calculated using the IncuCyte software with graph metrics set to red mean intensity object average normalized to time 0(%), thus providing a measurement of the red (quenched fluor) mean intensity per cell at a given time point normalized to the data obtained at time 0. Normalized mean red intensity per cell was fit to a single exponential “one-phase association” equation using Graphpad Prism (San Diego, Calif.) to determine apparent “half-time” (t½) values for the activation of the quenched fluorophore, a proxy for the internalization and endolysosomal trafficking of SGN-B7H4V.

### Results

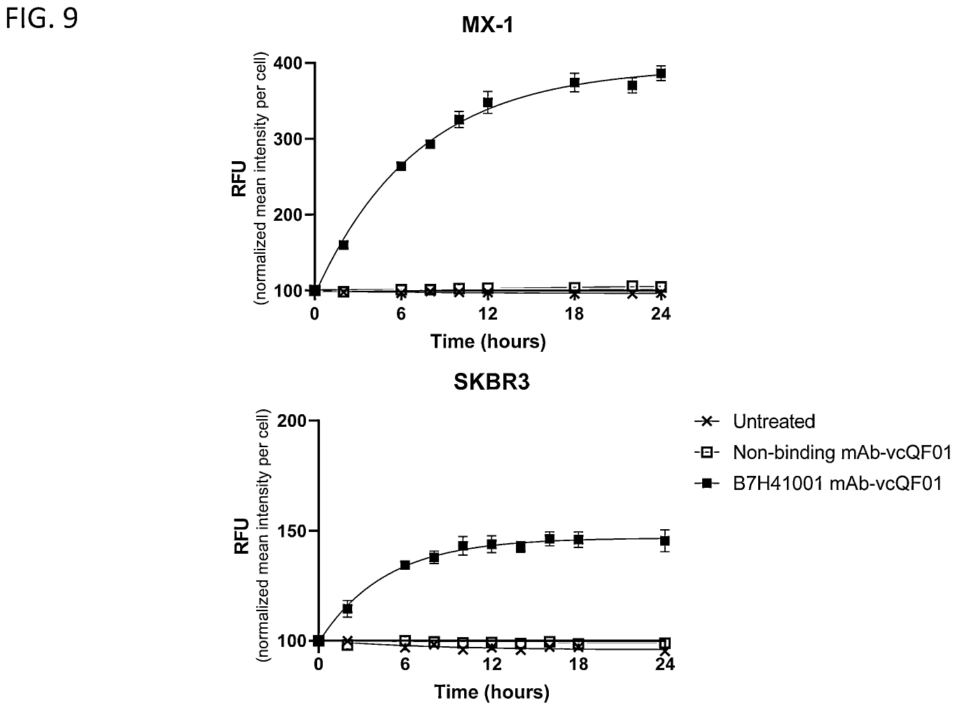

The B7H41001 mAb quenched fluorophore conjugate was incubated with cell lines that express B7-H4 endogenously (SKBR3 and MX-1). Fluorescence was then quantified by imaging cells every 2 to 6 hours and calculating the mean red fluorescence intensity per cell. The fluorescent signal in this assay increased with an apparent half-life ranging from approximately 3.2 to 4.9 hours depending upon the cell line (FIG. 9). The maximal fluorescent signal was higher on MX-1 cells, consistent with higher surface expression of B7-H4 on MX-1 cells compared to SKBR3 cells (see Table 9). Importantly, internalization was dependent on binding to B7-H4; minimal fluorescence was detected when cells were incubated with the non-binding mAb quenched fluorophore conjugate.

### Example 8: In Vitro Cytotoxicity of SGN-B7H4V

The ability of SGN-B7H4V to elicit cytotoxicity in a 96-hour in vitro assay was determined in three B7-H4-expressing cell lines (SKBR3, MX1, and MDA-MB-468) and one non-B7-H4-expressing cell line (MDA-MB-231). The cytotoxicity was evaluated when cells were grown in 3D spheroid (round bottom, ultra-low attachment plates) conditions.

Cancer cell lines expressing B7-H4 (SKBR3, MX-1, and MDA-MB-468) as well as a non-B7-H4-expressing cell line (MDA-MB-231) were thawed from cryovials stored at −210° C. into complete growth media and allowed to grow and recover from thaw at 37° C. and 5% CO2 until cell viability determined by Vi-CELL XR (Beckman Coulter, Indianapolis, Ind.) were above 90%. Cells were then counted and plated at 2000, 2200, 2200, and 2200 cells/well respectively. Cells were plated in 150 μL complete growth media in round bottom, black walled, ultra-low attachment, 96-well plates (Corning 4520). Cell plates were placed at 37° C. and 5% CO2 overnight to allow cells to adhere. ADCs were thawed and 4×8-point serial dilutions were prepared (final dose range 1000-0.061 ng/mL) in RPMI 1640+20% FBS. Fifty μL of each dilution were then added to each cell plate in triplicate. Cells were then left to incubate at 37° C. and 5% CO2 for 96 hours. Cell plates were then removed from the incubator and allowed to cool to room temperature for 30 minutes. CellTiter-Glo® luminescent assay (Promega Corporation, Madison, Wis.) was prepared according to Promega's protocol. One hundred uL CellTiter-Glo® were added to assay plates using a Formulatrix Tempest liquid handler (Formulatrix, Inc.) and plates were heat sealed using an ALPS300 automated microplate heat sealer (Thermo Scientific) and protected from light for 30 minutes at room temperature. The luminescence of each plate was then determined using an EnVision Multilabel plate reader (Perkin Elmer, Waltham, Mass.). For spheroid culture plates, after 15 minutes of incubation with CellTiter-Glo®, wells were mixed using a multichannel pipette to ensure complete lysis of spheroids. One hundred uL of lysed spheroid suspension was then transferred to a black walled, flat bottom 96 well plate and read on the EnVision Multilabel plate reader (Perkin Elmer, Waltham, Mass.). Raw data were then analyzed in Graphpad Prism (San Diego, Calif.) using a non-linear, 4-parameter curve fit model (Y=Bottom+[Top-Bottom]/[1+10\hat{ }[[Log EC50-X]×HillSlope]]). Results are reported as X50 values defined as the concentration of ADC required to reduce cell viability to 50%.

### Results

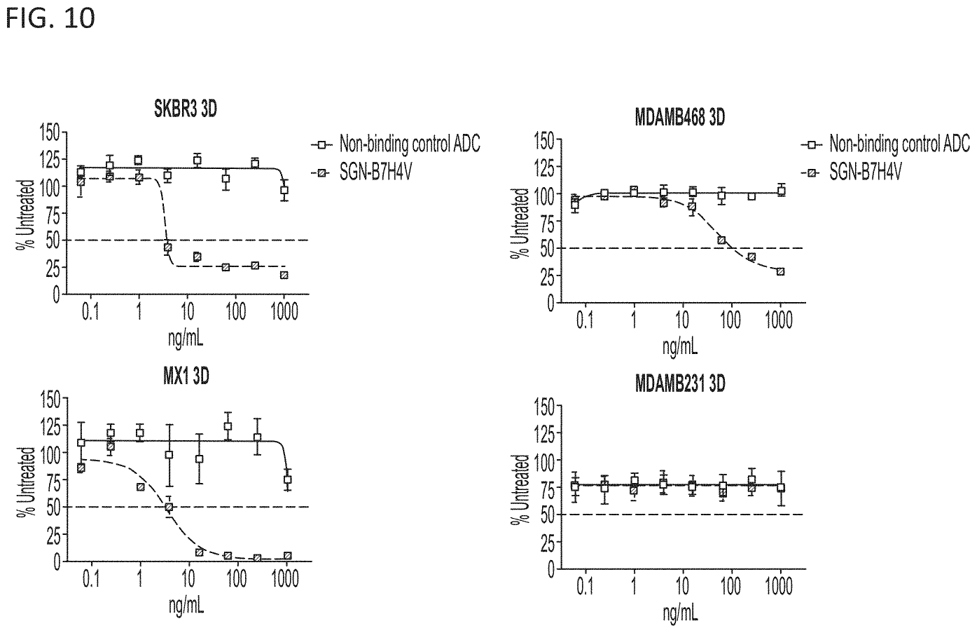

As shown in FIG. 10, all three of the B7-H4-expressing cell lines exhibited sensitivity to SGN-B7H4V, but not the non-binding control ADC with ×50 values ranging from 3 to 105 ng/mL (Table 12).

Here, we evaluated the internalization properties of the unconjugated antibody component as well as the cytotoxic activity of SGN-B7H4V. We found that the B7H41001 mAb binds to B7-H4-expressing tumors cells and internalizes into the intracellular compartment. SGN-B7H4V exerts potent cytotoxic activity on B7-H4-expressing cells in vitro. Collectively, these results suggest that SGN-B7H4V can deliver the cytotoxic payload MMAE to cells that express B7-H4 and support its further evaluation as a therapeutic in solid tumors that express the antigen.

TABLE 12
In vitro cytotoxicity of SGN-B7H4V (×50 values)

| | ×50 (ng/mL) | | | |
|---|---|---|---|---|
| | SKBR3 | MX1 | MDA-MB-468 | MDA-MB-231 |
| SGN-B7H4V | 4 | 3 | 105 | >1000 |
| Non-binding | >1000 | >1000 | >1000 | >1000 |
| control ADC | | | | |

### Example 9: SGN-B7H4V Fc Effector Function

Human activating FcγRs are divided into three types, FcγRI (CD64), FcγRIIa (CD32a), and FcγRIII (CD16). Upon interaction of the Fc region of the IgG1 antibody backbone with activating FcγRs on innate immune cells, such as monocytes and macrophages, a signaling cascade is triggered to elicit effector functions including ADCC, ADCP, and CDC. NK cells mediate ADCC via FcγRIII, while monocytes/macrophages are thought to mediate ADCP primarily via FcγRI/IIa. To characterize the ability of SGN-B7H4V and the unconjugated mAb B7H41001 to induce Fc effector functions, FcγR binding and cellular FcγR signaling were measured. The ability of SGN-B7H4V and B7H41001 mAb to elicit ADCC, ADCP, and CDC was also evaluated directly in primary cell-based assays.

### BLI Binding Assay

Binding kinetics with hFcγRI, hFcγRIIa H131, hFcγRIIa R131, hFcγRIIIa F158, hFcγRIIIa V158, and hscFcRN were assessed by BLI. Biotinylated avi-tagged human Fc Receptors fused with monomeric Fc (designed and expressed at Seagen, Inc) were loaded onto high precision streptavidin biosensors (from ForteBio) to responses around 0.4 nm for all receptors except for hFcγR1 with responses around 1.2 nm. An initial baseline was completed in immobilization buffer (0.1% BSA, 0.02% Tween20, 1×PBS pH 7.4) followed by a second baseline in kinetic buffer (1% casein, 0.2% Tween20, 1×PBS pH 7.4 for hFcγRI, IIa, IIIa, and IIb interactions and 1% BSA+0.2% Tween20, Phosphate Citrate pH 6.0 for hscFcRN interactions). Titrated SGN-B7H4V, B7H41001 mAb, and positive control mAb samples were associated and dissociated for: 600 s and 1000 s for hFcγRI, 10 s and 50 s for hFcγRIIa and hFcγRIIb, 60 s and 200 s for hFcγRIIIa, and 50 s and 200 s for hscFcRN in kinetic buffer. Sensorgrams were generated on an Octet HTX system (ForteBio) at 30° C. and globally fitted with the 1:1 kinetic Langmuir isotherm model (Rmax unlinked) after a reference subtraction of the antigen-loaded, 0 nM analyte sensor. Negative controls with the highest concentration of antibodies and ADCs (20 μM) with no Fc receptor immobilized were also performed to verify the absence of nonspecific binding of the analyte to the streptavidin biosensors themselves. Specific loading concentrations and times of each receptor to the streptavidin sensors, and concentrations of titrated analytes are listed (Table 13, Table 14).

TABLE 13
Immobilization concentrations and
times onto streptavidin biosensors

| | |
|---|---|
| hFcγI hmFc AAG A avi Biotin | (3.0 ug/mL, 400 s load) |
| hFcγRIIa H131 hmFc AAG avi Biotin | (0.7 ug/mL, 300 s load) |
| hFcγRIIa R131 hmFc AAG avi Biotin | (1.7 ug/mL, 300 s load) |
| hFcγRIIIa F158 hmFc AAG avi Biotin | (4.0 ug/mL), 300 s load) |
| hFcγRIIIa V158 hmFc AAG avi Biotin | (3.0 ug/mL, 300 s load) |
| hFcγRIIb hmFc AAG avi Biotin | (2.0 ug/mL, 300 s load) |
| hscFcRN hmFc IHH A avi Biotin | (7.0 ug/mL, 300 s load) |

TABLE 14
Concentrations of analytes

| | |
|---|---|
| B7H41001 mAb, SGN-B7H4V, and | 66.7, 22.2, 7.4, 2.47, 0.82, |
| Positive Control mAb with hFcγRI | 0.27 nM |
| B7H41001 mAb, SGN-B7H4V, and | 20, 8.57, 3.67, 1.57, 0.67, |
| Positive Control mAb with hFcγRIIa, | 0.29, 0.12 μM |
| IIIa, and IIb | |
| B7H41001 mAb, SGN-B7H4V, and | 500, 184.2, 67.9, 25, 9.21, |
| Positive Control mAb with hFcRN | 3.39, 1.25 nM |

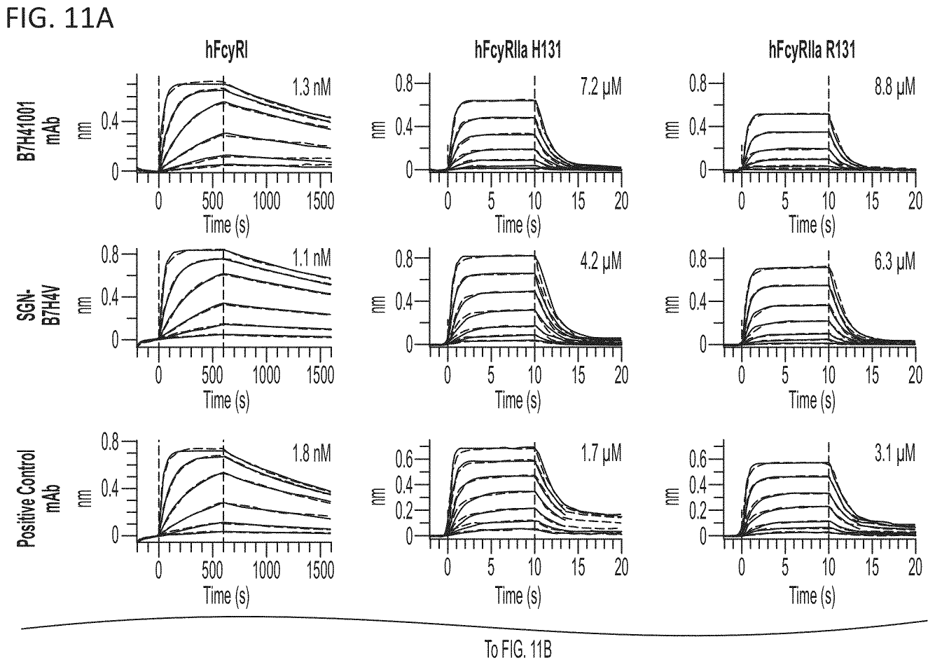

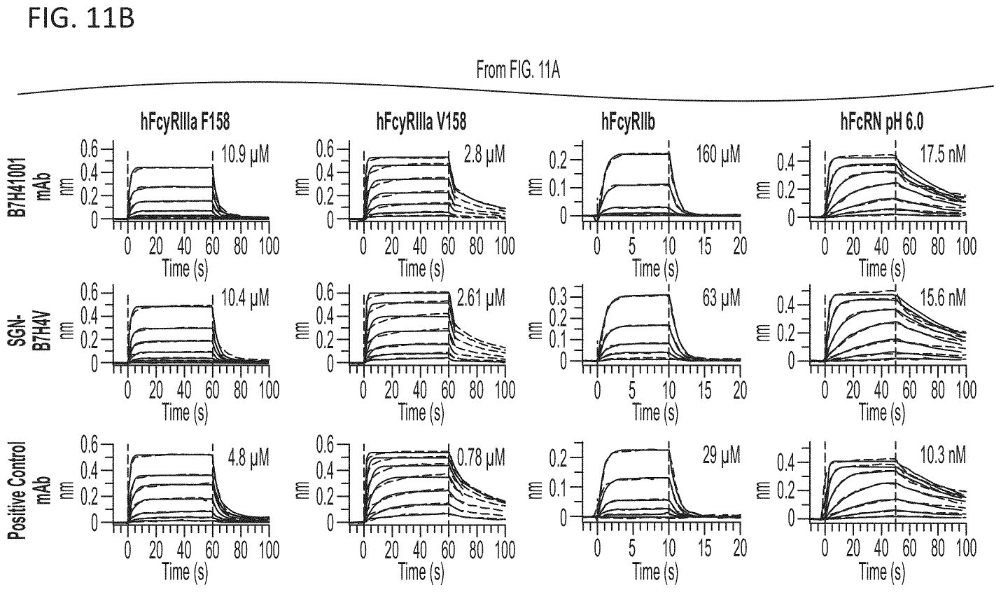

As shown in FIG. 11A and FIG. 11B, SGN-B7H4V and B7H41001 mAb bound similarly to all human Fc receptors tested. hFcγRI had the tightest affinity, around 1 nM, and hFcRN had the second tightest affinity at an average of 17 nM. The affinities for hFcγRIIIa and hFcγRIIa variants ranged from 2.6-10.9 μM and hFcγRIIb showed the weakest affinity. The data quality for hFcγRIIb interactions were low, which caused high degrees of variability due its inability to reach above 50% saturation even with the highest concentration set at 20 μM because the affinities were too weak. Even with the lowered data quality, the similar appearance of the sensorgrams infer that the affinities between the B7H41001 mAb and SGN-B7H4V were similar. Compared to the positive control mAb (the anti-CD70 mAb h1F6), SGN-B7H4V and B7H41001 mAb had slightly weaker affinities for Fc receptors, except for hFcγR1, for which they had similar binding as the positive control.

### FcγR Jurkat Reporter Cell Assay

Cellular FcγR signaling was measured in a cell-based assay that uses SKBR3 target cells that endogenously express B7-H4 and Jurkat effector cells engineered to express FcγRI, RIIa, or RIII and an NFAT (nuclear factor of activated T cells) driven luciferase reporter gene. Binding of the B7H41001 mAb Fab domain to B7-H4 on the target cells and the Fc domain to FcγR on the effector cells results in the induction of a luciferase signal. The luciferase signal is proportional to the degree of FcγR-induced effector cell activation and serves as a surrogate for ADCC (FcγRIII) or ADCP (FcγRI/IIa).

Specifically, Jurkat effector cells engineered to express FcγRI, RIIa, or RIII and an NFAT (nuclear factor of activated T cells) driven luciferase reporter gene were thawed and cultured in NFAT reporter cell medium (RPMI 1640 Medium (Gibco #11875-093) supplemented with 4% HyClone™ Fetal Bovine Serum, Super Low IgG (Gibco #A33819-01), 1× Penicillin-Streptomycin (Gibco #15140-122), 1×MEM nonessential amino acids (Gibco #11140-050), 1× L-Glutamine (Gibco #25030-081), 1× Sodium pyruvate (Gibco #11360-070), Hygromycin (Invitrogen #10687), Antibiotic G-418 sulfate solution (Promega #V8091), HEPES (Gibco #15630)). The Jurkat FcγR signaling assay was then performed as follows:

The day before the assay, target SKBR3 cells were lifted using Versene (Gibco #15040-066), washed twice with PBS, and plated at 1.2×10⁴ cells per well in 90 μL RPMI 1640 containing 10% low IgG FBS and penicillin/streptomycin in black-walled 96-well plates (Corning #3603). The following day, NFAT assay buffer was prepared by adding 4 mL low IgG serum to 100 mL of RPMI 1640, mixed, and warmed to 37° C. NFAT assay buffer was used to resuspend all cells and antibody dilutions. Stock dilution plates (10×) of each antibody were prepared at 100, 30, 10, 3, 1, 0.3, 0.1, 0.03, 0.01, and 0.003 μg/mL. 10 μL of each dilution was added to appropriate wells in plate with target cells (1:10 dilution). Cells were incubated at ambient temperature for 30 minutes. During incubation, cultured effector cells (Jurkats—FcγRI, FcγRIIa, or FcγRIII NFAT reporter cells) were washed twice in 1×PBS. Effector cells were counted, washed twice in PBS, and resuspended at 1×106 cells/mL in NFAT assay buffer. Assay buffer and antibody dilutions on target cells were carefully aspirated. 75 μL of effector cells were pipetted into each well (7.5×104 cells per well). The reporter cell assay plates were incubated at 37° C., 5% CO2 for 14-16 hours (FcγRI and FcγRIIa reporter cells) or 7 hours (FcγRIII reporter cells). After incubation, plates were equilibrated to ambient temperature for 15-30 minutes. Bio-Glo (G7941, Promega) reagent was thawed and resuspended according to manufacturer's instructions. 75 μL of Bio-Glo (Bio-Glo™ Luciferase Assay System, Promega #G7940) was added to each well, including to 3 wells with only medium for background control. Luminescence was measured for all samples with the Envision 96 CTG protocol (Envision plate reader, PerkinElmer) after mixing on a shaker (covered with foil) for at least 5 minutes. Background (no cells) signal was subtracted from the raw luminescence signal prior to graphing.

### Results

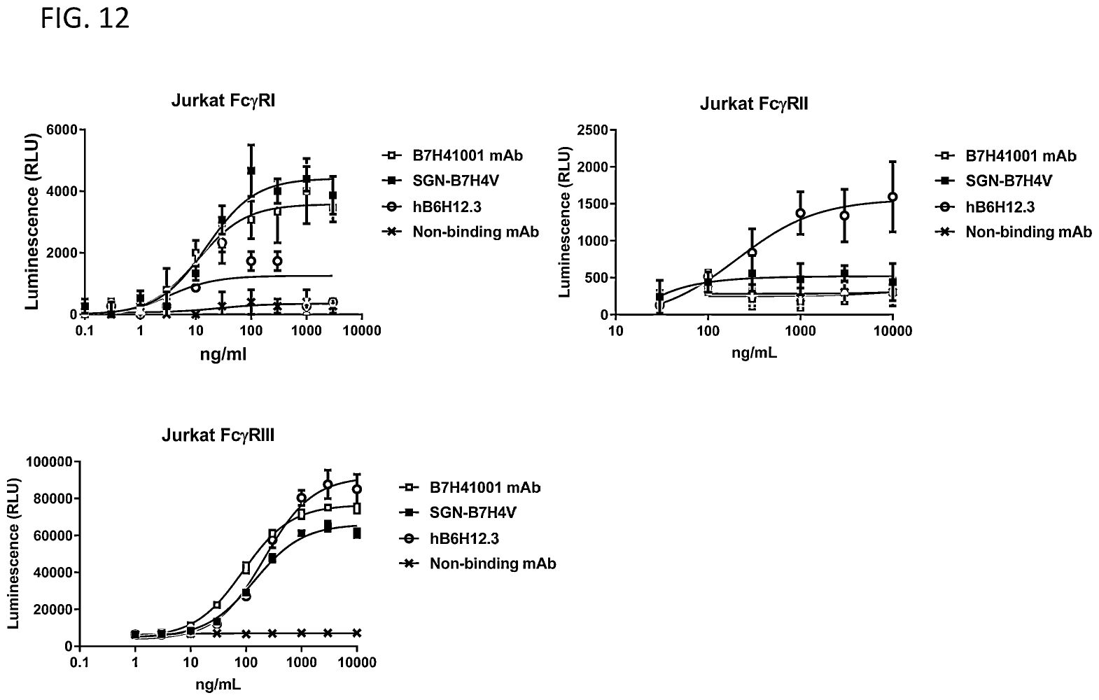

As shown in FIG. 12, SGN-B7H4V and B7H41001 mAb induced a dose-dependent increase in FcγRI and FcγRIII, but not FcγRIIa-mediated luciferase activity.

### ADCC

Antibodies directed to cell surface antigens can elicit direct killing of antibody-coated cells, including induction of ADCC. The ability of an antibody to drive ADCC is reliant on the antibody backbone, with human IgG1 antibodies being most active. Natural killer (NK) cell-mediated ADCC by SGN-B7H4V, the unconjugated B7H41001 mAb, as well as a non-binding control ADC and mAb were evaluated using the human B7-H4-expressing cell lines SKBR3, MX-1, and 293T-B7-H4.

Human PBMCs were thawed into pre-warmed R10+ Media (RPMI 1640 (Gibco #11875-093) with 10% HI-FBS (Gibco #16140-071), 1× sodium pyruvate (Gibco #11360-070), and 1× GlutaMax (Gibco #35050-061)) and then an EasySep NK isolation kit (StemCell #17955) was used to purify NK cells. Target tumor cells were lifted off using TrypLE Express (Gibco #12604-021) and then plated into a U-bottom plate (Falcon #353077) at 40,000 cells/well (50 μL/well) for MX-1 and 293T-B7-H4 (HEK 293T cells engineered to express human B7-H4) and 20,000 cells/well (50 μL/well) for SKBR3 cell line. SGN-B7H4V and B7H41001 mAbs were titrated with a 10× dilution ranging from 2000 ng/mL down to 0.02 ng/mL and then plated into the U-bottom plate at 50 uL/well. (NOTE: actual starting concentration was 6000 ng/mL to account for the inherent 3× dilution of the assay.) Then 200,000 cells/well (50 μL/well) of isolated NK cells were plated into the U-bottom plate (effector: tumor ratios were as follows: NK:SKBR3 cells 10:1; NK:MX1 cells 5:1, NK:293T-B7-H4 cells 5:1). Appropriate controls included: positive control mAb and ADC and negative non-binding control mAb and ADC, NK cells only control, target cells only control, target cells maximum lysis control, and media only control. The assay plate was incubated for 4 hours at 37° C. in a humidity-controlled incubator; 45 minutes prior to the ending of the incubation, lysis solution (from cytotoxicity kit below) was added to target cells maximum lysis control wells. Then, the assay plate was spun down and 50 uL of supernatant from each well was transferred to a new F-bottom clear plate (VWR #29442-058/3598). A CytoTox 96 Non-Radioactive Cytotoxicity Assay kit (Promega #G1780) was used to develop the signal, which was read using 490 nm wavelength on a SpectraMax 190 instrument.

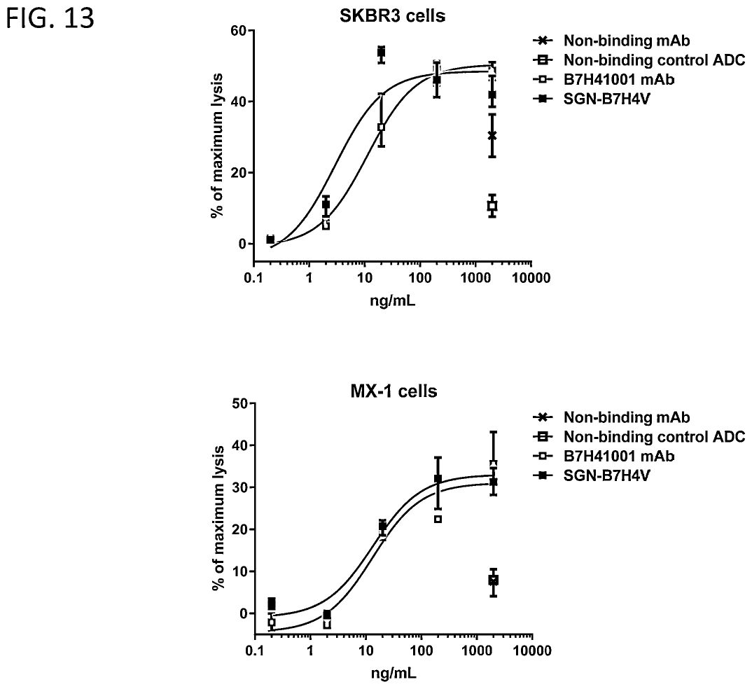

As shown in FIG. 13, both SGN-B7H4V and the unconjugated antibody B7H41001 mAb, but not the non-binding control ADC and mAb, elicited comparable ADCC responses.

### ADCP

The B7-H4-expressing cell line SKBR3 and primary monocytes/macrophages were used to evaluate SGN-B7H4V-mediated ADCP. SKBR3 cells were pre-incubated with increasing concentrations of SGN-B7H4V, the unconjugated B7H41001 mAb, a non-binding control mAb, or a positive control mAb to CD47 and then co-cultured with monocytes/macrophages. Phagocytosis of opsonized cells was assessed by flow cytometry.

SKBR3 tumor cells were fluorescently labeled with PKH26 (PKH26 Red Fluorescent Cell Membrane Labeling Kit, Sigma-Aldrich #PKH26GL-1KT), according to manufacturer instructions: SKBR3 cells were harvested with Versene (Gibco #15040-066) for 10 minutes and washed once with PBS. Cell were resuspended in 1 mL Diluent C (included in the PKH26 Red Fluorescent Cell Membrane Labeling Kit, Sigma-Aldrich #PKH26GL-1KT). In a separate tube, 1 mL Diluent C was mixed with 4 μL PKH26 dye by pipetting up and down. The dye solution was transferred to resuspended cells, quickly mixed by pipetting up and down several times, and incubated at room temperature for 5 minutes. Labeling reaction was stopped by adding 2 mL FBS (0.05-0.2 EU/ml Endotoxin, R&D Systems #S11550H). Cells were then washed once with RPMI 1640 (Gibco #11875-093) containing 10% FBS. Cells were resuspended in PBS at concentration of 0.8×106 cells/mL and transferred (300 μL/well) to a 96-well U-bottom plate (Falcon #353227). Target cells were treated with test articles as follows: A serial dilution (1:10) of test articles was prepared in PBS in a 96-well U-bottom plate from 0.0001-100 μg/mL (note that working concentration will be 0.001-10 μg/mL). Test articles (33 μL/well) were added to appropriate wells of cells in U-bottom plate and incubated at room temperature for 30 minutes. Cells were washed twice with 200 μL/well RPMI 1640 media containing 10% FBS. Finally, cells were resuspended in 330 μL/well RPMI 1640 media containing 10% FBS. PBMCs from two healthy donors were thawed and plated as follows: One day −1, cells were thawed at 37° C. and transferred to RPMI 1640 containing 10% FBS (0.05-0.2 EU/ml Endotoxin, R&D Systems #S11550H). PBMCs were plated at 0.7×106 cells/well in a flat-bottom 48-well plate (Falcon #353230) to allow monocytes to adhere to plates overnight. The next day, media was aspirated to remove the majority of non-adherent lymphocytes and replaced with 200 uL fresh RPMI 1640 containing 10% FBS. Then, treated target cells (100 uL) were transferred into corresponding wells 48-well plates containing monocytes/macrophages and incubated at 37° C. overnight for 14-18 hours. After 14-18 hours, cells were harvested and stained for flow cytometric analysis. All cells were harvested from each well by collecting cells in the supernatent, cells removed by a PBS wash, and cells lifted from the plate with Versene (Gibco #15040-066). Macrophages were then stained as follows: Target cells and macrophages were resuspended in 50 μL BD stain buffer (BD Pharmingen, #554657) containing human Fc fragment blocking agent (1:20 dilution, Millipore #401104) in a 96-well U-bottom plate and incubated on ice for 30 minutes. Next, 50 μL of anti-CD14-BV421 (clone M5E2, Biolegend #301830) and anti-CD45-APC-Cy7 (clone 2D1, Biolegend #368516) antibodies diluted 1:50 in BD stain buffer (BD Pharmingen, #554657) was added to each well and incubated on ice in the dark for 30 minutes. Finally, cells were washed twice with BD stain buffer, and resuspended in 150 uL PBS (Gibco #10010023) for subsequent flow cytometric analysis on an Attune N×T flow cytometer. Unstained wells were included as a negative control to aid in gating of CD14/CD45+ cells. Phagocytosis is reported as the PKH26 geometric mean fluorescence intensity (gMFI) of gated CD14+/CD45+ cells analyzed using Flowjo. Values were exported to Excel for further analysis and plotted using GraphPad Prism.

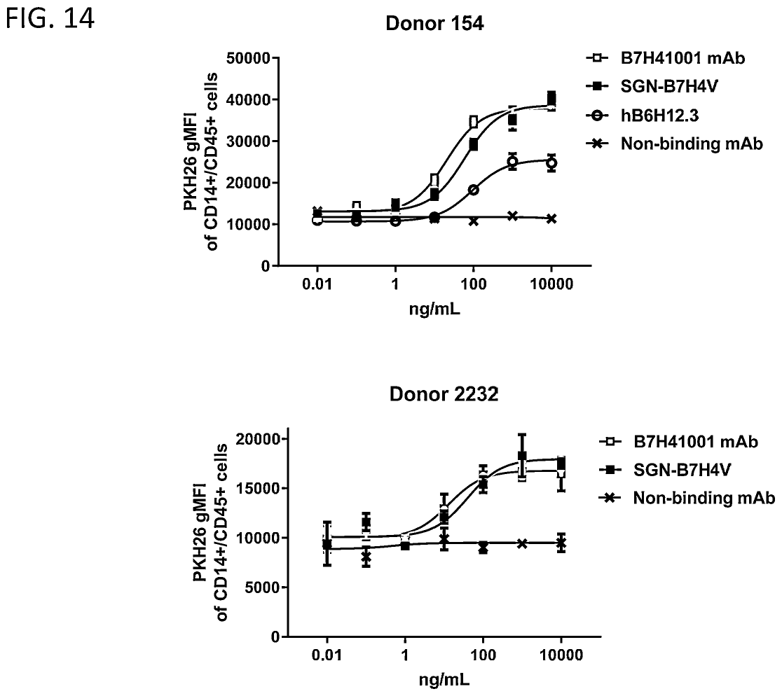

As shown in FIG. 14, both SGN-B7H4V and B7H41001 mAb demonstrated ADCP activity above that seen with the positive control, while the non-binding control mAb elicited minimal ADCP activity in 2 independent donors.

### CDC

Antibodies have the ability to recruit complement proteins triggering complement-dependent cytotoxicity (CDC). The ability of SGN-B7H4V, the unconjugated B7H41001 mAb as well as a non-binding control ADC and mAb to mediate CDC was tested using WIL2S and RAJI cells transduced to express human B7-H4.

Tumor target cells were counted and pre-blocked with 10 μg/mL of mAbs against complement regulatory protein (anti-human CD46 (Biolegend #352404), anti-human CD55 (R&D Systems #MAB2009), anti-human CD59 (BIO-RAD #MCA715G) in RPMI (Gibco #11875-093) containing 1% HI-FBS (Gibco #16140-071) to prevent inhibition of the complement pathway. Cells were incubated at room temperature for 20 minutes and then washed twice with RPMI. Target cells were resuspended at 1 million cells/mL in RPMI containing Sytox Green reagent (Life Technologies #S7020) at final dilution of 1:1000 and 100,000 cells/well (100 μL) were plated into clear F-bottom black plates. Using complement media (RPMI containing 10% human serum (Complement Technology, Inc. #NHS)), test antibodies and controls were titrated with a 3-fold dilution ranging from 50-0.02 μg/mL and 100 μL/well of the titrations were plated over the target cells. To measure total cell death, 2% Triton X (EMD Millipore Corp. #648463-50ML) was used as a positive control. Test plate was incubated for 2 hours at 37° C. in a humidity-controlled incubator and then Sytox Green fluorescence was read on an Envision plate reader.

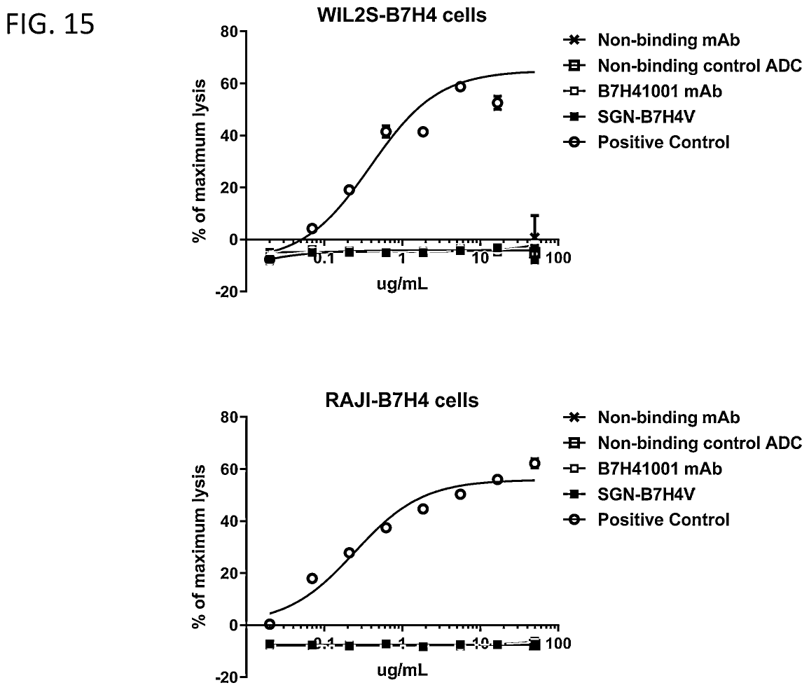

As shown in FIG. 15, treatment with SGN-B7H4V or B7H41001 mAb in the presence of complement (10% human serum) did not induce lysis. The functionality of the complement source used in the assay was confirmed by treating cells with a positive control mAb (the anti-CD70 mAb h1F6), which is known to elicit CDC.

### Results

Nonclinical data suggested that the antitumor activity of SGN-B7H4V was due to the binding of the ADC to B7-H4-expressing tumor cells, followed by internalization of the immune checkpoint ligand B7-H4 and release of MMAE via proteolytic cleavage. MMAE disrupts the microtubule network of actively dividing cells, leading to cell cycle arrest and apoptotic cell death in a manner consistent with immunogenic cell death. The preclinical data of this example suggested that the SGN-B7H4V antibody also binds and signals through Fc receptors and has Fc effector ADCC and ADCP functionality.

### Example 10: SGN-B7H4V In Vivo Efficacy

The objective of these studies was to evaluate the anti-tumor activity of SGN-B7H4V in vivo in a variety of xenograft models that express B7-H4 including two models of triple negative breast cancer (TNBC; MX-1 and MDA-MB-468), one model of Her2+ breast cancer (HCC1569), and one model of high grade serous ovarian adenocarcinoma (OVCAR3). In vivo, tumors exhibited uniform, high expression of B7-H4 by immunohistochemistry (IHC), except OVCAR3, which was heterogenous for B7-H4 staining. We evaluated SGN-B7H4V in all models at a single standard dose (3 mg/kg, 3 weekly doses). In the MDA-MB-468 model, we also evaluated SGN-B7H4V at a range of doses (0.3-3 mg/kg, 3 weekly doses) to determine the dose-response relationship.

### Study MX1-2

Animals were dosed with SGN-B7H4V or non-binding control ADC (N=5 mice per group, 3 mg/kg for three weekly doses) when tumors were 100 mm3. Tumor volume was measured twice weekly until study day 84.

Female SCID mice were implanted with 5×10⁵ MX1 tumor cells in 25% Matrigel H C (Corning #354248) subcutaneously. Once tumor volumes reached 100 mm³, mice were randomized into treatment groups of 5 mice each and dosed with 3 mg/kg of ADC every seven days for three total doses (q7dx3). Tumor volumes were measured twice per week, and animals were euthanized when tumor volume reached 700-1000 mm³. Stock concentrations of ADC were diluted to a desired concentration (with 0.01% Tween20 in PBS) and injected i.p. into each treatment group.

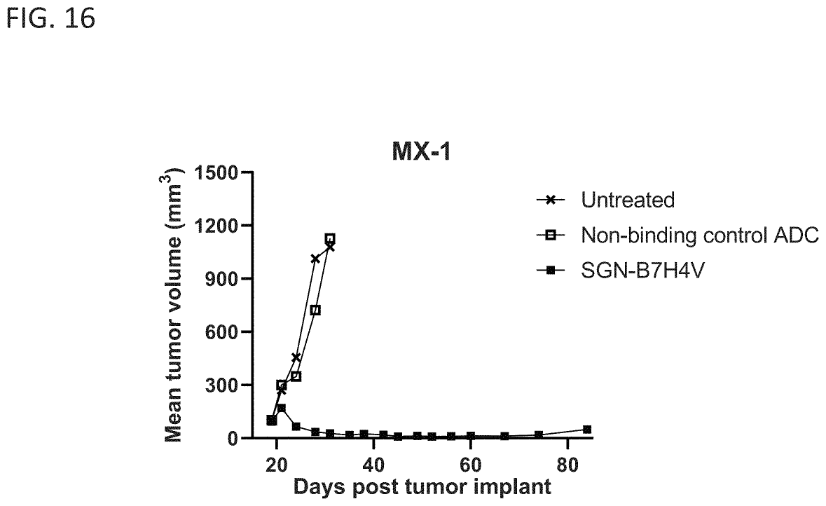

As shown in FIG. 16, treatment with SGN-B7H4V caused regression of tumor volumes in all mice, with sustained regression in 4 of the 5 mice. In contrast, the non-binding control ADC had minimal anti-tumor activity.

### Study MDAMB468-6

Animals were dosed with SGN-B7H4V or non-binding control ADC (N=5 mice per group, 3 mg/kg for three weekly doses) when tumors were 100 mm³. Tumor volume was measured twice weekly until study day 80.

Female NSG mice were implanted with 1×106 MDA-MB-468 cells in 25% Matrigel H C (Corning #354248) subcutaneously. Once tumor volumes reached 100 mm³, mice were randomized into treatment groups of 5 mice each and dosed with 0.3, 1, or 3 mg/kg every seven days for three total doses (q7dx3). Twenty-four hours prior to receiving each ADC dose, each animal was treated with 10 mg/kg hIVIG (Grifolds). Tumor volumes were measured twice per week, animals were euthanized when tumor volume reached 700-1000 mm³. Stock concentrations of ADC were diluted to desired concentration (with 0.01% Tween20 in PBS) and injected i.p. into each treatment group

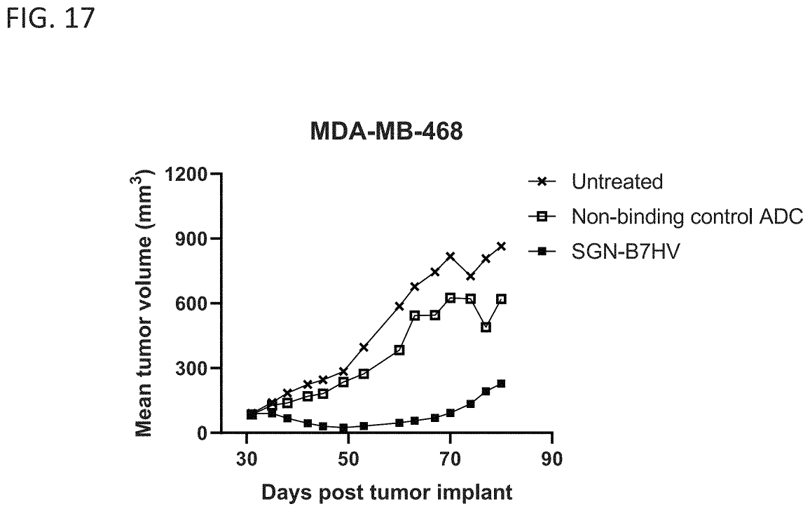

As shown in FIG. 17, treatment with SGN-B7H4V led to transient regression of tumor volumes in all mice. In contrast, the non-binding control ADC had minimal anti-tumor activity.

### Study MDAMB468-9.

Animals were dosed with SGN-B7H4V, the unconjugated mAb B7H41001, or non-binding control ADC (N=5 mice per group, 0.3, 1, and/or 3 mg/kg for three weekly doses) when tumors were 100 mm³. Tumor volume was measured twice weekly until study day 85.

Female NSG mice were implanted with 1×10⁶MDA-MB-468 cells in 25% Matrigel H C (Corning #354248) subcutaneously. Once tumor volumes reached 100 mm³, mice were randomized into treatment groups of 5 mice each and dosed with 0.3, 1, or 3 mg/kg every seven days for three total doses (q7dx3). Twenty-four hours prior to receiving each ADC dose, each animal was treated with 10 mg/kg hIVIG (Grifolds). Tumor volumes were measured twice per week, animals were euthanized when tumor volume reached 700-1000 mm³. Stock concentrations of ADC were diluted to desired concentration (with 0.01% Tween20 in PBS) and injected i.p. into each treatment group.

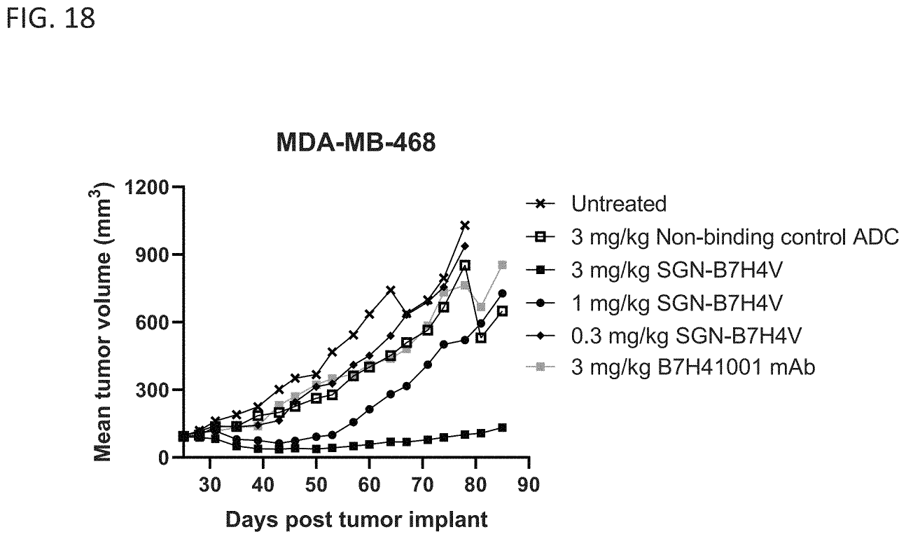

As shown in FIG. 18, treatment with both 1 and 3 mg/kg SGN-B7H4V led to transient regression of tumor volumes in all mice. In contrast, treatment with 0.3 mg/kg SGN-B7H4V, 3 mg/kg B7H41001 mAb, or 3 mg/kg non-binding control ADC had minimal anti-tumor activity.

### Study HCC1569-2.

Animals were dosed with SGN-B7H4V or non-binding control ADC (N=5 mice per group, 3 mg/kg for three weekly doses) when tumors were 100 mm³. Tumor volume was measured once or twice weekly until study day 71.

Female NSG mice were implanted with 1×10⁶HCC1569 tumor cells in 25% Matrigel H C (Corning #354248) subcutaneously. Once tumor volumes reached 100 mm³, mice were randomized into treatment groups of 5 mice each and dosed with 3 mg/kg of ADC every seven days for three total doses (q7dx3). Each mouse was treated with 10 mg/kg hIVIG (Grifolds) twenty-four hours prior to receiving each ADC dose. Tumor volumes were measured 1-3 times per week, and animals were euthanized when tumor volume reached 700-1000 mm³. Stock concentrations of ADC were diluted to a desired concentration (with 0.01% Tween20 in PBS) and injected i.p. into each treatment group.

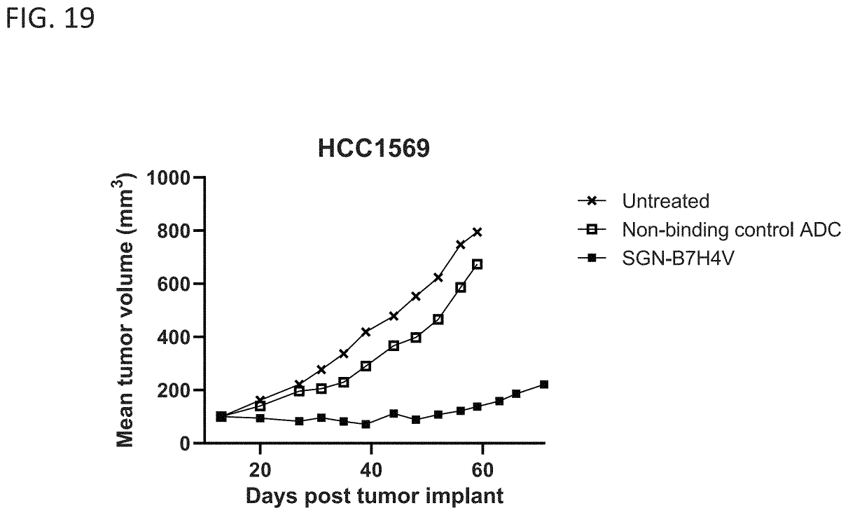

As shown in FIG. 19, treatment with SGN-B7H4V led robust tumor growth delay; we observed a reduction in mean tumor volume in the SGN-B7H4V treatment group compared to the untreated and non-binding control ADC treatment group.

Study OVCAR3-e314

Animals were dosed with SGN-B7H4V or non-binding control ADC (N=8 mice per group, 3 mg/kg for three weekly doses) when tumors were 150-200 mm³. Tumor volume was measured twice weekly until study day 60.

Female SCID mice were implanted with OVCAR3 tumor fragments (˜1 mm³) subcutaneously at Charles River Discovery Services (NC). Once tumor volumes reached 150-200 mm³, mice were dosed with 3 mg/kg of ADC every seven days for three total doses (q7dx3). Tumor volumes were measured twice weekly, and animals were euthanized when tumor volume reached 1000 mm³. Stock concentrations of ADC were diluted to a desired concentration (with PBS) and injected i.v. into each treatment group.

Tumor growth inhibition (TGI) for each treatment group compared to the untreated group was analyzed on study day 26 as follows: % TGI=[1−((MTV_{untreated}−MTV_{treated})/MTV_{untreated})]*100, where MTV is the mean tumor volume. Statistical significance was determined using the Mann-Whitney U test (two-tailed).

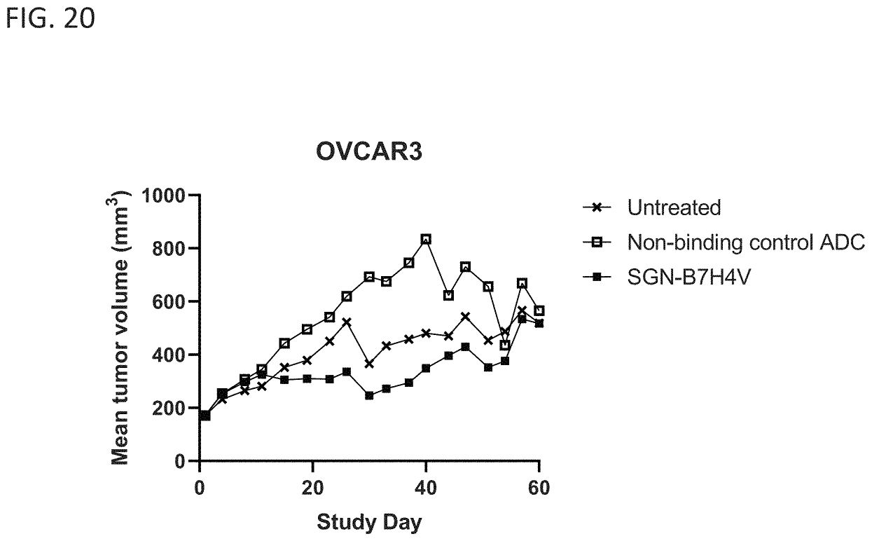

As shown in FIG. 20, treatment with SGN-B7H4V (but not the non-binding control ADC) led to a reduction in mean tumor volume compared to the untreated group. Tumor growth inhibition (TGI) on study day 26 for the SGN-B7H4V treatment group was 48% compared to the untreated group.

### Example 11: Immunohistochemical Evaluation of B7-H4 Expression by Xenograft Tumors

Immunohistochemical staining for B7-H4 was performed on untreated MX-1 and HCC1569 tumors as follows:

Samples were processed on a Bond-III™ autostainer (Leica Microsystems Inc., Buffalo Grove Ill.) at ambient temperature following the manufacturer's instructions. FFPE sections on glass slides were de-paraffinized using Bond™ Dewax solution (Leica, cat #AR9222) at 58-60° C. Antigen retrieval was performed using EDTA-based pH 9 Bond™ Epitope Retrieval Solution 2 (Leica, cat #AR9640) for 20 min at 98-100° C. The Peroxide Block was applied for 10 minutes followed by blocking of nonspecific background with Protein Block (Dako, cat #X090930) for 20 minutes. All antibodies were diluted to working concentration in BOND Primary Antibody Diluent (Leica, cat #AR9352). Isotype-matched rabbit IgG (Abcam, clone EPR25a cat #ab172730) was used as a negative control for background staining. For automated IHC staining we used the Bond™ Polymer Refine Detection (DAB) kit (Leica, cat #DS9800). Slides were incubated with rabbit monoclonal primary antibody against B7-H4 for 45 minutes at 5 μg/mL (primary antibody was dispensed twice for a total of 300 uL per slide). Detection of HRP was done with DAB refine chromogen incubated for 10 minutes. Sections were counterstained with hematoxylin for 7 minutes.

Upon protocol completion on the autostainer, the slides were immediately removed and placed in deionized (DI) water before going through a series of dehydration steps (70% EtOH, 70% EtOH, 95% EtOH, 95% EtOH, 100% EtOH, 100% EtOH, 100% EtOH, Xylenes×3) to allow for cover slipping (Leica, CV5030) using Surgipath mounting medium (Leica, Surgipath Micromount, cat #3801731). Images were captured using a slide scanner (Leica, Aperio AT2).

Immunohistochemical staining for B7-H4 was also performed on untreated MDA-MB-468 tumors (˜500 mm³), untreated OVCAR3 tumors (150-200 mm³) as well as OVCAR3 tumors (˜1000 mm³) following treatment with SGN-B7H4V or the non-binding control ADC as follows: Tumors were deparaffinized by incubation in: Xylene for 3 minutes (twice), 100% EtOH for 2 minutes (twice), 90% EtOH for 2 minutes, 700% EtOH for 2 minutes.

Antigen retrieval was performed using a Decloaking Chamber NxGen Biocare and the 110° C. program with Diva Retrieval solution. Slides were stained on the IntelliPath Automated stainer per the following protocol: Remove hot containers from the decloaking chamber and rinse in deionized water. Place slides in TBST prior to adding to IntelliPath. Hydrate slides on stainer prior to automation Add 300 uL/slide Peroxidaze (Biocare #PX968) and incubate 10 minutes. Wash slides in TBST. Add 300 uL/slide Sniper block (Biocare #B5966) and incubate 10 minutes. Blot off blocking solution. Add 300 uL/slide primary antibody and incubate 1 hour. Wash slides twice in TBST. Add 300 uL/slide HRP polymer and incubate 30 minutes. Wash slides twice in TBST. Add 300 uL/slide DAB (Vector #sk-4103) and incubate 5 minutes. Wash in TBST. Add 300 uL/slide hematoxylin and incubate 2 minutes. Wash in deionized water. Dehydrate slides by placing them in the oven for 30 minutes. Place coverslip on slides. Images were analyzed using Halo image analysis software (Indica Labs). The H-score was calculated according to the following equation: (3×% Strong Signal)+(2×% Moderate Signal)+(Weak Signal). Where applicable, biochemical followed by cells followed by animal (lowest to highest).

### Results

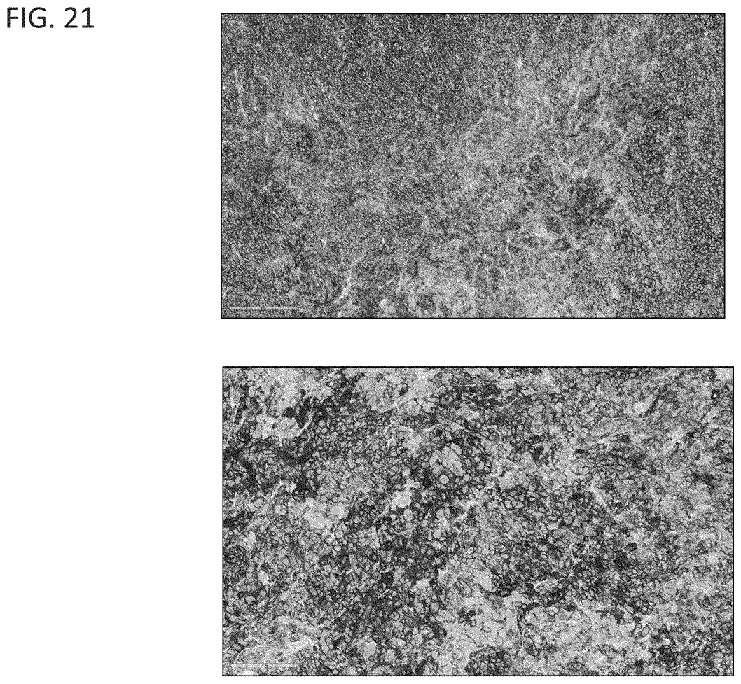

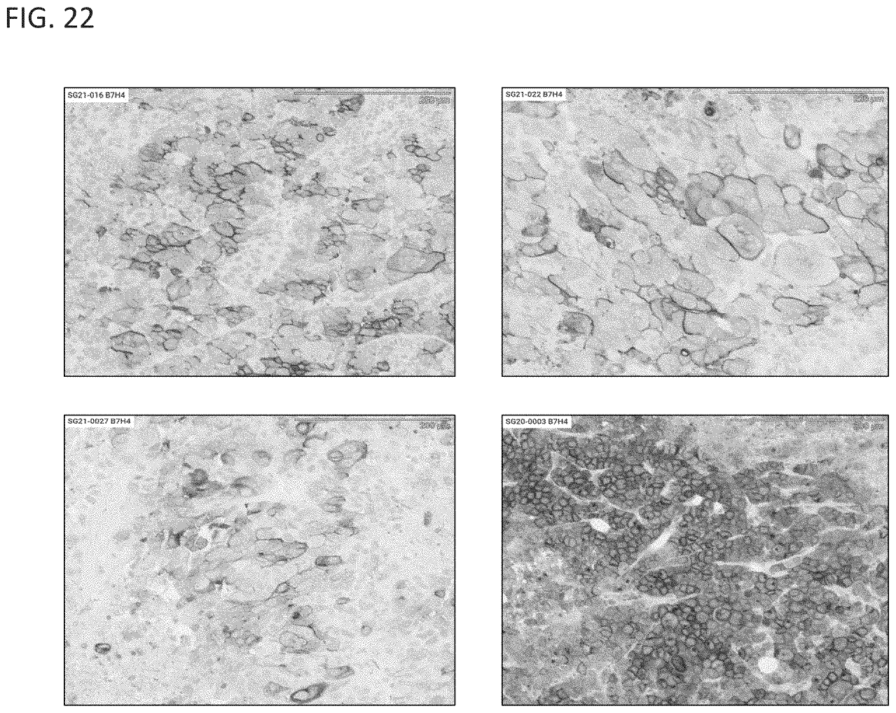

B7-H4 expression on untreated MX-1, MDA-MB-468, and HCC1569 tumors was evaluated by immunohistochemical staining. As shown in FIG. 21 and FIG. 22, all three tumors exhibited uniform, high expression of B7-H4

To characterize B7-H4 expression on OVCAR3 tumors prior to and following treatment with SGN-B7H4V, immunohistochemical staining for B7-H4 was performed on: Two untreated satellite OVCAR3 tumors (150-200 mm³); Six SGN-B7H4V-treated OVCAR3 tumors (˜1000 mm³); and Eight non-binding control ADC-treated OVCAR3 tumors (˜1000 mm³).

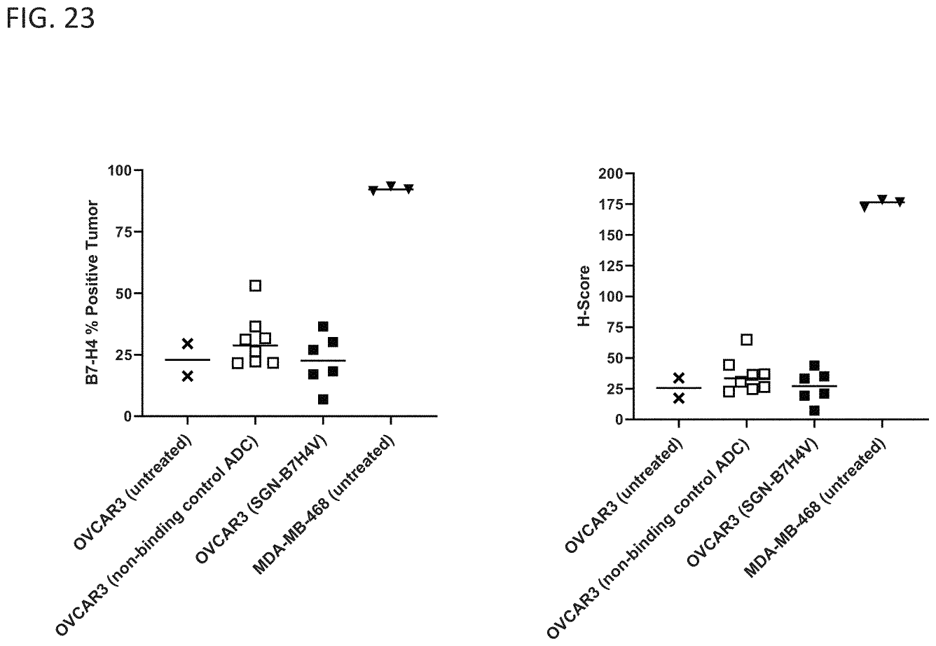

B7-H4 staining on untreated OVCAR3 tumors was heterogenous: ˜25% of tumor tissue was B7-H4+ with an average H-score of 26. Treatment with SGN-B7H4V or the non-binding control ADC did not have a significant impact tumor B7-H4 expression (FIGS. 22 and 23). In contrast, B7-H4 staining was uniformly high on untreated MDA-MB-468 tumors: >90% of tumor tissue was B7-H4+ with an average H-score of 176 (FIG. 23).

In this example, the effect of SGN-B7H4V in vivo was evaluated in several xenograft models of ovarian and breast cancer. In vivo, tumors exhibited uniform, high expression of B7-H4 by immunohistochemistry (IHC), except OVCAR3, which was heterogenous for B7-H4 staining. SGN-B7H4V demonstrated robust antitumor activity at the 3 mg/kg dose level (3 weekly doses) in three xenograft models of human breast cancer (MX-1, MDA-MB-468, and HCC1569), including durable tumor regression in the MX-1 model of triple negative breast cancer (TNBC). Transient tumor regression was observed in the MDA-MB-468 model following treatment with both 1 and 3 mg/kg of SGN-B7H4V. In the OVCAR3 xenograft model of high grade serous ovarian adenocarcinoma, 3 mg/kg SGN-B7H4V (3 weekly doses) demonstrated modest tumor growth delay. Altogether, these data support the evaluation of SGN-B7H4V in a phase 1 clinical trial.

### Example 12: Evaluation of SGN-B7H4V in PDX Models

The objective of these studies was to evaluate the anti-tumor activity of SGN-B7H4V in vivo in a variety of patient-derived xenograft (PDX) models of breast and ovarian cancer. PDX models were selected with a range of B7-H4 expression levels and included both naïve and heavily pre-treated tumors. We evaluated SGN-B7H4V in all models at a single standard dose (3 mg/kg, 3 weekly doses).

### Activity of SGN-B7H4V in 11 TNBC PDX Models

Animals were dosed with hIVIG followed by SGN-B7H4V or non-binding control ADC (3 mg/kg for three weekly doses) when tumors were 150-300 mm³. Tumor size and body weight were measured twice weekly, and the study was terminated when tumors in the control group reached 1500 mm³ or up to Day 28, whichever occurred first, or maximum up to Day 60.

Specifically, stock mice were bilaterally implanted with fragments from one of the Champions TumorGraft® models representing human triple negative breast cancer. After the tumors reached 1000-1500 mm³, they were harvested, and the tumor fragments were implanted subcutaneously (s.c.) in the left flank of the female study mice. Each animal was implanted with a specific passage lot and documented. Tumor growth was monitored twice a week using digital calipers, and the tumor volume (TV) was calculated using the formula (0.52×[length×width²]). When the TV reached approximately 150-300 mm³, animals were matched by tumor size and assigned into control (untreated) or treatment groups (n=1-3 animals/group). Each animal in the treatment groups was dosed with 10 mg/kg hIVIG (Grifolds), followed by 3 mg/kg of ADC every seven days for three total doses (q7dx3). Tumor size and body weight were measured twice weekly, and the study was terminated when tumors in the control group reached 1500 mm³ or up to Day 28, whichever occurred first, or maximum up to Day 60.

Inhibition of tumor growth was determined by calculating the percent TGI on the day at which animals in the control group were terminated (100%×[1−(final MTV−initial MTV of a treated group)/(final MTV−initial MTV of the control group)]). Treatment started on Day 0.

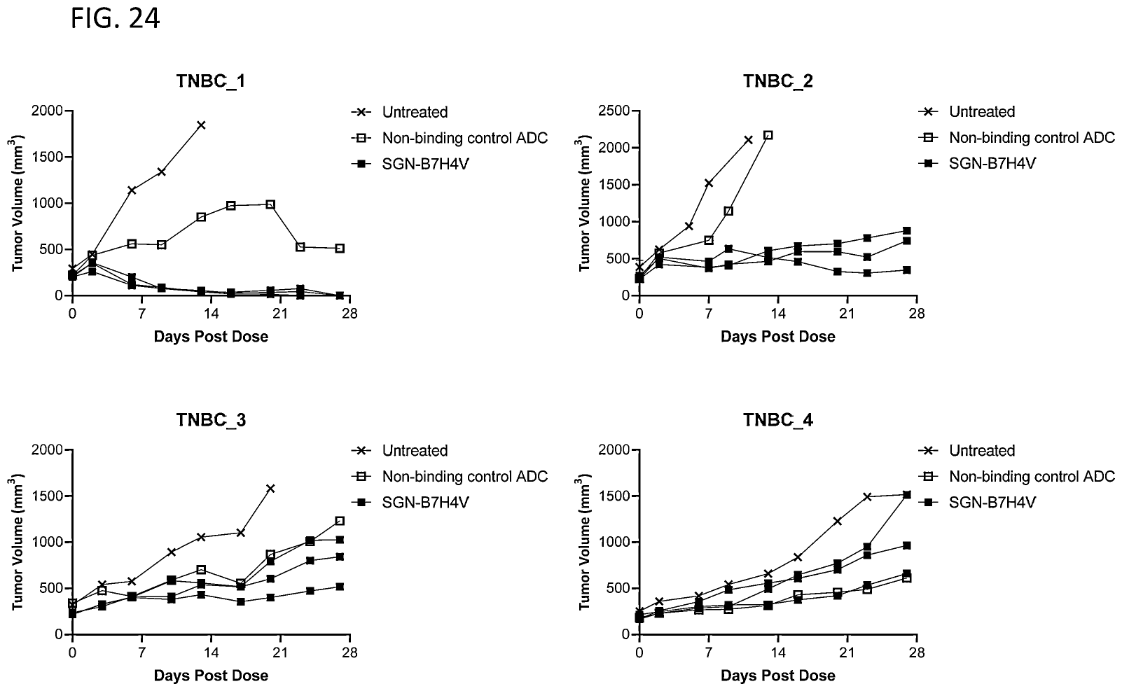

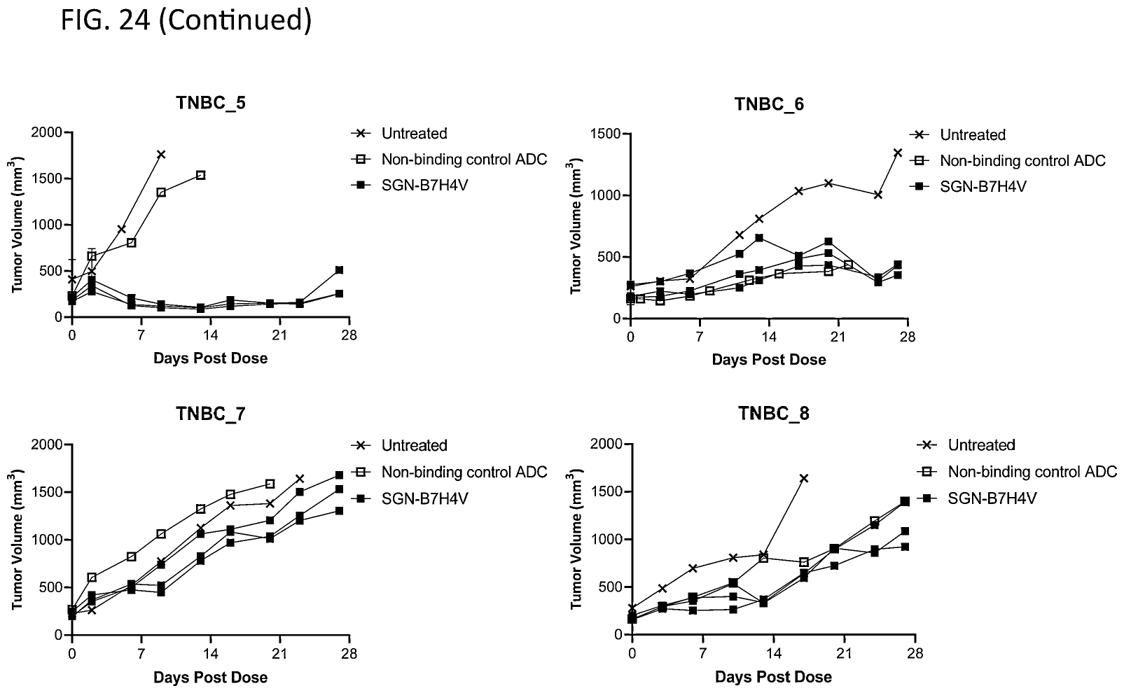

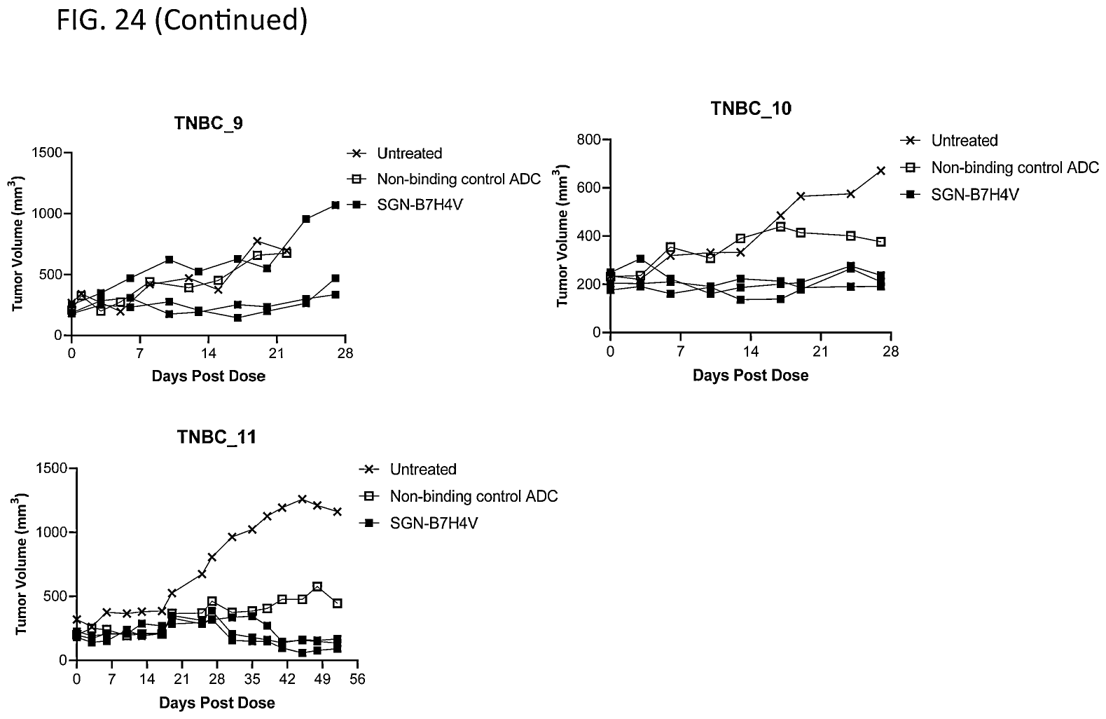

As shown in Table 15 and FIG. 24, treatment with SGN-B7H4V led to tumor regression or tumor growth delay (e.g. TGI>50%) in 9/11 PDX models of TNBC, including durable tumor regression in model TNBC_1 and transient tumor regression in models TNBC_5, TNBC_10, and TNBC_11.

TABLE 15
Activity of SGN-B7H4V in 11 TNBC PDX models

| Model | VTCN1 | | | |
|---|---|---|---|---|
| Number | mRNA (TPM) | Treatment History | Day | % TGI |
| TNBC_1 | 44 | Not available | 13 | 111 |
| TNBC_2 | 16 | 5-Fluorouracil/Epirubicin/Cyclophosphamide; | 7 | 86 |
| | | Carboplatin/Docetaxel | | |
| TNBC_3 | 69 | Doxorubicin/Cyclophosphamide/Paclitaxel; | 20 | 71 |
| | | Carboplatin/Gemcitabine; Capecitabine/Docetaxel; | | |
| | | Capecitabine; Tivantinib; Eribulin; Liposomal | | |
| | | doxorubicin | | |
| TNBC_4 | 169 | Doxorubicin/Cyclophosphamide/Paclitaxel; | 20 | 55 |
| | | Liposomal doxorubicin; Capecitabine; Cisplatin/ | | |
| | | Gemcitabine; Eribulin | | |
| TNBC_5 | 325 | No prior treatment | 9 | 106 |
| TNBC_6 | 158 | Doxorubicin/Cyclophosphamide/Paclitaxel; | 20 | 62 |
| | | Cyclophosphamide/Methotrexate/5-Fluorouracil; | | |
| | | Eribulin; Carboplatin/Gemcitabine; Carboplatin | | |
| TNBC_7 | 66 | Doxorubicin/Cyclophosphamide/Paclitaxel | 20 | 25 |
| TNBC_8 | 47 | No prior treatment | 17 | 67 |
| TNBC_9 | 15 | Paclitaxel | 22 | 26 |
| TNBC_10 | n.d. | No prior treatment | 27 | 100 |
| TNBC_11 | n.d. | Doxorubicin/Cyclophosphamide/Gosorelin; | 52 | 109 |
| | | Carboplatin/Paclitaxel; Talazoparib; Capecitabine; | | |
| | | Vinorelbine | | |

Description of TNBC patient-derived xenograft (PDX) models. VTCN1 (B7-H4) expression data anc patient treatment history were obtained from Champions Oncology's database. The % tumor growth inhibition (TGI) was calculated at the indicated day for the SGN-B7H4V-treated compared to untreated group.

### Activity of SGN-B7H4V in HR+ Breast PDX Models and Ovarian PDX Models

Animals were dosed SGN-B7H4V or non-binding control ADC (3 mg/kg for three weekly doses) when tumors were 150-300 mm³. Tumor size and body weight were measured twice weekly, and the study was terminated when tumors in the control group reached 1500 mm³ or up to Day 28, whichever occurred first, or maximum up to Day 60.

Specifically, Stock mice were bilaterally implanted with fragments from one of the Champions TumorGraft® models representing human HR⁺ BC or ovarian cancer. After the tumors reached 1000-1500 mm³, they were harvested, and the tumor fragments were implanted subcutaneously (s.c.) in the left flank of the female study mice. Each animal was implanted with a specific passage lot and documented. Tumor growth was monitored twice a week using digital calipers, and the tumor volume (TV) was calculated using the formula (0.52×[length×width²]). When the TV reached approximately 150-300 mm³, animals were matched by tumor size and assigned into control (untreated) or treatment groups (n=3 animals/group). Each animal in the treatment groups was dosed with 3 mg/kg of ADC every seven days for three total doses (q7dx3). Tumor size and body weight were measured twice weekly, and the study was terminated when tumors in the control group reached 1500 mm³ or up to Day 28, whichever occurred first, or maximum up to Day 60.

Inhibition of tumor growth was determined by calculating the percent TGI on the day at which animals in the control group were terminated (100%×[1−(final MTV−initial MTV of a treated group)/(final MTV−initial MTV of the control group)]). Treatment started on Day 0.

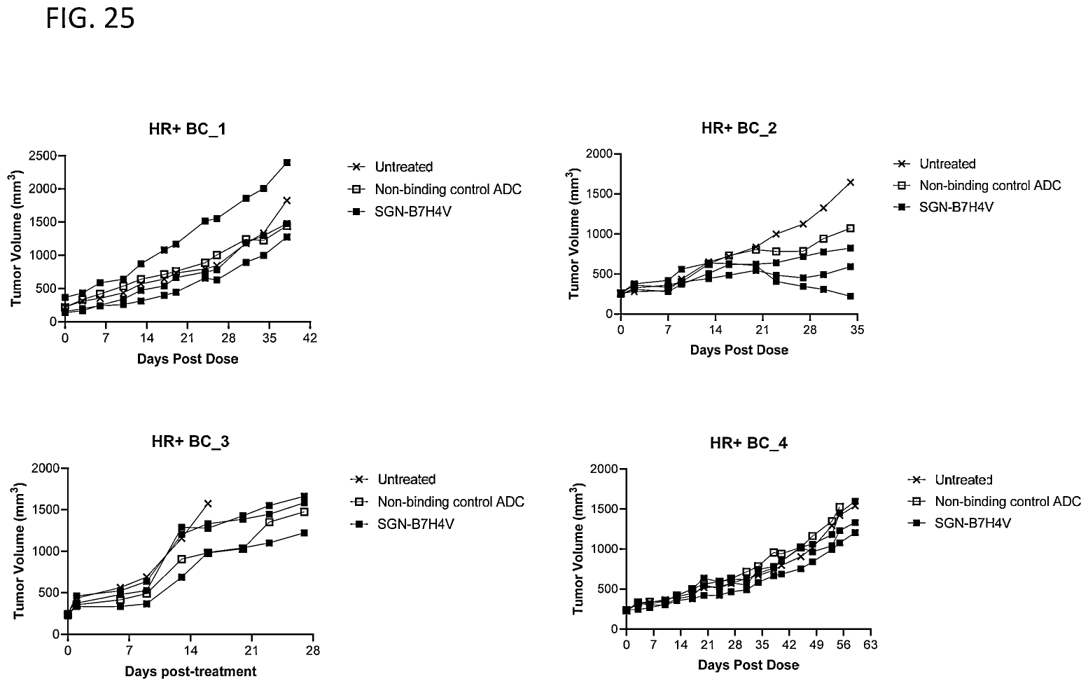

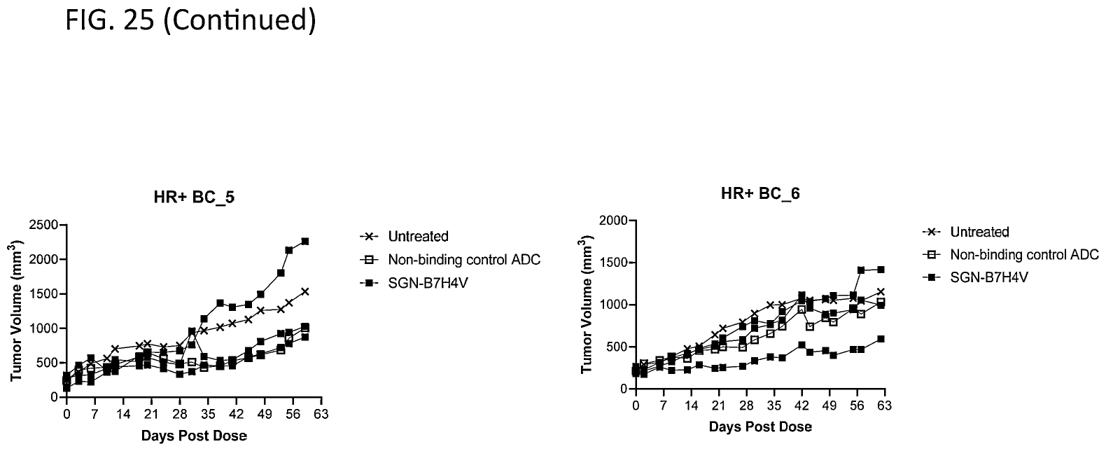

As shown in Table 16 and FIG. 25, treatment with SGN-B7H4V led to tumor growth delay (e.g. TGI>50%) in the HR_BC_2 PDX model of HR⁺ BC. Minimal anti-tumor activity was observed in the other 5 models evaluated.

TABLE 16
Activity of SGN-B7H4V in 6 HR⁺ BC PDX models

| Model | VTCN1 | Tumor | | | |
|---|---|---|---|---|---|
| Number | mRNA (TPM) | Status | Treatment History | Day | % TGI |
| /HR + | 28 | Primary | No prior treatment | 38 | 7 |
| BC_1 | | | | | |
| HR + | 16 | Primary | No prior treatment | 34 | 79 |
| BC_2 | | | | | |
| HR + | 45 | Primary | Doxorubicin/Cyclophosphamide/Paclitaxel | 16 | 28 |
| BC_3 | | | | | |
| HR + | 57 | Metastatic | Doxorubicin/Cyclophosphamide/Paclitaxel; | 59 | 12 |
| BC_4 | | | Capecitabine; Letrozole; Letrozole/ | | |
| | | | Palbociclib; Carboplatin/Gemcitabine; | | |
| | | | Carboplatin | | |
| HR+ | 10 | Metastatic | Fulvestrant; Palbociclib; | 59 | 11 |
| BC_5 | | | Everolimus/Exemestane | | |
| HR+ | 84 | Metastatic | Palbociclib; Everolimus; Capecitabine | 62 | 16 |
| BC_6 | | | | | |

Description of HR⁺ BC patient-derived xenograft (PDX) models in Study 1267-142. VTCNI (B7-H4) expression data and patient treatment history were obtained from Champions Oncology's database. The % tumor growth inhibition (TGI) was calculated at the indicated day for the SGN-B7H4V-treated compared to untreated group.

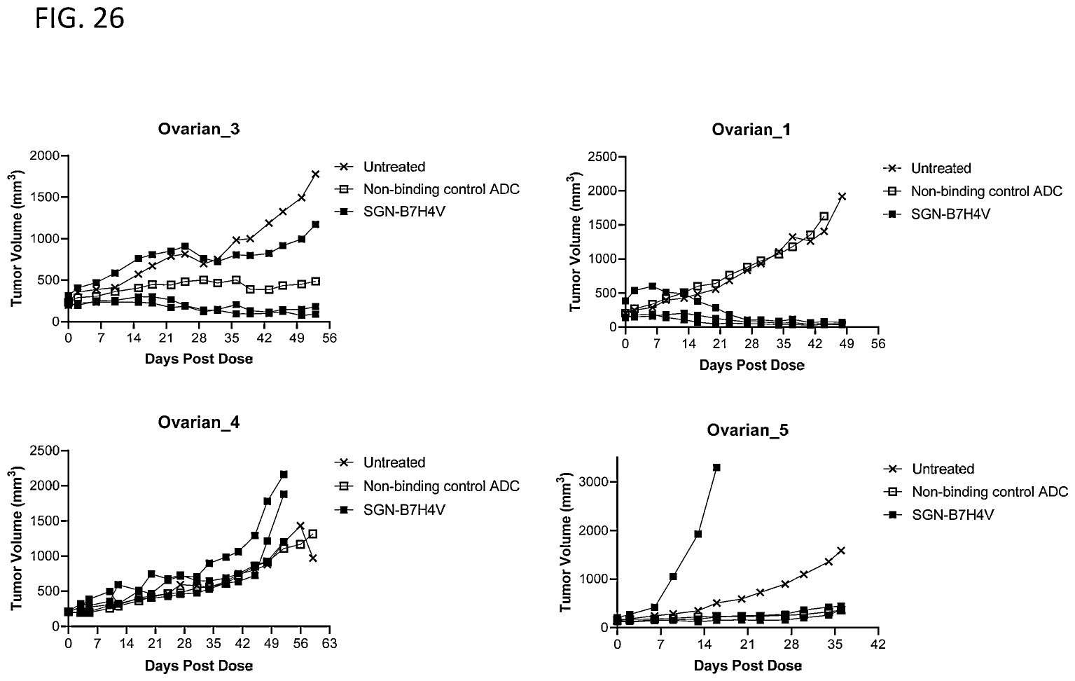

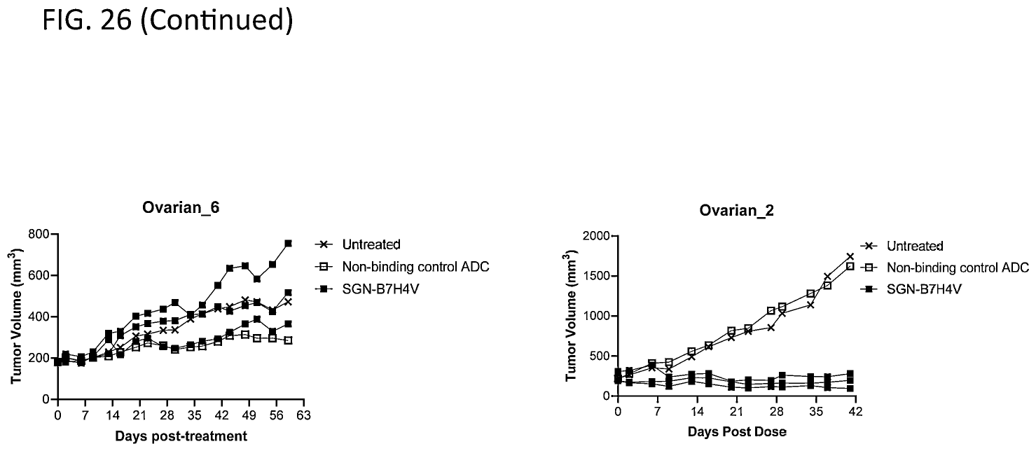

As shown in Table 17 and FIG. 26, treatment with SGN-B7H4V led to tumor regression or tumor growth delay (e.g. TGI>50%) in 4/6 PDX models of ovarian carcinoma, including durable tumor regression in model Ovarian_1 and transient tumor regression in model Ovarian 2, a xenograft model derived from a heavily pre-treated metastatic serous ovarian carcinoma tumor.

TABLE 17
Activity of SGN-B7H4V in 6 Ovarian carcinoma PDX models

| Model | VTCN1 | Tumor Status/ | | | |
|---|---|---|---|---|---|
| Number | mRNA (TPM) | Histology | Treatment History | Day | % TGI |
| Ovarian_3 | 59 | Metastatic/Papillary, | Carboplatin/Paclitaxel; Topotecan; | 53 | 84 |
| | | serous origin | Gemcitabine | | |
| Ovarian_1 | 274 | Metastatic/serous | No prior treatment | 48 | 110 |
| Ovarian_4 | 11 | Metastatic/serous | Carboplatin/Paclitaxel | 52 | −57 |
| Ovarian_5 | 29 | Primary/serous | No prior treatment | 36 | 83 |
| Ovarian_6 | 128 | Primary/serous | Carboplatin/Paclitaxel | 23 | −33 |
| | | cyst-adeno | | | |
| Ovarian_2 | 288 | Metastatic/serous | Cisplatin/Docetaxel; Bevacizumab | 41 | 103 |
| | | | (maintenance); Carboplatin/Paclitaxel; | | |
| | | | Cisplatin/Paclitaxel; Paclitaxel | | |

Description of ovarian carcinoma patient-derived xenograft (PDX) models in Study 1267-142. VTCN1 (B7-H4) expression data and patient treatment history were obtained from Champions Oncology's database. The % tumor growth inhibition (TGI) was calculated at the indicated day for the SGN-B7H4V-treated compared to untreated group.

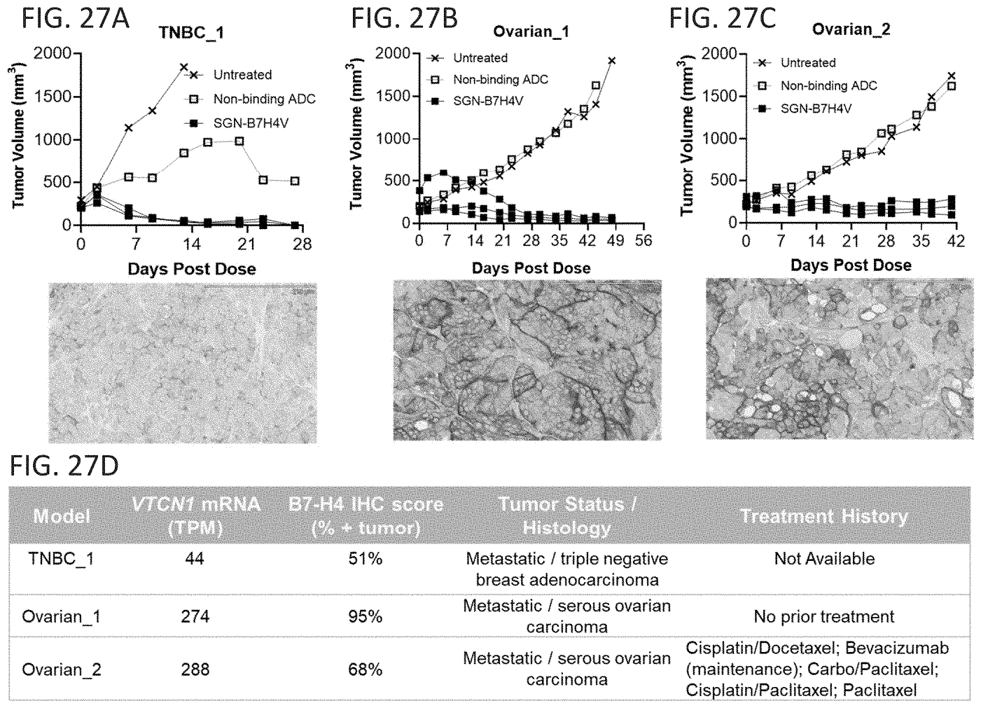

In this example, the effect of SGN-B7H4V in vivo was evaluated in several patient-derived xenograft models of breast and ovarian cancer. Tumor models were selected to with a range of VTCN1 (B7-H4) expression levels. SGN-B7H4V demonstrated antitumor activity at the 3 mg/kg dose level (3 weekly doses) in 9/11 models of TNBC, ⅙ models of HR⁺ BC, and 4/6 models of ovarian carcinoma. Activity was observed across a range of B7-H4 expression levels, including tumors with very low VTCN1 mRNA (TPM<20) and in both treatment naïve and heavily pretreated metastatic tumors (FIGS. 27A-D). Altogether, these data support the evaluation of SGN-B7H4V in a phase 1 clinical trial.

### Example 13: B7-H4 Expression on Hematopoietic and Immune Cells

### Gene Expression of VTCN1 (B7-H4) in Hematopoietic and Immune Cells

Blueprint RNA-seq data were quantified using a standardized pipeline by Qiagen/OmicSoft to produce gene-level normalized counts (Transcripts Per Kilobase Million, TPM) and exported from Qiagen OncoLand client on May 24, 2019 (https://digitalinsights.qiagen.com/products-overview/discovery-insights-portfolio/content-exploration-and-databases/qiagen-oncoland/). Gene-level expression values, subsequent analysis and visualization steps were performed in the R computing environment. Expression of B7 family members were analyzed for AML (Acute Myeloid Leukemia), APL (Acute Promyelocytic Leukemia), CLP (Common Lymphoid Progenitor), CMP (Common Myeloid Progenitor), GMP (Granulocyte myeloid progenitor), HMPC (Human Peritoneal Mesothelial Cells), HSC (Hematopoietic Stem Cell), MEP (Megakaryocyte-Erythroid Progenitor), MM (Multiple Myeloma), MSC (Mesenchymal Stem Cell), TPLL (T-cell Prolymphocytic Leukemia), MCL (Mantle Cell Lymphoma), CLL (Chronic Lymphocytic Leukemia).

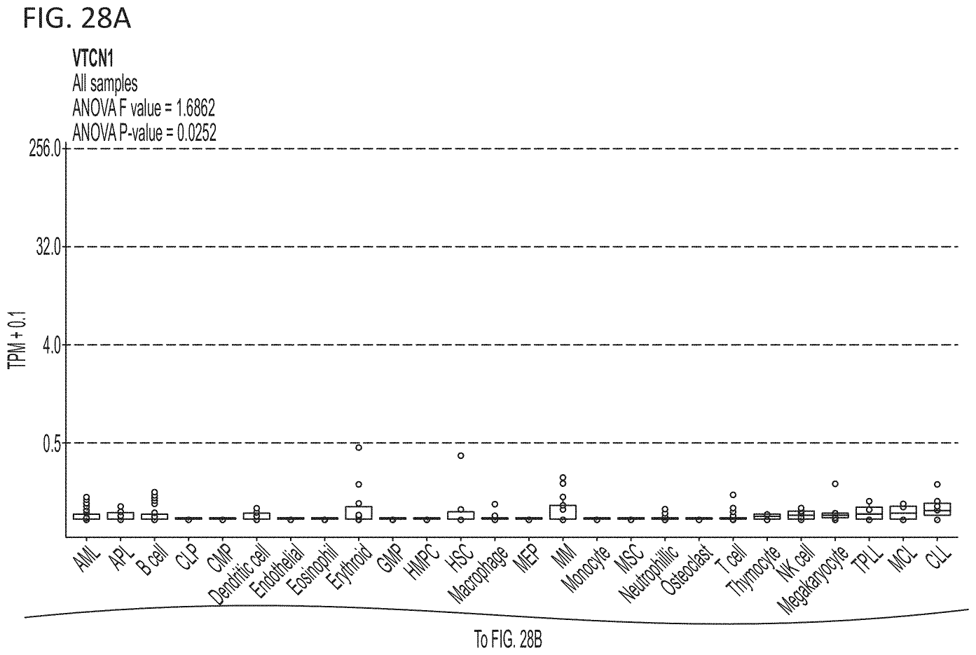

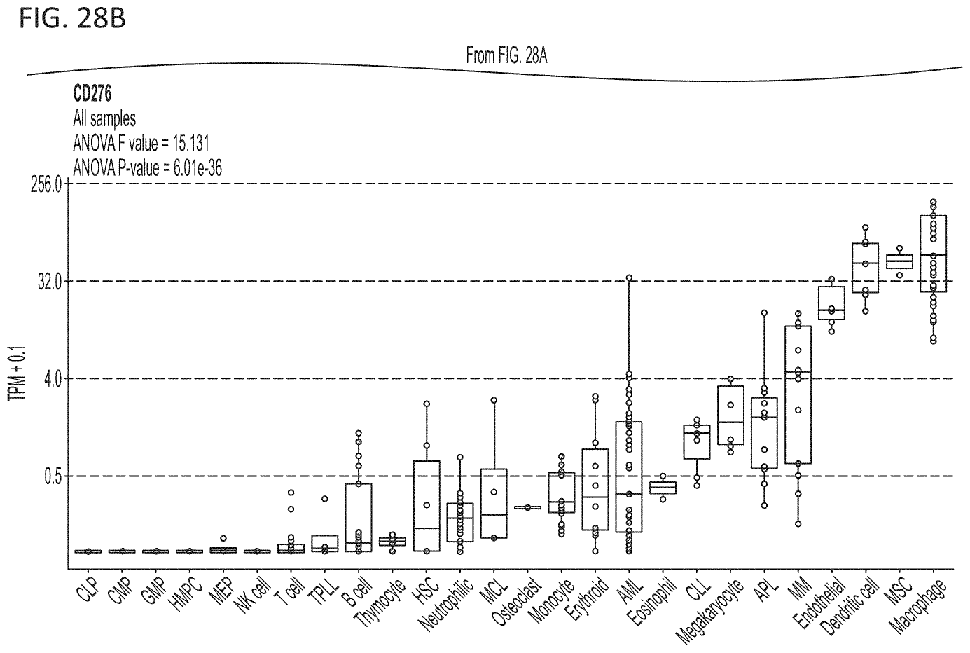

Expression levels of VTCN1 (B7-H4) compared to another B7 family member CD276 (B7-H3) in human hematopoietic cells are shown in FIG. 28A and FIG. 28B. Expression of VTCN1 was extremely low (<0.5 TPM) (FIG. 28A), while CD276 was expressed at high levels (>30 TPM) (FIG. 28B) in several myeloid cell types, including macrophages and dendritic cells. This data suggests that expression of B7-H4 is very low in hematopoietic and immune cells.

Expression of B7-H4 on Human Peripheral Blood Monocytes and Differentiated Macrophages

Expression levels of B7-H4 compared to B7-H3 (another B7 family member) was analyzed by flow cytometry on peripheral blood monocytes and differentiated macrophages from 6 donors. B7-H4 expression was analyzed with the antibody component of SGN-B7H4V, B7H41001 mAb, as well as the commercially available B7-H4 mAb clone MIH43.

Specifically, PBMCs were thawed and plated in complete “myeloid” medium (RPMI (Invitrogen #11875-093) with 10% FBS (Atlanta Biologicals #511550H), 1× Penicillin/Streptomycin (Gibco #15140-148), 1× Glutamax (Gibco #35050-061), and 10 μg/mL Ciprofloxacin (Corning #MT-61-277-RF)) in 6-well polystyrene plates (Fisher Scientific, 353046). Cells were incubated for 16 hours at 37° C. and 5% CO2 and then non-adherent cells were aspirated. For macrophage differentiation, adherent cells (monocytes) were grown in complete myeloid medium supplemented with 100 ng/mL M-CSF (R&D #216-MC-025/CF). Macrophages (MO) were harvested after 5 days. For further differentiation into TAM-like macrophages, macrophages (MO) were cultured in myeloid medium supplemented with 20 ng/mL M-CSF (R&D #216-MC-025/CF) and 100 ng/mL IL-10 (R&D #1064-IL-010/CF) for 3 additional days. For further differentiation into inflammatory (M1) macrophages, macrophages (MO) were cultured in myeloid medium supplemented with 30 ng/mL IFNg (R&D #285-IF-100/CF) for 2 additional days. For dendritic cell differentiation, adherent cells (monocytes) were grown in myeloid medium supplemented with 200 ng/mL GM-CSF (R&D #215-GM-010/CF) for 7 days. Dendritic cells were harvested at day 7 (immature) and after two additional days of culture with 100 ng/mL TNFα (mature, R&D #10291-TA-050).

All cells were harvested with 1× Versene (Gibco #15040-066) after rinsing culture plates with 1×PBS. Cells were stained with LIVE/DEAD Aqua Dead Cell stain (Invitrogen #L34957) according to manufacturer's instructions and then blocked for 20 minutes on ice with 100 ug/mL human Fc (EMD-Millipore #401104). An equal volume of antibodies diluted in BD FACS stain buffer (BD #554656) was then added and cells were incubated for 30 minutes on ice. Cells were stained with the macrophage or dendritic panels described in Table 18 and Table 19 including AF647 or APC-labelled anti-B7-H4 mAbs (clone MIH43 (BD #562787) or B7H41001 mAb (Seagen)), an anti-B7-H3 mAb (clone 7-517 (eBioscience #17-2769-42)), an anti-4-1BBL mAb (clone 5F4, (Biolegend #311506)) or non-binding isotype controls (“isotype FMO”-fluorescence minus one control). Macrophages were also stained with single positive control antibody fluorophores (e.g. single-stained controls) to set compensation values for the flow cytometry analysis. All cells were washed, centrifuged, and aspirated twice with 200 uL FACS Stain buffer before fixing in 1% PFA (Electron Microscopy Sciences #15710) in 1×PBS for flow cytometry analysis on an Attune cytometer. Alongside the myeloid cells, the B7-H4-expressing SKBR3 cell line was stained for B7-H4 surface expression as a positive control.

TABLE 18
Macrophage flow panel antibodies

| | | Cytometer | |
|---|---|---|---|
| Macrophage Panel | Purpose | Channel | Dilution |
| LIVE/DEAD Aqua | Live cell gate | VL2 | 1/100 |
| anti-CD3-BV421 | T cell dump gate | VL1 | 1/100 |
| anti-CD19-BV421 | B cell dump gate | VL1 | 1/100 |
| anti-HLADR-AF700 | myeloid marker | RL2 | 1/100 |
| anti-CD14-AF488 | monocytes/macrophages | FL1 | 1.5/100 |
| anti-CD163-PE | macrophage marker, | YL1 | 1/25 |
| | upregulated on TAM- | | |
| | like macrophages | | |
| anti-CD16-APC- | monocytes/macrophages | RL3 | 1.5/100 |
| Cy7 | | | |
| anti-B7-H4-APC | commercial antibody | RL1 | 1/50 |
| (MIH43) | for B7-H4 | | |
| Non-binding human | isotype control for | RL1 | 7 ug/mL |
| IgG1- AF647 | therapeutic | | |
| B7H41001 mAb- | SGN-B7H4V therapeutic | RL1 | 7 ug/mL |
| AF647 | antibody without | | |
| | drug linker | | |
| Non-binding mouse | Isotype control for | RL1 | 1/50 |
| IgG1- APC | commercial B7-H4 and | | |
| | B7-H3 mAb | | |
| anti-B7-H3-APC | commercial mAb for | RL1 | 1/150 |
| (7-517) | B7-H3 (positive | | |
| | control) | | |

TABLE 19
Dendritic cell flow panel antibodies

| Dendritic Cell | | Cytometer | |
|---|---|---|---|
| Panel | purpose | Channel | Dilution |
| LIVE/DEAD Aqua | Live cell gate | VL2 | 1/100 |
| anti-CD3-BV421 | T cell dump gate | VL1 | 1/100 |
| anti-CD19-BV421 | B cell dump gate | VL1 | 1/100 |
| anti-HLA-DR-AF700 | myeloid marker | RL2 | 1/100 |
| anti-CD14-AF488 | monocytes/macrophages | FL1 | 1.5/100 |
| anti-CD16-APC- | monocytes/macrophages | YL2 | 1.5/100 |
| Cy7 | | | |
| anti-CD123-PE | dendritic cell marker | YL1 | 1.5/100 |
| anti-CD11c-PE- | dendritic cell marker | RL1 | 1.5/100 |
| Cy7 | | | |
| anti 4-1BBL-APC | positive control for | RL1 | 1/50 |
| | dendritic cells | | |
| Non-binding human | isotype control for | RL1 | 7 ug/mL |
| IgG1- AF647 | therapeutic | | |
| B7H41001 mAb- | SGN-B7H4V therapeutic | RL1 | 7 ug/mL |
| AF647 | antibody without | | |
| | drug linker | | |
| Non-binding mouse | Isotype control for | RL1 | 1/50 |
| IgG1- APC | commercial 4-1 BBL | | |
| | mAb | | |

Macrophage, lymphocyte, and dendritic cell gates were applied using FCS files in FlowJo. Geometric mean of fluorescence intensities (gMFI) of all HLA-DR⁺CD19⁻CD3⁻ (monocytes and MO macrophages), HLA-DR⁺SSC-A^{hi} subsets (TAM-like and M1 macrophages), or HLA-DR⁺CD19⁻CD3⁻CD11c⁺CD123⁺ were exported to Excel where fold over isotype control was calculated and transferred to GraphPad Prism 8 where graphs were plotted.

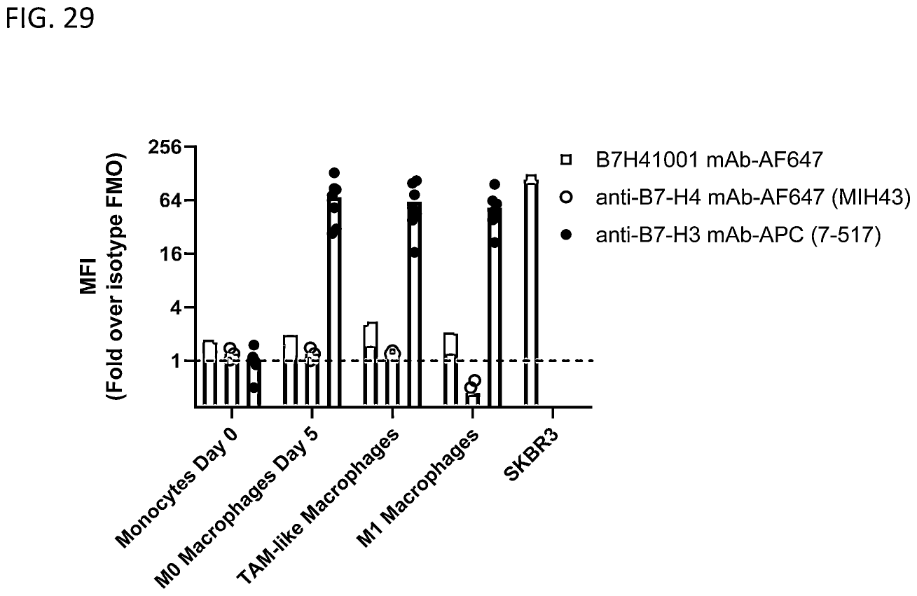

As shown in FIG. 29, Expression of B7-H4 was low on all monocyte and macrophage subsets (average<2-fold over isotype FMO). In contrast, expression of B7-H3 was high on all three differentiated macrophage subsets (average˜50-fold over isotype FMO). Expression of B7-H4 was high on SKBR3 cells, a human breast cancer cell line that endogenously expresses B7-H4 that was stained as a positive control.

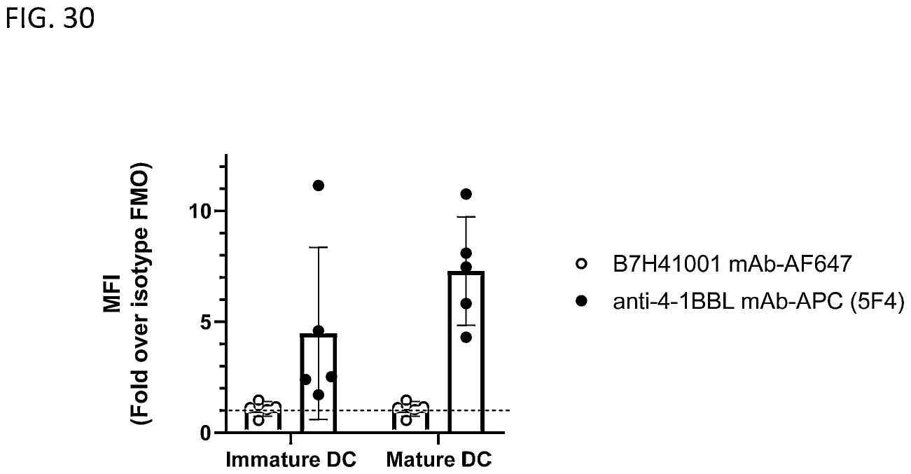

Expression levels of B7-H4 on monocyte-derived dendritic cell (DC) subsets from 5 donors was also analyzed by flow cytometry. As shown in FIG. 30, expression of B7-H4 on both immature DCs and TNFα-treated mature DCs was similar to the isotype FMO. In contrast, expression of 4-1BBL, a co-stimulatory molecule expressed on DCs (Futagawa et al., 2002, nt Immunol 14, 275-286; Hurtado et al., 1995, J Immunol 155, 3360-3367), was expressed at moderate levels on both DC subsets (˜4-7-fold over isotype FMO).

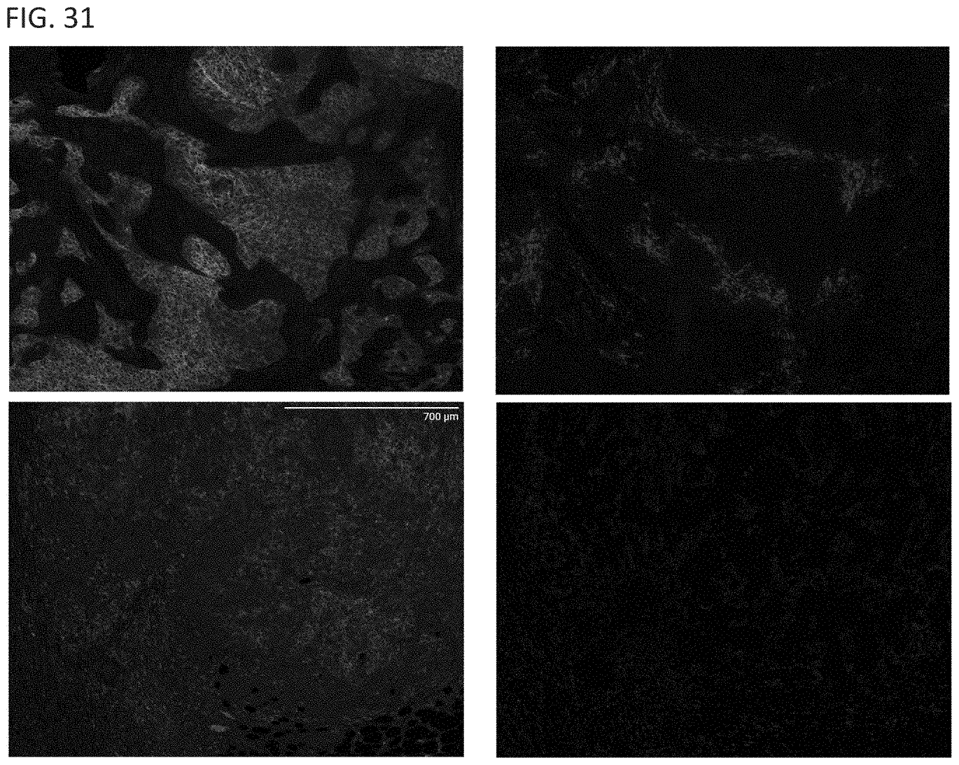

B7-H4 expression on CD163+ macrophages in human tumors was also examined by dual immunofluorescent staining for B7-H4 and CD163. No co-expression of B7-H4 and CD163 was observed on 14 tumor samples examined. A representative example of co-staining is shown in FIG. 31.

Altogether, this data suggests that, consistent with the RNA expression data, expression of B7-H4 was low or absent on myeloid immune cell subsets, including monocytes, macrophages, and dendritic cells.

### Example 14: Non-Human Primate (NHP) and Rat Tox Study

The nonclinical safety profile of SGN-B7H4V supports the proposed initial clinical development plan. SGN-B7H4V was tolerated in the rat and cynomolgus monkey with a dosing regimen that established the highest non-severely toxic dose (HNSTD) in both rat and cynomolgus monkey as well as a significantly toxic dose in 10% of the rat (STD10). Findings from pivotal GLP and non-GLP studies suggest that the primary target organs of SGN-B7H4V-related toxicity are the hematological system, testes, and ovaries. The hematologic toxicity is consistent with the mechanism of action (MOA) for MMAE. SGN-B7H4V is tolerated in rat and non-human primate (NHP) toxicity studies at doses consistent with approved vedotin ADCs.

### Example 15: SGN-B7H4V Induces Hallmarks of ICD In Vitro (ATP, HMGB1, and Calreticulin) and Elicits Immune Cell Activation In Vitro

Tubulin destabilization driven by the vedotin payload MMAE induces ER stress, which results in induction of immunogenic cell death (ICD), a form of cell death characterized by release of immune-stimulatory molecules that may activate an innate and subsequent adaptive immune response. Hallmarks of immunogenic cell death include release of the immunostimulatory molecules ATP and HMGB1 as well as surface exposure of calreticulin, which may drive innate and subsequent adaptive immune responses (Chaput et al., 2007; Kepp et al., 2014). Here, we evaluated the ability of SGN-B7H4V (antibody-drug conjugate, or “ADC” hereafter) to elicit these early hallmarks of ICD.

### Methods

SKBR3 cells were cultured in RPMI containing 10% fetal bovine serum (FBS) and penicillin/streptomycin (P/S) and passaged every 3-4 days at ˜1:5 dilution. On day 0, cells were collected with 0.05% Trypsin-EDTA (Gibco #25300-054), resuspended in complete media, and ˜120,000 cells in 1 mL media were added to each well of a 12-well plate (ThermoFisher Scientific #150628). The next day, the media from each well was removed and replaced with 1 mL of fresh media containing 1 μg/mL ADC or mAb or 100 nM MMAE. On day 3, 48 hours after treatment, media from each well of the 12-well plate was transferred to a 96-well, 2 ml plate (USA Scientific #18962800) and spun at 1500 rpm for 5 minutes (min) to remove cell debris and non-adherent “floating” cells. Then 200 mL of supernatants were transferred to a standard 96-well, round-bottom plate (ThermoFisher Scientific #163320). These supernatants were used immediately for the ATP assay (described below) or frozen at −20° C. for the HMGB1 assay (described below). Finally, 500 μL of non-enzymatic dissociation buffer (ThermoFisher Scientific/Gibco #13151-014) was added to each well to remove remaining adherent cells. Harvested adherent cells were combined with the pelleted “floating” cells collected above and stained for flow cytometry as described below.

### ATP and HMGB1 Release

ATP release was evaluated as follows immediately after collection of supernatants as described above. The CellTiter Glo reagents (Promega #G755A) were brought to room temperature (RT) before use, and 50 μL of supernatant was transferred to a black-walled, clear-bottom 96-well plate in duplicate and combined with 50 μL of reconstituted CellTiter Glo reagent. The plates were mixed briefly, sealed, and analyzed on an Envision plate reader within 20 minutes following the addition of the CellTiter Glo. HMGB1 release was evaluated using the HMGB1 Express ELISA kit according to the manufacturer's protocol (Tecan #30164033).

### Calreticulin Exposure

Calreticulin exposure was evaluated by flow cytometric staining as follows. Live/Dead (L/D) staining buffer [ThermoFisher Scientific #L10119] was prepared by reconstituting the dye in 50 μL of DMSO and transferring to a 50 mL conical containing 50 mL of PBS. The cells collected above were resuspended in 1 mL of freshly prepared L/D staining buffer and incubated for 20 min at room temperature (RT). Next, the cells were pelleted and washed twice with FACS buffer (PBS containing 2% FBS). Annexin V/Calreticulin/PI staining solution was prepared as follows:
FACS buffer (2% FBS/PBS)—90 μL per well
10× Annexin V binding buffer (Fisher Scientific #BDB556454)—10 μL per well
Annexin V-FITC (Fisher Scientific #BDB556419)—2 μL per well
Calreticulin-A647 (Abcam #ab196159)—2 μL per well
PI (Propidium Iodide, ThermoFisher Scientific #R37169)—5 μL per well

Cells in each well were resuspended in 100 μL staining solution and incubated for 20 min at RT. Next, the cells were washed twice with 1 mL of FACS/Annexin V binding buffer (1×), resuspended in 250 μL FACS/Annexin V staining buffer, and analyzed on an Attune flow cytometer (ThermoFisher Scientific).

### Tumor/PBMC Co-Culture Assay

Tumor cells were transfected with Incucyte® Cytolight red lentivirus per manufacturer's instructions and stable polyclonal cell populations expressing mKate2 (red fluorescent protein, RFP) were generated under puromycin selection. Live-cell killing assays were performed by seeding RFP+ MDA-MB-468 tumor cells in 96-well flat bottom plates (Corning #3603) at a variety of densities (3,750-10,000 cells/well) and grown overnight. The following day, PBMCs isolated from healthy donors were added at 15:1 or 25:1 effector to target (E:T) ratios and cultures were treated with the indicated small molecule drugs or ADCs. To evaluate immune cell activation, supernatants were harvested between 120 and 144 hours post treatment and MIP-1β production was evaluated by Milliplex MAP Human cytokine/chemokine/growth factor panel A (12-plex) Immunology Multiple Assay (Millipore Sigma #HCYTA-60K-12C). Cells on duplicate plates were dissociated (TrypLE Express, Gibco) between 48 and 86 hours post treatment and dead cells were stained with LIVE/DEAD Fixable Near-IR Dead Cell Stain Kit (ThermoFisher, L34994) per manufacturer's instructions. After the viability stain, Fc-gamma receptors were blocked using human TruStain FcX (Biolegend, 422302). Cells were washed 1× with cell staining buffer (BD, 554657) and subsequently stained (30 min, at 4° C.) with antibodies for detection of surface antigens. The following antibodies were used: CD8 eFluor 450 (clone OKT8, ThermoFisher), CD14 BV650 (clone M5E2, Biolegend), HLA-DR BV785 (clone L243, Biolegend), CD19 APC-eFluor 780 (clone HIB19, ThermoFisher), CD3 FITC (clone OKT3, ThermoFisher), CD56 PerCP-eF710 (clone TULY56, ThermoFisher), CD69 PE-Cy7 (clone FN50, Biolegend), CD86 APC (clone IT2.2, ThermoFisher), and CD45 A700 (clone 2D1, Biolegend). Following the final wash, cell pellets were resuspended in 200 μL staining buffer and analyzed on an NXT Attune flow cytometer (ThermoFisher). Flow cytometry data were analyzed using FlowJo software and monocyte activation was quantified by measuring the MFI of CD86 staining on CD14+ monocytes within the RFP−/live/CD19−/CD3−/CD14+ gate.

### Results—SGN-B7H4V Induces Release of ATP and HMGB1 as Well as Calreticulin Exposure and Elicits Immune Cell Activation In Vitro

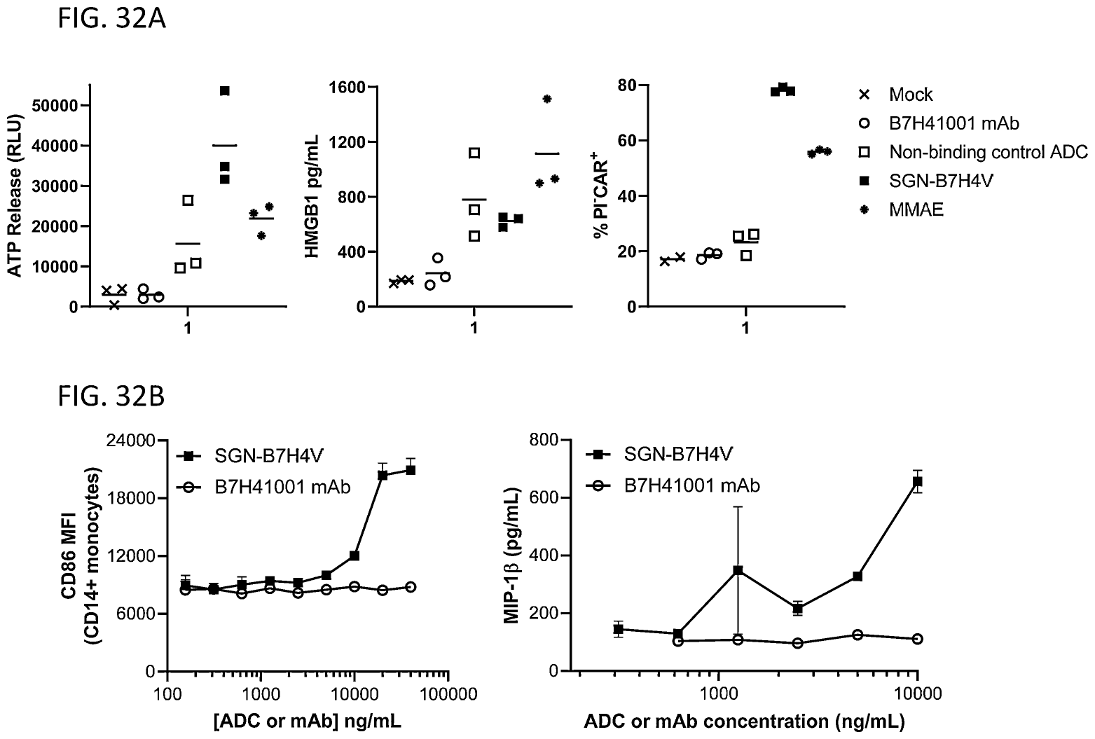

Treatment of SKBR3 breast cancer cells, which endogenously express B7-H4, with SGN-B7H4V or MMAE free drug led to the release of ATP and HMGB1 as well as surface exposure of calreticulin (FIG. 32A). In contrast, this was not observed following treatment with the unconjugated mAb backbone B7H41001. Moreover, in tumor/immune cell co-culture experiments, treatment of co-cultures with SGN-B7H4V, but not the unconjugated mAb backbone B7H41001, led to upregulation of the costimulatory molecule CD86 on CD14+ monocytes (FIG. 32B, left panel). In addition, SGN-B7H4V treatment of tumor/immune cell co-cultures (but not tumor cells alone, data not shown) led to the release of the chemoattractant MIP-1β/CCL4 (FIG. 32B, right panel). This suggests that, similar to other vedotin ADCs, SGN-B7H4V induces hallmarks of immunogenic cell death (Klussman K, 2020) and drives innate immune cell activation in vitro (Gray et al SITC 2020 poster).

### Example 16: SGN-B7H4V Induces Immunomodulatory Changes in the MDA-MB-468 Xenograft Tumor Model of Triple Negative Breast Cancer (TNBC) that are Distinct from Other Microtubule Disrupting Payloads

The ability of SGN-B7H4V to induce immunomodulatory changes in vivo in the MDA-MB-468 xenograft model of triple-negative breast cancer (TNBC) was then evaluated. B7-H4-expressing human MDA-MB-468 xenograft tumors were treated i.p. with a single 3 mg/kg dose of the vehicle control, unconjugated B7H41001 mAb, or SGN-B7H4V (antibody-drug conjugate, or “ADC” hereafter). Tumors were harvested 7 days after treatment, cut in half, and processed for RNA-seq or IHC. To compare the immunomodulatory changes following treatment with B7H41001 mAb conjugated to the vedotin payload (SGN-B7H4V) versus other microtubule disrupting payloads, MDA-MB-468 xenograft tumors were also treated i.p. with a single dose of B7H41001 mAb conjugated to DM1/emtansine (6 mg/kg) or DM4/ravtansine (6 mg/kg). Tumors were harvested 7 days after treatment, cut in half, and processed for RNA-seq or IHC.

Specifically, female NSG mice were implanted with 1×10⁶ MDA-MB-468 cells in 25% Matrigel H C subcutaneously. Once tumor volumes reached ˜250-300 mm³, mice were randomized into treatment groups of 6 mice each and treated with a single 3 mg/kg dose of ADC or mAb, or vehicle control (20 mM Histidine buffer, pH 6.0) injected intraperitoneally (i.p.). Stock concentrations of ADC were diluted to desired concentration with 20 mM Histidine buffer, pH 6.0 or and stock concentration of mAb were diluted to desired concentration with 0.01% Tween20 in PBS. Twenty-four hours prior to dosing, each animal was treated with 10 mg/kg hIVIG. One week following treatment, tumors were harvested, cut in half, and processed for RNAseq (frozen at −80° C.) or formalin-fixed and paraffin-embedded for immunohistochemical (IHC) analysis.

For RNAseq, RNA was extracted from tumors and library preparation for Illuminex sequencing was performed using PolyA selection. RNA was sequenced using the Illumina HiSeq platform 2×150 bp configuration. RNA extraction, library preparation, and sequencing were performed by GENEWIZ. Adaptors were trimmed using cutadapt version 1.16, reads were aligned to a composite (GRCh38(h38)/GRCm38(mm10)) genome using STAR version 2.5.2. Transcripts and genes were quantitated via RSEM version 1.2.31. Quantitative and statistical differences were determined between groups of samples using DESeq2 version 1.28.1. GO term and gene set enrichment analysis was performed with clusterProfiler version 3.16.1, msigdbr version 7.1.1, AnnotationDbi version 1.50.3, org.Mm.eg.db version 3.11.4, and org.Hs.eg.db version 3.11.4. For GO term analysis, genes were selected which had an unadjusted p-value 0.05 or lower. Up and down regulated genes in the mouse or human component were each analyzed independently for all pairwise comparisons of vehicle, non-binding ADC, B7H41001 mAb, and/or SGN-B7H4V-treated samples. Only GO terms with a p-value and q-value of 0.05 or less were retained. Figures were replotted using GraphPad Prism. The raw fastq files as well as the mapped BAM files are stored on the Seagen linux storage location: /fc1/jobs/research/RNASeq/. One vehicle control tumor was excluded from analysis due to degradation.

For IHC, FFPE blocks were sectioned at 4 μm and sections were placed on charged slides. Slides were baked for 1 hr at 60° C. Slides were deparaffinized and rehydrated by immersing the slides through the following solutions: two times xylene 3 minutes, two times 100% EtOH for 2 minutes, 90% EtOH 2 minutes, 70% EtOH 2 minutes followed by deionized water. Heat induced antigen retrieval (HIER) was performed using DIVA Decloaking solution (Biocare, cat #DV2004MX) in a Nx Gen Decloaking Chamber (Biocare) using the default setting. Slides were cooled down with deionized water and placed in TBST wash buffer prior to immunohistochemistry (IHC). All samples were processed on an IntelliPath autostainer (Biocare, Pacheco, Calif.) at R.T. Peroxidazed 1 (Biocare, cat #PX968) was applied for 10 minutes to all slides. Avidin and Biotin blocking reagent (Vector labs, cat #SP-2001) were applied to CD86 and Granzyme B slides for 15 minutes each. Background Sniper (Biocare, cat #BS966MM) was applied for 10 minutes to all slides to block nonspecific background. All antibodies were diluted to working concentration in DaVinci Green diluent (Biocare, cat #PD900M). Isotype-matched rabbit IgG (Jackson Immunoresearch, cat #011-000-003), rat IgG2a (BD Pharmingen, cat #555841) and rat IgG (Invitrogen, cat #16-4301-85) was used as a negative control for background staining. Slides were incubated with CD3 (BioRad, cat #MCA1477) at 1:500, CD8 (Cell Signaling, #98941) at 1:200, CD11c (Cell Signaling, cat #97585) at 1:200, F4/80 (BioRad, cat #MCA497) at 1:200, CD163 (Abcam, cat #ab182422) at 1:100, CD206 (Cell Signaling, cat #24595S) at 1:2500, CD86 (Invitrogen, cat #MA1-10299) at 1:1000, PD-L1 (Cell Signaling, cat #64988S) at 1:200, CD4 (Abcam, cat #ab183685) at 1:1000, PD-1 (Cell Signaling, cat #84651S) at 1:40, Ki-67 (Abcam, cat #ab15580) at 1:2000, Granzyme B (Invitrogen, cat #PA1-26616) at 1:1500, Chitinase 3-like 3 (R&D, cat #MAB2446) at 1:100 or appropriate isotype control antibody for 1 hour.

Slides were rinsed twice with TBST (Biocare, cat #TWB945M). Slides labeled with CD8, F4/80, CD11c, CD163, CD206, PD-L1, CD4, PD-1, Ki-67 and Granzyme B were incubated 30 minutes with Rabbit Envision HRP polymer (Dako, cat #K4001). Slides labeled with CD3, CD86 and Chitinase 3-like 3 were incubated 30 minutes with Rat Polymer HRP (Vector labs, cat #MP-7404-50). Slides were washed twice in TBST. Slides labeled with CD86 and Granzyme B were amplified the signals by applying TSA (PerkinElmer, cat #SAT700001KT) at 1:100 for 5 minutes followed with SA-HRP (PerkinElmer, cat #NEL750001) at 1:1000 for 30 minutes. Detection of HRP was done with ImmPact DAB (Vector labs, cat #SK-4103) to all slides for 5 minutes. Sections were counterstained with hematoxylin, diluted 1:10 in deionized water, for 5 minutes (Surgipath, cat #3801570). Upon protocol completion on the autostainer, the slides were immediately removed and placed in deionized (DI) water before going through a series of dehydration steps (70% EtOH, 70% EtOH, 95% EtOH, 95% EtOH, 100% EtOH, 100% EtOH, 100% EtOH, Xylenes×3) to allow for cover slipping (Tissue-Tek g2) using Surgipath mounting medium (Leica, cat #3801731). Images were captured using a slides scanner (Leica, Aperio AT2 or Vectra, Polaris) and reviewed by an ACVP board-certified veterinary pathologist.

Scanned images were analyzed with Halo image analysis software v. 3.1.1076 (Indica Labs), using the area quantification algorithm for CD11c and F4/80 and the cytonuclear algorithm for all other antibodies. A classifier was trained to allow the software to determine tumor, stroma, and glass. The classifier was added to the algorithm. The algorithm was optimized based on staining intensity and background staining. Percent area of positive tissue, tumor and stroma were determined.

### SGN-B7H4V Exhibited Anti-Tumor Activity in the MDA-MB-468 Xenograft Model of TNBC

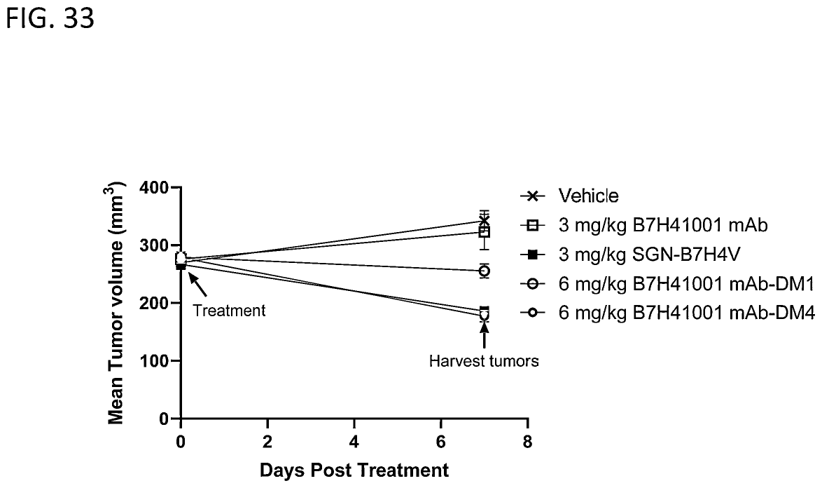

As shown in FIG. 33, treatment of NSG mice bearing MDA-MB-468 xenograft tumors with a single 3 mg/kg dose of SGN-B7H4V, but not the vehicle or B7H41001 mAb, led to a reduction in tumor volume. Treatment with a two-fold higher dose (6 mg/kg) of B7H41001 mAb-DM1 and B7H41001 mAb-DM4 conjugates also led to a reduction in tumor volume, compared to vehicle and mAb alone though the DM1 conjugate elicited less antitumor activity compared to SGN-B7H4V.

### SGN-B7H4V Recruited Mouse Macrophages to a Xenograft Tumor

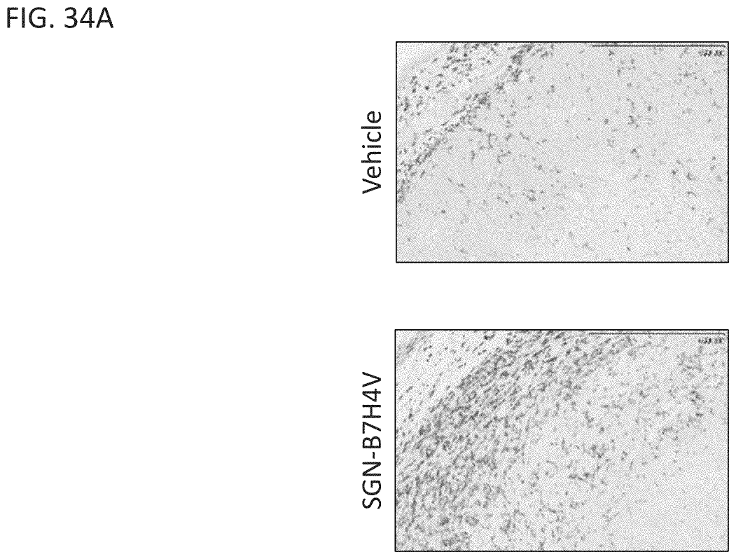

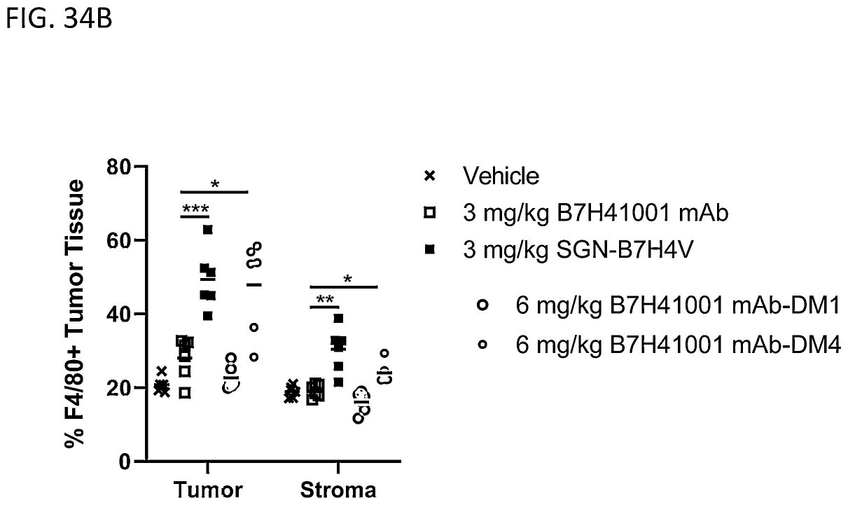

The ability of SGN-B7H4V to elicit recruitment of mouse innate immune cells to the tumor site was then evaluated by immunohistochemical staining for macrophage marker F4/80+ (Anti Mouse F4/80 Antibody from BioRad, cat #MCA497). Quantification of stained tumor sections revealed an increase in the proportion of F4/80+ macrophages in SGN-B7H4V-treated tumors (FIGS. 34A and 34B)—including an increase in the proportion of F4/80+ macrophages in the tumor nest as well as the surrounding tumor stroma, suggesting that SGN-B7H4V promoted recruitment of mouse innate immune cells to xenograft tumors. This is consistent with the ability of other vedotin ADCs to induce recruitment of immune cells to tumors in both preclinical models (Gray, 2020; Liu, 2020) and in patients (Pusztai, 2020). In contrast, treatment with B7H41001 mAb-DM1 conjugates did not elicit an increase in F4/80+ macrophages in the tumor nest or stroma; treatment with B7H41001 mAb-DM4 conjugates led to an increase in F4/80+ macrophages in the tumor nest and stroma but to a lesser extent than following treatment with SGN-B7H4V.

### SGN-B7H4V Induced Upregulation of Cytokines and Type I Interferon Response Genes by Human Tumors Cells

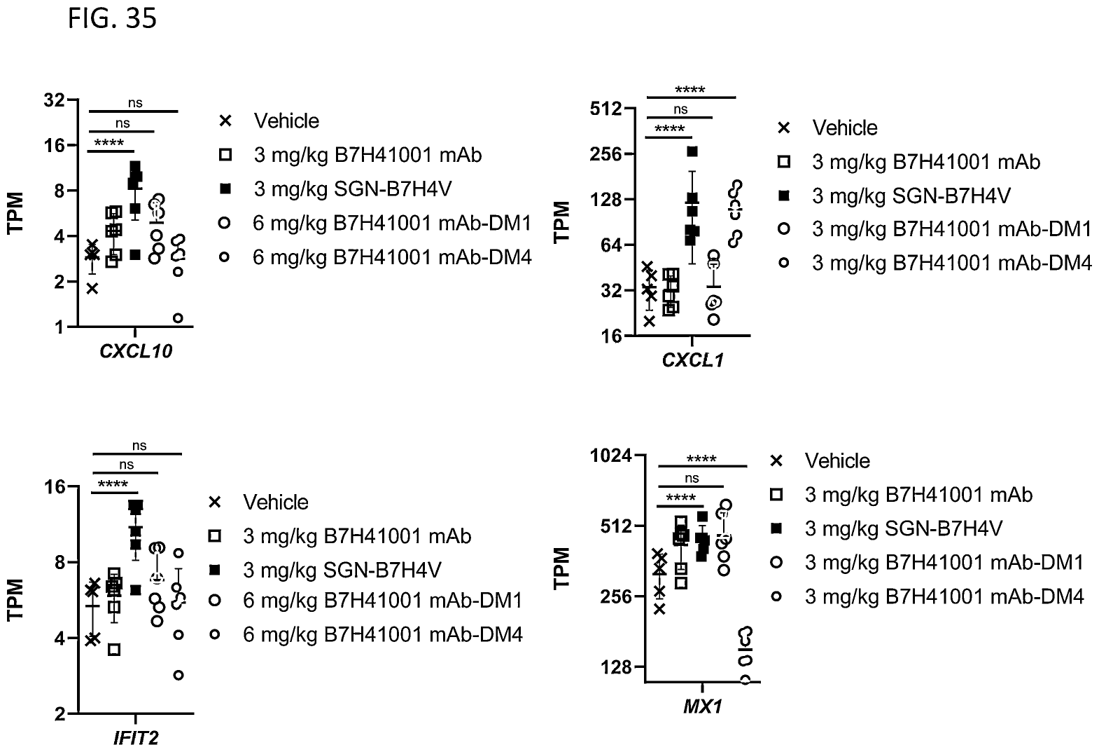

RNAseq analysis was also performed (Illumina HiSeq platform) and transcript reads were mapped to the human and mouse genomes to determine gene expression changes induced by SGN-B7H4V in the human MDA-MB-468 tumor cells or mouse immune and stromal cells, respectively. Human transcripts encoding cytokines (CXCL10 and CXCL1) and type I interferon (IFN) response genes (IFIT2 and MX1) were significantly upregulated (˜2-3 fold and ˜1.5 fold, respectively) in SGN-B7H4V-treated tumors compared to vehicle control (FIG. 35). The expression of these genes could promote immune cell activation and recruitment to tumors.

In contrast, treatment with B7H41001 mAb-DM1 conjugates did not elicit an increase in CXCL10, CXCL1, IFIT2, or MX1 (FIG. 35). While B7H41001 mAb-DM4 conjugates drove a similar inflammatory response as SGN-B7H4V (e.g. CXCL1 upregulation), treatment with B7H41001 mAb-DM4 conjugates did not elicit an increase in CXCL10 or IFIT2, and surprisingly elicited a decrease in MHC class I molecules (HLA-A, HLA-B, HLA-C, and B2M, Table 20) and the type I IFN response gene MX1 (FIG. 35). Analysis of several additional type I IFN response genes including IFIT1, IFIT3, ISG15, OAS2, and RSAD2 revealed an increase following treatment with SGN-B7H4V which was not seen with B7H41001 mAb-DM1 treatment and, conversely, decreased following treatment with B7H41001 mAb-DM4 (Table 20). Similarly, genes involved in MHC class I antigen presentation including B2M, HLA-A, HLA-B, HLA-C were significantly decreased following treatment with B7H41001 mAb-DM4, but not SGN-B7H4V or B7H41001 mAb-DM1 treatment (Table 20). Of the microtubule disrupting ADCs tested only SGN-B7H4V drove macrophage infiltration into the tumor and the stoma, elicited both an inflammatory and type 1 interferon response, and led to elevation in the antigen presentation machinery TAP1 and TAP2 (Table 20).

TABLE 20
RNAseq Analysis of expression of additional genes of interest by SGN-B7H4V,
B7H41001 mAb-DM1, and B7H41001 mAb-DM4-treated MDA-MB-468 tumors

| | | | Adjusted | Fold |
|---|---|---|---|---|
| GeneSymbol | Species | Comparison | P-value | Change |
| B2M | human | B7H41001 mAb-DM1 vs. vehicle | 0.995 | 0.999 |
| B2M | human | B7H41001 mAb-DM4 vs. vehicle | 0.000566 | 0.826 |
| B2M | human | SGN-B7H4V vs. vehicle | 0.375 | 1.06 |
| CCL20 | human | B7H41001 mAb-DM1 vs. vehicle | 0.823 | 1.00 |
| CCL20 | human | B7H41001 mAb-DM4 vs. vehicle | 0.000222 | 1.62 |
| CCL20 | human | SGN-B7H4V vs. vehicle | 0.00837 | 1.44 |
| CD274 | human | B7H41001 mAb-DM1 vs. vehicle | 0.682 | 1.00 |
| CD274 | human | B7H41001 mAb-DM4 vs. vehicle | 3.47E−19 | 6.54 |
| CD274 | human | SGN-B7H4V vs. vehicle | 5.75E−11 | 3.37 |
| CD276 | human | B7H41001 mAb-DM1 vs. vehicle | 0.982 | 0.998 |
| CD276 | human | B7H41001 mAb-DM4 vs. vehicle | 0.00784 | 1.15 |
| CD276 | human | SGN-B7H4V vs. vehicle | 0.528 | 0.972 |
| CXCL1 | human | B7H41001 mAb-DM1 vs. vehicle | 0.961 | 0.999 |
| CXCL1 | human | B7H41001 mAb-DM4 vs. vehicle | 1.97E−11 | 3.17 |
| CXCL1 | human | SGN-B7H4V vs. vehicle | 0.00000709 | 2.47 |
| CXCL10 | human | B7H41001 mAb-DM1 vs. vehicle | 0.124 | 1.25 |
| CXCL10 | human | B7H41001 mAb-DM4 vs. vehicle | 0.648 | 0.934 |
| CXCL10 | human | SGN-B7H4V vs. vehicle | 4.33E−10 | 2.39 |
| CXCL8 | human | B7H41001 mAb-DM1 vs. vehicle | 0.92 | 1.01 |
| CXCL8 | human | B7H41001 mAb-DM4 vs. vehicle | 1.27E−09 | 3.88 |
| CXCL8 | human | SGN-B7H4V vs. vehicle | 1.76E−07 | 3.33 |
| CXCL9 | human | B7H41001 mAb-DM1 vs. vehicle | | 1.00 |
| CXCL9 | human | B7H41001 mAb-DM4 vs. vehicle | 0.8 | 1.02 |
| CXCL9 | human | SGN-B7H4V vs. vehicle | 0.954 | 1.00 |
| HLA-A | human | B7H41001 mAb-DM1 vs. vehicle | 0.915 | 0.995 |
| HLA-A | human | B7H41001 mAb-DM4 vs. vehicle | 0.000457 | 0.785 |
| HLA-A | human | SGN-B7H4V vs. vehicle | 0.814 | 0.981 |
| HLA-B | human | B7H41001 mAb-DM1 vs. vehicle | 0.978 | 1.00 |
| HLA-B | human | B7H41001 mAb-DM4 vs. vehicle | 0.0492 | 0.801 |
| HLA-B | human | SGN-B7H4V vs. vehicle | 0.391 | 1.09 |
| HLA-C | human | B7H41001 mAb-DM1 vs. vehicle | 0.957 | 1.00 |
| HLA-C | human | B7H41001 mAb-DM4 vs. vehicle | 0.00202 | 0.798 |
| HLA-C | human | SGN-B7H4V vs. vehicle | 0.356 | 1.07 |
| HLA-DMA | human | B7H41001 mAb-DM1 vs. vehicle | 0.729 | 0.994 |
| HLA-DMA | human | B7H41001 mAb-DM4 vs. vehicle | 0.00323 | 0.627 |
| HLA-DMA | human | SGN-B7H4V vs. vehicle | 0.42 | 0.912 |
| HLA-DRB1 | human | B7H41001 mAb-DM1 vs. vehicle | | 0.997 |
| HLA-DRB1 | human | B7H41001 mAb-DM4 vs. vehicle | 0.44 | 1.10 |
| HLA-DRB1 | human | SGN-B7H4V vs. vehicle | 0.586 | 0.954 |
| IFIT1 | human | B7H41001 mAb-DM1 vs. vehicle | 0.251 | 1.01 |
| IFIT1 | human | B7H41001 mAb-DM4 vs. vehicle | 1.46E−17 | 0.399 |
| IFIT1 | human | SGN-B7H4V vs. vehicle | 0.000135 | 1.59 |
| IFIT2 | human | B7H41001 mAb-DM1 vs. vehicle | 0.403 | 1.01 |
| IFIT2 | human | B7H41001 mAb-DM4 vs. vehicle | 0.592 | 1.07 |
| IFIT2 | human | SGN-B7H4V vs. vehicle | 2.68E−11 | 1.95 |
| IFIT3 | human | B7H41001 mAb-DM1 vs. vehicle | 0.253 | 1.01 |
| IFIT3 | human | B7H41001 mAb-DM4 vs. vehicle | 0.00000148 | 0.6 |
| IFIT3 | human | SGN-B7H4V vs. vehicle | 0.00000231 | 1.73 |
| ISG15 | human | B7H41001 mAb-DM1 vs. vehicle | 0.27 | 1.01 |
| ISG15 | human | B7H41001 mAb-DM4 vs. vehicle | 0.00178 | 0.654 |
| ISG15 | human | SGN-B7H4V vs. vehicle | 1.51E−07 | 1.83 |
| MX1 | human | B7H41001 mAb-DM1 vs. vehicle | 0.0639 | 1.28 |
| MX1 | human | B7H41001 mAb-DM4 vs. vehicle | 4.37E−15 | 0.516 |
| MX1 | human | SGN-B7H4V vs. vehicle | 0.0000225 | 1.42 |
| OAS2 | human | B7H41001 mAb-DM1 vs. vehicle | 0.0687 | 1.23 |
| OAS2 | human | B7H41001 mAb-DM4 vs. vehicle | 3.50E−18 | 0.436 |
| OAS2 | human | SGN-B7H4V vs. vehicle | 0.0000126 | 1.42 |
| RSAD2 | human | B7H41001 mAb-DM1 vs. vehicle | 0.0981 | 1.24 |
| RSAD2 | human | B7H41001 mAb-DM4 vs. vehicle | 9.65E−09 | 0.525 |
| RSAD2 | human | SGN-B7H4V vs. vehicle | 0.00000283 | 1.63 |
| TAP1 | human | B7H41001 mAb-DM1 vs. vehicle | 0.142 | 1.12 |
| TAP1 | human | B7H41001 mAb-DM4 vs. vehicle | 0.252 | 1.08 |
| TAP1 | human | SGN-B7H4V vs. vehicle | 3.40E−07 | 1.35 |
| TAP2 | human | B7H41001 mAb-DM1 vs. vehicle | 0.422 | 1.03 |
| TAP2 | human | B7H41001 mAb-DM4 vs. vehicle | 9.93E−20 | 1.67 |
| TAP2 | human | SGN-B7H4V vs. vehicle | 2.64E−18 | 1.62 |
| TREX1 | human | B7H41001 mAb-DM1 vs. vehicle | 0.895 | 0.995 |
| TREX1 | human | B7H41001 mAb-DM4 vs. vehicle | 0.987 | 0.998 |
| TREX1 | human | SGN-B7H4V vs. vehicle | 0.0834 | 1.11 |
| VTCN1 | human | B7H41001 mAb-DM1 vs. vehicle | 0.783 | 0.991 |
| VTCN1 | human | B7H41001 mAb-DM4 vs. vehicle | 6.48E−55 | 0.395 |
| VTCN1 | human | SGN-B7H4V vs. vehicle | 2.89E−30 | 0.489 |

Gene ontology (GO) term analysis was also performed and results for B7H41001 mAb-DM1 compared to SGN-B7H4V (Table 21) and B7H41001 mAb-DM4 compared to SGN-B7H4V (Table 22) were filtered based on a cutoff of adjusted p-value<0.01 and Biological Process (BP) ontology. Human gene categories related to apoptosis/programmed cell death pathways (e.g. apoptotic mitochondrial changes, regulation of cysteine-type endopeptidase activity involved in apoptotic process, and positive regulation of programmed cell death) were elevated with SGN-B7H4V treatment compared to B7H41001 mAb-DM1 (Table 21), consistent with more robust tumor shrinkage with SGN-B7H4V. Moreover, several mouse immune-related GO terms were increased following treatment with SGN-B7H4V compared to B7H41001 mAb-DM1 (e.g. response to virus, antigen processing and presentation, positive regulation of cytokine production, cellular response to interferon-beta, regulation of tumor necrosis family superfamily cytokine production, macrophage activation, positive regulation of innate immune response, regulation of leukocyte chemotaxis, myeloid leukocyte migration) (Table 21). Overall, these findings suggest that SGN-B7H4V drives more immunomodulatory changes to the tumor microenvironment than B7H41001 mAb-DM1, consistent with both IHC and RNAseq analysis which show a superior ability of SGN-B7H4V compared to B7H41001 mAb-DM1 to recruit F4/80+ macrophages to the tumor nest as well as the surrounding tumor stroma (FIG. 34). Human gene categories related to the immune response were also elevated following treatment with SGN-B7H4V compared to B7H41001 mAb-DM4 (e.g. defense response to virus, antigen processing and presentation of endogenous peptide antigen via MHC class I, positive regulation of type I interferon production, response to type I interferon, regulation of innate immune response) (Table 22). This points to potential differences in the immunomodulatory effects of the two maytansinoid based ADCs, with vedotin based SGN-B7H4V driving more robust immune changes in the human tumor cells. Moreover, this is consistent the observation that SGN-B7H4V elicited increased type I interferon response gene expression, while B7H41001 mAb-DM4 elicited no change (CXCL10 and IFIT2) or a significant decrease (MX-1) in type I IFN response gene expression. Also, several mouse immune-related gene categories were elevated following treatment with SGN-B7H4V compared to B7H41001 mAb-DM4 (Table 22). These include leukocyte migration, positive regulation of cytokine production, response to interferon-beta, myeloid leukocyte migration, and regulation of chemotaxis (Table 22). Altogether, this suggests that SGN-B7H4V drives more robust immunomodulatory changes to both the human tumor cells and mouse immune cells compared to B7H41001 mAb-DM4. Altogether, this body of data suggests that treatment with SGN-B7H4V results in robust immunomodulatory changes in vivo that are distinct from other microtubule disrupting payloads.

TABLE 21
Gene Ontology (GO) analysis comparing gene expression changes
following treatment with B7H41001 mAb-DM1 versus SGN-B7H4V

| | | | | | | Adjusted |
|---|---|---|---|---|---|---|
| Species | Comparison | Direction | ONTOLOGY | ID | Description | p-value |
| human | B7H41001 mAb-DM1 | Down.in.Test/ | BP | GO:0042254 | ribosome biogenesis | 4.24E−12 |
| | vs. SGN-B7H4V | Control | | | | |
| human | B7H41001 mAb-DM1 | Down.in.Test/ | BP | GO:0034470 | ncRNA processing | 3.92E−10 |
| | vs. SGN-B7H4V | Control | | | | |
| human | B7H41001 mAb-DM1 | Down.in.Test/ | BP | GO:0016072 | rRNA metabolic process | 4.98E−10 |
| | vs. SGN-B7H4V | Control | | | | |
| human | B7H41001 mAb-DM1 | Down.in.Test/ | BP | GO:0006364 | rRNA processing | 4.98E−10 |
| | vs. SGN-B7H4V | Control | | | | |
| human | B7H41001 mAb-DM1 | Down.in.Test/ | BP | GO:0034660 | ncRNA metabolic process | 5.78E−09 |
| | vs. SGN-B7H4V | Control | | | | |
| human | B7H41001 mAb-DM1 | Down.in.Test/ | BP | GO:0022613 | ribonucleoprotein complex | 1.61E−08 |
| | vs. SGN-B7H4V | Control | | | biogenesis | |
| human | B7H41001 mAb-DM1 | Down.in.Test/ | BP | GO:0007005 | mitochondrion organization | 4.03E−07 |
| | vs. SGN-B7H4V | Control | | | | |
| human | B7H41001 mAb-DM1 | Down.in.Test/ | BP | GO:0140014 | mitotic nuclear division | 4.75E−07 |
| | vs. SGN-B7H4V | Control | | | | |
| human | B7H41001 mAb-DM1 | Down.in.Test/ | BP | GO:0006457 | protein folding | 9.49E−06 |
| | vs. SGN-B7H4V | Control | | | | |
| human | B7H41001 mAb-DM1 | Down.in.Test/ | BP | GO:0008637 | apoptotic mitochondrial changes | 7.58E−05 |
| | vs. SGN-B7H4V | Control | | | | |
| human | B7H41001 mAb-DM1 | Down.in.Test/ | BP | GO:0006839 | mitochondrial transport | 1.96E−04 |
| | vs. SGN-B7H4V | Control | | | | |
| human | B7H41001 mAb-DM1 | Down.in.Test/ | BP | GO:1902850 | microtubule cytoskeleton | 2.59E−04 |
| | vs. SGN-B7H4V | Control | | | organization involved in mitosis | |
| human | B7H41001 mAb-DM1 | Down.in.Test/ | BP | GO:0042273 | ribosomal large subunit | 2.59E−04 |
| | vs. SGN-B7H4V | Control | | | biogenesis | |
| human | B7H41001 mAb-DM1 | Down.in.Test/ | BP | GO:0000280 | nuclear division | 4.63E−04 |
| | vs. SGN-B7H4V | Control | | | | |
| human | B7H41001 mAb-DM1 | Down.in.Test/ | BP | GO:0007059 | chromosome segregation | 4.64E−04 |
| | vs. SGN-B7H4V | Control | | | | |
| human | B7H41001 mAb-DM1 | Down.in.Test/ | BP | GO:0006626 | protein targeting to | 4.64E−04 |
| | vs. SGN-B7H4V | Control | | | mitochondrion | |
| human | B7H41001 mAb-DM1 | Down.in.Test/ | BP | GO:0043281 | regulation of cysteine-type | 5.67E−04 |
| | vs. SGN-B7H4V | Control | | | endopeptidase activity involved | |
| | | | | | in apoptotic process | |
| human | B7H41001 mAb-DM1 | Down.in.Test/ | BP | GO:0070585 | protein localization to | 5.67E−04 |
| | vs. SGN-B7H4V | Control | | | mitochondrion | |
| human | B7H41001 mAb-DM1 | Down.in.Test/ | BP | GO:0000070 | mitotic sister chromatid | 5.67E−04 |
| | vs. SGN-B7H4V | Control | | | segregation | |
| human | B7H41001 mAb-DM1 | Down.in.Test/ | BP | GO:0007052 | mitotic spindle organization | 5.67E−04 |
| | vs. SGN-B7H4V | Control | | | | |
| human | B7H41001 mAb-DM1 | Down.in.Test/ | BP | GO:0061077 | chaperone-mediated protein | 6.31E−04 |
| | vs. SGN-B7H4V | Control | | | folding | |
| human | B7H41001 mAb-DM1 | Down.in.Test/ | BP | GO:0072655 | establishment of protein | 6.32E−04 |
| | vs. SGN-B7H4V | Control | | | localization to mitochondrion | |
| human | B7H41001 mAb-DM1 | Down.in.Test/ | BP | GO:0048285 | organelle fission | 6.93E−04 |
| | vs. SGN-B7H4V | Control | | | | |
| human | B7H41001 mAb-DM1 | Down.in.Test/ | BP | GO:0009408 | response to heat | 9.44E−04 |
| | vs. SGN-B7H4V | Control | | | | |
| human | B7H41001 mAb-DM1 | Down.in.Test/ | BP | GO:0050000 | chromosome localization | 9.53E−04 |
| | vs. SGN-B7H4V | Control | | | | |
| human | B7H41001 mAb-DM1 | Down.in.Test/ | BP | GO:0140053 | mitochondrial gene expression | 1.02E−03 |
| | vs. SGN-B7H4V | Control | | | | |
| human | B7H41001 mAb-DM1 | Down.in.Test/ | BP | GO:0000819 | sister chromatid segregation | 1.02E−03 |
| | vs. SGN-B7H4V | Control | | | | |
| human | B7H41001 mAb-DM1 | Down.in.Test/ | BP | GO:0001836 | release of cytochrome c from | 1.02E−03 |
| | vs. SGN-B7H4V | Control | | | mitochondria | |
| human | B7H41001 mAb-DM1 | Down.in.Test/ | BP | GO:0022904 | respiratory electron transport | 1.05E−03 |
| | vs. SGN-B7H4V | Control | | | chain | |
| human | B7H41001 mAb-DM1 | Down.in.Test/ | BP | GO:0007051 | spindle organization | 1.30E−03 |
| | vs. SGN-B7H4V | Control | | | | |
| human | B7H41001 mAb-DM1 | Down.in.Test/ | BP | GO:0071816 | tail-anchored membrane protein | 1.48E−03 |
| | vs. SGN-B7H4V | Control | | | insertion into ER membrane | |
| human | B7H41001 mAb-DM1 | Up.in.Test/ | BP | GO:0016126 | sterol biosynthetic process | 1.53E−03 |
| | vs. SGN-B7H4V | Control | | | | |
| human | B7H41001 mAb-DM1 | Up.in.Test/ | BP | GO:0006695 | cholesterol biosynthetic process | 1.53E−03 |
| | vs. SGN-B7H4V | Control | | | | |
| human | B7H41001 mAb-DM1 | Up.in.Test/ | BP | GO:1902653 | secondary alcohol biosynthetic | 1.53E−03 |
| | vs. SGN-B7H4V | Control | | | process | |
| human | B7H41001 mAb-DM1 | Down.in.Test/ | BP | GO:0051303 | establishment of chromosome | 1.61E−03 |
| | vs. SGN-B7H4V | Control | | | localization | |
| human | B7H41001 mAb-DM1 | Down.in.Test/ | BP | GO:0051310 | metaphase plate congression | 1.61E−03 |
| | vs. SGN-B7H4V | Control | | | | |
| human | B7H41001 mAb-DM1 | Up.in.Test/ | BP | GO:0016052 | carbohydrate catabolic process | 1.65E−03 |
| | vs. SGN-B7H4V | Control | | | | |
| human | B7H41001 mAb-DM1 | Down.in.Test/ | BP | GO:0045048 | protein insertion into ER | 2.03E−03 |
| | vs. SGN-B7H4V | Control | | | membrane | |
| human | B7H41001 mAb-DM1 | Down.in.Test/ | BP | GO:0045333 | cellular respiration | 2.52E−03 |
| | vs. SGN-B7H4V | Control | | | | |
| human | B7H41001 mAb-DM1 | Down.in.Test/ | BP | GO:0007088 | regulation of mitotic nuclear | 2.56E−03 |
| | vs. SGN-B7H4V | Control | | | division | |
| human | B7H41001 mAb-DM1 | Down.in.Test/ | BP | GO:0022900 | electron transport chain | 2.63E−03 |
| | vs. SGN-B7H4V | Control | | | | |
| human | B7H41001 mAb-DM1 | Down.in.Test/ | BP | GO:0097193 | intrinsic apoptotic signaling | 2.71E−03 |
| | vs. SGN-B7H4V | Control | | | pathway | |
| human | B7H41001 mAb-DM1 | Down.in.Test/ | BP | GO:0007080 | mitotic metaphase plate | 2.90E−03 |
| | vs. SGN-B7H4V | Control | | | congression | |
| human | B7H41001 mAb-DM1 | Down.in.Test/ | BP | GO:0016073 | snRNA metabolic process | 3.05E−03 |
| | vs. SGN-B7H4V | Control | | | | |
| human | B7H41001 mAb-DM1 | Down.in.Test/ | BP | GO:0052547 | regulation of peptidase activity | 3.30E−03 |
| | vs. SGN-B7H4V | Control | | | | |
| human | B7H41001 mAb-DM1 | Down.in.Test/ | BP | GO:2000116 | regulation of cysteine-type | 3.39E−03 |
| | vs. SGN-B7H4V | Control | | | endopeptidase activity | |
| human | B7H41001 mAb-DM1 | Down.in.Test/ | BP | GO:0043068 | positive regulation of | 3.39E−03 |
| | vs. SGN-B7H4V | Control | | | programmed cell death | |
| human | B7H41001 mAb-DM1 | Down.in.Test/ | BP | GO:1903533 | regulation of protein targeting | 3.84E−03 |
| | vs. SGN-B7H4V | Control | | | | |
| human | B7H41001 mAb-DM1 | Down.in.Test/ | BP | GO:0032543 | mitochondrial translation | 3.94E−03 |
| | vs. SGN-B7H4V | Control | | | | |
| human | B7H41001 mAb-DM1 | Down.in.Test/ | BP | GO:0043154 | negative regulation of cysteine- | 3.94E−03 |
| | vs. SGN-B7H4V | Control | | | type endopeptidase activity | |
| | | | | | involved in apoptotic process | |
| human | B7H41001 mAb-DM1 | Down.in.Test/ | BP | GO:0051205 | protein insertion into membrane | 5.55E−03 |
| | vs. SGN-B7H4V | Control | | | | |
| human | B7H41001 mAb-DM1 | Down.in.Test/ | BP | GO:0043065 | positive regulation of apoptotic | 5.74E−03 |
| | vs. SGN-B7H4V | Control | | | process | |
| human | B7H41001 mAb-DM1 | Down.in.Test/ | BP | GO:0052548 | regulation of endopeptidase | 5.82E−03 |
| | vs. SGN-B7H4V | Control | | | activity | |
| human | B7H41001 mAb-DM1 | Down.in.Test/ | BP | GO:2000117 | negative regulation of cysteine- | 6.43E−03 |
| | vs. SGN-B7H4V | Control | | | type endopeptidase activity | |
| human | B7H41001 mAb-DM1 | Down.in.Test/ | BP | GO:0034698 | response to gonadotropin | 6.61E−03 |
| | vs. SGN-B7H4V | Control | | | | |
| human | B7H41001 mAb-DM1 | Up.in.Test/ | BP | GO:0016125 | sterol metabolic process | 8.65E−03 |
| | vs. SGN-B7H4V | Control | | | | |
| human | B7H41001 mAb-DM1 | Up.in.Test/ | BP | GO:0006694 | steroid biosynthetic process | 8.65E−03 |
| | vs. SGN-B7H4V | Control | | | | |
| human | B7H41001 mAb-DM1 | Up.in.Test/ | BP | GO:0048871 | multicellular organismal | 8.65E−03 |
| | vs. SGN-B7H4V | Control | | | homeostasis | |
| human | B7H41001 mAb-DM1 | Up.in.Test/ | BP | GO:1902652 | secondary alcohol metabolic | 8.65E−03 |
| | vs. SGN-B7H4V | Control | | | process | |
| human | B7H41001 mAb-DM1 | Up.in.Test/ | BP | GO:0044283 | small molecule biosynthetic | 8.65E−03 |
| | vs. SGN-B7H4V | Control | | | process | |
| human | B7H41001 mAb-DM1 | Up.in.Test/ | BP | GO:0008299 | isoprenoid biosynthetic process | 8.88E−03 |
| | vs. SGN-B7H4V | Control | | | | |
| human | B7H41001 mAb-DM1 | Up.in.Test/ | BP | GO:0008203 | cholesterol metabolic process | 8.88E−03 |
| | vs. SGN-B7H4V | Control | | | | |
| human | B7H41001 mAb-DM1 | Down.in.Test/ | BP | GO:2001242 | regulation of intrinsic apoptotic | 9.64E−03 |
| | vs. SGN-B7H4V | Control | | | signaling pathway | |
| human | B7H41001 mAb-DM1 | Down.in.Test/ | BP | GO:0000470 | maturation of LSU-rRNA | 9.64E−03 |
| | vs. SGN-B7H4V | Control | | | | |
| mouse | B7H41001 mAb-DM1 | Down.in.Test/ | BP | GO:0009615 | response to virus | 2.98E−21 |
| | vs. SGN-B7H4V | Control | | | | |
| mouse | B7H41001 mAb-DM1 | Down.in.Test/ | BP | GO:0051607 | defense response to virus | 1.94E−20 |
| | vs. SGN-B7H4V | Control | | | | |
| mouse | B7H41001 mAb-DM1 | Down.in.Test/ | BP | GO:0140546 | defense response to symbiont | 1.94E−20 |
| | vs. SGN-B7H4V | Control | | | | |
| mouse | B7H41001 mAb-DM1 | Down.in.Test/ | BP | GO:0002764 | immune response-regulating | 1.76E−17 |
| | vs. SGN-B7H4V | Control | | | signaling pathway | |
| mouse | B7H41001 mAb-DM1 | Down.in.Test/ | BP | GO:0002250 | adaptive immune response | 8.26E−14 |
| | VS. SGN-B7H4V | Control | | | | |
| mouse | B7H41001 mAb-DM1 | Down.in.Test/ | BP | GO:0045088 | regulation of innate immune | 8.26E−14 |
| | vs. SGN-B7H4V | Control | | | response | |
| mouse | B7H41001 mAb-DM1 | Down.in.Test/ | BP | GO:0002831 | regulation of response to biotic | 9.69E−14 |
| | vs. SGN-B7H4V | Control | | | stimulus | |
| mouse | B7H41001 mAb-DM1 | Down.in.Test/ | BP | GO:0019882 | antigen processing and | 1.12E−13 |
| | vs. SGN-B7H4V | Control | | | presentation | |
| mouse | B7H41001 mAb-DM1 | Down.in.Test/ | BP | GO:0001819 | positive regulation of cytokine | 1.26E−13 |
| | vs. SGN-B7H4V | Control | | | production | |
| mouse | B7H41001 mAb-DM1 | Down.in.Test/ | BP | GO:0002443 | leukocyte mediated immunity | 2.09E−13 |
| | vs. SGN-B7H4V | Control | | | | |
| mouse | B7H41001 mAb-DM1 | Down.in.Test/ | BP | GO:0002460 | adaptive immune response based | 5.14E−13 |
| | vs. SGN-B7H4V | Control | | | on somatic recombination of | |
| | | | | | immune receptors built from | |
| | | | | | immunoglobulin superfamily | |
| | | | | | domains | |
| mouse | B7H41001 mAb-DM1 | Down.in.Test/ | BP | GO:0002221 | pattern recognition receptor | 5.14E−13 |
| | vs. SGN-B7H4V | Control | | | signaling pathway | |
| mouse | B7H41001 mAb-DM1 | Down.in.Test/ | BP | GO:0002697 | regulation of immune effector | 4.07E−12 |
| | vs. SGN-B7H4V | Control | | | process | |
| mouse | B7H41001 mAb-DM1 | Down.in.Test/ | BP | GO:0048002 | antigen processing and | 1.22E−11 |
| | vs. SGN-B7H4V | Control | | | presentation of peptide antigen | |
| mouse | B7H41001 mAb-DM1 | Down.in.Test/ | BP | GO:0051249 | regulation of lymphocyte | 1.59E−11 |
| | vs. SGN-B7H4V | Control | | | activation | |
| mouse | B7H41001 mAb-DM1 | Down.in.Test/ | BP | GO:0002449 | lymphocyte mediated immunity | 1.64E−11 |
| | vs. SGN-B7H4V | Control | | | | |
| mouse | B7H41001 mAb-DM1 | Down.in.Test/ | BP | GO:0035456 | response to interferon-beta | 2.99E−11 |
| | vs. SGN-B7H4V | Control | | | | |
| mouse | B7H41001 mAb-DM1 | Down.in.Test/ | BP | GO:0035458 | cellular response to interferon- | 3.51E−11 |
| | vs. SGN-B7H4V | Control | | | beta | |
| mouse | B7H41001 mAb-DM1 | Down.in.Test/ | BP | GO:0006909 | phagocytosis | 1.38E−10 |
| | vs. SGN-B7H4V | Control | | | | |
| mouse | B7H41001 mAb-DM1 | Down.in.Test/ | BP | GO:0032680 | regulation of tumor necrosis | 1.38E−10 |
| | vs. SGN-B7H4V | Control | | | factor production | |
| mouse | B7H41001 mAb-DM1 | Down.in.Test/ | BP | GO:1903555 | regulation of tumor necrosis | 2.00E−10 |
| | vs. SGN-B7H4V | Control | | | factor superfamily cytokine | |
| | | | | | production | |
| mouse | B7H41001 mAb-DM1 | Down.in.Test/ | BP | GO:0032640 | tumor necrosis factor production | 2.52E−10 |
| | vs. SGN-B7H4V | Control | | | | |
| mouse | B7H41001 mAb-DM1 | Down.in.Test/ | BP | GO:0019221 | cytokine-mediated signaling | 2.71E−10 |
| | vs. SGN-B7H4V | Control | | | pathway | |
| mouse | B7H41001 mAb-DM1 | Down.in.Test/ | BP | GO:0071706 | tumor necrosis factor superfamily | 3.47E−10 |
| | vs. SGN-B7H4V | Control | | | cytokine production | |
| mouse | B7H41001 mAb-DM1 | Down.in.Test/ | BP | GO:0002703 | regulation of leukocyte mediated | 4.18E−10 |
| | vs. SGN-B7H4V | Control | | | immunity | |
| mouse | B7H41001 mAb-DM1 | Down.in.Test/ | BP | GO:0002683 | negative regulation of immune | 4.28E−10 |
| | vs. SGN-B7H4V | Control | | | system process | |
| mouse | B7H41001 mAb-DM1 | Down.in.Test/ | BP | GO:0002822 | regulation of adaptive immune | 5.98E−10 |
| | vs. SGN-B7H4V | Control | | | response based on somatic | |
| | | | | | recombination of immune | |
| | | | | | receptors built from | |
| | | | | | immunoglobulin superfamily | |
| | | | | | domains | |
| mouse | B7H41001 mAb-DM1 | Down.in.Test/ | BP | GO:0002819 | regulation of adaptive immune | 7.54E−10 |
| | vs. SGN-B7H4V | Control | | | response | |
| mouse | B7H41001 mAb-DM1 | Down.in.Test/ | BP | GO:0001906 | cell killing | 2.21E−09 |
| | vs. SGN-B7H4V | Control | | | | |
| mouse | B7H41001 mAb-DM1 | Down.in.Test/ | BP | GO:0032103 | positive regulation of response to | 2.74E−09 |
| | vs. SGN-B7H4V | Control | | | external stimulus | |
| mouse | B7H41001 mAb-DM1 | Down.in.Test/ | BP | GO:0001818 | negative regulation of cytokine | 2.89E−09 |
| | vs. SGN-B7H4V | Control | | | production | |
| mouse | B7H41001 mAb-DM1 | Down.in.Test/ | BP | GO:0002274 | myeloid leukocyte activation | 4.29E−09 |
| | vs. SGN-B7H4V | Control | | | | |
| mouse | B7H41001 mAb-DM1 | Down.in.Test/ | BP | GO:0050764 | regulation of phagocytosis | 4.71E−09 |
| | vs. SGN-B7H4V | Control | | | | |
| mouse | B7H41001 mAb-DM1 | Down.in.Test/ | BP | GO:0002474 | antigen processing and | 4.96E−09 |
| | vs. SGN-B7H4V | Control | | | presentation of peptide antigen | |
| | | | | | via MHC class I | |
| mouse | B7H41001 mAb-DM1 | Down.in.Test/ | BP | GO:0032481 | positive regulation of type I | 1.14E−08 |
| | vs. SGN-B7H4V | Control | | | interferon production | |
| mouse | B7H41001 mAb-DM1 | Down.in.Test/ | BP | GO:0050867 | positive regulation of cell | 1.50E−08 |
| | vs. SGN-B7H4V | Control | | | activation | |
| mouse | B7H41001 mAb-DM1 | Down.in.Test/ | BP | GO:0050670 | regulation of lymphocyte | 3.46E−08 |
| | vs. SGN-B7H4V | Control | | | proliferation | |
| mouse | B7H41001 mAb-DM1 | Down.in.Test/ | BP | GO:0001909 | leukocyte mediated cytotoxicity | 3.86E−08 |
| | vs. SGN-B7H4V | Control | | | | |
| mouse | B7H41001 mAb-DM1 | Down.in.Test/ | BP | GO:0032944 | regulation of mononuclear cell | 4.82 E−08 |
| | vs. SGN-B7H4V | Control | | | proliferation | |
| mouse | B7H41001 mAb-DM1 | Down.in.Test/ | BP | GO:0032479 | regulation of type I interferon | 4.82 E−08 |
| | vs. SGN-B7H4V | Control | | | production | |
| mouse | B7H41001 mAb-DM1 | Down.in.Test/ | BP | GO:0042742 | defense response to bacterium | 4.82E−08 |
| | vs. SGN-B7H4V | Control | | | | |
| mouse | B7H41001 mAb-DM1 | Down.in.Test/ | BP | GO:0046651 | lymphocyte proliferation | 5.17E−08 |
| | vs. SGN-B7H4V | Control | | | | |
| mouse | B7H41001 mAb-DM1 | Down.in.Test/ | BP | GO:0002224 | toll-like receptor signaling | 5.17E−08 |
| | vs. SGN-B7H4V | Control | | | pathway | |
| mouse | B7H41001 mAb-DM1 | Down.in.Test/ | BP | GO:0002706 | regulation of lymphocyte | 5.53E−08 |
| | vs. SGN-B7H4V | Control | | | mediated immunity | |
| mouse | B7H41001 mAb-DM1 | Down.in.Test/ | BP | GO:0002696 | positive regulation of leukocyte | 6.00E−08 |
| | vs. SGN-B7H4V | Control | | | activation | |
| mouse | B7H41001 mAb-DM1 | Down.in.Test/ | BP | GO:0032606 | type I interferon production | 6.00E−08 |
| | vs. SGN-B7H4V | Control | | | | |
| mouse | B7H41001 mAb-DM1 | Down.in.Test/ | BP | GO:0032943 | mononuclear cell proliferation | 6.53E−08 |
| | vs. SGN-B7H4V | Control | | | | |
| mouse | B7H41001 mAb-DM1 | Down.in.Test/ | BP | GO:0032728 | positive regulation of interferon- | 8.86E−08 |
| | vs. SGN-B7H4V | Control | | | beta production | |
| mouse | B7H41001 mAb-DM1 | Down.in.Test/ | BP | GO:0070661 | leukocyte proliferation | 1.01E−07 |
| | vs. SGN-B7H4V | Control | | | | |
| mouse | B7H41001 mAb-DM1 | Down.in.Test/ | BP | GO:0060759 | regulation of response to cytokine | 1.42E−07 |
| | vs. SGN-B7H4V | Control | | | stimulus | |
| mouse | B7H41001 mAb-DM1 | Down.in.Test/ | BP | GO:0070663 | regulation of leukocyte | 1.57E−07 |
| | vs. SGN-B7H4V | Control | | | proliferation | |
| mouse | B7H41001 mAb-DM1 | Down.in.Test/ | BP | GO:0007159 | leukocyte cell-cell adhesion | 1.66E−07 |
| | vs. SGN-B7H4V | Control | | | | |
| mouse | B7H41001 mAb-DM1 | Down.in.Test/ | BP | GO:0031349 | positive regulation of defense | 1.66E−07 |
| | vs. SGN-B7H4V | Control | | | response | |
| mouse | B7H41001 mAb-DM1 | Down.in.Test/ | BP | GO:0002755 | MyD88-dependent toll-like | 1.76E−07 |
| | vs. SGN-B7H4V | Control | | | receptor signaling pathway | |
| mouse | B7H41001 mAb-DM1 | Down.in.Test/ | BP | GO:0034341 | response to interferon-gamma | 1.96E−07 |
| | vs. SGN-B7H4V | Control | | | | |
| mouse | B7H41001 mAb-DM1 | Down.in.Test/ | BP | GO:0050863 | regulation of T cell activation | 2.14E−07 |
| | vs. SGN-B7H4V | Control | | | | |
| mouse | B7H41001 mAb-DM1 | Down.in.Test/ | BP | GO:0002699 | positive regulation of immune | 3.73E−07 |
| | vs. SGN-B7H4V | Control | | | effector process | |
| mouse | B7H41001 mAb-DM1 | Down.in.Test/ | BP | GO:0002705 | positive regulation of leukocyte | 4.11E−07 |
| | vs. SGN-B7H4V | Control | | | mediated immunity | |
| mouse | B7H41001 mAb-DM1 | Down.in.Test/ | BP | GO:0071216 | cellular response to biotic | 4.11E−07 |
| | vs. SGN-B7H4V | Control | | | stimulus | |
| mouse | B7H41001 mAb-DM1 | Down.in.Test/ | BP | GO:0045824 | negative regulation of innate | 4.35E−07 |
| | vs. SGN-B7H4V | Control | | | immune response | |
| mouse | B7H41001 mAb-DM1 | Down.in.Test/ | BP | GO:0051251 | positive regulation of lymphocyte | 4.88E−07 |
| | vs. SGN-B7H4V | Control | | | activation | |
| mouse | B7H41001 mAb-DM1 | Down.in.Test/ | BP | GO:0002824 | positive regulation of adaptive | 5.38E−07 |
| | vs. SGN-B7H4V | Control | | | immune response based on | |
| | | | | | somatic recombination of immune | |
| | | | | | receptors built from | |
| | | | | | immunoglobulin superfamily | |
| | | | | | domains | |
| mouse | B7H41001 mAb-DM1 | Down.in.Test/ | BP | GO:0031348 | negative regulation of defense | 6.78E−07 |
| | vs. SGN-B7H4V | Control | | | response | |
| mouse | B7H41001 mAb-DM1 | Down.in.Test/ | BP | GO:0002456 | T cell mediated immunity | 6.78E−07 |
| | vs. SGN-B7H4V | Control | | | | |
| mouse | B7H41001 mAb-DM1 | Down.in.Test/ | BP | GO:0050727 | regulation of inflammatory | 6.93E−07 |
| | vs. SGN-B7H4V | Control | | | response | |
| mouse | B7H41001 mAb-DM1 | Down.in.Test/ | BP | GO:0060337 | type I interferon signaling | 9.79E−07 |
| | vs. SGN-B7H4V | Control | | | pathway | |
| mouse | B7H41001 mAb-DM1 | Down.in.Test/ | BP | GO:0071357 | cellular response to type I | 9.79E−07 |
| | vs. SGN-B7H4V | Control | | | interferon | |
| mouse | B7H41001 mAb-DM1 | Down.in.Test/ | BP | GO:0001959 | regulation of cytokine-mediated | 1.16E−06 |
| | vs. SGN-B7H4V | Control | | | signaling pathway | |
| mouse | B7H41001 mAb-DM1 | Down.in.Test/ | BP | GO:0002478 | antigen processing and | 1.16E−06 |
| | vs. SGN-B7H4V | Control | | | presentation of exogenous | |
| | | | | | peptide antigen | |
| mouse | B7H41001 mAb-DM1 | Down.in.Test/ | BP | GO:0002821 | positive regulation of adaptive | 1.17E−06 |
| | vs. SGN-B7H4V | Control | | | immune response | |
| mouse | B7H41001 mAb-DM1 | Down.in.Test/ | BP | GO:1903039 | positive regulation of leukocyte | 1.37E−06 |
| | vs. SGN-B7H4V | Control | | | cell-cell adhesion | |
| mouse | B7H41001 mAb-DM1 | Down.in.Test/ | BP | GO:1903037 | regulation of leukocyte cell-cell | 1.59E−06 |
| | vs. SGN-B7H4V | Control | | | adhesion | |
| mouse | B7H41001 mAb-DM1 | Down.in.Test/ | BP | GO:0042590 | antigen processing and | 1.72E−06 |
| | vs. SGN-B7H4V | Control | | | presentation of exogenous | |
| | | | | | peptide antigen via MHC class 1 | |
| mouse | B7H41001 mAb-DM1 | Down.in.Test/ | BP | GO:0032675 | regulation of interleu kin-6 | 1.72E−06 |
| | vs. SGN-B7H4V | Control | | | production | |
| mouse | B7H41001 mAb-DM1 | Down.in.Test/ | BP | GO:0032755 | positive regulation of interleukin- | 1.89E−06 |
| | vs. SGN-B7H4V | Control | | | 6 production | |
| mouse | B7H41001 mAb-DM1 | Down.in.Test/ | BP | GO:0032102 | negative regulation of response to | 1.96E−06 |
| | vs. SGN-B7H4V | Control | | | external stimulus | |
| mouse | B7H41001 mAb-DM1 | Down.in.Test/ | BP | GO:0045071 | negative regulation of viral | 2.21E−06 |
| | vs. SGN-B7H4V | Control | | | genome replication | |
| mouse | B7H41001 mAb-DM1 | Down.in.Test/ | BP | GO:0050766 | positive regulation of | 2.69E−06 |
| | vs. SGN-B7H4V | Control | | | phagocytosis | |
| mouse | B7H41001 mAb-DM1 | Down.in.Test/ | BP | GO:0034340 | response to type I interferon | 2.69E−06 |
| | vs. SGN-B7H4V | Control | | | | |
| mouse | B7H41001 mAb-DM1 | Down.in.Test/ | BP | GO:0046631 | alpha-beta T cell activation | 2.72E−06 |
| | vs. SGN-B7H4V | Control | | | | |
| mouse | B7H41001 mAb-DM1 | Down.in.Test/ | BP | GO:0002708 | positive regulation of lymphocyte | 3.07E−06 |
| | vs. SGN-B7H4V | Control | | | mediated immunity | |
| mouse | B7H41001 mAb-DM1 | Down.in.Test/ | BP | GO:0071219 | cellular response to molecule of | 3.36E−06 |
| | vs. SGN-B7H4V | Control | | | bacterial origin | |
| mouse | B7H41001 mAb-DM1 | Down.in.Test/ | BP | GO:0032635 | interleukin-6 production | 3.66E−06 |
| | vs. SGN-B7H4V | Control | | | | |
| mouse | B7H41001 mAb-DM1 | Down.in.Test/ | BP | GO:0032608 | interferon-beta production | 3.90E−06 |
| | vs. SGN-B7H4V | Control | | | | |
| mouse | B7H41001 mAb-DM1 | Down.in.Test/ | BP | GO:0032648 | regulation of interferon-beta | 3.90E−06 |
| | vs. SGN-B7H4V | Control | | | production | |
| mouse | B7H41001 mAb-DM1 | Down.in.Test/ | BP | GO:0002753 | cytoplasmic pattern recognition | 3.97E−06 |
| | vs. SGN-B7H4V | Control | | | receptor signaling pathway | |
| mouse | B7H41001 mAb-DM1 | Down.in.Test/ | BP | GO:0031341 | regulation of cell killing | 4.12E−06 |
| | vs. SGN-B7H4V | Control | | | | |
| mouse | B7H41001 mAb-DM1 | Down.in.Test/ | BP | GO:0002237 | response to molecule of bacterial | 4.93E−06 |
| | vs. SGN-B7H4V | Control | | | origin | |
| mouse | B7H41001 mAb-DM1 | Down.in.Test/ | BP | GO:0002366 | leukocyte activation involved in | 4.93E−06 |
| | vs. SGN-B7H4V | Control | | | immune response | |
| mouse | B7H41001 mAb-DM1 | Down.in.Test/ | BP | GO:0035455 | response to interferon-alpha | 4.93E−06 |
| | vs. SGN-B7H4V | Control | | | | |
| mouse | B7H41001 mAb-DM1 | Down.in.Test/ | BP | GO:0031343 | positive regulation of cell killing | 5.32E−06 |
| | vs. SGN-B7H4V | Control | | | | |
| mouse | B7H41001 mAb-DM1 | Down.in.Test/ | BP | GO:1903706 | regulation of hemopoiesis | 5.32E−06 |
| | vs. SGN-B7H4V | Control | | | | |
| mouse | B7H41001 mAb-DM1 | Down.in.Test/ | BP | GO:0032760 | positive regulation of tumor | 5.43E−06 |
| | vs. SGN-B7H4V | Control | | | necrosis factor production | |
| mouse | B7H41001 mAb-DM1 | Down.in.Test/ | BP | GO:0002263 | cell activation involved in immune | 5.82E−06 |
| | vs. SGN-B7H4V | Control | | | response | |
| mouse | B7H41001 mAb-DM1 | Down.in.Test/ | BP | GO:0002440 | production of molecular mediator | 5.91E−06 |
| | vs. SGN-B7H4V | Control | | | of immune response | |
| mouse | B7H41001 mAb-DM1 | Down.in.Test/ | BP | GO:1902105 | regulation of leukocyte | 6.09E−06 |
| | vs. SGN-B7H4V | Control | | | differentiation | |
| mouse | B7H41001 mAb-DM1 | Down.in.Test/ | BP | GO:0048525 | negative regulation of viral | 6.18E−06 |
| | vs. SGN-B7H4V | Control | | | process | |
| mouse | B7H41001 mAb-DM1 | Down.in.Test/ | BP | GO:0019884 | antigen processing and | 6.55E−06 |
| | vs. SGN-B7H4V | Control | | | presentation of exogenous | |
| | | | | | antigen | |
| mouse | B7H41001 mAb-DM1 | Down.in.Test/ | BP | GO:1903557 | positive regulation of tumor | 6.55E−06 |
| | vs. SGN-B7H4V | Control | | | necrosis factor superfamily | |
| | | | | | cytokine production | |
| mouse | B7H41001 mAb-DM1 | Down.in.Test/ | BP | GO:0006914 | autophagy | 6.55E−06 |
| | vs. SGN-B7H4V | Control | | | | |
| mouse | B7H41001 mAb-DM1 | Down.in.Test/ | BP | GO:0061919 | process utilizing autophagic | 6.55E−06 |
| | vs. SGN-B7H4V | Control | | | mechanism | |
| mouse | B7H41001 mAb-DM1 | Down.in.Test/ | BP | GO:0042116 | macrophage activation | 6.77E−06 |
| | vs. SGN-B7H4V | Control | | | | |
| mouse | B7H41001 mAb-DM1 | Down.in.Test/ | BP | GO:0007033 | vacuole organization | 7.35E−O6 |
| | vs. SGN-B7H4V | Control | | | | |
| mouse | B7H41001 mAb-DM1 | Down.in.Test/ | BP | GO:0030098 | lymphocyte differentiation | 7.40E−06 |
| | vs. SGN-B7H4V | Control | | | | |
| mouse | B7H41001 mAb-DM1 | Down.in.Test/ | BP | GO:0002709 | regulation of T cell mediated | 7.57E−06 |
| | vs. SGN-B7H4V | Control | | | immunity | |
| mouse | B7H41001 mAb-DM1 | Down.in.Test/ | BP | GO:0032607 | interferon-alpha production | 7.99E−06 |
| | vs. SGN-B7H4V | Control | | | | |
| mouse | B7H41001 mAb-DM1 | Down.in.Test/ | BP | GO:0050870 | positive regulation of T cell | 8.34E−06 |
| | vs. SGN-B7H4V | Control | | | activation | |
| mouse | B7H41001 mAb-DM1 | Down.in.Test/ | BP | GO:0042129 | regulation of T cell proliferation | 8.51E−06 |
| | vs. SGN-B7H4V | Control | | | | |
| mouse | B7H41001 mAb-DM1 | Down.in.Test/ | BP | GO:0002768 | immune response-regulating cell | 9.09E−06 |
| | vs. SGN-B7H4V | Control | | | surface receptor signaling | |
| | | | | | pathway | |
| mouse | B7H41001 mAb-DM1 | Down.in.Test/ | BP | GO:1903131 | mononuclear cell differentiation | 9.34E−06 |
| | vs. SGN-B7H4V | Control | | | | |
| mouse | B7H41001 mAb-DM1 | Down.in.Test/ | BP | GO:0022409 | positive regulation of cell-cell | 9.34E−06 |
| | vs. SGN-B7H4V | Control | | | adhesion | |
| mouse | B7H41001 mAb-DM1 | Down.in.Test/ | BP | GO:0032651 | regulation of interleukin-1 beta | 1.09E−05 |
| | vs. SGN-B7H4V | Control | | | production | |
| mouse | B7H41001 mAb-DM1 | Down.in.Test/ | BP | GO:0060339 | negative regulation of type I | 1.09E−05 |
| | vs. SGN-B7H4V | Control | | | interferon-mediated signaling | |
| | | | | | pathway | |
| mouse | B7H41001 mAb-DM1 | Down.in.Test/ | BP | GO:0045089 | positive regulation of innate | 1.09E−05 |
| | vs. SGN-B7H4V | Control | | | immune response | |
| mouse | B7H41001 mAb-DM1 | Down.in.Test/ | BP | GO:0060760 | positive regulation of response to | 1.14E−05 |
| | vs. SGN-B7H4V | Control | | | cytokine stimulus | |
| mouse | B7H41001 mAb-DM1 | Down.in.Test/ | BP | GO:0042098 | T cell proliferation | 1.17E−05 |
| | vs. SGN-B7H4V | Control | | | | |
| mouse | B7H41001 mAb-DM1 | Down.in.Test/ | BP | GO:0001910 | regulation of leukocyte mediated | 1.17E−05 |
| | vs. SGN-B7H4V | Control | | | cytotoxicity | |
| mouse | B7H41001 mAb-DM1 | Down.in.Test/ | BP | GO:0050900 | leukocyte migration | 1.34E−05 |
| | vs. SGN-B7H4V | Control | | | | |
| mouse | B7H41001 mAb-DM1 | Down.in.Test/ | BP | GO:1900017 | positive regulation of cytokine | 1.48E−05 |
| | vs. SGN-B7H4V | Control | | | production involved in | |
| | | | | | inflammatory response | |
| mouse | B7H41001 mAb-DM1 | Down.in.Test/ | BP | GO:0050777 | negative regulation of immune | 1.55E−05 |
| | vs. SGN-B7H4V | Control | | | response | |
| mouse | B7H41001 mAb-DM1 | Down.in.Test/ | BP | GO:0032720 | negative regulation of tumor | 1.81E−05 |
| | vs. SGN-B7H4V | Control | | | necrosis factor production | |
| mouse | B7H41001 mAb-DM1 | Down.in.Test/ | BP | GO:0071222 | cellular response to | 1.90E−05 |
| | vs. SGN-B7H4V | Control | | | lipopolysaccharide | |
| mouse | B7H41001 mAb-DM1 | Down.in.Test/ | BP | GO:0002685 | regulation of leukocyte migration | 1.96E−05 |
| | vs. SGN-B7H4V | Control | | | | |
| mouse | B7H41001 mAb-DM1 | Down.in.Test/ | BP | GO:1903556 | negative regulation of tumor | 2.10E−05 |
| | vs. SGN-B7H4V | Control | | | necrosis factor superfamily | |
| | | | | | cytokine production | |
| mouse | B7H41001 mAb-DM1 | Down.in.Test/ | BP | GO:0019883 | antigen processing and | 2.13E−05 |
| | vs. SGN-B7H4V | Control | | | presentation of endogenous | |
| | | | | | antigen | |
| mouse | B7H41001 mAb-DM1 | Down.in.Test/ | BP | GO:0032611 | interleukin-1 beta production | 2.23E−05 |
| | vs. SGN-B7H4V | Control | | | | |
| mouse | B7H41001 mAb-DM1 | Down.in.Test/ | BP | GO:0062207 | regulation of pattern recognition | 2.23E−05 |
| | vs. SGN-B7H4V | Control | | | receptor signaling pathway | |
| mouse | B7H41001 mAb-DM1 | Down.in.Test/ | BP | GO:0002832 | negative regulation of response to | 2.29E−05 |
| | vs. SGN-B7H4V | Control | | | biotic stimulus | |
| mouse | B7H41001 mAb-DM1 | Down.in.Test/ | BP | GO:0002833 | positive regulation of response to | 2.37E−05 |
| | vs. SGN-B7H4V | Control | | | biotic stimulus | |
| mouse | B7H41001 mAb-DM1 | Down.in.Test/ | BP | GO:0032652 | regulation of interleukin-1 | 2.55E−05 |
| | vs. SGN-B7H4V | Control | | | production | |
| mouse | B7H41001 mAb-DM1 | Down.in.Test/ | BP | GO:0060338 | regulation of type I interferon- | 2.64E−05 |
| | vs. SGN-B7H4V | Control | | | mediated signaling pathway | |
| mouse | B7H41001 mAb-DM1 | Down.in.Test/ | BP | GO:1903900 | regulation of viral life cycle | 3.29E−05 |
| | vs. SGN-B7H4V | Control | | | | |
| mouse | B7H41001 mAb-DM1 | Down.in.Test/ | BP | GO:0022407 | regulation of cell-cell adhesion | 3.83E−05 |
| | vs. SGN-B7H4V | Control | | | | |
| mouse | B7H41001 mAb-DM1 | Down.in.Test/ | BP | GO:0032496 | response to lipopolysaccharide | 3.92E−05 |
| | vs. SGN-B7H4V | Control | | | | |
| mouse | B7H41001 mAb-DM1 | Down.in.Test/ | BP | GO:0001912 | positive regulation of leukocyte | 4.31E−05 |
| | vs. SGN-B7H4V | Control | | | mediated cytotoxicity | |
| mouse | B7H41001 mAb-DM1 | Down.in.Test/ | BP | GO:0032647 | regulation of interferon-alpha | 4.61E−05 |
| | vs. SGN-B7H4V | Control | | | production | |
| mouse | B7H41001 mAb-DM1 | Down.in.Test/ | BP | GO:0002429 | immune response-activating cell | 4.61E−05 |
| | vs. SGN-B7H4V | Control | | | surface receptor signaling | |
| | | | | | pathway | |
| mouse | B7H41001 mAb-DM1 | Down.in.Test/ | BP | GO:0050792 | regulation of viral process | 4.71E−05 |
| | vs. SGN-B7H4V | Control | | | | |
| mouse | B7H41001 mAb-DM1 | Down.in.Test/ | BP | GO:0032612 | interleukin-1 production | 4.81E−05 |
| | vs. SGN-B7H4V | Control | | | | |
| mouse | B7H41001 mAb-DM1 | Down.in.Test/ | BP | GO:0045453 | bone resorption | 4.84E−05 |
| | vs. SGN-B7H4V | Control | | | | |
| mouse | B7H41001 mAb-DM1 | Down.in.Test/ | BP | GO:0002757 | immune response-activating | 4.84E−05 |
| | vs. SGN-B7H4V | Control | | | signal transduction | |
| mouse | B7H41001 mAb-DM1 | Down.in.Test/ | BP | GO:0019058 | viral life cycle | 4.94E−05 |
| | vs. SGN-B7H4V | Control | | | | |
| mouse | B7H41001 mAb-DM1 | Down.in.Test/ | BP | GO:0001960 | negative regulation of cytokine- | 4.94E−05 |
| | vs. SGN-B7H4V | Control | | | mediated signaling pathway | |
| mouse | B7H41001 mAb-DM1 | Down.in.Test/ | BP | GO:0097530 | granulocyte migration | 4.94E−05 |
| | vs. SGN-B7H4V | Control | | | | |
| mouse | B7H41001 mAb-DM1 | Down.in.Test/ | BP | GO:0001562 | response to protozoan | 4.95E−05 |
| | vs. SGN-B7H4V | Control | | | | |
| mouse | B7H41001 mAb-DM1 | Down.in.Test/ | BP | GO:0002377 | immunoglobulin production | 6.28E−05 |
| | vs. SGN-B7H4V | Control | | | | |
| mouse | B7H41001 mAb-DM1 | Down.in.Test/ | BP | GO:0002253 | activation of immune response | 6.58E−05 |
| | vs. SGN-B7H4V | Control | | | | |
| mouse | B7H41001 mAb-DM1 | Down.in.Test/ | BP | GO:0002688 | regulation of leukocyte | 6.97E−05 |
| | vs. SGN-B7H4V | Control | | | chemotaxis | |
| mouse | B7H41001 mAb-DM1 | Down.in.Test/ | BP | GO:1990266 | neutrophil migration | 6.97E−05 |
| | vs. SGN-B7H4V | Control | | | | |
| mouse | B7H41001 mAb-DM1 | Down.in.Test/ | BP | GO:0060761 | negative regulation of response to | 7.76E−05 |
| | vs. SGN-B7H4V | Control | | | cytokine stimulus | |
| mouse | B7H41001 mAb-DM1 | Down.in.Test/ | BP | GO:0032642 | regulation of chemokine | 8.35E−05 |
| | vs. SGN-B7H4V | Control | | | production | |
| mouse | B7H41001 mAb-DM1 | Down.in.Test/ | BP | GO:0050671 | positive regulation of lymphocyte | 8.89 E−05 |
| | vs. SGN-B7H4V | Control | | | proliferation | |
| mouse | B7H41001 mAb-DM1 | Down.in.Test/ | BP | GO:1900015 | regulation of cytokine production | 8.89 E−05 |
| | vs. SGN-B7H4V | Control | | | involved in inflammatory | |
| | | | | | response | |
| mouse | B7H41001 mAb-DM1 | Down.in.Test/ | BP | GO:0016032 | viral process | 8.89E−05 |
| | vs. SGN-B7H4V | Control | | | | |
| mouse | B7H41001 mAb-DM1 | Down.in.Test/ | BP | GO:0071346 | cellular response to interferon- | 9.00E−05 |
| | vs. SGN-B7H4V | Control | | | gamma | |
| mouse | B7H41001 mAb-DM1 | Down.in.Test/ | BP | GO:0002483 | antigen processing and | 9.00E−05 |
| | vs. SGN-B7H4V | Control | | | presentation of endogenous | |
| | | | | | peptide antigen | |
| mouse | B7H41001 mAb-DM1 | Down.in.Test/ | BP | GO:0019885 | antigen processing and | 9.00E−05 |
| | vs. SGN-B7H4V | Control | | | presentation of endogenous | |
| | | | | | peptide antigen via MHC class I | |
| mouse | B7H41001 mAb-DM1 | Down.in.Test/ | BP | GO:0042119 | neutrophil activation | 9.00E−05 |
| | vs. SGN-B7H4V | Control | | | | |
| mouse | B7H41001 mAb-DM1 | Down.in.Test/ | BP | GO:0060326 | cell chemotaxis | 9.33E−05 |
| | vs. SGN-B7H4V | Control | | | | |
| mouse | B7H41001 mAb-DM1 | Down.in.Test/ | BP | GO:0048771 | tissue remodeling | 9.74E−05 |
| | vs. SGN-B7H4V | Control | | | | |
| mouse | B7H41001 mAb-DM1 | Down.in.Test/ | BP | GO:0032732 | positive regulation of interleukin- | 9.89E−05 |
| | vs. SGN-B7H4V | Control | | | 1 production | |
| mouse | B7H41001 mAb-DM1 | Down.in.Test/ | BP | GO:0032731 | positive regulation of interleukin- | 1.01E−04 |
| | vs. SGN-B7H4V | Control | | | 1 beta production | |
| mouse | B7H41001 mAb-DM1 | Down.in.Test/ | BP | GO:0032946 | positive regulation of | 1.01E−04 |
| | vs. SGN-B7H4V | Control | | | mononuclear cell proliferation | |
| mouse | B7H41001 mAb-DM1 | Down.in.Test/ | BP | GO:0002486 | antigen processing and | 1.16E−04 |
| | vs. SGN-B7H4V | Control | | | presentation of endogenous | |
| | | | | | peptide antigen via MHC class I via | |
| | | | | | ER pathway, TAP-independent | |
| mouse | B7H41001 mAb-DM1 | Down.in.Test/ | BP | GO:0032727 | positive regulation of interferon- | 1.16E−04 |
| | vs. SGN-B7H4V | Control | | | alpha production | |
| mouse | B7H41001 mAb-DM1 | Down.in.Test/ | BP | GO:0032602 | chemokine production | 1.19E−04 |
| | vs. SGN-B7H4V | Control | | | | |
| mouse | B7H41001 mAb-DM1 | Down.in.Test/ | BP | GO:0045069 | regulation of viral genome | 1.25E−04 |
| | vs. SGN-B7H4V | Control | | | replication | |
| mouse | B7H41001 mAb-DM1 | Down.in.Test/ | BP | GO:0045851 | pH reduction | 1.25E−04 |
| | vs. SGN-B7H4V | Control | | | | |
| mouse | B7H41001 mAb-DM1 | Down.in.Test/ | BP | GO:0034121 | regulation of toll-like receptor | 1.27E−04 |
| | vs. SGN-B7H4V | Control | | | signaling pathway | |
| mouse | B7H41001 mAb-DM1 | Down.in.Test/ | BP | GO:0098586 | cellular response to virus | 1.27E−04 |
| | vs. SGN-B7H4V | Control | | | | |
| mouse | B7H41001 mAb-DM1 | Down.in.Test/ | BP | GO:0030217 | T cell differentiation | 1.34E−04 |
| | vs. SGN-B7H4V | Control | | | | |
| mouse | B7H41001 mAb-DM1 | Down.in.Test/ | BP | GO:0002534 | cytokine production involved in | 1.34E−04 |
| | vs. SGN-B7H4V | Control | | | inflammatory response | |
| mouse | B7H41001 mAb-DM1 | Down.in.Test/ | BP | GO:0050830 | defense response to Gram- | 1.37E−04 |
| | vs. SGN-B7H4V | Control | | | positive bacterium | |
| mouse | B7H41001 mAb-DM1 | Down.in.Test/ | BP | GO:0097529 | myeloid leukocyte migration | 1.45E−04 |
| | vs. SGN-B7H4V | Control | | | | |
| mouse | B7H41001 mAb-DM1 | Down.in.Test/ | BP | GO:1902622 | regulation of neutrophil migration | 1.46E−04 |
| | vs. SGN-B7H4V | Control | | | | |
| mouse | B7H41001 mAb-DM1 | Down.in.Test/ | BP | GO:0002476 | antigen processing and | 1.46E−04 |
| | vs. SGN-B7H4V | Control | | | presentation of endogenous | |
| | | | | | peptide antigen via MHC class Ib | |
| mouse | B7H41001 mAb-DM1 | Down.in.Test/ | BP | GO:0002484 | antigen processing and | 1.46E−04 |
| | vs. SGN-B7H4V | Control | | | presentation of endogenous | |
| | | | | | peptide antigen via MHC class I via | |
| | | | | | ER pathway | |
| mouse | B7H41001 mAb-DM1 | Down.in.Test/ | BP | GO:0002700 | regulation of production of | 1.54E−04 |
| | vs. SGN-B7H4V | Control | | | molecular mediator of immune | |
| | | | | | response | |
| mouse | B7H41001 mAb-DM1 | Down.in.Test/ | BP | GO:0001961 | positive regulation of cytokine- | 1.73E−04 |
| | vs. SGN-B7H4V | Control | | | mediated signaling pathway | |
| mouse | B7H41001 mAb-DM1 | Down.in.Test/ | BP | GO:0046849 | bone remodeling | 1.86E−04 |
| | vs. SGN-B7H4V | Control | | | | |
| mouse | B7H41001 mAb-DM1 | Down.in.Test/ | BP | GO:0002428 | antigen processing and | 1.90E−04 |
| | vs. SGN-B7H4V | Control | | | presentation of peptide antigen | |
| | | | | | via MHC class Ib | |
| mouse | B7H41001 mAb-DM1 | Down.in.Test/ | BP | GO:0042832 | defense response to protozoan | 2.02E−04 |
| | vs. SGN-B7H4V | Control | | | | |
| mouse | B7H41001 mAb-DM1 | Down.in.Test/ | BP | GO:0001914 | regulation of T cell mediated | 2.04E−04 |
| | vs. SGN-B7H4V | Control | | | cytotoxicity | |
| mouse | B7H41001 mAb-DM1 | Down.in.Test/ | BP | GO:0001913 | T cell mediated cytotoxicity | 2.04E−04 |
| | vs. SGN-B7H4V | Control | | | | |
| mouse | B7H41001 mAb-DM1 | Down.in.Test/ | BP | GO:0030595 | leukocyte chemotaxis | 2.07E−04 |
| | vs. SGN-B7H4V | Control | | | | |
| mouse | B7H41001 mAb-DM1 | Down.in.Test/ | BP | GO:0034154 | toll-like receptor 7 signaling | 2.07E−04 |
| | vs. SGN-B7H4V | Control | | | pathway | |
| mouse | B7H41001 mAb-DM1 | Down.in.Test/ | BP | GO:0035457 | cellular response to interferon- | 2.10E−04 |
| | vs. SGN-B7H4V | Control | | | alpha | |
| mouse | B7H41001 mAb-DM1 | Down.in.Test/ | BP | GO:0002532 | production of molecular mediator | 2.10E−04 |
| | vs. SGN-B7H4V | Control | | | involved in inflammatory | |
| | | | | | response | |
| mouse | B7H41001 mAb-DM1 | Down.in.Test/ | BP | GO:0070665 | positive regulation of leukocyte | 2.36E−04 |
| | vs. SGN-B7H4V | Control | | | proliferation | |
| mouse | B7H41001 mAb-DM1 | Down.in.Test/ | BP | GO:0002285 | lymphocyte activation involved in | 2.68E−04 |
| | vs. SGN-B7H4V | Control | | | immune response | |
| mouse | B7H41001 mAb-DM1 | Down.in.Test/ | BP | GO:0036230 | granulocyte activation | 2.94E−04 |
| | vs. SGN-B7H4V | Control | | | | |
| mouse | B7H41001 mAb-DM1 | Down.in.Test/ | BP | GO:1902600 | proton transmembrane transport | 2.94E−04 |
| | vs. SGN-B7H4V | Control | | | | |
| mouse | B7H41001 mAb-DM1 | Down.in.Test/ | BP | GO:0051452 | intracellular pH reduction | 3.58E−04 |
| | vs. SGN-B7H4V | Control | | | | |
| mouse | B7H41001 mAb-DM1 | Up.in.Test/ | BP | GO:0010035 | response to inorganic substance | 3.75E−04 |
| | vs. SGN-B7H4V | Control | | | | |
| mouse | B7H41001 mAb-DM1 | Down.in.Test/ | BP | GO:0045785 | positive regulation of cell | 3.97E−04 |
| | vs. SGN-B7H4V | Control | | | adhesion | |
| mouse | B7H41001 mAb-DM1 | Down.in.Test/ | BP | GO:0098543 | detection of other organism | 4.01E−04 |
| | vs. SGN-B7H4V | Control | | | | |
| mouse | B7H41001 mAb-DM1 | Down.in.Test/ | BP | GO:0006801 | superoxide metabolic process | 4.03E−04 |
| | vs. SGN-B7H4V | Control | | | | |
| mouse | B7H41001 mAb-DM1 | Down.in.Test/ | BP | GO:0032609 | interferon-gamma production | 4.10E−04 |
| | vs. SGN-B7H4V | Control | | | | |
| mouse | B7H41001 mAb-DM1 | Down.in.Test/ | BP | GO:0001916 | positive regulation of T cell | 4.22E−04 |
| | vs. SGN-B7H4V | Control | | | mediated cytotoxicity | |
| mouse | B7H41001 mAb-DM1 | Down.in.Test/ | BP | GO:0050691 | regulation of defense response to | 4.22E−04 |
| | vs. SGN-B7H4V | Control | | | virus by host | |
| mouse | B7H41001 mAb-DM1 | Down.in.Test/ | BP | GO:1901136 | carbohydrate derivative catabolic | 4.66E−04 |
| | vs. SGN-B7H4V | Control | | | process | |
| mouse | B7H41001 mAb-DM1 | Down.in.Test/ | BP | GO:0030641 | regulation of cellular pH | 4.68E−04 |
| | vs. SGN-B7H4V | Control | | | | |
| mouse | B7H41001 mAb-DM1 | Down.in.Test/ | BP | GO:0002475 | antigen processing and | 4.75E−04 |
| | vs. SGN-B7H4V | Control | | | presentation via MHC class Ib | |
| mouse | B7H41001 mAb-DM1 | Down.in.Test/ | BP | GO:0010950 | positive regulation of | 4.94E−04 |
| | vs. SGN-B7H4V | Control | | | endopeptidase activity | |
| mouse | B7H41001 mAb-DM1 | Down.in.Test/ | BP | GO:0097696 | receptor signaling pathway via | 4.94E−04 |
| | vs. SGN-B7H4V | Control | | | STAT | |
| mouse | B7H41001 mAb-DM1 | Down.in.Test/ | BP | GO:0032722 | positive regulation of chemokine | 5.18E−04 |
| | vs. SGN-B7H4V | Control | | | production | |
| mouse | B7H41001 mAb-DM1 | Down.in.Test/ | BP | GO:0060333 | interferon-gamma-mediated | 5.18E−04 |
| | vs. SGN-B7H4V | Control | | | signaling pathway | |
| mouse | B7H41001 mAb-DM1 | Down.in.Test/ | BP | GO:0052548 | regulation of endopeptidase | 5.18E−04 |
| | vs. SGN-B7H4V | Control | | | activity | |
| mouse | B7H41001 mAb-DM1 | Up.in.Test/ | BP | GO:0015850 | organic hydroxy compound | 5.23E−04 |
| | vs. SGN-B7H4V | Control | | | transport | |
| mouse | B7H41001 mAb-DM1 | Down.in.Test/ | BP | GO:1902107 | positive regulation of leukocyte | 5.36E−04 |
| | vs. SGN-B7H4V | Control | | | differentiation | |
| mouse | B7H41001 mAb-DM1 | Down.in.Test/ | BP | GO:1903708 | positive regulation of hemopoiesis | 5.36E−04 |
| | vs. SGN-B7H4V | Control | | | | |
| mouse | B7H41001 mAb-DM1 | Down.in.Test/ | BP | GO:0050729 | positive regulation of | 5.53E−04 |
| | vs. SGN-B7H4V | Control | | | inflammatory response | |
| mouse | B7H41001 mAb-DM1 | Down.in.Test/ | BP | GO:0032649 | regulation of interferon-gamma | 5.55E−04 |
| | vs. SGN-B7H4V | Control | | | production | |
| mouse | B7H41001 mAb-DM1 | Down.in.Test/ | BP | GO:0044403 | biological process involved in | 5.79E−04 |
| | vs. SGN-B7H4V | Control | | | symbiotic interaction | |
| mouse | B7H41001 mAb-DM1 | Down.in.Test/ | BP | GO:0030101 | natural killer cell activation | 6.31E−04 |
| | vs. SGN-B7H4V | Control | | | | |
| mouse | B7H41001 mAb-DM1 | Down.in.Test/ | BP | GO:0042102 | positive regulation of T cell | 6.37E−04 |
| | vs. SGN-B7H4V | Control | | | proliferation | |
| mouse | B7H41001 mAb-DM1 | Down.in.Test/ | BP | GO:0016064 | immunoglobulin mediated | 6.54E−04 |
| | vs. SGN-B7H4V | Control | | | immune response | |
| mouse | B7H41001 mAb-DM1 | Down.in.Test/ | BP | GO:0007035 | vacuolar acidification | 6.58E−04 |
| | vs. SGN-B7H4V | Control | | | | |
| mouse | B7H41001 mAb-DM1 | Down.in.Test/ | BP | GO:0140632 | inflammasome complex assembly | 6.58E−04 |
| | vs. SGN-B7H4V | Control | | | | |
| mouse | B7H41001 mAb-DM1 | Down.in.Test/ | BP | GO:0062208 | positive regulation of pattern | 6.72E−04 |
| | vs. SGN-B7H4V | Control | | | recognition receptor signaling | |
| | | | | | pathway | |
| mouse | B7H41001 mAb-DM1 | Down.in.Test/ | BP | GO:0070431 | nucleotide-binding | 7.04E−04 |
| | vs. SGN-B7H4V | Control | | | oligomerization domain | |
| | | | | | containing 2 signaling pathway | |
| mouse | B7H41001 mAb-DM1 | Down.in.Test/ | BP | GO:0002711 | positive regulation of T cell | 7.04E−04 |
| | vs. SGN-B7H4V | Control | | | mediated immunity | |
| mouse | B7H41001 mAb-DM1 | Down.in.Test/ | BP | GO:0050920 | regulation of chemotaxis | 7.15E−04 |
| | vs. SGN-B7H4V | Control | | | | |
| mouse | B7H41001 mAb-DM1 | Up.in.Test/ | BP | GO:0061138 | morphogenesis of a branching | 7.53E−04 |
| | vs. SGN-B7H4V | Control | | | epithelium | |
| mouse | B7H41001 mAb-DM1 | Up.in.Test/ | BP | GO:0050873 | brown fat cell differentiation | 7.53E−04 |
| | vs. SGN-B7H4V | Control | | | | |
| mouse | B7H41001 mAb-DM1 | Up.in.Test/ | BP | GO:0045765 | regulation of angiogenesis | 7.53E−04 |
| | vs. SGN-B7H4V | Control | | | | |
| mouse | B7H41001 mAb-DM1 | Up.in.Test/ | BP | GO:0006939 | smooth muscle contraction | 7.53E−04 |
| | vs. SGN-B7H4V | Control | | | | |
| mouse | B7H41001 mAb-DM1 | Up.in.Test/ | BP | GO:0048762 | mesenchymal cell differentiation | 7.53E−04 |
| | vs. SGN-B7H4V | Control | | | | |
| mouse | B7H41001 mAb-DM1 | Up.in.Test/ | BP | GO:1901342 | regulation of vasculature | 7.53E−04 |
| | vs. SGN-B7H4V | Control | | | development | |
| mouse | B7H41001 mAb-DM1 | Down.in.Test/ | BP | GO:0019079 | viral genome replication | 7.69E−04 |
| | vs. SGN-B7H4V | Control | | | | |
| mouse | B7H41001 mAb-DM1 | Up.in.Test/ | BP | GO:0048754 | branching morphogenesis of an | 7.85E−04 |
| | vs. SGN-B7H4V | Control | | | epithelial tube | |
| mouse | B7H41001 mAb-DM1 | Up.in.Test/ | BP | GO:0050673 | epithelial cell proliferation | 7.85E−04 |
| | vs. SGN-B7H4V | Control | | | | |
| mouse | B7H41001 mAb-DM1 | Down.in.Test/ | BP | GO:0019724 | B cell mediated immunity | 7.98E−04 |
| | vs. SGN-B7H4V | Control | | | | |
| mouse | B7H41001 mAb-DM1 | Down.in.Test/ | BP | GO:0007249 | I-kappaB kinase/NF-kappaB | 7.98E−04 |
| | vs. SGN-B7H4V | Control | | | signaling | |
| mouse | B7H41001 mAb-DM1 | Up.in.Test/ | BP | GO:0055074 | calcium ion homeostasis | 8.34E−04 |
| | vs. SGN-B7H4V | Control | | | | |
| mouse | B7H41001 mAb-DM1 | Down.in.Test/ | BP | GO:0006879 | cellular iron ion homeostasis | 8.37E−04 |
| | vs. SGN-B7H4V | Control | | | | |
| mouse | B7H41001 mAb-DM1 | Down.in.Test/ | BP | GO:0050864 | regulation of B cell activation | 8.53E−04 |
| | vs. SGN-B7H4V | Control | | | | |
| mouse | B7H41001 mAb-DM1 | Down.in.Test/ | BP | GO:0010952 | positive regulation of peptidase | 8.72E−04 |
| | vs. SGN-B7H4V | Control | | | activity | |
| mouse | B7H41001 mAb-DM1 | Down.in.Test/ | BP | GO:0043506 | regulation of JUN kinase activity | 8.74E−04 |
| | vs. SGN-B7H4V | Control | | | | |
| mouse | B7H41001 mAb-DM1 | Down.in.Test/ | BP | GO:0042113 | B cell activation | 9.23E−04 |
| | vs. SGN-B7H4V | Control | | | | |
| mouse | B7H41001 mAb-DM1 | Up.in.Test/ | BP | GO:0001763 | morphogenesis of a branching | 9.44E−04 |
| | vs. SGN-B7H4V | Control | | | structure | |
| mouse | B7H41001 mAb-DM1 | Down.in.Test/ | BP | GO:0007259 | receptor signaling pathway via | 9.76E−04 |
| | vs. SGN-B7H4V | Control | | | JAK-STAT | |
| mouse | B7H41001 mAb-DM1 | Down.in.Test/ | BP | GO:0034162 | toll-like receptor 9 signaling | 9.76E−04 |
| | vs. SGN-B7H4V | Control | | | pathway | |
| mouse | B7H41001 mAb-DM1 | Down.in.Test/ | BP | GO:0060700 | regulation of ribonuclease activity | 9.76E−04 |
| | vs. SGN-B7H4V | Control | | | | |
| mouse | B7H41001 mAb-DM1 | Down.in.Test/ | BP | GO:0001779 | natural killer cell differentiation | 9.80E−04 |
| | vs. SGN-B7H4V | Control | | | | |
| mouse | B7H41001 mAb-DM1 | Down.in.Test/ | BP | GO:2000107 | negative regulation of leukocyte | 1.07E−03 |
| | vs. SGN-B7H4V | Control | | | apoptotic process | |
| mouse | B7H41001 mAb-DM1 | Up.in.Test/ | BP | GO:0062013 | positive regulation of small | 1.07E−03 |
| | vs. SGN-B7H4V | Control | | | molecule metabolic process | |
| mouse | B7H41001 mAb-DM1 | Up.in.Test/ | BP | GO:0060347 | heart trabecula formation | 1.07E−03 |
| | vs. SGN-B7H4V | Control | | | | |
| mouse | B7H41001 mAb-DM1 | Down.in.Test/ | BP | GO:0032677 | regulation of interleukin-8 | 1.08E−03 |
| | vs. SGN-B7H4V | Control | | | production | |
| mouse | B7H41001 mAb-DM1 | Up.in.Test/ | BP | GO:1903707 | negative regulation of | 1.15E−03 |
| | vs. SGN-B7H4V | Control | | | hemopoiesis | |
| mouse | B7H41001 mAb-DM1 | Up.in.Test/ | BP | GO:0072006 | nephron development | 1.15E−03 |
| | vs. SGN-B7H4V | Control | | | | |
| mouse | B7H41001 mAb-DM1 | Up.in.Test/ | BP | GO:0062012 | regulation of small molecule | 1.16E−03 |
| | vs. SGN-B7H4V | Control | | | metabolic process | |
| mouse | B7H41001 mAb-DM1 | Up.in.Test/ | BP | GO:0001667 | ameboidal-type cell migration | 1.16E−03 |
| | vs. SGN-B7H4V | Control | | | | |
| mouse | B7H41001 mAb-DM1 | Down.in.Test/ | BP | GO:0009595 | detection of biotic stimulus | 1.18E−03 |
| | vs. SGN-B7H4V | Control | | | | |
| mouse | B7H41001 mAb-DM1 | Down.in.Test/ | BP | GO:0002637 | regulation of immunoglobulin | 1.20E−03 |
| | vs. SGN-B7H4V | Control | | | production | |
| mouse | B7H41001 mAb-DM1 | Down.in.Test/ | BP | GO:0032637 | interleukin-8 production | 1.20E−03 |
| | vs. SGN-B7H4V | Control | | | | |
| mouse | B7H41001 mAb-DM1 | Up.in.Test/ | BP | GO:0060485 | mesenchyme development | 1.22E−03 |
| | vs. SGN-B7H4V | Control | | | | |
| mouse | B7H41001 mAb-DM1 | Up.in.Test/ | BP | GO:0072009 | nephron epithelium development | 1.31E−03 |
| | vs. SGN-B7H4V | Control | | | | |
| mouse | B7H41001 mAb-DM1 | Down.in.Test/ | BP | GO:0070423 | nucleotide-binding | 1.34E−03 |
| | vs. SGN-B7H4V | Control | | | oligomerization domain | |
| | | | | | containing signaling pathway | |
| mouse | B7H41001 mAb-DM1 | Down.in.Test/ | BP | GO:0032655 | regulation of interleukin-12 | 1.36E−03 |
| | vs. SGN-B7H4V | Control | | | production | |
| mouse | B7H41001 mAb-DM1 | Down.in.Test/ | BP | GO:0002286 | T cell activation involved in | 1.37E−03 |
| | vs. SGN-B7H4V | Control | | | immune response | |
| mouse | B7H41001 mAb-DM1 | Up.in.Test/ | BP | GO:0071241 | cellular response to inorganic | 1.40E−03 |
| | vs. SGN-B7H4V | Control | | | substance | |
| mouse | B7H41001 mAb-DM1 | Down.in.Test/ | BP | GO:0002367 | cytokine production involved in | 1.49E−03 |
| | vs. SGN-B7H4V | Control | | | immune response | |
| mouse | B7H41001 mAb-DM1 | Down.in.Test/ | BP | GO:0072593 | reactive oxygen species metabolic | 1.63E−03 |
| | vs. SGN-B7H4V | Control | | | process | |
| mouse | B7H41001 mAb-DM1 | Down.in.Test/ | BP | GO:0098581 | detection of external biotic | 1.63E−03 |
| | vs. SGN-B7H4V | Control | | | stimulus | |
| mouse | B7H41001 mAb-DM1 | Down.in.Test/ | BP | GO:0032615 | interleukin-12 production | 1.72E−03 |
| | vs. SGN-B7H4V | Control | | | | |
| mouse | B7H41001 mAb-DM1 | Down.in.Test/ | BP | GO:0002690 | positive regulation of leukocyte | 1.76E−03 |
| | vs. SGN-B7H4V | Control | | | chemotaxis | |
| mouse | B7H41001 mAb-DM1 | Down.in.Test/ | BP | GO:0002718 | regulation of cytokine production | 1.76E−03 |
| | vs. SGN-B7H4V | Control | | | involved in immune response | |
| mouse | B7H41001 mAb-DM1 | Up.in.Test/ | BP | GO:0060326 | cell chemotaxis | 1.78E−03 |
| | vs. SGN-B7H4V | Control | | | | |
| mouse | B7H41001 mAb-DM1 | Up.in.Test/ | BP | GO:0072503 | cellular divalent inorganic cation | 1.78E−03 |
| | vs. SGN-B7H4V | Control | | | homeostasis | |
| mouse | B7H41001 mAb-DM1 | Up.in.Test/ | BP | GO:0072080 | nephron tubule development | 1.78E−03 |
| | vs. SGN-B7H4V | Control | | | | |
| mouse | B7H41001 mAb-DM1 | Up.in.Test/ | BP | GO:0045834 | positive regulation of lipid | 1.78E−03 |
| | vs. SGN-B7H4V | Control | | | metabolic process | |
| mouse | B7H41001 mAb-DM1 | Up.in.Test/ | BP | GO:0051051 | negative regulation of transport | 1.78E−03 |
| | vs. SGN-B7H4V | Control | | | | |
| mouse | B7H41001 mAb-DM1 | Up.in.Test/ | BP | GO:0002040 | sprouting angiogenesis | 1.78E−03 |
| | vs. SGN-B7H4V | Control | | | | |
| mouse | B7H41001 mAb-DM1 | Up.in.Test/ | BP | GO:0006874 | cellular calcium ion homeostasis | 1.78E−03 |
| | vs. SGN-B7H4V | Control | | | | |
| mouse | B7H41001 mAb-DM1 | Up.in.Test/ | BP | GO:0006936 | muscle contraction | 1.78E−03 |
| | vs. SGN-B7H4V | Control | | | | |
| mouse | B7H41001 mAb-DM1 | Up.in.Test/ | BP | GO:1902105 | regulation of leukocyte | 1.78E−03 |
| | vs. SGN-B7H4V | Control | | | differentiation | |
| mouse | B7H41001 mAb-DM1 | Up.in.Test/ | BP | GO:0045444 | fat cell differentiation | 1.78E−03 |
| | vs. SGN-B7H4V | Control | | | | |
| mouse | B7H41001 mAb-DM1 | Up.in.Test/ | BP | GO:0050920 | regulation of chemotaxis | 1.78E−03 |
| | vs. SGN-B7H4V | Control | | | | |
| mouse | B7H41001 mAb-DM1 | Up.in.Test/ | BP | GO:0010906 | regulation of glucose metabolic | 1.78E−03 |
| | vs. SGN-B7H4V | Control | | | process | |
| mouse | B7H41001 mAb-DM1 | Up.in.Test/ | BP | GO:0070875 | positive regulation of glycogen | 1.78E−03 |
| | vs. SGN-B7H4V | Control | | | metabolic process | |
| mouse | B7H41001 mAb-DM1 | Down.in.Test/ | BP | GO:0002698 | negative regulation of immune | 1.78E−03 |
| | vs. SGN-B7H4V | Control | | | effector process | |
| mouse | B7H41001 mAb-DM1 | Down.in.Test/ | BP | GO:0035872 | nucleotide-binding domain, | 1.78E−03 |
| | vs. SGN-B7H4V | Control | | | leucine rich repeat containing | |
| | | | | | receptor signaling pathway | |
| mouse | B7H41001 mAb-DM1 | Up.in.Test/ | BP | GO:0050921 | positive regulation of chemotaxis | 1.83E−03 |
| | vs. SGN-B7H4V | Control | | | | |
| mouse | B7H41001 mAb-DM1 | Up.in.Test/ | BP | GO:0072088 | nephron epithelium | 1.83E−03 |
| | vs. SGN-B7H4V | Control | | | morphogenesis | |
| mouse | B7H41001 mAb-DM1 | Up.in.Test/ | BP | GO:0072073 | kidney epithelium development | 1.84E−03 |
| | vs. SGN-B7H4V | Control | | | | |
| mouse | B7H41001 mAb-DM1 | Up.in.Test/ | BP | GO:0006641 | triglyceride metabolic process | 1.84E−03 |
| | vs. SGN-B7H4V | Control | | | | |
| mouse | B7H41001 mAb-DM1 | Up.in.Test/ | BP | GO:0061326 | renal tubule development | 1.85E−03 |
| | vs. SGN-B7H4V | Control | | | | |
| mouse | B7H41001 mAb-DM1 | Up.in.Test/ | BP | GO:0009914 | hormone transport | 1.85E−03 |
| | vs. SGN-B7H4V | Control | | | | |
| mouse | B7H41001 mAb-DM1 | Up.in.Test/ | BP | GO:0050678 | regulation of epithelial cell | 1.85E−03 |
| | vs. SGN-B7H4V | Control | | | proliferation | |
| mouse | B7H41001 mAb-DM1 | Up.in.Test/ | BP | GO:0046888 | negative regulation of hormone | 1.85E−03 |
| | vs. SGN-B7H4V | Control | | | secretion | |
| mouse | B7H41001 mAb-DM1 | Up.in.Test/ | BP | GO:0072028 | nephron morphogenesis | 1.85E−03 |
| | vs. SGN-B7H4V | Control | | | | |
| mouse | B7H41001 mAb-DM1 | Up.in.Test/ | BP | GO:0008217 | regulation of blood pressure | 1.85E−03 |
| | vs. SGN-B7H4V | Control | | | | |
| mouse | B7H41001 mAb-DM1 | Down.in.Test/ | BP | GO:0051250 | negative regulation of lymphocyte | 1.86E−03 |
| | vs. SGN-B7H4V | Control | | | activation | |
| mouse | B7H41001 mAb-DM1 | Up.in.Test/ | BP | GO:0030856 | regulation of epithelial cell | 1.98E−03 |
| | vs. SGN-B7H4V | Control | | | differentiation | |
| mouse | B7H41001 mAb-DM1 | Up.in.Test/ | BP | GO:0001666 | response to hypoxia | 1.98E−03 |
| | vs. SGN-B7H4V | Control | | | | |
| mouse | B7H41001 mAb-DM1 | Down.in.Test/ | BP | GO:2000116 | regulation of cysteine-type | 1.99E−03 |
| | vs. SGN-B7H4V | Control | | | endopeptidase activity | |
| mouse | B7H41001 mAb-DM1 | Down.in.Test/ | BP | GO:0055072 | iron ion homeostasis | 1.99E−03 |
| | vs. SGN-B7H4V | Control | | | | |
| mouse | B7H41001 mAb-DM1 | Up.in.Test/ | BP | GO:0061333 | renal tubule morphogenesis | 2.05E−03 |
| | vs. SGN-B7H4V | Control | | | | |
| mouse | B7H41001 mAb-DM1 | Up.in.Test/ | BP | GO:1902106 | negative regulation of leukocyte | 2.05E−03 |
| | vs. SGN-B7H4V | Control | | | differentiation | |
| mouse | B7H41001 mAb-DM1 | Up.in.Test/ | BP | GO:0010675 | regulation of cellular | 2.10E−03 |
| | vs. SGN-B7H4V | Control | | | carbohydrate metabolic process | |
| mouse | B7H41001 mAb-DM1 | Up.in.Test/ | BP | GO:0006006 | glucose metabolic process | 2.15E−03 |
| | vs. SGN-B7H4V | Control | | | | |
| mouse | B7H41001 mAb-DM1 | Up.in.Test/ | BP | GO:0002761 | regulation of myeloid leukocyte | 2.16E−03 |
| | vs. SGN-B7H4V | Control | | | differentiation | |
| mouse | B7H41001 mAb-DM1 | Up.in.Test/ | BP | GO:0045637 | regulation of myeloid cell | 2.30E−03 |
| | vs. SGN-B7H4V | Control | | | differentiation | |
| mouse | B7H41001 mAb-DM1 | Down.in.Test/ | BP | GO:1900225 | regulation of NLRP3 | 2.37E−03 |
| | vs. SGN-B7H4V | Control | | | inflammasome complex assembly | |
| mouse | B7H41001 mAb-DM1 | Up.in.Test/ | BP | GO:0002027 | regulation of heart rate | 2.37E−03 |
| | vs. SGN-B7H4V | Control | | | | |
| mouse | B7H41001 mAb-DM1 | Up.in.Test/ | BP | GO:0036293 | response to decreased oxygen | 2.46E−03 |
| | vs. SGN-B7H4V | Control | | | levels | |
| mouse | B7H41001 mAb-DM1 | Down.in.Test/ | BP | GO:0006885 | regulation of pH | 2.49E−03 |
| | vs. SGN-B7H4V | Control | | | | |
| mouse | B7H41001 mAb-DM1 | Down.in.Test/ | BP | GO:0030593 | neutrophil chemotaxis | 2.49E−03 |
| | vs. SGN-B7H4V | Control | | | | |
| mouse | B7H41001 mAb-DM1 | Down.in.Test/ | BP | GO:1905037 | autophagosome organization | 2.49E−03 |
| | vs. SGN-B7H4V | Control | | | | |
| mouse | B7H41001 mAb-DM1 | Up.in.Test/ | BP | GO:0050900 | leukocyte migration | 2.49E−03 |
| | vs. SGN-B7H4V | Control | | | | |
| mouse | B7H41001 mAb-DM1 | Up.in.Test/ | BP | GO:0060562 | epithelial tube morphogenesis | 2.49E−03 |
| | vs. SGN-B7H4V | Control | | | | |
| mouse | B7H41001 mAb-DM1 | Up.in.Test/ | BP | GO:0001935 | endothelial cell proliferation | 2.49E−03 |
| | vs. SGN-B7H4V | Control | | | | |
| mouse | B7H41001 mAb-DM1 | Down.in.Test/ | BP | GO:0030522 | intracellular receptor signaling | 2.54E−03 |
| | vs. SGN-B7H4V | Control | | | pathway | |
| mouse | B7H41001 mAb-DM1 | Down.in.Test/ | BP | GO:0052547 | regulation of peptidase activity | 2.57E−03 |
| | vs. SGN-B7H4V | Control | | | | |
| mouse | B7H41001 mAb-DM1 | Down.in.Test/ | BP | GO:0038061 | NIK/NF-kappaB signaling | 2.57E−03 |
| | vs. SGN-B7H4V | Control | | | | |
| mouse | B7H41001 mAb-DM1 | Down.in.Test/ | BP | GO:0001774 | microglial cell activation | 2.60E−03 |
| | vs. SGN-B7H4V | Control | | | | |
| mouse | B7H41001 mAb-DM1 | Down.in.Test/ | BP | GO:0050688 | regulation of defense response to | 2.63E−03 |
| | vs. SGN-B7H4V | Control | | | virus | |
| mouse | B7H41001 mAb-DM1 | Up.in.Test/ | BP | GO:0006109 | regulation of carbohydrate | 2.64E−03 |
| | vs. SGN-B7H4V | Control | | | metabolic process | |
| mouse | B7H41001 mAb-DM1 | Up.in.Test/ | BP | GO:0097529 | myeloid leukocyte migration | 2.64E−03 |
| | vs. SGN-B7H4V | Control | | | | |
| mouse | B7H41001 mAb-DM1 | Up.in.Test/ | BP | GO:0003073 | regulation of systemic arterial | 2.66E−03 |
| | vs. SGN-B7H4V | Control | | | blood pressure | |
| mouse | B7H41001 mAb-DM1 | Up.in.Test/ | BP | GO:0042440 | pigment metabolic process | 2.88E−03 |
| | vs. SGN-B7H4V | Control | | | | |
| mouse | B7H41001 mAb-DM1 | Down.in.Test/ | BP | GO:0002230 | positive regulation of defense | 2.90E−03 |
| | vs. SGN-B7H4V | Control | | | response to virus by host | |
| mouse | B7H41001 mAb-DM1 | Up.in.Test/ | BP | GO:0071248 | cellular response to metal ion | 2.94E−03 |
| | vs. SGN-B7H4V | Control | | | | |
| mouse | B7H41001 mAb-DM1 | Up.in.Test/ | BP | GO:0014910 | regulation of smooth muscle cell | 2.97E−03 |
| | vs. SGN-B7H4V | Control | | | migration | |
| mouse | B7H41001 mAb-DM1 | Down.in.Test/ | BP | GO:0007039 | protein catabolic process in the | 3.01E−03 |
| | vs. SGN-B7H4V | Control | | | vacuole | |
| mouse | B7H41001 mAb-DM1 | Down.in.Test/ | BP | GO:0045619 | regulation of lymphocyte | 3.01E−03 |
| | vs. SGN-B7H4V | Control | | | differentiation | |
| mouse | B7H41001 mAb-DM1 | Up.in.Test/ | BP | GO:0046879 | hormone secretion | 3.19E−03 |
| | vs. SGN-B7H4V | Control | | | | |
| mouse | B7H41001 mAb-DM1 | Down.in.Test/ | BP | GO:0030888 | regulation of B cell proliferation | 3.25E−03 |
| | vs. SGN-B7H4V | Control | | | | |
| mouse | B7H41001 mAb-DM1 | Down.in.Test/ | BP | GO:2000146 | negative regulation of cell motility | 3.29E−03 |
| | vs. SGN-B7H4V | Control | | | | |
| mouse | B7H41001 mAb-DM1 | Up.in.Test/ | BP | GO:0018958 | phenol-containing compound | 3.33E−03 |
| | vs. SGN-B7H4V | Control | | | metabolic process | |
| mouse | B7H41001 mAb-DM1 | Down.in.Test/ | BP | GO:0043507 | positive regulation of JUN kinase | 3.33E−03 |
| | vs. SGN-B7H4V | Control | | | activity | |
| mouse | B7H41001 mAb-DM1 | Down.in.Test/ | BP | GO:0045058 | T cell selection | 3.33E−03 |
| | vs. SGN-B7H4V | Control | | | | |
| mouse | B7H41001 mAb-DM1 | Down.in.Test/ | BP | GO:0061635 | regulation of protein complex | 3.33E−03 |
| | vs. SGN-B7H4V | Control | | | stability | |
| mouse | B7H41001 mAb-DM1 | Down.in.Test/ | BP | GO:0140289 | protein mono-ADP-ribosylation | 3.33E−03 |
| | vs. SGN-B7H4V | Control | | | | |
| mouse | B7H41001 mAb-DM1 | Down.in.Test/ | BP | GO:1900227 | positive regulation of NLRP3 | 3.33E−03 |
| | vs. SGN-B7H4V | Control | | | inflammasome complex assembly | |
| mouse | B7H41001 mAb-DM1 | Up.in.Test/ | BP | GO:0014031 | mesenchymal cell development | 3.36E−03 |
| | vs. SGN-B7H4V | Control | | | | |
| mouse | B7H41001 mAb-DM1 | Up.in.Test/ | BP | GO:0030099 | myeloid cell differentiation | 3.36E−03 |
| | vs. SGN-B7H4V | Control | | | | |
| mouse | B7H41001 mAb-DM1 | Down.in.Test/ | BP | GO:0016236 | macroautophagy | 3.38E−03 |
| | vs. SGN-B7H4V | Control | | | | |
| mouse | B7H41001 mAb-DM1 | Down.in.Test/ | BP | GO:0032814 | regulation of natural killer cell | 3.39E−03 |
| | vs. SGN-B7H4V | Control | | | activation | |
| mouse | B7H41001 mAb-DM1 | Down.in.Test/ | BP | GO:0042554 | superoxide anion generation | 3.39E−03 |
| | vs. SGN-B7H4V | Control | | | | |
| mouse | B7H41001 mAb-DM1 | Up.in.Test/ | BP | GO:0001655 | urogenital system development | 3.41E−03 |
| | vs. SGN-B7H4V | Control | | | | |
| mouse | B7H41001 mAb-DM1 | Up.in.Test/ | BP | GO:0033344 | cholesterol efflux | 3.41E−03 |
| | vs. SGN-B7H4V | Control | | | | |
| mouse | B7H41001 mAb-DM1 | Up.in.Test/ | BP | GO:0070663 | regulation of leukocyte | 3.44E−03 |
| | vs. SGN-B7H4V | Control | | | proliferation | |
| mouse | B7H41001 mAb-DM1 | Up.in.Test/ | BP | GO:1905952 | regulation of lipid localization | 3.62E−03 |
| | vs. SGN-B7H4V | Control | | | | |
| mouse | B7H41001 mAb-DM1 | Up.in.Test/ | BP | GO:0010874 | regulation of cholesterol efflux | 3.62E−03 |
| | vs. SGN-B7H4V | Control | | | | |
| mouse | B7H41001 mAb-DM1 | Down.in.Test/ | BP | GO:0043280 | positive regulation of cysteine- | 3.63E−03 |
| | vs. SGN-B7H4V | Control | | | type endopeptidase activity | |
| | | | | | involved in apoptotic process | |
| mouse | B7H41001 mAb-DM1 | Down.in.Test/ | BP | GO:0071900 | regulation of protein | 3.64E−03 |
| | vs. SGN-B7H4V | Control | | | serine/threonine kinase activity | |
| mouse | B7H41001 mAb-DM1 | Down.in.Test/ | BP | GO:0044546 | NLRP3 inflammasome complex | 3.73E−03 |
| | vs. SGN-B7H4V | Control | | | assembly | |
| mouse | B7H41001 mAb-DM1 | Up.in.Test/ | BP | GO:1903706 | regulation of hemopoiesis | 3.83E−03 |
| | vs. SGN-B7H4V | Control | | | | |
| mouse | B7H41001 mAb-DM1 | Down.in.Test/ | BP | GO:0007040 | lysosome organization | 3.83E−03 |
| | vs. SGN-B7H4V | Control | | | | |
| mouse | B7H41001 mAb-DM1 | Down.in.Test/ | BP | GO:0051453 | regulation of intracellular pH | 3.83E−03 |
| | vs. SGN-B7H4V | Control | | | | |
| mouse | B7H41001 mAb-DM1 | Down.in.Test/ | BP | GO:0080171 | lytic vacuole organization | 3.83E−03 |
| | vs. SGN-B7H4V | Control | | | | |
| mouse | B7H41001 mAb-DM1 | Down.in.Test/ | BP | GO:0006865 | amino acid transport | 3.83E−03 |
| | vs. SGN-B7H4V | Control | | | | |
| mouse | B7H41001 mAb-DM1 | Down.in.Test/ | BP | GO:2001056 | positive regulation of cysteine- | 3.90E−03 |
| | vs. SGN-B7H4V | Control | | | type endopeptidase activity | |
| mouse | B7H41001 mAb-DM1 | Up.in.Test/ | BP | GO:0045913 | positive regulation of | 3.91E−03 |
| | vs. SGN-B7H4V | Control | | | carbohydrate metabolic process | |
| mouse | B7H41001 mAb-DM1 | Up.in.Test/ | BP | GO:0043405 | regulation of MAP kinase activity | 3.93E−03 |
| | vs. SGN-B7H4V | Control | | | | |
| mouse | B7H41001 mAb-DM1 | Up.in.Test/ | BP | GO:0010676 | positive regulation of cellular | 3.93E−03 |
| | vs. SGN-B7H4V | Control | | | carbohydrate metabolic process | |
| mouse | B7H41001 mAb-DM1 | Up.in.Test/ | BP | GO:0043473 | pigmentation | 4.00E−03 |
| | vs. SGN-B7H4V | Control | | | | |
| mouse | B7H41001 mAb-DM1 | Up.in.Test/ | BP | GO:0070482 | response to oxygen levels | 4.00E−03 |
| | vs. SGN-B7H4V | Control | | | | |
| mouse | B7H41001 mAb-DM1 | Up.in.Test/ | BP | GO:0003012 | muscle system process | 4.00E−03 |
| | vs. SGN-B7H4V | Control | | | | |
| mouse | B7H41001 mAb-DM1 | Down.in.Test/ | BP | GO:0043281 | regulation of cysteine-type | 4.01E−03 |
| | vs. SGN-B7H4V | Control | | | endopeptidase activity involved in | |
| | | | | | apoptotic process | |
| mouse | B7H41001 mAb-DM1 | Up.in.Test/ | BP | GO:0019216 | regulation of lipid metabolic | 4.09E−03 |
| | vs. SGN-B7H4V | Control | | | process | |
| mouse | B7H41001 mAb-DM1 | Up.in.Test/ | BP | GO:0014909 | smooth muscle cell migration | 4.09E−03 |
| | vs. SGN-B7H4V | Control | | | | |
| mouse | B7H41001 mAb-DM1 | Up.in.Test/ | BP | GO:0060993 | kidney morphogenesis | 4.09E−03 |
| | vs. SGN-B7H4V | Control | | | | |
| mouse | B7H41001 mAb-DM1 | Down.in.Test/ | BP | GO:0042157 | lipoprotein metabolic process | 4.10E−03 |
| | vs. SGN-B7H4V | Control | | | | |
| mouse | B7H41001 mAb-DM1 | Up.in.Test/ | BP | GO:0002687 | positive regulation of leukocyte | 4.14E−03 |
| | vs. SGN-B7H4V | Control | | | migration | |
| mouse | B7H41001 mAb-DM1 | Down.in.Test/ | BP | GO:0030004 | cellular monovalent inorganic | 4.15E−03 |
| | vs. SGN-B7H4V | Control | | | cation homeostasis | |
| mouse | B7H41001 mAb-DM1 | Down.in.Test/ | BP | GO:0043405 | regulation of MAP kinase activity | 4.20E−03 |
| | vs. SGN-B7H4V | Control | | | | |
| mouse | B7H41001 mAb-DM1 | Down.in.Test/ | BP | GO:0045860 | positive regulation of protein | 4.31E−03 |
| | vs. SGN-B7H4V | Control | | | kinase activity | |
| mouse | B7H41001 mAb-DM1 | Up.in.Test/ | BP | GO:0007204 | positive regulation of cytosolic | 4.34E−03 |
| | vs. SGN-B7H4V | Control | | | calcium ion concentration | |
| mouse | B7H41001 mAb-DM1 | Up.in.Test/ | BP | GO:0048608 | reproductive structure | 4.34E−03 |
| | vs. SGN-B7H4V | Control | | | development | |
| mouse | B7H41001 mAb-DM1 | Down.in.Test/ | BP | GO:0043123 | positive regulation of I-kappaB | 4.35E−03 |
| | vs. SGN-B7H4V | Control | | | kinase/NF-kappaB signaling | |
| mouse | B7H41001 mAb-DM1 | Down.in.Test/ | BP | GO:0071621 | granulocyte chemotaxis | 4.35E−03 |
| | vs. SGN-B7H4V | Control | | | | |
| mouse | B7H41001 mAb-DM1 | Down.in.Test/ | BP | GO:0030336 | negative regulation of cell | 4.40E−03 |
| | vs. SGN-B7H4V | Control | | | migration | |
| mouse | B7H41001 mAb-DM1 | Down.in.Test/ | BP | GO:0050730 | regulation of peptidyl-tyrosine | 4.40E−03 |
| | vs. SGN-B7H4V | Control | | | phosphorylation | |
| mouse | B7H41001 mAb-DM1 | Up.in.Test/ | BP | GO:0001503 | ossification | 4.41E−03 |
| | vs. SGN-B7H4V | Control | | | | |
| mouse | B7H41001 mAb-DM1 | Up.in.Test/ | BP | GO:0072078 | nephron tubule morphogenesis | 4.41E−03 |
| | vs. SGN-B7H4V | Control | | | | |
| mouse | B7H41001 mAb-DM1 | Down.in.Test/ | BP | GO:0051271 | negative regulation of cellular | 4.51E−03 |
| | vs. SGN-B7H4V | Control | | | component movement | |
| mouse | B7H41001 mAb-DM1 | Down.in.Test/ | BP | GO:0002281 | macrophage activation involved in | 4.51E−03 |
| | vs. SGN-B7H4V | Control | | | immune response | |
| mouse | B7H41001 mAb-DM1 | Down.in.Test/ | BP | GO:0050829 | defense response to Gram- | 4.51E−03 |
| | vs. SGN-B7H4V | Control | | | negative bacterium | |
| mouse | B7H41001 mAb-DM1 | Down.in.Test/ | BP | GO:0036037 | CD8-positive, alpha-beta T cell | 4.55E−03 |
| | vs. SGN-B7H4V | Control | | | activation | |
| mouse | B7H41001 mAb-DM1 | Down.in.Test/ | BP | GO:0071902 | positive regulation of protein | 4.55E−03 |
| | vs. SGN-B7H4V | Control | | | serine/threonine kinase activity | |
| mouse | B7H41001 mAb-DM1 | Down.in.Test/ | BP | GO:0046632 | alpha-beta T cell differentiation | 4.57E−03 |
| | vs. SGN-B7H4V | Control | | | | |
| mouse | B7H41001 mAb-DM1 | Up.in.Test/ | BP | GO:0006582 | melanin metabolic process | 4.67E−03 |
| | vs. SGN-B7H4V | Control | | | | |
| mouse | B7H41001 mAb-DM1 | Up.in.Test/ | BP | GO:0061458 | reproductive system development | 4.69E−03 |
| | vs. SGN-B7H4V | Control | | | | |
| mouse | B7H41001 mAb-DM1 | Down.in.Test/ | BP | GO:0032729 | positive regulation of interferon- | 4.92E−03 |
| | vs. SGN-B7H4V | Control | | | gamma production | |
| mouse | B7H41001 mAb-DM1 | Down.in.Test/ | BP | GO:2000379 | positive regulation of reactive | 4.92E−03 |
| | vs. SGN-B7H4V | Control | | | oxygen species metabolic process | |
| mouse | B7H41001 mAb-DM1 | Up.in.Test/ | BP | GO:0050679 | positive regulation of epithelial | 4.93E−03 |
| | vs. SGN-B7H4V | Control | | | cell proliferation | |
| mouse | B7H41001 mAb-DM1 | Up.in.Test/ | BP | GO:0032944 | regulation of mononuclear cell | 4.95E−03 |
| | vs. SGN-B7H4V | Control | | | proliferation | |
| mouse | B7H41001 mAb-DM1 | Up.in.Test/ | BP | GO:1901615 | organic hydroxy compound | 4.95E−03 |
| | vs. SGN-B7H4V | Control | | | metabolic process | |
| mouse | B7H41001 mAb-DM1 | Up.in.Test/ | BP | GO:0015849 | organic acid transport | 5.14E−03 |
| | vs. SGN-B7H4V | Control | | | | |
| mouse | B7H41001 mAb-DM1 | Up.in.Test/ | BP | GO:0070661 | leukocyte proliferation | 5.18E−03 |
| | vs. SGN-B7H4V | Control | | | | |
| mouse | B7H41001 mAb-DM1 | Up.in.Test/ | BP | GO:0016525 | negative regulation of | 5.18E−03 |
| | vs. SGN-B7H4V | Control | | | angiogenesis | |
| mouse | B7H41001 mAb-DM1 | Up.in.Test/ | BP | GO:0019318 | hexose metabolic process | 5.18E−03 |
| | vs. SGN-B7H4V | Control | | | | |
| mouse | B7H41001 mAb-DM1 | Up.in.Test/ | BP | GO:0070371 | ERK1 and ERK2 cascade | 5.18E−03 |
| | vs. SGN-B7H4V | Control | | | | |
| mouse | B7H41001 mAb-DM1 | Up.in.Test/ | BP | GO:0072001 | renal system development | 5.18E−03 |
| | vs. SGN-B7H4V | Control | | | | |
| mouse | B7H41001 mAb-DM1 | Up.in.Test/ | BP | GO:0051480 | regulation of cytosolic calcium ion | 5.18E−03 |
| | vs. SGN-B7H4V | Control | | | concentration | |
| mouse | B7H41001 mAb-DM1 | Up.in.Test/ | BP | GO:0098900 | regulation of action potential | 5.20E−03 |
| | vs. SGN-B7H4V | Control | | | | |
| mouse | B7H41001 mAb-DM1 | Up.in.Test/ | BP | GO:0010907 | positive regulation of glucose | 5.27E−03 |
| | vs. SGN-B7H4V | Control | | | metabolic process | |
| mouse | B7H41001 mAb-DM1 | Up.in.Test/ | BP | GO:0043410 | positive regulation of MAPK | 5.27E−03 |
| | vs. SGN-B7H4V | Control | | | cascade | |
| mouse | B7H41001 mAb-DM1 | Up.in.Test/ | BP | GO:0045638 | negative regulation of myeloid cell | 5.27E−03 |
| | vs. SGN-B7H4V | Control | | | differentiation | |
| mouse | B7H41001 mAb-DM1 | Up.in.Test/ | BP | GO:0070373 | negative regulation of ERK1 and | 5.27E−03 |
| | vs. SGN-B7H4V | Control | | | ERK2 cascade | |
| mouse | B7H41001 mAb-DM1 | Up.in.Test/ | BP | GO:0010634 | positive regulation of epithelial | 5.27E−03 |
| | vs. SGN-B7H4V | Control | | | cell migration | |
| mouse | B7H41001 mAb-DM1 | Up.in.Test/ | BP | GO:0010631 | epithelial cell migration | 5.27E−03 |
| | vs. SGN-B7H4V | Control | | | | |
| mouse | B7H41001 mAb-DM1 | Up.in.Test/ | BP | GO:2000181 | negative regulation of blood | 5.28E−03 |
| | vs. SGN-B7H4V | Control | | | vessel morphogenesis | |
| mouse | B7H41001 mAb-DM1 | Up.in.Test/ | BP | GO:0001658 | branching involved in ureteric bud | 5.28E−03 |
| | vs. SGN-B7H4V | Control | | | morphogenesis | |
| mouse | B7H41001 mAb-DM1 | Up.in.Test/ | BP | GO:0001755 | neural crest cell migration | 5.28E−03 |
| | vs. SGN-B7H4V | Control | | | | |
| mouse | B7H41001 mAb-DM1 | Up.in.Test/ | BP | GO:0090132 | epithelium migration | 5.28E−03 |
| | vs. SGN-B7H4V | Control | | | | |
| mouse | B7H41001 mAb-DM1 | Down.in.Test/ | BP | GO:0040013 | negative regulation of locomotion | 5.29E−03 |
| | vs. SGN-B7H4V | Control | | | | |
| mouse | B7H41001 mAb-DM1 | Down.in.Test/ | BP | GO:0071402 | cellular response to lipoprotein | 5.32E−03 |
| | vs. SGN-B7H4V | Control | | | particle stimulus | |
| mouse | B7H41001 mAb-DM1 | Down.in.Test/ | BP | GO:0000045 | autophagosome assembly | 5.35E−03 |
| | vs. SGN-B7H4V | Control | | | | |
| mouse | B7H41001 mAb-DM1 | Down.in.Test/ | BP | GO:0042158 | lipoprotein biosynthetic process | 5.35E−03 |
| | vs. SGN-B7H4V | Control | | | | |
| mouse | B7H41001 mAb-DM1 | Up.in.Test/ | BP | GO:0060343 | trabecula formation | 5.37E−03 |
| | vs. SGN-B7H4V | Control | | | | |
| mouse | B7H41001 mAb-DM1 | Up.in.Test/ | BP | GO:0090130 | tissue migration | 5.38E−03 |
| | vs. SGN-B7H4V | Control | | | | |
| mouse | B7H41001 mAb-DM1 | Down.in.Test/ | BP | GO:0045639 | positive regulation of myeloid cell | 5.40E−03 |
| | vs. SGN-B7H4V | Control | | | differentiation | |
| mouse | B7H41001 mAb-DM1 | Up.in.Test/ | BP | GO:1901343 | negative regulation of vasculature | 5.46E−03 |
| | vs. SGN-B7H4V | Control | | | development | |
| mouse | B7H41001 mAb-DM1 | Up.in.Test/ | BP | GO:0001659 | temperature homeostasis | 5.75E−03 |
| | vs. SGN-B7H4V | Control | | | | |
| mouse | B7H41001 mAb-DM1 | Down.in.Test/ | BP | GO:0050921 | positive regulation of chemotaxis | 5.84E−03 |
| | vs. SGN-B7H4V | Control | | | | |
| mouse | B7H41001 mAb-DM1 | Down.in.Test/ | BP | GO:0002687 | positive regulation of leukocyte | 6.22E−03 |
| | vs. SGN-B7H4V | Control | | | migration | |
| mouse | B7H41001 mAb-DM1 | Up.in.Test/ | BP | GO:0043542 | endothelial cell migration | 6.22E−03 |
| | vs. SGN-B7H4V | Control | | | | |
| mouse | B7H41001 mAb-DM1 | Down.in.Test/ | BP | GO:0035710 | CD4-positive, alpha-beta T cell | 6.26E−03 |
| | vs. SGN-B7H4V | Control | | | activation | |
| mouse | B7H41001 mAb-DM1 | Up.in.Test/ | BP | GO:0002685 | regulation of leukocyte migration | 6.36E−03 |
| | vs. SGN-B7H4V | Control | | | | |
| mouse | B7H41001 mAb-DM1 | Up.in.Test/ | BP | GO:0030324 | lung development | 6.36E−03 |
| | vs. SGN-B7H4V | Control | | | | |
| mouse | B7H41001 mAb-DM1 | Up.in.Test/ | BP | GO:0071900 | regulation of protein | 6.36E−03 |
| | vs. SGN-B7H4V | Control | | | serine/threonine kinase activity | |
| mouse | B7H41001 mAb-DM1 | Up.in.Test/ | BP | GO:0044262 | cellular carbohydrate metabolic | 6.36E−03 |
| | vs. SGN-B7H4V | Control | | | process | |
| mouse | B7H41001 mAb-DM1 | Up.in.Test/ | BP | GO:0016051 | carbohydrate biosynthetic process | 6.36E−03 |
| | vs. SGN-B7H4V | Control | | | | |
| mouse | B7H41001 mAb-DM1 | Up.in.Test/ | BP | GO:0060541 | respiratory system development | 6.36E−03 |
| | vs. SGN-B7H4V | Control | | | | |
| mouse | B7H41001 mAb-DM1 | Up.in.Test/ | BP | GO:0006639 | acylglycerol metabolic process | 6.39E−03 |
| | vs. SGN-B7H4V | Control | | | | |
| mouse | B7H41001 mAb-DM1 | Down.in.Test/ | BP | GO:0043122 | regulation of I-kappaB kinase/NF- | 6.40E−03 |
| | vs. SGN-B7H4V | Control | | | kappaB signaling | |
| mouse | B7H41001 mAb-DM1 | Up.in.Test/ | BP | GO:0030595 | leukocyte chemotaxis | 6.67E−03 |
| | vs. SGN-B7H4V | Control | | | | |
| mouse | B7H41001 mAb-DM1 | Down.in.Test/ | BP | GO:0006516 | glycoprotein catabolic process | 6.70E−03 |
| | vs. SGN-B7H4V | Control | | | | |
| mouse | B7H41001 mAb-DM1 | Down.in.Test/ | BP | GO:0050765 | negative regulation of | 6.70E−03 |
| | vs. SGN-B7H4V | Control | | | phagocytosis | |
| mouse | B7H41001 mAb-DM1 | Down.in.Test/ | BP | GO:0043406 | positive regulation of MAP kinase | 6.73E−03 |
| | vs. SGN-B7H4V | Control | | | activity | |
| mouse | B7H41001 mAb-DM1 | Down.in.Test/ | BP | GO:0043410 | positive regulation of MAPK | 6.73E−03 |
| | vs. SGN-B7H4V | Control | | | cascade | |
| mouse | B7H41001 mAb-DM1 | Down.in.Test/ | BP | GO:1901222 | regulation of NIK/NF-kappaB | 6.73E−03 |
| | vs. SGN-B7H4V | Control | | | signaling | |
| mouse | B7H41001 mAb-DM1 | Up.in.Test/ | BP | GO:0046883 | regulation of hormone secretion | 6.87E−03 |
| | vs. SGN-B7H4V | Control | | | | |
| mouse | B7H41001 mAb-DM1 | Up.in.Test/ | BP | GO:0030323 | respiratory tube development | 6.87E−03 |
| | vs. SGN-B7H4V | Control | | | | |
| mouse | B7H41001 mAb-DM1 | Down.in.Test/ | BP | GO:0010506 | regulation of autophagy | 6.89E−03 |
| | vs. SGN-B7H4V | Control | | | | |
| mouse | B7H41001 mAb-DM1 | Down.in.Test/ | BP | GO:0045806 | negative regulation of endocytosis | 6.89E−03 |
| | vs. SGN-B7H4V | Control | | | | |
| mouse | B7H41001 mAb-DM1 | Down.in.Test/ | BP | GO:0030433 | ubiquitin-dependent ERAD | 6.91E−03 |
| | vs. SGN-B7H4V | Control | | | pathway | |
| mouse | B7H41001 mAb-DM1 | Up.in.Test/ | BP | GO:0006638 | neutral lipid metabolic process | 6.97E−03 |
| | vs. SGN-B7H4V | Control | | | | |
| mouse | B7H41001 mAb-DM1 | Up.in.Test/ | BP | GO:0048469 | cell maturation | 6.97E−03 |
| | vs. SGN-B7H4V | Control | | | | |
| mouse | B7H41001 mAb-DM1 | Up.in.Test/ | BP | GO:0001818 | negative regulation of cytokine | 7.14E−03 |
| | vs. SGN-B7H4V | Control | | | production | |
| mouse | B7H41001 mAb-DM1 | Up.in.Test/ | BP | GO:0014812 | muscle cell migration | 7.14E−03 |
| | vs. SGN-B7H4V | Control | | | | |
| mouse | B7H41001 mAb-DM1 | Up.in.Test/ | BP | GO:1905954 | positive regulation of lipid | 7.14E−03 |
| | vs. SGN-B7H4V | Control | | | localization | |
| mouse | B7H41001 mAb-DM1 | Up.in.Test/ | BP | GO:0001822 | kidney development | 7.14E−03 |
| | vs. SGN-B7H4V | Control | | | | |
| mouse | B7H41001 mAb-DM1 | Up.in.Test/ | BP | GO:0006940 | regulation of smooth muscle | 7.14E−03 |
| | vs. SGN-B7H4V | Control | | | contraction | |
| mouse | B7H41001 mAb-DM1 | Up.in.Test/ | BP | GO:1903531 | negative regulation of secretion | 7.24E−03 |
| | vs. SGN-B7H4V | Control | | | by cell | |
| mouse | B7H41001 mAb-DM1 | Up.in.Test/ | BP | GO:0046460 | neutral lipid biosynthetic process | 7.36E−03 |
| | vs. SGN-B7H4V | Control | | | | |
| mouse | B7H41001 mAb-DM1 | Up.in.Test/ | BP | GO:0046463 | acylglycerol biosynthetic process | 7.36E−03 |
| | vs. SGN-B7H4V | Control | | | | |
| mouse | B7H41001 mAb-DM1 | Down.in.Test/ | BP | GO:0050731 | positive regulation of peptidyl- | 7.36E−03 |
| | vs. SGN-B7H4V | Control | | | tyrosine phosphorylation | |
| mouse | B7H41001 mAb-DM1 | Up.in.Test/ | BP | GO:0005996 | monosaccharide metabolic | 7.56E−03 |
| | vs. SGN-B7H4V | Control | | | process | |
| mouse | B7H41001 mAb-DM1 | Up.in.Test/ | BP | GO:0010038 | response to metal ion | 7.56E−03 |
| | vs. SGN-B7H4V | Control | | | | |
| mouse | B7H41001 mAb-DM1 | Up.in.Test/ | BP | GO:0001656 | metanephros development | 7.61E−03 |
| | vs. SGN-B7H4V | Control | | | | |
| mouse | B7H41001 mAb-DM1 | Down.in.Test/ | BP | GO:0002888 | positive regulation of myeloid | 7.98E−03 |
| | vs. SGN-B7H4V | Control | | | leukocyte mediated immunity | |
| mouse | B7H41001 mAb-DM1 | Down.in.Test/ | BP | GO:0030970 | retrograde protein transport, ER | 7.98E−03 |
| | vs. SGN-B7H4V | Control | | | to cytosol | |
| mouse | B7H41001 mAb-DM1 | Down.in.Test/ | BP | GO:0045730 | respiratory burst | 7.98E−03 |
| | vs. SGN-B7H4V | Control | | | | |
| mouse | B7H41001 mAb-DM1 | Down.in.Test/ | BP | GO:1903513 | endoplasmic reticulum to cytosol | 7.98E−03 |
| | vs. SGN-B7H4V | Control | | | transport | |
| mouse | B7H41001 mAb-DM1 | Down.in.Test/ | BP | GO:0031640 | killing of cells of other organism | 7.98E−03 |
| | vs. SGN-B7H4V | Control | | | | |
| mouse | B7H41001 mAb-DM1 | Down.in.Test/ | BP | GO:0051701 | biological process involved in | 7.99E−03 |
| | vs. SGN-B7H4V | Control | | | interaction with host | |
| mouse | B7H41001 mAb-DM1 | Up.in.Test/ | BP | GO:0001508 | action potential | 8.02E−03 |
| | vs. SGN-B7H4V | Control | | | | |
| mouse | B7H41001 mAb-DM1 | Down.in.Test/ | BP | GO:0018212 | peptidyl-tyrosine modification | 8.04E−03 |
| | vs. SGN-B7H4V | Control | | | | |
| mouse | B7H41001 mAb-DM1 | Up.in.Test/ | BP | GO:0060675 | ureteric bud morphogenesis | 8.09E−03 |
| | vs. SGN-B7H4V | Control | | | | |
| mouse | B7H41001 mAb-DM1 | Up.in.Test/ | BP | GO:0033674 | positive regulation of kinase | 8.09E−03 |
| | vs. SGN-B7H4V | Control | | | activity | |
| mouse | B7H41001 mAb-DM1 | Down.in.Test/ | BP | GO:0006911 | phagocytosis, engulfment | 8.10E−03 |
| | vs. SGN-B7H4V | Control | | | | |
| mouse | B7H41001 mAb-DM1 | Up.in.Test/ | BP | GO:0045785 | positive regulation of cell | 8.17E−03 |
| | vs. SGN-B7H4V | Control | | | adhesion | |
| mouse | B7H41001 mAb-DM1 | Up.in.Test/ | BP | GO:0002573 | myeloid leukocyte differentiation | 8.17E−03 |
| | vs. SGN-B7H4V | Control | | | | |
| mouse | B7H41001 mAb-DM1 | Up.in.Test/ | BP | GO:0010885 | regulation of cholesterol storage | 8.22E−03 |
| | vs. SGN-B7H4V | Control | | | | |
| mouse | B7H41001 mAb-DM1 | Down.in.Test/ | BP | GO:0046916 | cellular transition metal ion | 8.26E−03 |
| | vs. SGN-B7H4V | Control | | | homeostasis | |
| mouse | B7H41001 mAb-DM1 | Down.in.Test/ | BP | GO:2000106 | regulation of leukocyte apoptotic | 8.26E−03 |
| | vs. SGN-B7H4V | Control | | | process | |
| mouse | B7H41001 mAb-DM1 | Up.in.Test/ | BP | GO:0090287 | regulation of cellular response to | 8.30E−03 |
| | vs. SGN-B7H4V | Control | | | growth factor stimulus | |
| mouse | B7H41001 mAb-DM1 | Down.in.Test/ | BP | GO:0035966 | response to topologically | 8.42E−03 |
| | vs. SGN-B7H4V | Control | | | incorrect protein | |
| mouse | B7H41001 mAb-DM1 | Up.in.Test/ | BP | GO:0072171 | mesonephric tubule | 8.44E−03 |
| | vs. SGN-B7H4V | Control | | | morphogenesis | |
| mouse | B7H41001 mAb-DM1 | Up.in.Test/ | BP | GO:0001936 | regulation of endothelial cell | 8.44E−03 |
| | vs. SGN-B7H4V | Control | | | proliferation | |
| mouse | B7H41001 mAb-DM1 | Up.in.Test/ | BP | GO:0015833 | peptide transport | 8.44E−03 |
| | vs. SGN-B7H4V | Control | | | | |
| mouse | B7H41001 mAb-DM1 | Down.in.Test/ | BP | GO:0002444 | myeloid leukocyte mediated | 8.52E−03 |
| | vs. SGN-B7H4V | Control | | | immunity | |
| mouse | B7H41001 mAb-DM1 | Down.in.Test/ | BP | GO:0002695 | negative regulation of leukocyte | 8.80E−03 |
| | vs. SGN-B7H4V | Control | | | activation | |
| mouse | B7H41001 mAb-DM1 | Up.in.Test/ | BP | GO:0002690 | positive regulation of leukocyte | 8.83E−03 |
| | vs. SGN-B7H4V | Control | | | chemotaxis | |
| mouse | B7H41001 mAb-DM1 | Down.in.Test/ | BP | GO:0042100 | B cell proliferation | 8.85E−03 |
| | vs. SGN-B7H4V | Control | | | | |
| mouse | B7H41001 mAb-DM1 | Down.in.Test/ | BP | GO:2000377 | regulation of reactive oxygen | 8.94E−03 |
| | vs. SGN-B7H4V | Control | | | species metabolic process | |
| mouse | B7H41001 mAb-DM1 | Up.in.Test/ | BP | GO:0042129 | regulation of T cell proliferation | 9.10E−03 |
| | vs. SGN-B7H4V | Control | | | | |
| mouse | B7H41001 mAb-DM1 | Up.in.Test/ | BP | GO:0050670 | regulation of lymphocyte | 9.10E−03 |
| | vs. SGN-B7H4V | Control | | | proliferation | |
| mouse | B7H41001 mAb-DM1 | Up.in.Test/ | BP | GO:0002688 | regulation of leukocyte | 9.10E−03 |
| | vs. SGN-B7H4V | Control | | | chemotaxis | |
| mouse | B7H41001 mAb-DM1 | Up.in.Test/ | BP | GO:0002762 | negative regulation of myeloid | 9.10E−03 |
| | vs. SGN-B7H4V | Control | | | leukocyte differentiation | |
| mouse | B7H41001 mAb-DM1 | Up.in.Test/ | BP | GO:0050886 | endocrine process | 9.19E−03 |
| | vs. SGN-B7H4V | Control | | | | |
| mouse | B7H41001 mAb-DM1 | Up.in.Test/ | BP | GO:0071674 | mononuclear cell migration | 9.32E−03 |
| | vs. SGN-B7H4V | Control | | | | |
| mouse | B7H41001 mAb-DM1 | Up.in.Test/ | BP | GO:0051249 | regulation of lymphocyte | 9.32E−03 |
| | vs. SGN-B7H4V | Control | | | activation | |
| mouse | B7H41001 mAb-DM1 | Down.in.Test/ | BP | GO:0032816 | positive regulation of natural killer | 9.38E−03 |
| | vs. SGN-B7H4V | Control | | | cell activation | |
| mouse | B7H41001 mAb-DM1 | Down.in.Test/ | BP | GO:0001894 | tissue homeostasis | 9.40E−03 |
| | vs. SGN-B7H4V | Control | | | | |
| mouse | B7H41001 mAb-DM1 | Up.in.Test/ | BP | GO:0008015 | blood circulation | 9.42E−03 |
| | vs. SGN-B7H4V | Control | | | | |
| mouse | B7H41001 mAb-DM1 | Down.in.Test/ | BP | GO:1904894 | positive regulation of receptor | 9.43E−03 |
| | vs. SGN-B7H4V | Control | | | signaling pathway via STAT | |
| mouse | B7H41001 mAb-DM1 | Down.in.Test/ | BP | GO:0036503 | ERAD pathway | 9.45E−03 |
| | vs. SGN-B7H4V | Control | | | | |
| mouse | B7H41001 mAb-DM1 | Up.in.Test/ | BP | GO:0048568 | embryonic organ development | 9.51E−03 |
| | vs. SGN-B7H4V | Control | | | | |
| mouse | B7H41001 mAb-DM1 | Up.in.Test/ | BP | GO:0015918 | sterol transport | 9.51E−03 |
| | vs. SGN-B7H4V | Control | | | | |
| mouse | B7H41001 mAb-DM1 | Up.in.Test/ | BP | GO:0030301 | cholesterol transport | 9.51E−03 |
| | vs. SGN-B7H4V | Control | | | | |
| mouse | B7H41001 mAb-DM1 | Up.in.Test/ | BP | GO:0061005 | cell differentiation involved in | 9.62E−03 |
| | vs. SGN-B7H4V | Control | | | kidney development | |
| mouse | B7H41001 mAb-DM1 | Down.in.Test/ | BP | GO:0050866 | negative regulation of cell | 9.84E−03 |
| | vs. SGN-B7H4V | Control | | | activation | |
| mouse | B7H41001 mAb-DM1 | Down.in.Test/ | BP | GO:0002468 | dendritic cell antigen processing | 9.96E−03 |
| | vs. SGN-B7H4V | Control | | | and presentation | |
| mouse | B7H41001 mAb-DM1 | Down.in.Test/ | BP | GO:0002730 | regulation of dendritic cell | 9.96E−03 |
| | vs. SGN-B7H4V | Control | | | cytokine production | |
| mouse | B7H41001 mAb-DM1 | Down.in.Test/ | BP | GO:0060330 | regulation of response to | 9.96E−03 |
| | vs. SGN-B7H4V | Control | | | interferon-gamma | |
| mouse | B7H41001 mAb-DM1 | Down.in.Test/ | BP | GO:0060334 | regulation of interferon-gamma- | 9.96E−03 |
| | vs. SGN-B7H4V | Control | | | mediated signaling pathway | |

TABLE 22
Gene Ontology (GO) analysis comparing gene expression changes following treatment with B7H41001 mAb-DM4 versus SGN-B7H4V

| | | | | | | Adjusted |
|---|---|---|---|---|---|---|
| Species | Comparison | Direction | ONTOLOGY | ID | Description | p-value |
| human | B7H41001 mAb-DM4 | Down.in.Test/ | BP | GO:0051607 | defense response to virus | 2.35E−17 |
| | vs. SGN-B7H4V | Control | | | | |
| human | B7H41001 mAb-DM4 | Down.in.Test/ | BP | GO:0140546 | defense response to symbiont | 2.35E−17 |
| | vs. SGN-B7H4V | Control | | | | |
| human | B7H41001 mAb-DM4 | Down.in.Test/ | BP | GO:0009615 | response to virus | 9.71E−16 |
| | vs. SGN-B7H4V | Control | | | | |
| human | B7H41001 mAb-DM4 | Down.in.Test/ | BP | GO:0050792 | regulation of viral process | 6.05E−14 |
| | vs. SGN-B7H4V | Control | | | | |
| human | B7H41001 mAb-DM4 | Down.in.Test/ | BP | GO:1903900 | regulation of viral life cycle | 8.83E−14 |
| | vs. SGN-B7H4V | Control | | | | |
| human | B7H41001 mAb-DM4 | Up.in.Test/ | BP | GO:0007015 | actin filament organization | 9.90E−14 |
| | vs. SGN-B7H4V | Control | | | | |
| human | B7H41001 mAb-DM4 | Down.in.Test/ | BP | GO:0048525 | negative regulation of viral process | 2.18E−13 |
| | vs. SGN-B7H4V | Control | | | | |
| human | B7H41001 mAb-DM4 | Down.in.Test/ | BP | GO:0045071 | negative regulation of viral genome | 4.12E−11 |
| | vs. SGN-B7H4V | Control | | | replication | |
| human | B7H41001 mAb-DM4 | Down.in.Test/ | BP | GO:0002831 | regulation of response to biotic stimulus | 3.72E−10 |
| | vs. SGN-B7H4V | Control | | | | |
| human | B7H41001 mAb-DM4 | Down.in.Test/ | BP | GO:0045824 | negative regulation of innate immune | 6.29E−10 |
| | vs. SGN-B7H4V | Control | | | response | |
| human | B7H41001 mAb-DM4 | Down.in.Test/ | BP | GO:0019885 | antigen processing and presentation of | 9.69E−10 |
| | vs. SGN-B7H4V | Control | | | endogenous peptide antigen via MHC class I | |
| human | B7H41001 mAb-DM4 | Down.in.Test/ | BP | GO:0019079 | viral genome replication | 1.25E−09 |
| | vs. SGN-B7H4V | Control | | | | |
| human | B7H41001 mAb-DM4 | Down.in.Test/ | BP | GO:0034340 | response to type I interferon | 2.01E−09 |
| | vs. SGN-B7H4V | Control | | | | |
| human | B7H41001 mAb-DM4 | Down.in.Test/ | BP | GO:0045069 | regulation of viral genome replication | 2.01E−09 |
| | vs. SGN-B7H4V | Control | | | | |
| human | B7H41001 mAb-DM4 | Up.in.Test/ | BP | GO:0022604 | regulation of cell morphogenesis | 2.13E−09 |
| | vs. SGN-B7H4V | Control | | | | |
| human | B7H41001 mAb-DM4 | Down.in.Test/ | BP | GO:0002832 | negative regulation of response to biotic | 2.45E−09 |
| | vs. SGN-B7H4V | Control | | | stimulus | |
| human | B7H41001 mAb-DM4 | Up.in.Test/ | BP | GO:0045785 | positive regulation of cell adhesion | 2.47E−09 |
| | vs. SGN-B7H4V | Control | | | | |
| human | B7H41001 mAb-DM4 | Up.in.Test/ | BP | GO:0030335 | positive regulation of cell migration | 3.10E−09 |
| | vs. SGN-B7H4V | Control | | | | |
| human | B7H41001 mAb-DM4 | Down.in.Test/ | BP | GO:0071357 | cellular response to type I interferon | 3.29E−09 |
| | vs. SGN-B7H4V | Control | | | | |
| human | B7H41001 mAb-DM4 | Down.in.Test/ | BP | GO:0002474 | antigen processing and presentation of | 3.30E−09 |
| | vs. SGN-B7H4V | Control | | | peptide antigen via MHC class I | |
| human | B7H41001 mAb-DM4 | Down.in.Test/ | BP | GO:0002483 | antigen processing and presentation of | 3.85E−09 |
| | vs. SGN-B7H4V | Control | | | endogenous peptide antigen | |
| human | B7H41001 mAb-DM4 | Up.in.Test/ | BP | GO:2000147 | positive regulation of cell motility | 7.98E−09 |
| | vs. SGN-B7H4V | Control | | | | |
| human | B7H41001 mAb-DM4 | Down.in.Test/ | BP | GO:0060337 | type I interferon signaling pathway | 1.59E−08 |
| | vs. SGN-B7H4V | Control | | | | |
| human | B7H41001 mAb-DM4 | Down.in.Test/ | BP | GO:0045088 | regulation of innate immune response | 5.72E−08 |
| | vs. SGN-B7H4V | Control | | | | |
| human | B7H41001 mAb-DM4 | Down.in.Test/ | BP | GO:0019883 | antigen processing and presentation of | 6.22E−08 |
| | vs. SGN-B7H4V | Control | | | endogenous antigen | |
| human | B7H41001 mAb-DM4 | Down.in.Test/ | BP | GO:0098586 | cellular response to virus | 2.87E−07 |
| | vs. SGN-B7H4V | Control | | | | |
| human | B7H41001 mAb-DM4 | Down.in.Test/ | BP | GO:0016032 | viral process | 3.28E−07 |
| | vs. SGN-B7H4V | Control | | | | |
| human | B7H41001 mAb-DM4 | Down.in.Test/ | BP | GO:0019058 | viral life cycle | 4.59E−07 |
| | vs. SGN-B7H4V | Control | | | | |
| human | B7H41001 mAb-DM4 | Up.in.Test/ | BP | GO:0042110 | T cell activation | 5.04E−07 |
| | vs. SGN-B7H4V | Control | | | | |
| human | B7H41001 mAb-DM4 | Up.in.Test/ | BP | GO:0008360 | regulation of cell shape | 5.04E−07 |
| | vs. SGN-B7H4V | Control | | | | |
| human | B7H41001 mAb-DM4 | Up.in.Test/ | BP | GO:0050851 | antigen receptor-mediated signaling pathway | 5.04E−07 |
| | vs. SGN-B7H4V | Control | | | | |
| human | B7H41001 mAb-DM4 | Up.in.Test/ | BP | GO:0051493 | regulation of cytoskeleton organization | 5.08E−07 |
| | vs. SGN-B7H4V | Control | | | | |
| human | B7H41001 mAb-DM4 | Up.in.Test/ | BP | GO:0022407 | regulation of cell-cell adhesion | 5.13E−07 |
| | vs. SGN-B7H4V | Control | | | | |
| human | B7H41001 mAb-DM4 | Up.in.Test/ | BP | GO:0050867 | positive regulation of cell activation | 5.59E−07 |
| | vs. SGN-B7H4V | Control | | | | |
| human | B7H41001 mAb-DM4 | Up.in.Test/ | BP | GO:0002694 | regulation of leukocyte activation | 6.38E−07 |
| | vs. SGN-B7H4V | Control | | | | |
| human | B7H41001 mAb-DM4 | Up.in.Test/ | BP | GO:0022409 | positive regulation of cell-cell adhesion | 8.01E−07 |
| | vs. SGN-B7H4V | Control | | | | |
| human | B7H41001 mAb-DM4 | Up.in.Test/ | BP | GO:0032970 | regulation of actin filament-based process | 1.08E−06 |
| | vs. SGN-B7H4V | Control | | | | |
| human | B7H41001 mAb-DM4 | Up.in.Test/ | BP | GO:0002696 | positive regulation of leukocyte activation | 1.34E−06 |
| | vs. SGN-B7H4V | Control | | | | |
| human | B7H41001 mAb-DM4 | Up.in.Test/ | BP | GO:0032956 | regulation of actin cytoskeleton organization | 1.39E−06 |
| | vs. SGN-B7H4V | Control | | | | |
| human | B7H41001 mAb-DM4 | Up.in.Test/ | BP | GO:0050852 | T cell receptor signaling pathway | 1.65E−06 |
| | vs. SGN-B7H4V | Control | | | | |
| human | B7H41001 mAb-DM4 | Up.in.Test/ | BP | GO:0007159 | leukocyte cell-cell adhesion | 1.65E−06 |
| | vs. SGN-B7H4V | Control | | | | |
| human | B7H41001 mAb-DM4 | Up.in.Test/ | BP | GO:1902532 | negative regulation of intracellular signal | 1.89E−06 |
| | vs. SGN-B7H4V | Control | | | transduction | |
| human | B7H41001 mAb-DM4 | Up.in.Test/ | BP | GO:0051249 | regulation of lymphocyte activation | 2.14E−06 |
| | vs. SGN-B7H4V | Control | | | | |
| human | B7H41001 mAb-DM4 | Up.in.Test/ | BP | GO:0002429 | immune response-activating cell surface | 2.89E−06 |
| | vs. SGN-B7H4V | Control | | | receptor signaling pathway | |
| human | B7H41001 mAb-DM4 | Up.in.Test/ | BP | GO:0002757 | immune response-activating signal | 2.89E−06 |
| | vs. SGN-B7H4V | Control | | | transduction | |
| human | B7H41001 mAb-DM4 | Up.in.Test/ | BP | GO:0001667 | ameboidal-type cell migration | 2.89E−06 |
| | vs. SGN-B7H4V | Control | | | | |
| human | B7H41001 mAb-DM4 | Up.in.Test/ | BP | GO:0042060 | wound healing | 5.03E−06 |
| | vs. SGN-B7H4V | Control | | | | |
| human | B7H41001 mAb-DM4 | Up.in.Test/ | BP | GO:0007264 | small GTPase mediated signal transduction | 7.19E−06 |
| | vs. SGN-B7H4V | Control | | | | |
| human | B7H41001 mAb-DM4 | Up.in.Test/ | BP | GO:1903039 | positive regulation of leukocyte cell-cell | 7.24E−06 |
| | vs. SGN-B7H4V | Control | | | adhesion | |
| human | B7H41001 mAb-DM4 | Down.in.Test/ | BP | GO:0035455 | response to interferon-alpha | 7.32E−06 |
| | vs. SGN-B7H4V | Control | | | | |
| human | B7H41001 mAb-DM4 | Up.in.Test/ | BP | GO:0050854 | regulation of antigen receptor-mediated | 1.06E−05 |
| | vs. SGN-B7H4V | Control | | | signaling pathway | |
| human | B7H41001 mAb-DM4 | Up.in.Test/ | BP | GO:0002768 | immune response-regulating cell surface | 1.06E−05 |
| | vs. SGN-B7H4V | Control | | | receptor signaling pathway | |
| human | B7H41001 mAb-DM4 | Up.in.Test/ | BP | GO:0018108 | peptidyl-tyrosine phosphorylation | 1.06E−05 |
| | vs. SGN-B7H4V | Control | | | | |
| human | B7H41001 mAb-DM4 | Up.in.Test/ | BP | GO:0018212 | peptidyl-tyrosine modification | 1.35E−05 |
| | vs. SGN-B7H4V | Control | | | | |
| human | B7H41001 mAb-DM4 | Up.in.Test/ | BP | GO:0031589 | cell-substrate adhesion | 1.36E−05 |
| | vs. SGN-B7H4V | Control | | | | |
| human | B7H41001 mAb-DM4 | Up.in.Test/ | BP | GO:0043254 | regulation of protein-containing complex | 1.36E−05 |
| | vs. SGN-B7H4V | Control | | | assembly | |
| human | B7H41001 mAb-DM4 | Up.in.Test/ | BP | GO:0009611 | response to wounding | 1.44E−05 |
| | vs. SGN-B7H4V | Control | | | | |
| human | B7H41001 mAb-DM4 | Up.in.Test/ | BP | GO:1903829 | positive regulation of cellular protein | 1.45E−05 |
| | vs. SGN-B7H4V | Control | | | localization | |
| human | B7H41001 mAb-DM4 | Up.in.Test/ | BP | GO:0051251 | positive regulation of lymphocyte activation | 1.72E−05 |
| | vs. SGN-B7H4V | Control | | | | |
| human | B7H41001 mAb-DM4 | Up.in.Test/ | BP | GO:0050900 | leukocyte migration | 2.07E−05 |
| | vs. SGN-B7H4V | Control | | | | |
| human | B7H41001 mAb-DM4 | Up.in.Test/ | BP | GO:0045936 | negative regulation of phosphate metabolic | 2.29E−05 |
| | vs. SGN-B7H4V | Control | | | process | |
| human | B7H41001 mAb-DM4 | Up.in.Test/ | BP | GO:0010563 | negative regulation of phosphorus metabolic | 2.41E−05 |
| | vs. SGN-B7H4V | Control | | | process | |
| human | B7H41001 mAb-DM4 | Up.in.Test/ | BP | GO:0018105 | peptidyl-serine phosphorylation | 2.48E−05 |
| | vs. SGN-B7H4V | Control | | | | |
| human | B7H41001 mAb-DM4 | Up.in.Test/ | BP | GO:1903827 | regulation of cellular protein localization | 2.70E−05 |
| | vs. SGN-B7H4V | Control | | | | |
| human | B7H41001 mAb-DM4 | Up.in.Test/ | BP | GO:0002764 | immune response-regulating signaling | 3.04E−05 |
| | vs. SGN-B7H4V | Control | | | pathway | |
| human | B7H41001 mAb-DM4 | Up.in.Test/ | BP | GO:1903037 | regulation of leukocyte cell-cell adhesion | 3.24E−05 |
| | vs. SGN-B7H4V | Control | | | | |
| human | B7H41001 mAb-DM4 | Up.in.Test/ | BP | GO:0050863 | regulation of T cell activation | 3.41E−05 |
| | vs. SGN-B7H4V | Control | | | | |
| human | B7H41001 mAb-DM4 | Up.in.Test/ | BP | GO:0050870 | positive regulation of T cell activation | 3.41E−05 |
| | vs. SGN-B7H4V | Control | | | | |
| human | B7H41001 mAb-DM4 | Up.in.Test/ | BP | GO:0043068 | positive regulation of programmed cell death | 3.53E−05 |
| | vs. SGN-B7H4V | Control | | | | |
| human | B7H41001 mAb-DM4 | Up.in.Test/ | BP | GO:0051017 | actin filament bundle assembly | 3.66E−05 |
| | vs. SGN-B7H4V | Control | | | | |
| human | B7H41001 mAb-DM4 | Up.in.Test/ | BP | GO:0061572 | actin filament bundle organization | 3.66E−05 |
| | vs. SGN-B7H4V | Control | | | | |
| human | B7H41001 mAb-DM4 | Up.in.Test/ | BP | GO:0018209 | peptidyl-serine modification | 3.96E−05 |
| | vs. SGN-B7H4V | Control | | | | |
| human | B7H41001 mAb-DM4 | Up.in.Test/ | BP | GO:1902107 | positive regulation of leukocyte differentiation | 3.96E−05 |
| | vs. SGN-B7H4V | Control | | | | |
| human | B7H41001 mAb-DM4 | Up.in.Test/ | BP | GO:1903708 | positive regulation of hemopoiesis | 3.96E−05 |
| | vs. SGN-B7H4V | Control | | | | |
| human | B7H41001 mAb-DM4 | Up.in.Test/ | BP | GO:0046651 | lymphocyte proliferation | 5.67E−05 |
| | vs. SGN-B7H4V | Control | | | | |
| human | B7H41001 mAb-DM4 | Up.in.Test/ | BP | GO:0043065 | positive regulation of apoptotic process | 6.11E−05 |
| | vs. SGN-B7H4V | Control | | | | |
| human | B7H41001 mAb-DM4 | Up.in.Test/ | BP | GO:1902903 | regulation of supramolecular fiber | 6.17E−05 |
| | vs. SGN-B7H4V | Control | | | organization | |
| human | B7H41001 mAb-DM4 | Up.in.Test/ | BP | GO:0030050 | vesicle transport along actin filament | 6.26E−05 |
| | vs. SGN-B7H4V | Control | | | | |
| human | B7H41001 mAb-DM4 | Up.in.Test/ | BP | GO:0032943 | mononuclear cell proliferation | 6.40E−05 |
| | vs. SGN-B7H4V | Control | | | | |
| human | B7H41001 mAb-DM4 | Up.in.Test/ | BP | GO:1902105 | regulation of leukocyte differentiation | 6.93E−05 |
| | vs. SGN-B7H4V | Control | | | | |
| human | B7H41001 mAb-DM4 | Up.in.Test/ | BP | GO:0050778 | positive regulation of immune response | 7.05E−05 |
| | vs. SGN-B7H4V | Control | | | | |
| human | B7H41001 mAb-DM4 | Up.in.Test/ | BP | GO:0070661 | leukocyte proliferation | 7.45E−05 |
| | vs. SGN-B7H4V | Control | | | | |
| human | B7H41001 mAb-DM4 | Down.in.Test/ | BP | GO:0060339 | negative regulation of type I interferon- | 7.70E−05 |
| | vs. SGN-B7H4V | Control | | | mediated signaling pathway | |
| human | B7H41001 mAb-DM4 | Down.in.Test/ | BP | GO:0048002 | antigen processing and presentation of | 7.79E−05 |
| | vs. SGN-B7H4V | Control | | | peptide antigen | |
| human | B7H41001 mAb-DM4 | Up.in.Test/ | BP | GO:0043087 | regulation of GTPase activity | 9.56E−05 |
| | vs. SGN-B7H4V | Control | | | | |
| human | B7H41001 mAb-DM4 | Up.in.Test/ | BP | GO:0031032 | actomyosin structure organization | 1.02E−04 |
| | vs. SGN-B7H4V | Control | | | | |
| human | B7H41001 mAb-DM4 | Up.in.Test/ | BP | GO:0008154 | actin polymerization or depolymerization | 1.03E−04 |
| | vs. SGN-B7H4V | Control | | | | |
| human | B7H41001 mAb-DM4 | Up.in.Test/ | BP | GO:0030099 | myeloid cell differentiation | 1.29E−04 |
| | vs. SGN-B7H4V | Control | | | | |
| human | B7H41001 mAb-DM4 | Up.in.Test/ | BP | GO:0050730 | regulation of peptidyl-tyrosine | 1.33E−04 |
| | vs. SGN-B7H4V | Control | | | phosphorylation | |
| human | B7H41001 mAb-DM4 | Up.in.Test/ | BP | GO:0099515 | actin filament-based transport | 1.50E−04 |
| | vs. SGN-B7H4V | Control | | | | |
| human | B7H41001 mAb-DM4 | Up.in.Test/ | BP | GO:0090130 | tissue migration | 1.58E−04 |
| | vs. SGN-B7H4V | Control | | | | |
| human | B7H41001 mAb-DM4 | Down.in.Test/ | BP | GO:0060338 | regulation of type I interferon-mediated | 1.68E−04 |
| | vs. SGN-B7H4V | Control | | | signaling pathway | |
| human | B7H41001 mAb-DM4 | Down.in.Test/ | BP | GO:0050777 | negative regulation of immune response | 1.68E−04 |
| | vs. SGN-B7H4V | Control | | | | |
| human | B7H41001 mAb-DM4 | Down.in.Test/ | BP | GO:0035456 | response to interferon-beta | 1.84E−04 |
| | vs. SGN-B7H4V | Control | | | | |
| human | B7H41001 mAb-DM4 | Up.in.Test/ | BP | GO:0030865 | cortical cytoskeleton organization | 1.93E−04 |
| | vs. SGN-B7H4V | Control | | | | |
| human | B7H41001 mAb-DM4 | Up.in.Test/ | BP | GO:0010631 | epithelial cell migration | 1.93E−04 |
| | vs. SGN-B7H4V | Control | | | | |
| human | B7H41001 mAb-DM4 | Up.in.Test/ | BP | GO:0042098 | T cell proliferation | 1.94E−04 |
| | vs. SGN-B7H4V | Control | | | | |
| human | B7H41001 mAb-DM4 | Up.in.Test/ | BP | GO:1903706 | regulation of hemopoiesis | 2.19E−04 |
| | vs. SGN-B7H4V | Control | | | | |
| human | B7H41001 mAb-DM4 | Up.in.Test/ | BP | GO:0031331 | positive regulation of cellular catabolic | 2.23E−04 |
| | vs. SGN-B7H4V | Control | | | process | |
| human | B7H41001 mAb-DM4 | Up.in.Test/ | BP | GO:0001819 | positive regulation of cytokine production | 2.23E−04 |
| | vs. SGN-B7H4V | Control | | | | |
| human | B7H41001 mAb-DM4 | Up.in.Test/ | BP | GO:0042326 | negative regulation of phosphorylation | 2.23E−04 |
| | vs. SGN-B7H4V | Control | | | | |
| human | B7H41001 mAb-DM4 | Up.in.Test/ | BP | GO:0050856 | regulation of T cell receptor signaling pathway | 2.23E−04 |
| | vs. SGN-B7H4V | Control | | | | |
| human | B7H41001 mAb-DM4 | Up.in.Test/ | BP | GO:0050671 | positive regulation of lymphocyte | 2.23E−04 |
| | vs. SGN-B7H4V | Control | | | proliferation | |
| human | B7H41001 mAb-DM4 | Up.in.Test/ | BP | GO:0090132 | epithelium migration | 2.25E−04 |
| | vs. SGN-B7H4V | Control | | | | |
| human | B7H41001 mAb-DM4 | Up.in.Test/ | BP | GO:0099518 | vesicle cytoskeletal trafficking | 2.28E−04 |
| | vs. SGN-B7H4V | Control | | | | |
| human | B7H41001 mAb-DM4 | Up.in.Test/ | BP | GO:0070665 | positive regulation of leukocyte proliferation | 2.36E−04 |
| | vs. SGN-B7H4V | Control | | | | |
| human | B7H41001 mAb-DM4 | Up.in.Test/ | BP | GO:0045860 | positive regulation of protein kinase activity | 2.36E−04 |
| | vs. SGN-B7H4V | Control | | | | |
| human | B7H41001 mAb-DM4 | Up.in.Test/ | BP | GO:0043122 | regulation of I-kappaB kinase/NF-kappaB | 2.36E−04 |
| | vs. SGN-B7H4V | Control | | | signaling | |
| human | B7H41001 mAb-DM4 | Up.in.Test/ | BP | GO:0032946 | positive regulation of mononuclear cell | 2.36E−04 |
| | vs. SGN-B7H4V | Control | | | proliferation | |
| human | B7H41001 mAb-DM4 | Up.in.Test/ | BP | GO:0002237 | response to molecule of bacterial origin | 2.37E−04 |
| | vs. SGN-B7H4V | Control | | | | |
| human | B7H41001 mAb-DM4 | Up.in.Test/ | BP | GO:0050670 | regulation of lymphocyte proliferation | 2.49E−04 |
| | vs. SGN-B7H4V | Control | | | | |
| human | B7H41001 mAb-DM4 | Up.in.Test/ | BP | GO:0110053 | regulation of actin filament organization | 2.62E−04 |
| | vs. SGN-B7H4V | Control | | | | |
| human | B7H41001 mAb-DM4 | Up.in.Test/ | BP | GO:0032944 | regulation of mononuclear cell proliferation | 2.69E−04 |
| | vs. SGN-B7H4V | Control | | | | |
| human | B7H41001 mAb-DM4 | Down.in.Test/ | BP | GO:0032728 | positive regulation of interferon-beta | 2.72E−04 |
| | vs. SGN-B7H4V | Control | | | production | |
| human | B7H41001 mAb-DM4 | Up.in.Test/ | BP | GO:0050857 | positive regulation of antigen receptor- | 2.81E−04 |
| | vs. SGN-B7H4V | Control | | | mediated signaling pathway | |
| human | B7H41001 mAb-DM4 | Up.in.Test/ | BP | GO:0001933 | negative regulation of protein | 2.83E−04 |
| | vs. SGN-B7H4V | Control | | | phosphorylation | |
| human | B7H41001 mAb-DM4 | Up.in.Test/ | BP | GO:0070663 | regulation of leukocyte proliferation | 3.66E−04 |
| | vs. SGN-B7H4V | Control | | | | |
| human | B7H41001 mAb-DM4 | Up.in.Test/ | BP | GO:0071706 | tumor necrosis factor superfamily cytokine | 3.66E−04 |
| | vs. SGN-B7H4V | Control | | | production | |
| human | B7H41001 mAb-DM4 | Up.in.Test/ | BP | GO:1903555 | regulation of tumor necrosis factor | 3.66E−04 |
| | vs. SGN-B7H4V | Control | | | superfamily cytokine production | |
| human | B7H41001 mAb-DM4 | Up.in.Test/ | BP | GO:0032496 | response to lipopolysaccharide | 3.72E−04 |
| | vs. SGN-B7H4V | Control | | | | |
| human | B7H41001 mAb-DM4 | Up.in.Test/ | BP | GO:0009896 | positive regulation of catabolic process | 4.35E−04 |
| | vs. SGN-B7H4V | Control | | | | |
| human | B7H41001 mAb-DM4 | Down.in.Test/ | BP | GO:0046596 | regulation of viral entry into host cell | 4.45E−04 |
| | vs. SGN-B7H4V | Control | | | | |
| human | B7H41001 mAb-DM4 | Up.in.Test/ | BP | GO:0032535 | regulation of cellular component size | 4.66E−04 |
| | vs. SGN-B7H4V | Control | | | | |
| human | B7H41001 mAb-DM4 | Up.in.Test/ | BP | GO:0071216 | cellular response to biotic stimulus | 4.84E−04 |
| | vs. SGN-B7H4V | Control | | | | |
| human | B7H41001 mAb-DM4 | Up.in.Test/ | BP | GO:0002573 | myeloid leukocyte differentiation | 5.06E−04 |
| | vs. SGN-B7H4V | Control | | | | |
| human | B7H41001 mAb-DM4 | Up.in.Test/ | BP | GO:0042176 | regulation of protein catabolic process | 5.06E−04 |
| | vs. SGN-B7H4V | Control | | | | |
| human | B7H41001 mAb-DM4 | Down.in.Test/ | BP | GO:0031348 | negative regulation of defense response | 5.31E−04 |
| | vs. SGN-B7H4V | Control | | | | |
| human | B7H41001 mAb-DM4 | Down.in.Test/ | BP | GO:0045953 | negative regulation of natural killer cell | 5.97E−04 |
| | vs. SGN-B7H4V | Control | | | mediated cytotoxicity | |
| human | B7H41001 mAb-DM4 | Up.in.Test/ | BP | GO:0007163 | establishment or maintenance of cell polarity | 6.43E−04 |
| | vs. SGN-B7H4V | Control | | | | |
| human | B7H41001 mAb-DM4 | Up.in.Test/ | BP | GO:0033674 | positive regulation of kinase activity | 6.43E−04 |
| | vs. SGN-B7H4V | Control | | | | |
| human | B7H41001 mAb-DM4 | Up.in.Test/ | BP | GO:0002253 | activation of immune response | 6.43E−04 |
| | vs. SGN-B7H4V | Control | | | | |
| human | B7H41001 mAb-DM4 | Up.in.Test/ | BP | GO:0051640 | organelle localization | 6.58E−04 |
| | vs. SGN-B7H4V | Control | | | | |
| human | B7H41001 mAb-DM4 | Up.in.Test/ | BP | GO:0042102 | positive regulation of T cell proliferation | 6.59E−04 |
| | vs. SGN-B7H4V | Control | | | | |
| human | B7H41001 mAb-DM4 | Up.in.Test/ | BP | GO:0002683 | negative regulation of immune system | 6.94E−04 |
| | vs. SGN-B7H4V | Control | | | process | |
| human | B7H41001 mAb-DM4 | Down.in.Test/ | BP | GO:0002683 | negative regulation of immune system | 7.40E−04 |
| | vs. SGN-B7H4V | Control | | | process | |
| human | B7H41001 mAb-DM4 | Up.in.Test/ | BP | GO:1901653 | cellular response to peptide | 7.48E−04 |
| | vs. SGN-B7H4V | Control | | | | |
| human | B7H41001 mAb-DM4 | Up.in.Test/ | BP | GO:0007162 | negative regulation of cell adhesion | 8.43E−04 |
| | vs. SGN-B7H4V | Control | | | | |
| human | B7H41001 mAb-DM4 | Down.in.Test/ | BP | GO:0002716 | negative regulation of natural killer cell | 8.60E−04 |
| | vs. SGN-B7H4V | Control | | | mediated immunity | |
| human | B7H41001 mAb-DM4 | Up.in.Test/ | BP | GO:0007249 | I-kappaB kinase/NF-kappaB signaling | 9.05E−04 |
| | vs. SGN-B7H4V | Control | | | | |
| human | B7H41001 mAb-DM4 | Up.in.Test/ | BP | GO:0007265 | Ras protein signal transduction | 9.22E−04 |
| | vs. SGN-B7H4V | Control | | | | |
| human | B7H41001 mAb-DM4 | Up.in.Test/ | BP | GO:0071219 | cellular response to molecule of bacterial | 9.22E−04 |
| | vs. SGN-B7H4V | Control | | | origin | |
| human | B7H41001 mAb-DM4 | Up.in.Test/ | BP | GO:0034109 | homotypic cell-cell adhesion | 9.35E−04 |
| | vs. SGN-B7H4V | Control | | | | |
| human | B7H41001 mAb-DM4 | Up.in.Test/ | BP | GO:0071375 | cellular response to peptide hormone | 1.01E−03 |
| | vs. SGN-B7H4V | Control | | | stimulus | |
| human | B7H41001 mAb-DM4 | Up.in.Test/ | BP | GO:2001233 | regulation of apoptotic signaling pathway | 1.01E−03 |
| | vs. SGN-B7H4V | Control | | | | |
| human | B7H41001 mAb-DM4 | Up.in.Test/ | BP | GO:0071674 | mononuclear cell migration | 1.01E−03 |
| | vs. SGN-B7H4V | Control | | | | |
| human | B7H41001 mAb-DM4 | Up.in.Test/ | BP | GO:0031098 | stress-activated protein kinase signaling | 1.01E−03 |
| | vs. SGN-B7H4V | Control | | | cascade | |
| human | B7H41001 mAb-DM4 | Up.in.Test/ | BP | GO:1905475 | regulation of protein localization to | 1.01E−03 |
| | vs. SGN-B7H4V | Control | | | membrane | |
| human | B7H41001 mAb-DM4 | Up.in.Test/ | BP | GO:0010810 | regulation of cell-substrate adhesion | 1.01E−03 |
| | vs. SGN-B7H4V | Control | | | | |
| human | B7H41001 mAb-DM4 | Up.in.Test/ | BP | GO:1901652 | response to peptide | 1.03E−03 |
| | vs. SGN-B7H4V | Control | | | | |
| human | B7H41001 mAb-DM4 | Up.in.Test/ | BP | GO:0071222 | cellular response to lipopolysaccharide | 1.06E−03 |
| | vs. SGN-B7H4V | Control | | | | |
| human | B7H41001 mAb-DM4 | Down.in.Test/ | BP | GO:0032608 | interferon-beta production | 1.11E−03 |
| | vs. SGN-B7H4V | Control | | | | |
| human | B7H41001 mAb-DM4 | Down.in.Test/ | BP | GO:0032648 | regulation of interferon-beta production | 1.11E−03 |
| | vs. SGN-B7H4V | Control | | | | |
| human | B7H41001 mAb-DM4 | Up.in.Test/ | BP | GO:0051258 | protein polymerization | 1.12E−03 |
| | vs. SGN-B7H4V | Control | | | | |
| human | B7H41001 mAb-DM4 | Up.in.Test/ | BP | GO:0050727 | regulation of inflammatory response | 1.13E−03 |
| | vs. SGN-B7H4V | Control | | | | |
| human | B7H41001 mAb-DM4 | Up.in.Test/ | BP | GO:0009612 | response to mechanical stimulus | 1.15E−03 |
| | vs. SGN-B7H4V | Control | | | | |
| human | B7H41001 mAb-DM4 | Up.in.Test/ | BP | GO:0045621 | positive regulation of lymphocyte | 1.15E−03 |
| | vs. SGN-B7H4V | Control | | | differentiation | |
| human | B7H41001 mAb-DM4 | Up.in.Test/ | BP | GO:0051897 | positive regulation of protein kinase B | 1.15E−03 |
| | vs. SGN-B7H4V | Control | | | signaling | |
| human | B7H41001 mAb-DM4 | Up.in.Test/ | BP | GO:0042129 | regulation of T cell proliferation | 1.15E−03 |
| | vs. SGN-B7H4V | Control | | | | |
| human | B7H41001 mAb-DM4 | Up.in.Test/ | BP | GO:0060326 | cell chemotaxis | 1.15E−03 |
| | vs. SGN-B7H4V | Control | | | | |
| human | B7H41001 mAb-DM4 | Up.in.Test/ | BP | GO:0051403 | stress-activated MAPK cascade | 1.16E−03 |
| | vs. SGN-B7H4V | Control | | | | |
| human | B7H41001 mAb-DM4 | Up.in.Test/ | BP | GO:1903131 | mononuclear cell differentiation | 1.23E−03 |
| | vs. SGN-B7H4V | Control | | | | |
| human | B7H41001 mAb-DM4 | Up.in.Test/ | BP | GO:0043434 | response to peptide hormone | 1.26E−03 |
| | vs. SGN-B7H4V | Control | | | | |
| human | B7H41001 mAb-DM4 | Up.in.Test/ | BP | GO:1903557 | positive regulation of tumor necrosis factor | 1.27E−03 |
| | vs. SGN-B7H4V | Control | | | superfamily cytokine production | |
| human | B7H41001 mAb-DM4 | Up.in.Test/ | BP | GO:0070527 | platelet aggregation | 1.34E−03 |
| | vs. SGN-B7H4V | Control | | | | |
| human | B7H41001 mAb-DM4 | Up.in.Test/ | BP | GO:0032640 | tumor necrosis factor production | 1.34E−03 |
| | vs. SGN-B7H4V | Control | | | | |
| human | B7H41001 mAb-DM4 | Up.in.Test/ | BP | GO:0032680 | regulation of tumor necrosis factor production | 1.34E−03 |
| | vs. SGN-B7H4V | Control | | | | |
| human | B7H41001 mAb-DM4 | Up.in.Test/ | BP | GO:0043547 | positive regulation of GTPase activity | 1.34E−03 |
| | vs. SGN-B7H4V | Control | | | | |
| human | B7H41001 mAb-DM4 | Up.in.Test/ | BP | GO:0007229 | integrin-mediated signaling pathway | 1.40E−03 |
| | vs. SGN-B7H4V | Control | | | | |
| human | B7H41001 mAb-DM4 | Up.in.Test/ | BP | GO:1905477 | positive regulation of protein localization to | 1.40E−03 |
| | vs. SGN-B7H4V | Control | | | membrane | |
| human | B7H41001 mAb-DM4 | Up.in.Test/ | BP | GO:0045862 | positive regulation of proteolysis | 1.42E−03 |
| | vs. SGN-B7H4V | Control | | | | |
| human | B7H41001 mAb-DM4 | Up.in.Test/ | BP | GO:0007596 | blood coagulation | 1.42E−03 |
| | vs. SGN-B7H4V | Control | | | | |
| human | B7H41001 mAb-DM4 | Up.in.Test/ | BP | GO:0043409 | negative regulation of MAPK cascade | 1.43E−03 |
| | vs. SGN-B7H4V | Control | | | | |
| human | B7H41001 mAb-DM4 | Up.in.Test/ | BP | GO:0046777 | protein autophosphorylation | 1.49E−03 |
| | vs. SGN-B7H4V | Control | | | | |
| human | B7H41001 mAb-DM4 | Up.in.Test/ | BP | GO:0071559 | response to transforming growth factor beta | 1.51E−03 |
| | vs. SGN-B7H4V | Control | | | | |
| human | B7H41001 mAb-DM4 | Up.in.Test/ | BP | GO:0002443 | leukocyte mediated immunity | 1.52E−03 |
| | vs. SGN-B7H4V | Control | | | | |
| human | B7H41001 mAb-DM4 | Up.in.Test/ | BP | GO:0150115 | cell-substrate junction organization | 1.52E−03 |
| | vs. SGN-B7H4V | Control | | | | |
| human | B7H41001 mAb-DM4 | Down.in.Test/ | BP | GO:0032481 | positive regulation of type I interferon | 1.58E−03 |
| | vs. SGN-B7H4V | Control | | | production | |
| human | B7H41001 mAb-DM4 | Up.in.Test/ | BP | GO:0007160 | cell-matrix adhesion | 1.58E−03 |
| | vs. SGN-B7H4V | Control | | | | |
| human | B7H41001 mAb-DM4 | Up.in.Test/ | BP | GO:0072659 | protein localization to plasma membrane | 1.59E−03 |
| | vs. SGN-B7H4V | Control | | | | |
| human | B7H41001 mAb-DM4 | Up.in.Test/ | BP | GO:0001818 | negative regulation of cytokine production | 1.59E−03 |
| | vs. SGN-B7H4V | Control | | | | |
| human | B7H41001 mAb-DM4 | Up.in.Test/ | BP | GO:0030041 | actin filament polymerization | 1.62E−03 |
| | vs. SGN-B7H4V | Control | | | | |
| human | B7H41001 mAb-DM4 | Up.in.Test/ | BP | GO:0002761 | regulation of myeloid leukocyte | 1.62E−03 |
| | vs. SGN-B7H4V | Control | | | differentiation | |
| human | B7H41001 mAb-DM4 | Up.in.Test/ | BP | GO:1902905 | positive regulation of supramolecular fiber | 1.65E−03 |
| | vs. SGN-B7H4V | Control | | | organization | |
| human | B7H41001 mAb-DM4 | Up.in.Test/ | BP | GO:0031400 | negative regulation of protein modification | 1.70E−03 |
| | vs. SGN-B7H4V | Control | | | process | |
| human | B7H41001 mAb-DM4 | Up.in.Test/ | BP | GO:0032233 | positive regulation of actin filament bundle | 1.73E−03 |
| | vs. SGN-B7H4V | Control | | | assembly | |
| human | B7H41001 mAb-DM4 | Down.in.Test/ | BP | GO:0052372 | modulation by symbiont of entry into host | 1.74E−03 |
| | vs. SGN-B7H4V | Control | | | | |
| human | B7H41001 mAb-DM4 | Up.in.Test/ | BP | GO:0007259 | receptor signaling pathway via JAK-STAT | 1.79E−03 |
| | vs. SGN-B7H4V | Control | | | | |
| human | B7H41001 mAb-DM4 | Up.in.Test/ | BP | GO:0050817 | coagulation | 1.83E−03 |
| | vs. SGN-B7H4V | Control | | | | |
| human | B7H41001 mAb-DM4 | Up.in.Test/ | BP | GO:0002250 | adaptive immune response | 1.86E−03 |
| | vs. SGN-B7H4V | Control | | | | |
| human | B7H41001 mAb-DM4 | Up.in.Test/ | BP | GO:0045582 | positive regulation of T cell differentiation | 1.86E−03 |
| | vs. SGN-B7H4V | Control | | | | |
| human | B7H41001 mAb-DM4 | Down.in.Test/ | BP | GO:0039528 | cytoplasmic pattern recognition receptor | 1.88E−03 |
| | vs. SGN-B7H4V | Control | | | signaling pathway in response to virus | |
| human | B7H41001 mAb-DM4 | Down.in.Test/ | BP | GO:0140374 | antiviral innate immune response | 1.96E−03 |
| | vs. SGN-B7H4V | Control | | | | |
| human | B7H41001 mAb-DM4 | Down.in.Test/ | BP | GO:0034341 | response to interferon-gamma | 1.96E−03 |
| | vs. SGN-B7H4V | Control | | | | |
| human | B7H41001 mAb-DM4 | Down.in.Test/ | BP | GO:0060759 | regulation of response to cytokine stimulus | 1.96E−03 |
| | vs. SGN-B7H4V | Control | | | | |
| human | B7H41001 mAb-DM4 | Up.in.Test/ | BP | GO:0007599 | hemostasis | 1.96E−03 |
| | vs. SGN-B7H4V | Control | | | | |
| human | B7H41001 mAb-DM4 | Up.in.Test/ | BP | GO:0072678 | T cell migration | 2.00E−03 |
| | vs. SGN-B7H4V | Control | | | | |
| human | B7H41001 mAb-DM4 | Up.in.Test/ | BP | GO:0071560 | cellular response to transforming growth | 2.01E−03 |
| | vs. SGN-B7H4V | Control | | | factor beta stimulus | |
| human | B7H41001 mAb-DM4 | Up.in.Test/ | BP | GO:0032609 | interferon-gamma production | 2.03E−03 |
| | vs. SGN-B7H4V | Control | | | | |
| human | B7H41001 mAb-DM4 | Up.in.Test/ | BP | GO:0032649 | regulation of interferon-gamma production | 2.03E−03 |
| | vs. SGN-B7H4V | Control | | | | |
| human | B7H41001 mAb-DM4 | Up.in.Test/ | BP | GO:0070371 | ERK1 and ERK2 cascade | 2.05E−03 |
| | vs. SGN-B7H4V | Control | | | | |
| human | B7H41001 mAb-DM4 | Up.in.Test/ | BP | GO:0032760 | positive regulation of tumor necrosis factor | 2.06E−03 |
| | vs. SGN-B7H4V | Control | | | production | |
| human | B7H41001 mAb-DM4 | Down.in.Test/ | BP | GO:0001916 | positive regulation of T cell mediated | 2.07E−03 |
| | vs. SGN-B7H4V | Control | | | cytotoxicity | |
| human | B7H41001 mAb-DM4 | Down.in.Test/ | BP | GO:0043903 | regulation of biological process involved in | 2.08E−03 |
| | vs. SGN-B7H4V | Control | | | symbiotic interaction | |
| human | B7H41001 mAb-DM4 | Up.in.Test/ | BP | GO:2001235 | positive regulation of apoptotic signaling | 2.14E−03 |
| | vs. SGN-B7H4V | Control | | | pathway | |
| human | B7H41001 mAb-DM4 | Up.in.Test/ | BP | GO:0006909 | phagocytosis | 2.46E−03 |
| | vs. SGN-B7H4V | Control | | | | |
| human | B7H41001 mAb-DM4 | Up.in.Test/ | BP | GO:0050920 | regulation of chemotaxis | 2.55E−03 |
| | vs. SGN-B7H4V | Control | | | | |
| human | B7H41001 mAb-DM4 | Up.in.Test/ | BP | GO:0050731 | positive regulation of peptidyl-tyrosine | 2.57E−03 |
| | vs. SGN-B7H4V | Control | | | phosphorylation | |
| human | B7H41001 mAb-DM4 | Down.in.Test/ | BP | GO:0000375 | RNA splicing, via transesterification reactions | 2.64E−03 |
| | vs. SGN-B7H4V | Control | | | | |
| human | B7H41001 mAb-DM4 | Up.in.Test/ | BP | GO:0030048 | actin filament-based movement | 2.65E−03 |
| | vs. SGN-B7H4V | Control | | | | |
| human | B7H41001 mAb-DM4 | Up.in.Test/ | BP | GO:0050866 | negative regulation of cell activation | 2.65E−03 |
| | vs. SGN-B7H4V | Control | | | | |
| human | B7H41001 mAb-DM4 | Up.in.Test/ | BP | GO:0090316 | positive regulation of intracellular protein | 2.70E−03 |
| | vs. SGN-B7H4V | Control | | | transport | |
| human | B7H41001 mAb-DM4 | Up.in.Test/ | BP | GO:0030595 | leukocyte chemotaxis | 2.70E−03 |
| | vs. SGN-B7H4V | Control | | | | |
| human | B7H41001 mAb-DM4 | Up.in.Test/ | BP | GO:0046631 | alpha-beta T cell activation | 2.71E−03 |
| | vs. SGN-B7H4V | Control | | | | |
| human | B7H41001 mAb-DM4 | Down.in.Test/ | BP | GO:0006397 | mRNA processing | 2.81E−03 |
| | vs. SGN-B7H4V | Control | | | | |
| human | B7H41001 mAb-DM4 | Down.in.Test/ | BP | GO:0001913 | T cell mediated cytotoxicity | 2.81E−03 |
| | vs. SGN-B7H4V | Control | | | | |
| human | B7H41001 mAb-DM4 | Up.in.Test/ | BP | GO:0030168 | platelet activation | 2.91E−03 |
| | vs. SGN-B7H4V | Control | | | | |
| human | B7H41001 mAb-DM4 | Up.in.Test/ | BP | GO:0046635 | positive regulation of alpha-beta T cell | 3.08E−03 |
| | vs. SGN-B7H4V | Control | | | activation | |
| human | B7H41001 mAb-DM4 | Up.in.Test/ | BP | GO:0030217 | T cell differentiation | 3.17E−03 |
| | vs. SGN-B7H4V | Control | | | | |
| human | B7H41001 mAb-DM4 | Up.in.Test/ | BP | GO:1904705 | regulation of vascular associated smooth | 3.18E−03 |
| | vs. SGN-B7H4V | Control | | | muscle cell proliferation | |
| human | B7H41001 mAb-DM4 | Up.in.Test/ | BP | GO:0030038 | contractile actin filament bundle assembly | 3.20E−03 |
| | vs. SGN-B7H4V | Control | | | | |
| human | B7H41001 mAb-DM4 | Up.in.Test/ | BP | GO:0043149 | stress fiber assembly | 3.20E−03 |
| | vs. SGN-B7H4V | Control | | | | |
| human | B7H41001 mAb-DM4 | Up.in.Test/ | BP | GO:0097696 | receptor signaling pathway via STAT | 3.20E−03 |
| | vs. SGN-B7H4V | Control | | | | |
| human | B7H41001 mAb-DM4 | Up.in.Test/ | BP | GO:0002685 | regulation of leukocyte migration | 3.25E−03 |
| | vs. SGN-B7H4V | Control | | | | |
| human | B7H41001 mAb-DM4 | Up.in.Test/ | BP | GO:0043491 | protein kinase B signaling | 3.27E−03 |
| | vs. SGN-B7H4V | Control | | | | |
| human | B7H41001 mAb-DM4 | Down.in.Test/ | BP | GO:0019882 | antigen processing and presentation | 3.31E−03 |
| | vs. SGN-B7H4V | Control | | | | |
| human | B7H41001 mAb-DM4 | Down.in.Test/ | BP | GO:0001914 | regulation of T cell mediated cytotoxicity | 3.31E−03 |
| | vs. SGN-B7H4V | Control | | | | |
| human | B7H41001 mAb-DM4 | Up.in.Test/ | BP | GO:0010632 | regulation of epithelial cell migration | 3.32E−03 |
| | vs. SGN-B7H4V | Control | | | | |
| human | B7H41001 mAb-DM4 | Down.in.Test/ | BP | GO:0001911 | negative regulation of leukocyte mediated | 3.38E−03 |
| | vs. SGN-B7H4V | Control | | | cytotoxicity | |
| human | B7H41001 mAb-DM4 | Up.in.Test/ | BP | GO:0007179 | transforming growth factor beta receptor | 3.38E−03 |
| | vs. SGN-B7H4V | Control | | | signaling pathway | |
| human | B7H41001 mAb-DM4 | Up.in.Test/ | BP | GO:0051222 | positive regulation of protein transport | 3.40E−03 |
| | vs. SGN-B7H4V | Control | | | | |
| human | B7H41001 mAb-DM4 | Up.in.Test/ | BP | GO:0030098 | lymphocyte differentiation | 3.51E−03 |
| | vs. SGN-B7H4V | Control | | | | |
| human | B7H41001 mAb-DM4 | Up.in.Test/ | BP | GO:0016358 | dendrite development | 3.51E−03 |
| | vs. SGN-B7H4V | Control | | | | |
| human | B7H41001 mAb-DM4 | Up.in.Test/ | BP | GO:0051056 | regulation of small GTPase mediated signal | 3.56E−03 |
| | vs. SGN-B7H4V | Control | | | transduction | |
| human | B7H41001 mAb-DM4 | Up.in.Test/ | BP | GO:1990874 | vascular associated smooth muscle cell | 3.56E−03 |
| | vs. SGN-B7H4V | Control | | | proliferation | |
| human | B7H41001 mAb-DM4 | Up.in.Test/ | BP | GO:0045058 | T cell selection | 3.56E−03 |
| | vs. SGN-B7H4V | Control | | | | |
| human | B7H41001 mAb-DM4 | Up.in.Test/ | BP | GO:0043123 | positive regulation of I-kappaB kinase/NF- | 3.56E−03 |
| | vs. SGN-B7H4V | Control | | | kappaB signaling | |
| human | B7H41001 mAb-DM4 | Up.in.Test/ | BP | GO:0051271 | negative regulation of cellular component | 3.73E−03 |
| | vs. SGN-B7H4V | Control | | | movement | |
| human | B7H41001 mAb-DM4 | Up.in.Test/ | BP | GO:0040013 | negative regulation of locomotion | 3.73E−03 |
| | vs. SGN-B7H4V | Control | | | | |
| human | B7H41001 mAb-DM4 | Down.in.Test/ | BP | GO:0001960 | negative regulation of cytokine-mediated | 3.75E−03 |
| | vs. SGN-B7H4V | Control | | | signaling pathway | |
| human | B7H41001 mAb-DM4 | Up.in.Test/ | BP | GO:0030336 | negative regulation of cell migration | 3.88E−03 |
| | vs. SGN-B7H4V | Control | | | | |
| human | B7H41001 mAb-DM4 | Up.in.Test/ | BP | GO:0032388 | positive regulation of intracellular transport | 3.90E−03 |
| | vs. SGN-B7H4V | Control | | | | |
| human | B7H41001 mAb-DM4 | Up.in.Test/ | BP | GO:0045637 | regulation of myeloid cell differentiation | 4.13E−03 |
| | vs. SGN-B7H4V | Control | | | | |
| human | B7H41001 mAb-DM4 | Up.in.Test/ | BP | GO:0051496 | positive regulation of stress fiber assembly | 4.18E−03 |
| | vs. SGN-B7H4V | Control | | | | |
| human | B7H41001 mAb-DM4 | Up.in.Test/ | BP | GO:0072676 | lymphocyte migration | 4.18E−03 |
| | vs. SGN-B7H4V | Control | | | | |
| human | B7H41001 mAb-DM4 | Up.in.Test/ | BP | GO:0097191 | extrinsic apoptotic signaling pathway | 4.18E−03 |
| | vs. SGN-B7H4V | Control | | | | |
| human | B7H41001 mAb-DM4 | Up.in.Test/ | BP | GO:0032801 | receptor catabolic process | 4.18E−03 |
| | vs. SGN-B7H4V | Control | | | | |
| human | B7H41001 mAb-DM4 | Down.in.Test/ | BP | GO:0032479 | regulation of type I interferon production | 4.38E−03 |
| | vs. SGN-B7H4V | Control | | | | |
| human | B7H41001 mAb-DM4 | Down.in.Test/ | BP | GO:0032606 | type I interferon production | 4.38E−03 |
| | vs. SGN-B7H4V | Control | | | | |
| human | B7H41001 mAb-DM4 | Down.in.Test/ | BP | GO:0000377 | RNA splicing, via transesterification reactions | 4.38E−03 |
| | vs. SGN-B7H4V | Control | | | with bulged adenosine as nucleophile | |
| human | B7H41001 mAb-DM4 | Down.in.Test/ | BP | GO:0000398 | mRNA splicing, via spliceosome | 4.38E−03 |
| | vs. SGN-B7H4V | Control | | | | |
| human | B7H41001 mAb-DM4 | Down.in.Test/ | BP | GO:0002753 | cytoplasmic pattern recognition receptor | 4.38E−03 |
| | vs. SGN-B7H4V | Control | | | signaling pathway | |
| human | B7H41001 mAb-DM4 | Up.in.Test/ | BP | GO:0050862 | positive regulation of T cell receptor signaling | 4.41E−03 |
| | vs. SGN-B7H4V | Control | | | pathway | |
| human | B7H41001 mAb-DM4 | Up.in.Test/ | BP | GO:2000146 | negative regulation of cell motility | 4.51E−03 |
| | vs. SGN-B7H4V | Control | | | | |
| human | B7H41001 mAb-DM4 | Up.in.Test/ | BP | GO:0051896 | regulation of protein kinase B signaling | 4.78E−03 |
| | vs. SGN-B7H4V | Control | | | | |
| human | B7H41001 mAb-DM4 | Up.in.Test/ | BP | GO:0045732 | positive regulation of protein catabolic | 4.79E−03 |
| | vs. SGN-B7H4V | Control | | | process | |
| human | B7H41001 mAb-DM4 | Up.in.Test/ | BP | GO:0051495 | positive regulation of cytoskeleton | 4.79E−03 |
| | vs. SGN-B7H4V | Control | | | organization | |
| human | B7H41001 mAb-DM4 | Down.in.Test/ | BP | GO:0002833 | positive regulation of response to biotic | 4.85E−03 |
| | vs. SGN-B7H4V | Control | | | stimulus | |
| human | B7H41001 mAb-DM4 | Up.in.Test/ | BP | GO:0042100 | B cell proliferation | 5.07E−03 |
| | vs. SGN-B7H4V | Control | | | | |
| human | B7H41001 mAb-DM4 | Up.in.Test/ | BP | GO:0050878 | regulation of body fluid levels | 5.09E−03 |
| | vs. SGN-B7H4V | Control | | | | |
| human | B7H41001 mAb-DM4 | Down.in.Test/ | BP | GO:0060761 | negative regulation of response to cytokine | 5.20E−03 |
| | vs. SGN-B7H4V | Control | | | stimulus | |
| human | B7H41001 mAb-DM4 | Up.in.Test/ | BP | GO:0045619 | regulation of lymphocyte differentiation | 5.20E−03 |
| | vs. SGN-B7H4V | Control | | | | |
| human | B7H41001 mAb-DM4 | Up.in.Test/ | BP | GO:0032147 | activation of protein kinase activity | 5.29E−03 |
| | vs. SGN-B7H4V | Control | | | | |
| human | B7H41001 mAb-DM4 | Down.in.Test/ | BP | GO:0019221 | cytokine-mediated signaling pathway | 5.42E−03 |
| | vs. SGN-B7H4V | Control | | | | |
| human | B7H41001 mAb-DM4 | Up.in.Test/ | BP | GO:0007044 | cell-substrate junction assembly | 5.42E−03 |
| | vs. SGN-B7H4V | Control | | | | |
| human | B7H41001 mAb-DM4 | Up.in.Test/ | BP | GO:0071356 | cellular response to tumor necrosis factor | 5.45E−03 |
| | vs. SGN-B7H4V | Control | | | | |
| human | B7H41001 mAb-DM4 | Up.in.Test/ | BP | GO:1990778 | protein localization to cell periphery | 5.47E−03 |
| | vs. SGN-B7H4V | Control | | | | |
| human | B7H41001 mAb-DM4 | Down.in.Test/ | BP | GO:0002221 | pattern recognition receptor signaling | 5.48E−03 |
| | vs. SGN-B7H4V | Control | | | pathway | |
| human | B7H41001 mAb-DM4 | Up.in.Test/ | BP | GO:0044089 | positive regulation of cellular component | 5.48E−03 |
| | vs. SGN-B7H4V | Control | | | biogenesis | |
| human | B7H41001 mAb-DM4 | Up.in.Test/ | BP | GO:0061097 | regulation of protein tyrosine kinase activity | 5.56E−03 |
| | vs. SGN-B7H4V | Control | | | | |
| human | B7H41001 mAb-DM4 | Up.in.Test/ | BP | GO:0034446 | substrate adhesion-dependent cell spreading | 5.56E−03 |
| | vs. SGN-B7H4V | Control | | | | |
| human | B7H41001 mAb-DM4 | Up.in.Test/ | BP | GO:0048008 | platelet-derived growth factor receptor | 5.56E−03 |
| | vs. SGN-B7H4V | Control | | | signaling pathway | |
| human | B7H41001 mAb-DM4 | Up.in.Test/ | BP | GO:0070373 | negative regulation of ERK1 and ERK2 cascade | 5.56E−03 |
| | vs. SGN-B7H4V | Control | | | | |
| human | B7H41001 mAb-DM4 | Up.in.Test/ | BP | GO:0046425 | regulation of receptor signaling pathway via | 5.57E−03 |
| | vs. SGN-B7H4V | Control | | | JAK-STAT | |
| human | B7H41001 mAb-DM4 | Up.in.Test/ | BP | GO:1903533 | regulation of protein targeting | 5.57E−03 |
| | vs. SGN-B7H4V | Control | | | | |
| human | B7H41001 mAb-DM4 | Up.in.Test/ | BP | GO:0032271 | regulation of protein polymerization | 5.65E−03 |
| | vs. SGN-B7H4V | Control | | | | |
| human | B7H41001 mAb-DM4 | Up.in.Test/ | BP | GO:0060627 | regulation of vesicle-mediated transport | 5.69E−03 |
| | vs. SGN-B7H4V | Control | | | | |
| human | B7H41001 mAb-DM4 | Up.in.Test/ | BP | GO:0032231 | regulation of actin filament bundle assembly | 5.77E−03 |
| | vs. SGN-B7H4V | Control | | | | |
| human | B7H41001 mAb-DM4 | Up.in.Test/ | BP | GO:0046634 | regulation of alpha-beta T cell activation | 5.77E−03 |
| | vs. SGN-B7H4V | Control | | | | |
| human | B7H41001 mAb-DM4 | Up.in.Test/ | BP | GO:0050853 | B cell receptor signaling pathway | 5.98E−03 |
| | vs. SGN-B7H4V | Control | | | | |
| human | B7H41001 mAb-DM4 | Up.in.Test/ | BP | GO:0043542 | endothelial cell migration | 5.99E−03 |
| | vs. SGN-B7H4V | Control | | | | |
| human | B7H41001 mAb-DM4 | Up.in.Test/ | BP | GO:0043368 | positive T cell selection | 6.04E−03 |
| | vs. SGN-B7H4V | Control | | | | |
| human | B7H41001 mAb-DM4 | Up.in.Test/ | BP | GO:0043372 | positive regulation of CD4-positive, alpha-beta | 6.26E−03 |
| | vs. SGN-B7H4V | Control | | | T cell differentiation | |
| human | B7H41001 mAb-DM4 | Up.in.Test/ | BP | GO:0046320 | regulation of fatty acid oxidation | 6.26E−03 |
| | vs. SGN-B7H4V | Control | | | | |
| human | B7H41001 mAb-DM4 | Up.in.Test/ | BP | GO:0032869 | cellular response to insulin stimulus | 6.26E−03 |
| | vs. SGN-B7H4V | Control | | | | |
| human | B7H41001 mAb-DM4 | Up.in.Test/ | BP | GO:0010761 | fibroblast migration | 6.26E−03 |
| | vs. SGN-B7H4V | Control | | | | |
| human | B7H41001 mAb-DM4 | Up.in.Test/ | BP | GO:0032873 | negative regulation of stress-activated MAPK | 6.26E−03 |
| | vs. SGN-B7H4V | Control | | | cascade | |
| human | B7H41001 mAb-DM4 | Up.in.Test/ | BP | GO:0070303 | negative regulation of stress-activated protein | 6.26E−03 |
| | vs. SGN-B7H4V | Control | | | kinase signaling cascade | |
| human | B7H41001 mAb-DM4 | Up.in.Test/ | BP | GO:1902041 | regulation of extrinsic apoptotic signaling | 6.26E−03 |
| | vs. SGN-B7H4V | Control | | | pathway via death domain receptors | |
| human | B7H41001 mAb-DM4 | Down.in.Test/ | BP | GO:0031342 | negative regulation of cell killing | 6.39E−03 |
| | vs. SGN-B7H4V | Control | | | | |
| human | B7H41001 mAb-DM4 | Up.in.Test/ | BP | GO:0071675 | regulation of mononuclear cell migration | 6.43E−03 |
| | vs. SGN-B7H4V | Control | | | | |
| human | B7H41001 mAb-DM4 | Up.in.Test/ | BP | GO:0007266 | Rho protein signal transduction | 6.43E−03 |
| | vs. SGN-B7H4V | Control | | | | |
| human | B7H41001 mAb-DM4 | Up.in.Test/ | BP | GO:0010811 | positive regulation of cell-substrate adhesion | 6.45E−03 |
| | vs. SGN-B7H4V | Control | | | | |
| human | B7H41001 mAb-DM4 | Down.in.Test/ | BP | GO:0001959 | regulation of cytokine-mediated signaling | 6.46E−03 |
| | vs. SGN-B7H4V | Control | | | pathway | |
| human | B7H41001 mAb-DM4 | Up.in.Test/ | BP | GO:0033673 | negative regulation of kinase activity | 6.56E−03 |
| | vs. SGN-B7H4V | Control | | | | |
| human | B7H41001 mAb-DM4 | Up.in.Test/ | BP | GO:0000910 | cytokinesis | 6.56E−03 |
| | vs. SGN-B7H4V | Control | | | | |
| human | B7H41001 mAb-DM4 | Up.in.Test/ | BP | GO:0030888 | regulation of B cell proliferation | 6.56E−03 |
| | vs. SGN-B7H4V | Control | | | | |
| human | B7H41001 mAb-DM4 | Up.in.Test/ | BP | GO:0051346 | negative regulation of hydrolase activity | 6.56E−03 |
| | vs. SGN-B7H4V | Control | | | | |
| human | B7H41001 mAb-DM4 | Up.in.Test/ | BP | GO:0051656 | establishment of organelle localization | 6.91E−03 |
| | vs. SGN-B7H4V | Control | | | | |
| human | B7H41001 mAb-DM4 | Up.in.Test/ | BP | GO:0018210 | peptidyl-threonine modification | 6.98E−03 |
| | vs. SGN-B7H4V | Control | | | | |
| human | B7H41001 mAb-DM4 | Up.in.Test/ | BP | GO:0046633 | alpha-beta T cell proliferation | 6.98E−03 |
| | vs. SGN-B7H4V | Control | | | | |
| human | B7H41001 mAb-DM4 | Up.in.Test/ | BP | GO:0046578 | regulation of Ras protein signal transduction | 6.98E−03 |
| | vs. SGN-B7H4V | Control | | | | |
| human | B7H41001 mAb-DM4 | Up.in.Test/ | BP | GO:0006469 | negative regulation of protein kinase activity | 6.98E−03 |
| | vs. SGN-B7H4V | Control | | | | |
| human | B7H41001 mAb-DM4 | Up.in.Test/ | BP | GO:0106027 | neuron projection organization | 7.11E−03 |
| | vs. SGN-B7H4V | Control | | | | |
| human | B7H41001 mAb-DM4 | Up.in.Test/ | BP | GO:0002697 | regulation of immune effector process | 7.11E−03 |
| | vs. SGN-B7H4V | Control | | | | |
| human | B7H41001 mAb-DM4 | Up.in.Test/ | BP | GO:1904951 | positive regulation of establishment of protein | 7.11E−03 |
| | vs. SGN-B7H4V | Control | | | localization | |
| human | B7H41001 mAb-DM4 | Up.in.Test/ | BP | GO:0045216 | cell-cell junction organization | 7.42E−03 |
| | vs. SGN-B7H4V | Control | | | | |
| human | B7H41001 mAb-DM4 | Up.in.Test/ | BP | GO:0043299 | leukocyte degranulation | 7.43E−03 |
| | vs. SGN-B7H4V | Control | | | | |
| human | B7H41001 mAb-DM4 | Up.in.Test/ | BP | GO:0018107 | peptidyl-threonine phosphorylation | 7.43E−03 |
| | vs. SGN-B7H4V | Control | | | | |
| human | B7H41001 mAb-DM4 | Up.in.Test/ | BP | GO:0002700 | regulation of production of molecular | 7.43E−03 |
| | vs. SGN-B7H4V | Control | | | mediator of immune response | |
| human | B7H41001 mAb-DM4 | Up.in.Test/ | BP | GO:0006887 | exocytosis | 7.43E−03 |
| | vs. SGN-B7H4V | Control | | | | |
| human | B7H41001 mAb-DM4 | Up.in.Test/ | BP | GO:0042692 | muscle cell differentiation | 7.43E−03 |
| | vs. SGN-B7H4V | Control | | | | |
| human | B7H41001 mAb-DM4 | Up.in.Test/ | BP | GO:2001236 | regulation of extrinsic apoptotic signaling | 7.68E−03 |
| | vs. SGN-B7H4V | Control | | | pathway | |
| human | B7H41001 mAb-DM4 | Up.in.Test/ | BP | GO:0071496 | cellular response to external stimulus | 7.68E−03 |
| | vs. SGN-B7H4V | Control | | | | |
| human | B7H41001 mAb-DM4 | Up.in.Test/ | BP | GO:1901214 | regulation of neuron death | 7.68E−03 |
| | vs. SGN-B7H4V | Control | | | | |
| human | B7H41001 mAb-DM4 | Up.in.Test/ | BP | GO:0001952 | regulation of cell-matrix adhesion | 8.08E−03 |
| | vs. SGN-B7H4V | Control | | | | |
| human | B7H41001 mAb-DM4 | Up.in.Test/ | BP | GO:0070372 | regulation of ERK1 and ERK2 cascade | 8.22E−03 |
| | vs. SGN-B7H4V | Control | | | | |
| human | B7H41001 mAb-DM4 | Down.in.Test/ | BP | GO:0002449 | lymphocyte mediated immunity | 8.41E−03 |
| | vs. SGN-B7H4V | Control | | | | |
| human | B7H41001 mAb-DM4 | Down.in.Test/ | BP | GO:0044403 | biological process involved in symbiotic | 8.49E−03 |
| | vs. SGN-B7H4V | Control | | | interaction | |
| human | B7H41001 mAb-DM4 | Up.in.Test/ | BP | GO:0110020 | regulation of actomyosin structure | 8.61E−03 |
| | vs. SGN-B7H4V | Control | | | organization | |
| human | B7H41001 mAb-DM4 | Up.in.Test/ | BP | GO:0042542 | response to hydrogen peroxide | 8.61E−03 |
| | vs. SGN-B7H4V | Control | | | | |
| human | B7H41001 mAb-DM4 | Up.in.Test/ | BP | GO:0051650 | establishment of vesicle localization | 8.61E−03 |
| | vs. SGN-B7H4V | Control | | | | |
| human | B7H41001 mAb-DM4 | Up.in.Test/ | BP | GO:0032872 | regulation of stress-activated MAPK cascade | 8.92E−03 |
| | vs. SGN-B7H4V | Control | | | | |
| human | B7H41001 mAb-DM4 | Up.in.Test/ | BP | GO:0002263 | cell activation involved in immune response | 8.97E−03 |
| | vs. SGN-B7H4V | Control | | | | |
| human | B7H41001 mAb-DM4 | Up.in.Test/ | BP | GO:0000423 | mitophagy | 9.03E−03 |
| | vs. SGN-B7H4V | Control | | | | |
| human | B7H41001 mAb-DM4 | Down.in.Test/ | BP | GO:0002456 | T cell mediated immunity | 9.14E−03 |
| | vs. SGN-B7H4V | Control | | | | |
| human | B7H41001 mAb-DM4 | Up.in.Test/ | BP | GO:0033157 | regulation of intracellular protein transport | 9.14E−03 |
| | vs. SGN-B7H4V | Control | | | | |
| human | B7H41001 mAb-DM4 | Up.in.Test/ | BP | GO:0016311 | dephosphorylation | 9.14E−03 |
| | vs. SGN-B7H4V | Control | | | | |
| human | B7H41001 mAb-DM4 | Up.in.Test/ | BP | GO:0051223 | regulation of protein transport | 9.22E−03 |
| | vs. SGN-B7H4V | Control | | | | |
| human | B7H41001 mAb-DM4 | Up.in.Test/ | BP | GO:0090066 | regulation of anatomical structure size | 9.22E−03 |
| | vs. SGN-B7H4V | Control | | | | |
| human | B7H41001 mAb-DM4 | Up.in.Test/ | BP | GO:0038066 | p38MAPK cascade | 9.46E−03 |
| | vs. SGN-B7H4V | Control | | | | |
| human | B7H41001 mAb-DM4 | Up.in.Test/ | BP | GO:0046638 | positive regulation of alpha-beta T cell | 9.46E−03 |
| | vs. SGN-B7H4V | Control | | | differentiation | |
| human | B7H41001 mAb-DM4 | Up.in.Test/ | BP | GO:0030866 | cortical actin cytoskeleton organization | 9.51E−03 |
| | vs. SGN-B7H4V | Control | | | | |
| human | B7H41001 mAb-DM4 | Up.in.Test/ | BP | GO:0034612 | response to tumor necrosis factor | 9.58E−03 |
| | vs. SGN-B7H4V | Control | | | | |
| human | B7H41001 mAb-DM4 | Up.in.Test/ | BP | GO:0032102 | negative regulation of response to external | 9.63E−03 |
| | vs. SGN-B7H4V | Control | | | stimulus | |
| human | B7H41001 mAb-DM4 | Up.in.Test/ | BP | GO:0051098 | regulation of binding | 9.64E−03 |
| | vs. SGN-B7H4V | Control | | | | |
| human | B7H41001 mAb-DM4 | Up.in.Test/ | BP | GO:0070997 | neuron death | 9.64E−03 |
| | vs. SGN-B7H4V | Control | | | | |
| human | B7H41001 mAb-DM4 | Up.in.Test/ | BP | GO:0010638 | positive regulation of organelle organization | 9.67E−03 |
| | vs. SGN-B7H4V | Control | | | | |
| mouse | B7H41001 mAb-DM4 | Up.in.Test/ | BP | GO:0006091 | generation of precursor metabolites and | 1.55E−16 |
| | vs. SGN-B7H4V | Control | | | energy | |
| mouse | B7H41001 mAb-DM4 | Up.in.Test/ | BP | GO:0015980 | energy derivation by oxidation of organic | 2.76E−16 |
| | vs. SGN-B7H4V | Control | | | compounds | |
| mouse | B7H41001 mAb-DM4 | Up.in.Test/ | BP | GO:0045333 | cellular respiration | 4.61E−15 |
| | vs. SGN-B7H4V | Control | | | | |
| mouse | B7H41001 mAb-DM4 | Up.in.Test/ | BP | GO:0006099 | tricarboxylic acid cycle | 4.61E−15 |
| | vs. SGN-B7H4V | Control | | | | |
| mouse | B7H41001 mAb-DM4 | Up.in.Test/ | BP | GO:0009060 | aerobic respiration | 2.62E−14 |
| | vs. SGN-B7H4V | Control | | | | |
| mouse | B7H41001 mAb-DM4 | Up.in.Test/ | BP | GO:0043648 | dicarboxylic acid metabolic process | 2.60E−13 |
| | vs. SGN-B7H4V | Control | | | | |
| mouse | B7H41001 mAb-DM4 | Up.in.Test/ | BP | GO:0046395 | carboxylic acid catabolic process | 1.76E−12 |
| | vs. SGN-B7H4V | Control | | | | |
| mouse | B7H41001 mAb-DM4 | Up.in.Test/ | BP | GO:0044282 | small molecule catabolic process | 1.76E−12 |
| | vs. SGN-B7H4V | Control | | | | |
| mouse | B7H41001 mAb-DM4 | Up.in.Test/ | BP | GO:0016054 | organic acid catabolic process | 1.80E−12 |
| | vs. SGN-B7H4V | Control | | | | |
| mouse | B7H41001 mAb-DM4 | Up.in.Test/ | BP | GO:0009062 | fatty acid catabolic process | 6.70E−11 |
| | vs. SGN-B7H4V | Control | | | | |
| mouse | B7H41001 mAb-DM4 | Up.in.Test/ | BP | GO:0072329 | monocarboxylic acid catabolic process | 1.45E−09 |
| | vs. SGN-B7H4V | Control | | | | |
| mouse | B7H41001 mAb-DM4 | Up.in.Test/ | BP | GO:0006635 | fatty acid beta-oxidation | 1.73E−09 |
| | vs. SGN-B7H4V | Control | | | | |
| mouse | B7H41001 mAb-DM4 | Up.in.Test/ | BP | GO:0019395 | fatty acid oxidation | 2.88E−09 |
| | vs. SGN-B7H4V | Control | | | | |
| mouse | B7H41001 mAb-DM4 | Up.in.Test/ | BP | GO:0009117 | nucleotide metabolic process | 5.12E−09 |
| | vs. SGN-B7H4V | Control | | | | |
| mouse | B7H41001 mAb-DM4 | Up.in.Test/ | BP | GO:0034440 | lipid oxidation | 6.75E−09 |
| | vs. SGN-B7H4V | Control | | | | |
| mouse | B7H41001 mAb-DM4 | Up.in.Test/ | BP | GO:0006753 | nucleoside phosphate metabolic process | 6.75E−09 |
| | vs. SGN-B7H4V | Control | | | | |
| mouse | B7H41001 mAb-DM4 | Up.in.Test/ | BP | GO:0006163 | purine nucleotide metabolic process | 6.75E−09 |
| | vs. SGN-B7H4V | Control | | | | |
| mouse | B7H41001 mAb-DM4 | Up.in.Test/ | BP | GO:0009150 | purine ribonucleotide metabolic process | 7.33E−09 |
| | vs. SGN-B7H4V | Control | | | | |
| mouse | B7H41001 mAb-DM4 | Up.in.Test/ | BP | GO:0072521 | purine-containing compound metabolic | 2.29E−08 |
| | vs. SGN-B7H4V | Control | | | process | |
| mouse | B7H41001 mAb-DM4 | Up.in.Test/ | BP | GO:0009259 | ribonucleotide metabolic process | 2.32E−08 |
| | vs. SGN-B7H4V | Control | | | | |
| mouse | B7H41001 mAb-DM4 | Up.in.Test/ | BP | GO:0019693 | ribose phosphate metabolic process | 4.68E−08 |
| | vs. SGN-B7H4V | Control | | | | |
| mouse | B7H41001 mAb-DM4 | Up.in.Test/ | BP | GO:0055086 | nucleobase-containing small molecule | 7.82E−08 |
| | vs. SGN-B7H4V | Control | | | metabolic process | |
| mouse | B7H41001 mAb-DM4 | Up.in.Test/ | BP | GO:0006103 | 2-oxoglutarate metabolic process | 1.91E−07 |
| | vs. SGN-B7H4V | Control | | | | |
| mouse | B7H41001 mAb-DM4 | Up.in.Test/ | BP | GO:0016042 | lipid catabolic process | 7.17E−07 |
| | vs. SGN-B7H4V | Control | | | | |
| mouse | B7H41001 mAb-DM4 | Down.in.Test/ | BP | GO:0010634 | positive regulation of epithelial cell migration | 1.20E−06 |
| | vs. SGN-B7H4V | Control | | | | |
| mouse | B7H41001 mAb-DM4 | Down.in.Test/ | BP | GO:0010632 | regulation of epithelial cell migration | 1.20E−06 |
| | vs. SGN-B7H4V | Control | | | | |
| mouse | B7H41001 mAb-DM4 | Down.in.Test/ | BP | GO:0010631 | epithelial cell migration | 1.20E−06 |
| | vs. SGN-B7H4V | Control | | | | |
| mouse | B7H41001 mAb-DM4 | Down.in.Test/ | BP | GO:0090132 | epithelium migration | 1.20E−06 |
| | vs. SGN-B7H4V | Control | | | | |
| mouse | B7H41001 mAb-DM4 | Down.in.Test/ | BP | GO:0090130 | tissue migration | 1.20E−06 |
| | vs. SGN-B7H4V | Control | | | | |
| mouse | B7H41001 mAb-DM4 | Down.in.Test/ | BP | GO:0010594 | regulation of endothelial cell migration | 1.26E−06 |
| | vs. SGN-B7H4V | Control | | | | |
| mouse | B7H41001 mAb-DM4 | Up.in.Test/ | BP | GO:0044242 | cellular lipid catabolic process | 1.44E−06 |
| | vs. SGN-B7H4V | Control | | | | |
| mouse | B7H41001 mAb-DM4 | Down.in.Test/ | BP | GO:0010595 | positive regulation of endothelial cell | 1.51E−06 |
| | vs. SGN-B7H4V | Control | | | migration | |
| mouse | B7H41001 mAb-DM4 | Down.in.Test/ | BP | GO:0050900 | leukocyte migration | 1.60E−06 |
| | vs. SGN-B7H4V | Control | | | | |
| mouse | B7H41001 mAb-DM4 | Up.in.Test/ | BP | GO:0006631 | fatty acid metabolic process | 2.51E−06 |
| | vs. SGN-B7H4V | Control | | | | |
| mouse | B7H41001 mAb-DM4 | Up.in.Test/ | BP | GO:0009165 | nucleotide biosynthetic process | 2.55E−06 |
| | vs. SGN-B7H4V | Control | | | | |
| mouse | B7H41001 mAb-DM4 | Up.in.Test/ | BP | GO:0030258 | lipid modification | 2.57E−06 |
| | vs. SGN-B7H4V | Control | | | | |
| mouse | B7H41001 mAb-DM4 | Up.in.Test/ | BP | GO:0006164 | purine nucleotide biosynthetic process | 2.57E−06 |
| | vs. SGN-B7H4V | Control | | | | |
| mouse | B7H41001 mAb-DM4 | Up.in.Test/ | BP | GO:1901293 | nucleoside phosphate biosynthetic process | 3.08E−06 |
| | vs. SGN-B7H4V | Control | | | | |
| mouse | B7H41001 mAb-DM4 | Up.in.Test/ | BP | GO:0009152 | purine ribonucleotide biosynthetic process | 3.48E−06 |
| | vs. SGN-B7H4V | Control | | | | |
| mouse | B7H41001 mAb-DM4 | Down.in.Test/ | BP | GO:0001819 | positive regulation of cytokine production | 3.71E−06 |
| | vs. SGN-B7H4V | Control | | | | |
| mouse | B7H41001 mAb-DM4 | Up.in.Test/ | BP | GO:0072522 | purine-containing compound biosynthetic | 3.92E−06 |
| | vs. SGN-B7H4V | Control | | | process | |
| mouse | B7H41001 mAb-DM4 | Up.in.Test/ | BP | GO:0007005 | mitochondrion organization | 4.10E−06 |
| | vs. SGN-B7H4V | Control | | | | |
| mouse | B7H41001 mAb-DM4 | Up.in.Test/ | BP | GO:0090407 | organophosphate biosynthetic process | 5.93E−06 |
| | vs. SGN-B7H4V | Control | | | | |
| mouse | B7H41001 mAb-DM4 | Up.in.Test/ | BP | GO:0006520 | cellular amino acid metabolic process | 7.26E−06 |
| | vs. SGN-B7H4V | Control | | | | |
| mouse | B7H41001 mAb-DM4 | Down.in.Test/ | BP | GO:0043542 | endothelial cell migration | 8.73E−06 |
| | vs. SGN-B7H4V | Control | | | | |
| mouse | B7H41001 mAb-DM4 | Up.in.Test/ | BP | GO:0009260 | ribonucleotide biosynthetic process | 8.81E−06 |
| | vs. SGN-B7H4V | Control | | | | |
| mouse | B7H41001 mAb-DM4 | Down.in.Test/ | BP | GO:1901342 | regulation of vasculature development | 1.54E−05 |
| | vs. SGN-B7H4V | Control | | | | |
| mouse | B7H41001 mAb-DM4 | Up.in.Test/ | BP | GO:0046390 | ribose phosphate biosynthetic process | 1.80E−05 |
| | vs. SGN-B7H4V | Control | | | | |
| mouse | B7H41001 mAb-DM4 | Up.in.Test/ | BP | GO:0033539 | fatty acid beta-oxidation using acyl-CoA | 1.93E−05 |
| | vs. SGN-B7H4V | Control | | | dehydrogenase | |
| mouse | B7H41001 mAb-DM4 | Up.in.Test/ | BP | GO:0006090 | pyruvate metabolic process | 2.05E−05 |
| | vs. SGN-B7H4V | Control | | | | |
| mouse | B7H41001 mAb-DM4 | Down.in.Test/ | BP | GO:0001667 | ameboidal-type cell migration | 2.19E−05 |
| | vs. SGN-B7H4V | Control | | | | |
| mouse | B7H41001 mAb-DM4 | Up.in.Test/ | BP | GO:0006107 | oxaloacetate metabolic process | 3.26E−05 |
| | vs. SGN-B7H4V | Control | | | | |
| mouse | B7H41001 mAb-DM4 | Up.in.Test/ | BP | GO:0009064 | glutamine family amino acid metabolic | 3.26E−05 |
| | vs. SGN-B7H4V | Control | | | process | |
| mouse | B7H41001 mAb-DM4 | Up.in.Test/ | BP | GO:0033865 | nucleoside bisphosphate metabolic process | 3.64E−05 |
| | vs. SGN-B7H4V | Control | | | | |
| mouse | B7H41001 mAb-DM4 | Up.in.Test/ | BP | GO:0033875 | ribonucleoside bisphosphate metabolic | 3.64E−05 |
| | vs. SGN-B7H4V | Control | | | process | |
| mouse | B7H41001 mAb-DM4 | Up.in.Test/ | BP | GO:0034032 | purine nucleoside bisphosphate metabolic | 3.64E−05 |
| | vs. SGN-B7H4V | Control | | | process | |
| mouse | B7H41001 mAb-DM4 | Up.in.Test/ | BP | GO:0006734 | NADH metabolic process | 4.05E−05 |
| | vs. SGN-B7H4V | Control | | | | |
| mouse | B7H41001 mAb-DM4 | Up.in.Test/ | BP | GO:1905952 | regulation of lipid localization | 4.15E−05 |
| | vs. SGN-B7H4V | Control | | | | |
| mouse | B7H41001 mAb-DM4 | Down.in.Test/ | BP | GO:0045765 | regulation of angiogenesis | 4.20E−05 |
| | vs. SGN-B7H4V | Control | | | | |
| mouse | B7H41001 mAb-DM4 | Up.in.Test/ | BP | GO:0006637 | acyl-CoA metabolic process | 5.80E−05 |
| | vs. SGN-B7H4V | Control | | | | |
| mouse | B7H41001 mAb-DM4 | Up.in.Test/ | BP | GO:0035383 | thioester metabolic process | 5.80E−05 |
| | vs. SGN-B7H4V | Control | | | | |
| mouse | B7H41001 mAb-DM4 | Up.in.Test/ | BP | GO:0006790 | sulfur compound metabolic process | 6.34E−05 |
| | vs. SGN-B7H4V | Control | | | | |
| mouse | B7H41001 mAb-DM4 | Up.in.Test/ | BP | GO:0033866 | nucleoside bisphosphate biosynthetic process | 6.72E−05 |
| | vs. SGN-B7H4V | Control | | | | |
| mouse | B7H41001 mAb-DM4 | Up.in.Test/ | BP | GO:0034030 | ribonucleoside bisphosphate biosynthetic | 6.72E−05 |
| | vs. SGN-B7H4V | Control | | | process | |
| mouse | B7H41001 mAb-DM4 | Up.in.Test/ | BP | GO:0034033 | purine nucleoside bisphosphate biosynthetic | 6.72E−05 |
| | vs. SGN-B7H4V | Control | | | process | |
| mouse | B7H41001 mAb-DM4 | Up.in.Test/ | BP | GO:0009063 | cellular amino acid catabolic process | 7.30E−05 |
| | vs. SGN-B7H4V | Control | | | | |
| mouse | B7H41001 mAb-DM4 | Up.in.Test/ | BP | GO:0006536 | glutamate metabolic process | 9.53E−05 |
| | vs. SGN-B7H4V | Control | | | | |
| mouse | B7H41001 mAb-DM4 | Down.in.Test/ | BP | GO:0060326 | cell chemotaxis | 1.11E−04 |
| | vs. SGN-B7H4V | Control | | | | |
| mouse | B7H41001 mAb-DM4 | Up.in.Test/ | BP | GO:0035384 | thioester biosynthetic process | 1.19E−04 |
| | vs. SGN-B7H4V | Control | | | | |
| mouse | B7H41001 mAb-DM4 | Up.in.Test/ | BP | GO:0071616 | acyl-CoA biosynthetic process | 1.19E−04 |
| | vs. SGN-B7H4V | Control | | | | |
| mouse | B7H41001 mAb-DM4 | Down.in.Test/ | BP | GO:0071674 | mononuclear cell migration | 1.20E−04 |
| | vs. SGN-B7H4V | Control | | | | |
| mouse | B7H41001 mAb-DM4 | Down.in.Test/ | BP | GO:0032103 | positive regulation of response to external | 1.20E−04 |
| | vs. SGN-B7H4V | Control | | | stimulus | |
| mouse | B7H41001 mAb-DM4 | Up.in.Test/ | BP | GO:1901605 | alpha-amino acid metabolic process | 1.49E−04 |
| | vs. SGN-B7H4V | Control | | | | |
| mouse | B7H41001 mAb-DM4 | Down.in.Test/ | BP | GO:0090287 | regulation of cellular response to growth | 1.85E−04 |
| | vs. SGN-B7H4V | Control | | | factor stimulus | |
| mouse | B7H41001 mAb-DM4 | Down.in.Test/ | BP | GO:0009615 | response to virus | 1.96E−04 |
| | vs. SGN-B7H4V | Control | | | | |
| mouse | B7H41001 mAb-DM4 | Down.in.Test/ | BP | GO:0043535 | regulation of blood vessel endothelial cell | 2.25E−04 |
| | vs. SGN-B7H4V | Control | | | migration | |
| mouse | B7H41001 mAb-DM4 | Down.in.Test/ | BP | GO:0019221 | cytokine-mediated signaling pathway | 2.26E−04 |
| | vs. SGN-B7H4V | Control | | | | |
| mouse | B7H41001 mAb-DM4 | Down.in.Test/ | BP | GO:0071396 | cellular response to lipid | 2.32E−04 |
| | vs. SGN-B7H4V | Control | | | | |
| mouse | B7H41001 mAb-DM4 | Up.in.Test/ | BP | GO:0033108 | mitochondrial respiratory chain complex | 2.74E−04 |
| | vs. SGN-B7H4V | Control | | | assembly | |
| mouse | B7H41001 mAb-DM4 | Down.in.Test/ | BP | GO:0014812 | muscle cell migration | 3.04E−04 |
| | vs. SGN-B7H4V | Control | | | | |
| mouse | B7H41001 mAb-DM4 | Down.in.Test/ | BP | GO:0030336 | negative regulation of cell migration | 3.12E−04 |
| | vs. SGN-B7H4V | Control | | | | |
| mouse | B7H41001 mAb-DM4 | Down.in.Test/ | BP | GO:0097529 | myeloid leukocyte migration | 3.12E−04 |
| | vs. SGN-B7H4V | Control | | | | |
| mouse | B7H41001 mAb-DM4 | Down.in.Test/ | BP | GO:0040013 | negative regulation of locomotion | 3.12E−04 |
| | vs. SGN-B7H4V | Control | | | | |
| mouse | B7H41001 mAb-DM4 | Up.in.Test/ | BP | GO:0006085 | acetyl-CoA biosynthetic process | 3.80E−04 |
| | vs. SGN-B7H4V | Control | | | | |
| mouse | B7H41001 mAb-DM4 | Down.in.Test/ | BP | GO:0030595 | eukocyte chemotaxis | 4.18E−04 |
| | vs. SGN-B7H4V | Control | | | | |
| mouse | B7H41001 mAb-DM4 | Down.in.Test/ | BP | GO:0001503 | ossification | 4.18E−04 |
| | vs. SGN-B7H4V | Control | | | | |
| mouse | B7H41001 mAb-DM4 | Down.in.Test/ | BP | GO:0071559 | response to transforming growth factor beta | 4.89E−04 |
| | vs. SGN-B7H4V | Control | | | | |
| mouse | B7H41001 mAb-DM4 | Down.in.Test/ | BP | GO:2000146 | negative regulation of cell motility | 5.18E−04 |
| | vs. SGN-B7H4V | Control | | | | |
| mouse | B7H41001 mAb-DM4 | Down.in.Test/ | BP | GO:0043536 | positive regulation of blood vessel endothelial | 5.18E−04 |
| | vs. SGN-B7H4V | Control | | | cell migration | |
| mouse | B7H41001 mAb-DM4 | Down.in.Test/ | BP | GO:0061041 | regulation of wound healing | 5.18E−04 |
| | vs. SGN-B7H4V | Control | | | | |
| mouse | B7H41001 mAb-DM4 | Down.in.Test/ | BP | GO:1903034 | regulation of response to wounding | 5.95E−04 |
| | vs. SGN-B7H4V | Control | | | | |
| mouse | B7H41001 mAb-DM4 | Down.in.Test/ | BP | GO:2000181 | negative regulation of blood vessel | 6.01E−04 |
| | vs. SGN-B7H4V | Control | | | morphogenesis | |
| mouse | B7H41001 mAb-DM4 | Down.in.Test/ | BP | GO:1901343 | negative regulation of vasculature | 6.45E−04 |
| | vs. SGN-B7H4V | Control | | | development | |
| mouse | B7H41001 mAb-DM4 | Down.in.Test/ | BP | GO:0045785 | positive regulation of cell adhesion | 6.46E−04 |
| | vs. SGN-B7H4V | Control | | | | |
| mouse | B7H41001 mAb-DM4 | Down.in.Test/ | BP | GO:0051271 | negative regulation of cellular component | 6.89E−04 |
| | vs. SGN-B7H4V | Control | | | movement | |
| mouse | B7H41001 mAb-DM4 | Up.in.Test/ | BP | GO:0006084 | acetyl-CoA metabolic process | 7.49E−04 |
| | vs. SGN-B7H4V | Control | | | | |
| mouse | B7H41001 mAb-DM4 | Down.in.Test/ | BP | GO:0003158 | endothelium development | 8.12E−04 |
| | vs. SGN-B7H4V | Control | | | | |
| mouse | B7H41001 mAb-DM4 | Up.in.Test/ | BP | GO:0010565 | regulation of cellular ketone metabolic | 8.47E−04 |
| | vs. SGN-B7H4V | Control | | | process | |
| mouse | B7H41001 mAb-DM4 | Up.in.Test/ | BP | GO:0009065 | glutamine family amino acid catabolic process | 1.05E−03 |
| | vs. SGN-B7H4V | Control | | | | |
| mouse | B7H41001 mAb-DM4 | Down.in.Test/ | BP | GO:0048010 | vascular endothelial growth factor receptor | 1.07E−03 |
| | vs. SGN-B7H4V | Control | | | signaling pathway | |
| mouse | B7H41001 mAb-DM4 | Down.in.Test/ | BP | GO:0035791 | platelet-derived growth factor receptor-beta | 1.07E−03 |
| | vs. SGN-B7H4V | Control | | | signaling pathway | |
| mouse | B7H41001 mAb-DM4 | Down.in.Test/ | BP | GO:0071560 | cellular response to transforming growth | 1.07E−03 |
| | vs. SGN-B7H4V | Control | | | factor beta stimulus | |
| mouse | B7H41001 mAb-DM4 | Down.in.Test/ | BP | GO:0043534 | blood vessel endothelial cell migration | 1.14E−03 |
| | vs. SGN-B7H4V | Control | | | | |
| mouse | B7H41001 mAb-DM4 | Up.in.Test/ | BP | GO:0032543 | mitochondrial translation | 1.14E−03 |
| | vs. SGN-B7H4V | Control | | | | |
| mouse | B7H41001 mAb-DM4 | Down.in.Test/ | BP | GO:0001649 | osteoblast differentiation | 1.34E−03 |
| | vs. SGN-B7H4V | Control | | | | |
| mouse | B7H41001 mAb-DM4 | Down.in.Test/ | BP | GO:0014909 | smooth muscle cell migration | 1.37E−03 |
| | vs. SGN-B7H4V | Control | | | | |
| mouse | B7H41001 mAb-DM4 | Up.in.Test/ | BP | GO:0009437 | carnitine metabolic process | 1.38E−03 |
| | vs. SGN-B7H4V | Control | | | | |
| mouse | B7H41001 mAb-DM4 | Down.in.Test/ | BP | GO:0017015 | regulation of transforming growth factor beta | 1.51E−03 |
| | vs. SGN-B7H4V | Control | | | receptor signaling pathway | |
| mouse | B7H41001 mAb-DM4 | Down.in.Test/ | BP | GO:0007179 | transforming growth factor beta receptor | 1.52E−03 |
| | vs. SGN-B7H4V | Control | | | signaling pathway | |
| mouse | B7H41001 mAb-DM4 | Down.in.Test/ | BP | GO:0001935 | endothelial cell proliferation | 1.53E−03 |
| | vs. SGN-B7H4V | Control | | | | |
| mouse | B7H41001 mAb-DM4 | Down.in.Test/ | BP | GO:1903844 | regulation of cellular response to transforming | 1.80E−03 |
| | vs. SGN-B7H4V | Control | | | growth factor beta stimulus | |
| mouse | B7H41001 mAb-DM4 | Down.in.Test/ | BP | GO:0090049 | regulation of cell migration involved in | 1.80E−03 |
| | vs. SGN-B7H4V | Control | | | sprouting angiogenesis | |
| mouse | B7H41001 mAb-DM4 | Down.in.Test/ | BP | GO:0016525 | negative regulation of angiogenesis | 1.93E−03 |
| | vs. SGN-B7H4V | Control | | | | |
| mouse | B7H41001 mAb-DM4 | Down.in.Test/ | BP | GO:0046883 | regulation of hormone secretion | 1.97E−03 |
| | vs. SGN-B7H4V | Control | | | | |
| mouse | B7H41001 mAb-DM4 | Down.in.Test/ | BP | GO:0045071 | negative regulation of viral genome | 1.97E−03 |
| | vs. SGN-B7H4V | Control | | | replication | |
| mouse | B7H41001 mAb-DM4 | Down.in.Test/ | BP | GO:0002237 | response to molecule of bacterial origin | 1.97E−03 |
| | vs. SGN-B7H4V | Control | | | | |
| mouse | B7H41001 mAb-DM4 | Down.in.Test/ | BP | GO:0045766 | positive regulation of angiogenesis | 1.97E−03 |
| | vs. SGN-B7H4V | Control | | | | |
| mouse | B7H41001 mAb-DM4 | Down.in.Test/ | BP | GO:1904018 | positive regulation of vasculature | 1.97E−03 |
| | vs. SGN-B7H4V | Control | | | development | |
| mouse | B7H41001 mAb-DM4 | Up.in.Test/ | BP | GO:0140053 | mitochondrial gene expression | 2.07E−03 |
| | vs. SGN-B7H4V | Control | | | | |
| mouse | B7H41001 mAb-DM4 | Down.in.Test/ | BP | GO:0051607 | defense response to virus | 2.15E−03 |
| | vs. SGN-B7H4V | Control | | | | |
| mouse | B7H41001 mAb-DM4 | Down.in.Test/ | BP | GO:0140546 | defense response to symbiont | 2.15E−03 |
| | vs. SGN-B7H4V | Control | | | | |
| mouse | B7H41001 mAb-DM4 | Up.in.Test/ | BP | GO:0050994 | regulation of lipid catabolic process | 2.16E−03 |
| | vs. SGN-B7H4V | Control | | | | |
| mouse | B7H41001 mAb-DM4 | Down.in.Test/ | BP | GO:0050729 | positive regulation of inflammatory response | 2.20E−03 |
| | vs. SGN-B7H4V | Control | | | | |
| mouse | B7H41001 mAb-DM4 | Down.in.Test/ | BP | GO:0043367 | CD4-positive, alpha-beta T cell differentiation | 2.20E−03 |
| | vs. SGN-B7H4V | Control | | | | |
| mouse | B7H41001 mAb-DM4 | Down.in.Test/ | BP | GO:0046879 | hormone secretion | 2.20E−03 |
| | vs. SGN-B7H4V | Control | | | | |
| mouse | B7H41001 mAb-DM4 | Up.in.Test/ | BP | GO:0072593 | reactive oxygen species metabolic process | 2.24E−03 |
| | vs. SGN-B7H4V | Control | | | | |
| mouse | B7H41001 mAb-DM4 | Down.in.Test/ | BP | GO:0042060 | wound healing | 2.26E−03 |
| | vs. SGN-B7H4V | Control | | | | |
| mouse | B7H41001 mAb-DM4 | Down.in.Test/ | BP | GO:0032496 | response to lipopolysaccharide | 2.30E−03 |
| | vs. SGN-B7H4V | Control | | | | |
| mouse | B7H41001 mAb-DM4 | Up.in.Test/ | BP | GO:1990845 | adaptive thermogenesis | 2.31E−03 |
| | vs. SGN-B7H4V | Control | | | | |
| mouse | B7H41001 mAb-DM4 | Down.in.Test/ | BP | GO:0002685 | regulation of leukocyte migration | 2.36E−03 |
| | vs. SGN-B7H4V | Control | | | | |
| mouse | B7H41001 mAb-DM4 | Up.in.Test/ | BP | GO:1901615 | organic hydroxy compound metabolic process | 2.36E−03 |
| | vs. SGN-B7H4V | Control | | | | |
| mouse | B7H41001 mAb-DM4 | Up.in.Test/ | BP | GO:0042180 | cellular ketone metabolic process | 2.36E−03 |
| | vs. SGN-B7H4V | Control | | | | |
| mouse | B7H41001 mAb-DM4 | Up.in.Test/ | BP | GO:0006577 | amino-acid betaine metabolic process | 2.42E−03 |
| | vs. SGN-B7H4V | Control | | | | |
| mouse | B7H41001 mAb-DM4 | Up.in.Test/ | BP | GO:0009083 | branched-chain amino acid catabolic process | 2.42E−03 |
| | vs. SGN-B7H4V | Control | | | | |
| mouse | B7H41001 mAb-DM4 | Down.in.Test/ | BP | GO:0090092 | regulation of transmembrane receptor protein | 2.63E−03 |
| | vs. SGN-B7H4V | Control | | | serine/threonine kinase signaling pathway | |
| mouse | B7H41001 mAb-DM4 | Down.in.Test/ | BP | GO:0014910 | regulation of smooth muscle cell migration | 2.70E−03 |
| | vs. SGN-B7H4V | Control | | | | |
| mouse | B7H41001 mAb-DM4 | Down.in.Test/ | BP | GO:0009914 | hormone transport | 2.70E−03 |
| | vs. SGN-B7H4V | Control | | | | |
| mouse | B7H41001 mAb-DM4 | Down.in.Test/ | BP | GO:0035710 | CD4-positive, alpha-beta T cell activation | 2.89E−03 |
| | vs. SGN-B7H4V | Control | | | | |
| mouse | B7H41001 mAb-DM4 | Down.in.Test/ | BP | GO:0050727 | regulation of inflammatory response | 2.99E−03 |
| | vs. SGN-B7H4V | Control | | | | |
| mouse | B7H41001 mAb-DM4 | Down.in.Test/ | BP | GO:0045123 | cellular extravasation | 3.06E−03 |
| | vs. SGN-B7H4V | Control | | | | |
| mouse | B7H41001 mAb-DM4 | Down.in.Test/ | BP | GO:1903706 | regulation of hemopoiesis | 3.06E−03 |
| | vs. SGN-B7H4V | Control | | | | |
| mouse | B7H41001 mAb-DM4 | Down.in.Test/ | BP | GO:0007178 | transmembrane receptor protein | 3.09E−03 |
| | vs. SGN-B7H4V | Control | | | serine/threonine kinase signaling pathway | |
| mouse | B7H41001 mAb-DM4 | Down.in.Test/ | BP | GO:0034341 | response to interferon-gamma | 3.10E−03 |
| | vs. SGN-B7H4V | Control | | | | |
| mouse | B7H41001 mAb-DM4 | Up.in.Test/ | BP | GO:0006167 | AMP biosynthetic process | 3.12E−03 |
| | vs. SGN-B7H4V | Control | | | | |
| mouse | B7H41001 mAb-DM4 | Up.in.Test/ | BP | GO:0006188 | IMP biosynthetic process | 3.12E−03 |
| | vs. SGN-B7H4V | Control | | | | |
| mouse | B7H41001 mAb-DM4 | Up.in.Test/ | BP | GO:0072350 | tricarboxylic acid metabolic process | 3.12E−03 |
| | vs. SGN-B7H4V | Control | | | | |
| mouse | B7H41001 mAb-DM4 | Down.in.Test/ | BP | GO:0006986 | response to unfolded protein | 3.12E−03 |
| | vs. SGN-B7H4V | Control | | | | |
| mouse | B7H41001 mAb-DM4 | Up.in.Test/ | BP | GO:0044272 | sulfur compound biosynthetic process | 3.32E−03 |
| | vs. SGN-B7H4V | Control | | | | |
| mouse | B7H41001 mAb-DM4 | Up.in.Test/ | BP | GO:0006839 | mitochondrial transport | 3.40E−03 |
| | vs. SGN-B7H4V | Control | | | | |
| mouse | B7H41001 mAb-DM4 | Up.in.Test/ | BP | GO:0032368 | regulation of lipid transport | 3.59E−03 |
| | vs. SGN-B7H4V | Control | | | | |
| mouse | B7H41001 mAb-DM4 | Up.in.Test/ | BP | GO:0046034 | ATP metabolic process | 3.60E−03 |
| | vs. SGN-B7H4V | Control | | | | |
| mouse | B7H41001 mAb-DM4 | Up.in.Test/ | BP | GO:1901606 | alpha-amino acid catabolic process | 3.65E−03 |
| | vs. SGN-B7H4V | Control | | | | |
| mouse | B7H41001 mAb-DM4 | Up.in.Test/ | BP | GO:1905954 | positive regulation of lipid localization | 3.65E−03 |
| | vs. SGN-B7H4V | Control | | | | |
| mouse | B7H41001 mAb-DM4 | Down.in.Test/ | BP | GO:0009611 | response to wounding | 3.67E−03 |
| | vs. SGN-B7H4V | Control | | | | |
| mouse | B7H41001 mAb-DM4 | Up.in.Test/ | BP | GO:0120161 | regulation of cold-induced thermogenesis | 3.84E−03 |
| | vs. SGN-B7H4V | Control | | | | |
| mouse | B7H41001 mAb-DM4 | Down.in.Test/ | BP | GO:0048008 | platelet-derived growth factor receptor | 3.89E−03 |
| | vs. SGN-B7H4V | Control | | | signaling pathway | |
| mouse | B7H41001 mAb-DM4 | Up.in.Test/ | BP | GO:0019318 | hexose metabolic process | 4.07E−03 |
| | vs. SGN-B7H4V | Control | | | | |
| mouse | B7H41001 mAb-DM4 | Down.in.Test/ | BP | GO:0071222 | cellular response to lipopolysaccharide | 4.18E−03 |
| | vs. SGN-B7H4V | Control | | | | |
| mouse | B7H41001 mAb-DM4 | Up.in.Test/ | BP | GO:0106106 | cold-induced thermogenesis | 4.21E−03 |
| | vs. SGN-B7H4V | Control | | | | |
| mouse | B7H41001 mAb-DM4 | Down.in.Test/ | BP | GO:0060393 | regulation of pathway-restricted SMAD | 4.25E−03 |
| | vs. SGN-B7H4V | Control | | | protein phosphorylation | |
| mouse | B7H41001 mAb-DM4 | Down.in.Test/ | BP | GO:0031032 | actomyosin structure organization | 4.25E−03 |
| | vs. SGN-B7H4V | Control | | | | |
| mouse | B7H41001 mAb-DM4 | Down.in.Test/ | BP | GO:0032640 | tumor necrosis factor production | 4.26E−03 |
| | vs. SGN-B7H4V | Control | | | | |
| mouse | B7H41001 mAb-DM4 | Up.in.Test/ | BP | GO:0006177 | GMP biosynthetic process | 4.62E−03 |
| | vs. SGN-B7H4V | Control | | | | |
| mouse | B7H41001 mAb-DM4 | Up.in.Test/ | BP | GO:0009081 | branched-chain amino acid metabolic process | 4.62E−03 |
| | vs. SGN-B7H4V | Control | | | | |
| mouse | B7H41001 mAb-DM4 | Up.in.Test/ | BP | GO:0006119 | oxidative phosphorylation | 4.62E−03 |
| | vs. SGN-B7H4V | Control | | | | |
| mouse | B7H41001 mAb-DM4 | Up.in.Test/ | BP | GO:0062012 | regulation of small molecule metabolic | 4.62E−03 |
| | vs. SGN-B7H4V | Control | | | process | |
| mouse | B7H41001 mAb-DM4 | Up.in.Test/ | BP | GO:0120162 | positive regulation of cold-induced | 4.88E−03 |
| | vs. SGN-B7H4V | Control | | | thermogenesis | |
| mouse | B7H41001 mAb-DM4 | Down.in.Test/ | BP | GO:0002040 | sprouting angiogenesis | 4.93E−03 |
| | vs. SGN-B7H4V | Control | | | | |
| mouse | B7H41001 mAb-DM4 | Down.in.Test/ | BP | GO:0071706 | tumor necrosis factor superfamily cytokine | 4.95E−03 |
| | vs. SGN-B7H4V | Control | | | production | |
| mouse | B7H41001 mAb-DM4 | Down.in.Test/ | BP | GO:0042093 | T-helper cell differentiation | 5.56E−03 |
| | vs. SGN-B7H4V | Control | | | | |
| mouse | B7H41001 mAb-DM4 | Down.in.Test/ | BP | GO:0060389 | pathway-restricted SMAD protein | 5.56E−03 |
| | vs. SGN-B7H4V | Control | | | phosphorylation | |
| mouse | B7H41001 mAb-DM4 | Down.in.Test/ | BP | GO:0035296 | regulation of tube diameter | 5.59E−03 |
| | vs. SGN-B7H4V | Control | | | | |
| mouse | B7H41001 mAb-DM4 | Down.in.Test/ | BP | GO:0097746 | blood vessel diameter maintenance | 5.59E−03 |
| | vs. SGN-B7H4V | Control | | | | |
| mouse | B7H41001 mAb-DM4 | Down.in.Test/ | BP | GO:0001936 | regulation of endothelial cell proliferation | 5.60E−03 |
| | vs. SGN-B7H4V | Control | | | | |
| mouse | B7H41001 mAb-DM4 | Down.in.Test/ | BP | GO:0035150 | regulation of tube size | 5.80E−03 |
| | vs. SGN-B7H4V | Control | | | | |
| mouse | B7H41001 mAb-DM4 | Down.in.Test/ | BP | GO:0002294 | CD4-positive, alpha-beta T cell differentiation | 5.85E−03 |
| | vs. SGN-B7H4V | Control | | | involved in immune response | |
| mouse | B7H41001 mAb-DM4 | Down.in.Test/ | BP | GO:0071219 | cellular response to molecule of bacterial | 5.97E−03 |
| | vs. SGN-B7H4V | Control | | | origin | |
| mouse | B7H41001 mAb-DM4 | Down.in.Test/ | BP | GO:0090303 | positive regulation of wound healing | 6.03E−03 |
| | vs. SGN-B7H4V | Control | | | | |
| mouse | B7H41001 mAb-DM4 | Down.in.Test/ | BP | GO:0002687 | positive regulation of leukocyte migration | 6.03E−03 |
| | vs. SGN-B7H4V | Control | | | | |
| mouse | B7H41001 mAb-DM4 | Down.in.Test/ | BP | GO:0001952 | regulation of cell-matrix adhesion | 6.16E−03 |
| | vs. SGN-B7H4V | Control | | | | |
| mouse | B7H41001 mAb-DM4 | Down.in.Test/ | BP | GO:0002293 | alpha-beta T cell differentiation involved in | 6.16E−03 |
| | vs. SGN-B7H4V | Control | | | immune response | |
| mouse | B7H41001 mAb-DM4 | Up.in.Test/ | BP | GO:2000378 | negative regulation of reactive oxygen species | 6.61E−03 |
| | vs. SGN-B7H4V | Control | | | metabolic process | |
| mouse | B7H41001 mAb-DM4 | Down.in.Test/ | BP | GO:0032102 | negative regulation of response to external | 6.63E−03 |
| | vs. SGN-B7H4V | Control | | | stimulus | |
| mouse | B7H41001 mAb-DM4 | Down.in.Test/ | BP | GO:0002287 | alpha-beta T cell activation involved in | 6.63E−03 |
| | vs. SGN-B7H4V | Control | | | immune response | |
| mouse | B7H41001 mAb-DM4 | Down.in.Test/ | BP | GO:1903036 | positive regulation of response to wounding | 6.63E−03 |
| | vs. SGN-B7H4V | Control | | | | |
| mouse | B7H41001 mAb-DM4 | Down.in.Test/ | BP | GO:0010810 | regulation of cell-substrate adhesion | 6.68E−03 |
| | vs. SGN-B7H4V | Control | | | | |
| mouse | B7H41001 mAb-DM4 | Down.in.Test/ | BP | GO:0007030 | Golgi organization | 6.68E−03 |
| | vs. SGN-B7H4V | Control | | | | |
| mouse | B7H41001 mAb-DM4 | Up.in.Test/ | BP | GO:0001659 | temperature homeostasis | 6.68E−03 |
| | vs. SGN-B7H4V | Control | | | | |
| mouse | B7H41001 mAb-DM4 | Up.in.Test/ | BP | GO:0009067 | aspartate family amino acid biosynthetic | 6.87E−03 |
| | vs. SGN-B7H4V | Control | | | process | |
| mouse | B7H41001 mAb-DM4 | Down.in.Test/ | BP | GO:0001763 | morphogenesis of a branching structure | 6.95E−03 |
| | vs. SGN-B7H4V | Control | | | | |
| mouse | B7H41001 mAb-DM4 | Down.in.Test/ | BP | GO:0002042 | cell migration involved in sprouting | 6.95E−03 |
| | vs. SGN-B7H4V | Control | | | angiogenesis | |
| mouse | B7H41001 mAb-DM4 | Down.in.Test/ | BP | GO:0140353 | lipid export from cell | 6.95E−03 |
| | vs. SGN-B7H4V | Control | | | | |
| mouse | B7H41001 mAb-DM4 | Up.in.Test/ | BP | GO:0005996 | monosaccharide metabolic process | 7.24E−03 |
| | vs. SGN-B7H4V | Control | | | | |
| mouse | B7H41001 mAb-DM4 | Down.in.Test/ | BP | GO:0050920 | regulation of chemotaxis | 7.44E−03 |
| | vs. SGN-B7H4V | Control | | | | |
| mouse | B7H41001 mAb-DM4 | Down.in.Test/ | BP | GO:0035456 | response to interferon-beta | 7.63E−03 |
| | vs. SGN-B7H4V | Control | | | | |
| mouse | B7H41001 mAb-DM4 | Down.in.Test/ | BP | GO:0050673 | epithelial cell proliferation | 7.63E−03 |
| | vs. SGN-B7H4V | Control | | | | |
| mouse | B7H41001 mAb-DM4 | Down.in.Test/ | BP | GO:0030198 | extracellular matrix organization | 7.90E−03 |
| | vs. SGN-B7H4V | Control | | | | |
| mouse | B7H41001 mAb-DM4 | Down.in.Test/ | BP | GO:0043062 | extracellular structure organization | 8.13E−03 |
| | vs. SGN-B7H4V | Control | | | | |
| mouse | B7H41001 mAb-DM4 | Down.in.Test/ | BP | GO:0031349 | positive regulation of defense response | 8.13E−03 |
| | vs. SGN-B7H4V | Control | | | | |
| mouse | B7H41001 mAb-DM4 | Up.in.Test/ | BP | GO:0046320 | regulation of fatty acid oxidation | 8.21E−03 |
| | vs. SGN-B7H4V | Control | | | | |
| mouse | B7H41001 mAb-DM4 | Up.in.Test/ | BP | GO:0031998 | regulation of fatty acid beta-oxidation | 8.21E−03 |
| | vs. SGN-B7H4V | Control | | | | |
| mouse | B7H41001 mAb-DM4 | Down.in.Test/ | BP | GO:0045229 | external encapsulating structure organization | 8.33E−03 |
| | vs. SGN-B7H4V | Control | | | | |
| mouse | B7H41001 mAb-DM4 | Down.in.Test/ | BP | GO:0031589 | cell-substrate adhesion | 8.45E−03 |
| | vs. SGN-B7H4V | Control | | | | |
| mouse | B7H41001 mAb-DM4 | Down.in.Test/ | BP | GO:0051222 | positive regulation of protein transport | 8.45E−03 |
| | vs. SGN-B7H4V | Control | | | | |
| mouse | B7H41001 mAb-DM4 | Down.in.Test/ | BP | GO:0032680 | regulation of tumor necrosis factor production | 8.45E−03 |
| | vs. SGN-B7H4V | Control | | | | |
| mouse | B7H41001 mAb-DM4 | Down.in.Test/ | BP | GO:0008217 | regulation of blood pressure | 8.45E−03 |
| | vs. SGN-B7H4V | Control | | | | |
| mouse | B7H41001 mAb-DM4 | Down.in.Test/ | BP | GO:0070555 | response to interleukin-1 | 8.45E−03 |
| | vs. SGN-B7H4V | Control | | | | |
| mouse | B7H41001 mAb-DM4 | Down.in.Test/ | BP | GO:0035966 | response to topologically incorrect protein | 8.62E−03 |
| | vs. SGN-B7H4V | Control | | | | |
| mouse | B7H41001 mAb-DM4 | Down.in.Test/ | BP | GO:0030512 | negative regulation of transforming growth | 9.10E−03 |
| | vs. SGN-B7H4V | Control | | | factor beta receptor signaling pathway | |
| mouse | B7H41001 mAb-DM4 | Up.in.Test/ | BP | GO:0010876 | lipid localization | 9.24E−03 |
| | vs. SGN-B7H4V | Control | | | | |
| mouse | B7H41001 mAb-DM4 | Down.in.Test/ | BP | GO:1903555 | regulation of tumor necrosis factor | 9.61E−03 |
| | vs. SGN-B7H4V | Control | | | superfamily cytokine production | |
| mouse | B7H41001 mAb-DM4 | Down.in.Test/ | BP | GO:0002548 | monocyte chemotaxis | 9.61E−03 |
| | vs. SGN-B7H4V | Control | | | | |
| mouse | B7H41001 mAb-DM4 | Down.in.Test/ | BP | GO:0002292 | T cell differentiation involved in immune | 9.75E−03 |
| | vs. SGN-B7H4V | Control | | | response | |
| mouse | B7H41001 mAb-DM4 | Down.in.Test/ | BP | GO:0045637 | regulation of myeloid cell differentiation | 9.78E−03 |
| | vs. SGN-B7H4V | Control | | | | |
| mouse | B7H41001 mAb-DM4 | Down.in.Test/ | BP | GO:1903532 | positive regulation of secretion by cell | 9.80E−03 |
| | vs. SGN-B7H4V | Control | | | | |
| mouse | B7H41001 mAb-DM4 | Down.in.Test/ | BP | GO:0009101 | glycoprotein biosynthetic process | 9.80E−03 |
| | vs. SGN-B7H4V | Control | | | | |
| mouse | B7H41001 mAb-DM4 | Down.in.Test/ | BP | GO:0048608 | reproductive structure development | 9.87E−03 |
| | vs. SGN-B7H4V | Control | | | | |
| mouse | B7H41001 mAb-DM4 | Up.in.Test/ | BP | GO:0022900 | electron transport chain | 9.96E−03 |
| | vs. SGN-B7H4V | Control | | | | |

### Example 17: SGN-B7H4V mIgG2a Elicits Robust Activity in the Immunocompetent Murine B7-H4-Expressing Renca Tumor Model

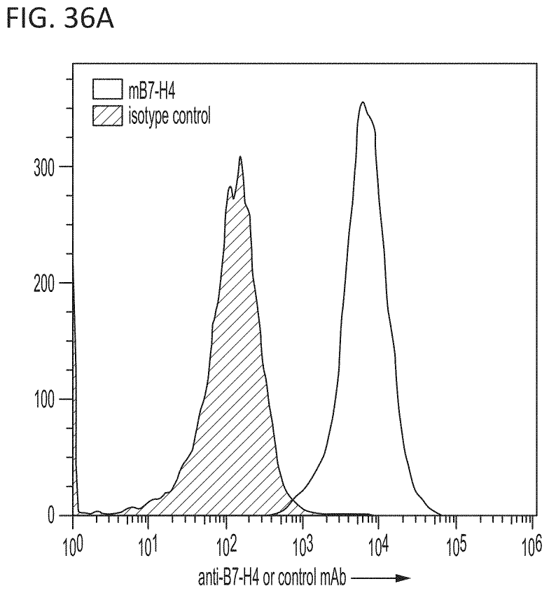

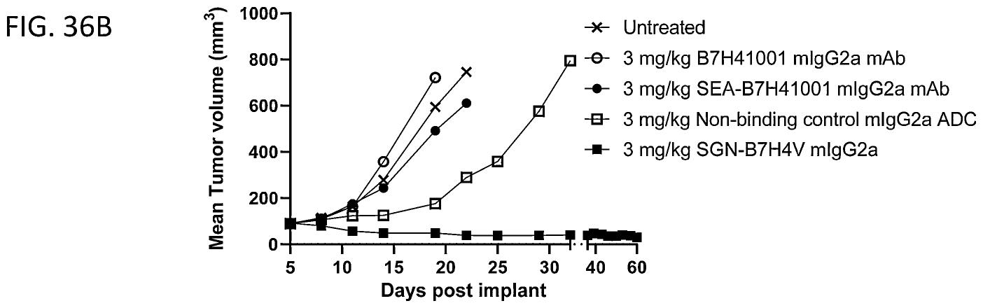

The activity of SGN-B7H4V mIgG2a (antibody-drug conjugate, or “ADC” hereafter) was then evaluated in an immunocompetent murine Renca tumor model engineered via lentiviral transduction to express murine B7-H4 (mB7-H4, FIG. 36A).

Murine B7-H4-expressing Renca cells were cultured in RPMI-1640 (ATCC) with 10% heat-inactivated fetal bovine serum, MEM non-essential amino acids (1×), sodium pyruvate (1 mM), and L-glutamine (2 mM). Renca cancer cells were implanted (2×10⁶ cells in 200 μL 25% Matrigel in RPMI-1640 medium) subcutaneously into Balb/c female mice. Once tumor volumes reached˜100 mm³, mice were randomized into treatment groups of 5-10 mice each.

mB7-H4-Renca tumor-bearing mice were treated with 3 weekly doses of 3 mg/kg unconjugated antibody or ADCs when tumor volumes reach 100 mm³. Antibodies and ADCs were prepared with a murine IgG2a (mIgG2a) backbone, rather than the human IgG1 (hIgG1) backbone used in the clinical therapeutic, to avoid elicitation of an anti-drug antibody response against the xenogeneic human antibody.

SGN-B7H4V mIgG2a Drove Robust Anti-Tumor Responses in an Immunocompetent Murine B7-H4-Expressing Renca Tumor Model

Treatment with SGN-B7H4V mIgG2a caused sustained tumor regression in all mice, while in contrast the non-binding control mIgG2a ADC elicited modest tumor growth delay. On the other hand, treatment with the unconjugated B7-H4-targeted antibodies B7H41001 mIgG2a (fucosylated Fc backbone) as well as SEA-B7H41001 mIgG2a (Fc effector function enhanced afucosylated Fc backbone) elicited minimal anti-tumor activity (FIG. 36B).

This demonstrates that enhanced anti-tumor activity was achieved with the targeted ADC approach, in which a B7-H4-targeted antibody was empowered with a vedotin payload.

### Example 18: SGN-B7H4V mIgG2a Recruits Multiple Immune Cell Types to Murine B7-H4-Expressing Renca Tumors

Murine B7-H4-expressing Renca-tumor bearing mice were then treated i.p. with a single 3 mg/kg dose of vehicle, the unconjugated B7H41001 mIgG2a mAb, the non-binding control mIgG2a ADC, or SGN-B7H4V mIgG2a. Tumors were harvested 6-7 days after treatment, cut in half, and processed for RNA-seq or IHC. The ability of SGN-B7H4V mIgG2a to elicit immunomodulatory changes, including recruitment of immune cells to the Renca tumor site, was evaluated by analysis of gene expression changes by RNA-seq as well as immunohistochemical staining as described above.

SGN-B7H4V mIgG2a Induces Upregulation of Cytokines and Type I IFN Response Genes in Murine B7-H4-Expressing Renca Tumors.

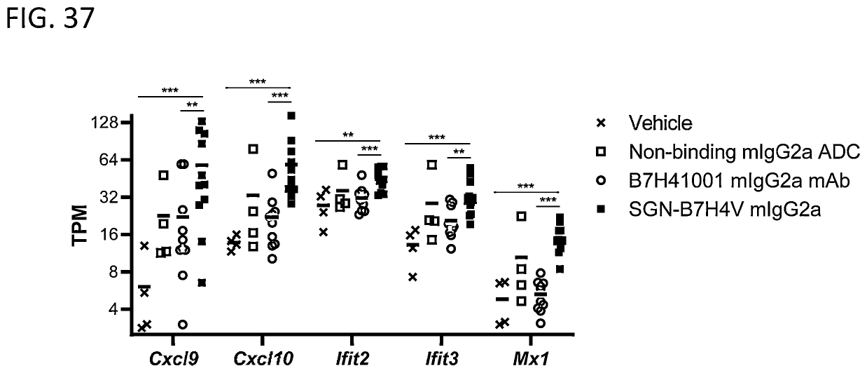

RNAseq analysis of SGN-B7H4V mIgG2a-treated tumors revealed a significant increase in transcripts encoding cytokines and type I IFN response genes (FIG. 37) following treatment with SGN-B7H4V mIgG2a compared to the unconjugated mAb B7H41001. The expression of these genes may promote immune cell activation and recruitment to tumors. There was a trend (though not statistically significant) towards an increase in some of these transcripts (e.g. Cxcl9) following treatment with B7H41001 mIgG2a mAb compared to vehicle; however, maximal changes occurred following treatment with SGN-B7H4V mIgG2a highlighting the advantage of a B7-H4-targeted antibody that is empowered with a vedotin payload.

SGN-B7H4V mIgG2a Elicited Recruitment of Antigen-Presenting Cells to Murine B7-H4-Expressing Renca Tumors as Well as Upregulation of MHC Class II and Costimulatory Molecules

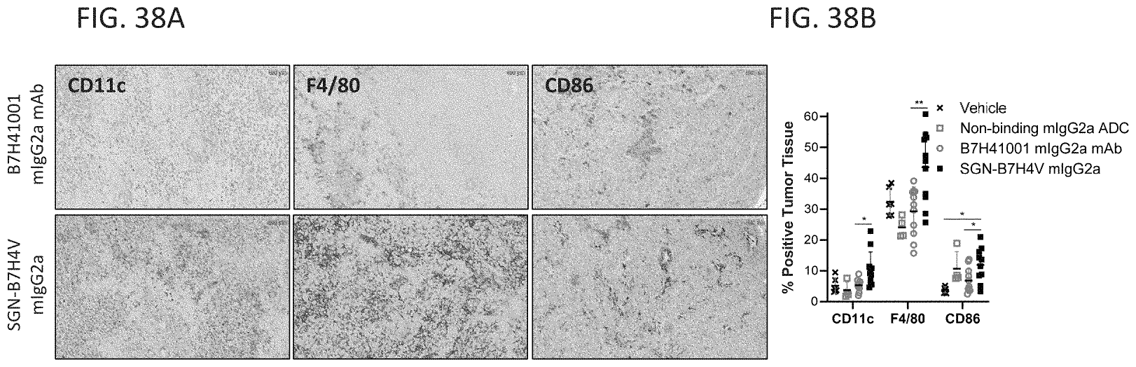

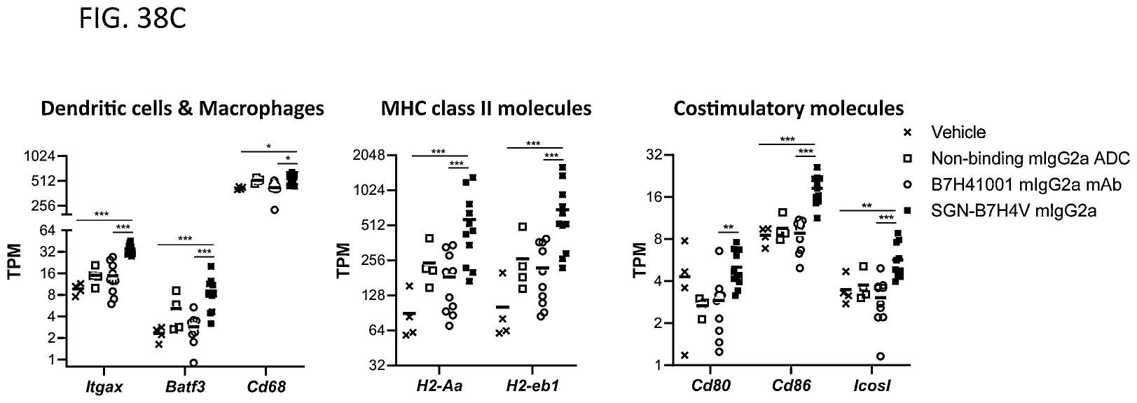

Quantification of stained tumor sections revealed an increase in the proportion of CD11c+ dendritic cells, F4/80+ macrophages, and cells expressing the co-stimulatory molecule CD86 in SGN-B7H4V mIgG2a-treated tumors (FIGS. 38A and 38B). This was corroborated by RNAseq analysis, which revealed a significant increase in Itgax (encodes CD11c). Batf3 (encodes BatF3, a transcription factor associated with antigen cross-presentation), Cd68 (encodes the macrophage marker CD68), H2-Aa & H2-eb1 (encode MHC class II molecules), and Cd80, Cd86, & Icos1 (encode costimulatory molecules) transcripts following treatment with SGN-B7H4V mIgG2a (FIG. 38C). This suggests that SGN-B7H4V mIgG2a can promote recruitment of innate antigen-presenting cells to tumors as well as upregulation of genes associated with antigen presentation to T cells including MHC class II molecules as well as multiple co-stimulatory molecules. There was a trend (though not statistically significant) towards an increase in some of these transcripts (e.g. H2-Aa and H2-eb1) following treatment with B7H41001 mIgG2a mAb compared to vehicle; however, maximal changes occurred following treatment with SGN-B7H4V mIgG2a highlighting the advantage of a B7-H4-targeted antibody that is empowered with a vedotin payload.

SGN-B7H4V mIgG2a Elicited Recruitment of CD4 and CD8 T Cells to Murine B7-H4-Expressing Renca Tumors

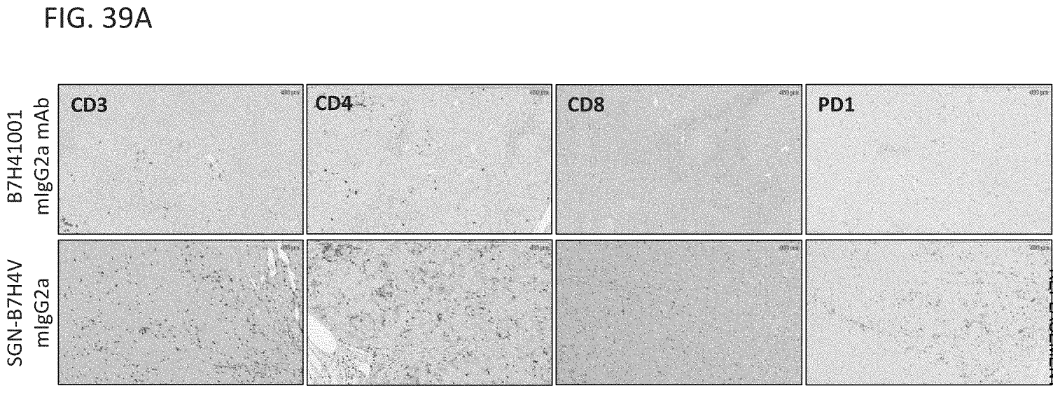

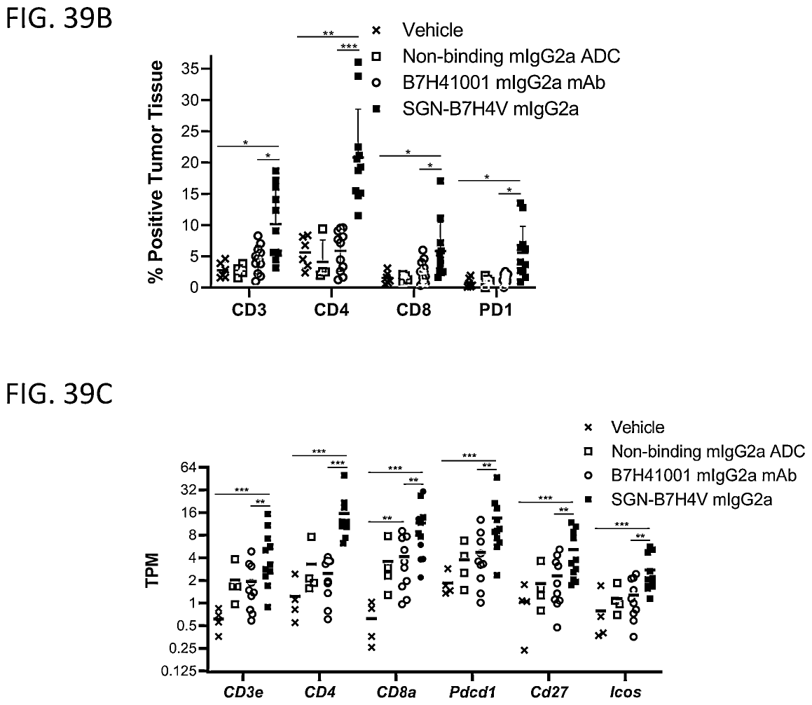

Quantification of stained tumor sections also revealed an increase in the proportion of CD3+, CD4+, and CD8+ T cells as well as cells expressing PD-1, the receptor for PD-L1 that is upregulated on newly activated T cells, in SGN-B7H4V mIgG2a-treated tumors (FIGS. 39A and 39B). This was corroborated by RNAseq analysis, which revealed an increase in Cd3e (which encodes the T cell marker CD3), Cd4 (which encodes the T cell marker CD4), Cd8a (which encodes the T cell marker CD8), Pdcd1 (which encodes PD-1), Cd27 (which encodes CD27), and Icos (which encodes ICOS) transcripts following treatment with SGN-B7H4V mIgG2a (FIG. 39C). Altogether, this suggests that SGN-B7H4V mIgG2a can promote recruitment of adaptive T cells to tumors and upregulation of genes associated with early T cell activation such as PD-1, CD27, and ICOS. There was a trend (though, with the exception of CD8a, not statistically significant) towards an increase in these transcripts following treatment with B7H41001 mIgG2a mAb compared to vehicle; however, maximal changes occurred following treatment with SGN-B7H4V mIgG2a highlighting the advantage of a B7-H4-targeted antibody that is empowered with a vedotin payload.

SGN-B7H4V mIgG2a Drives Upregulation of Genes that have been Associated Clinically with Response to Anti-PD(L)1 Agents in Murine B7-H4-Expressing Renca Tumors

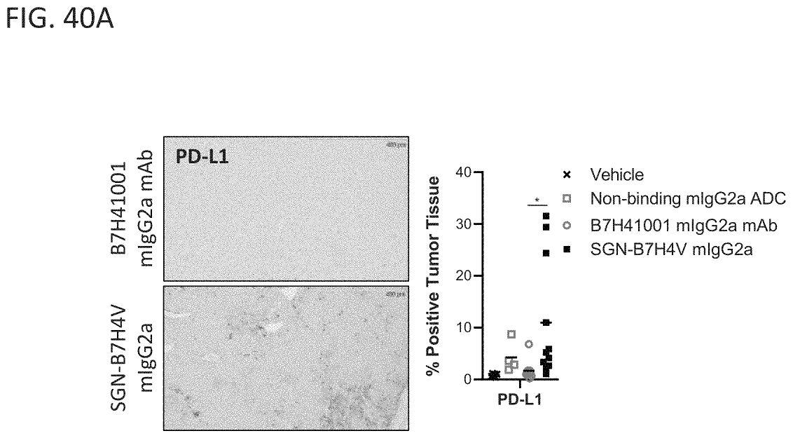

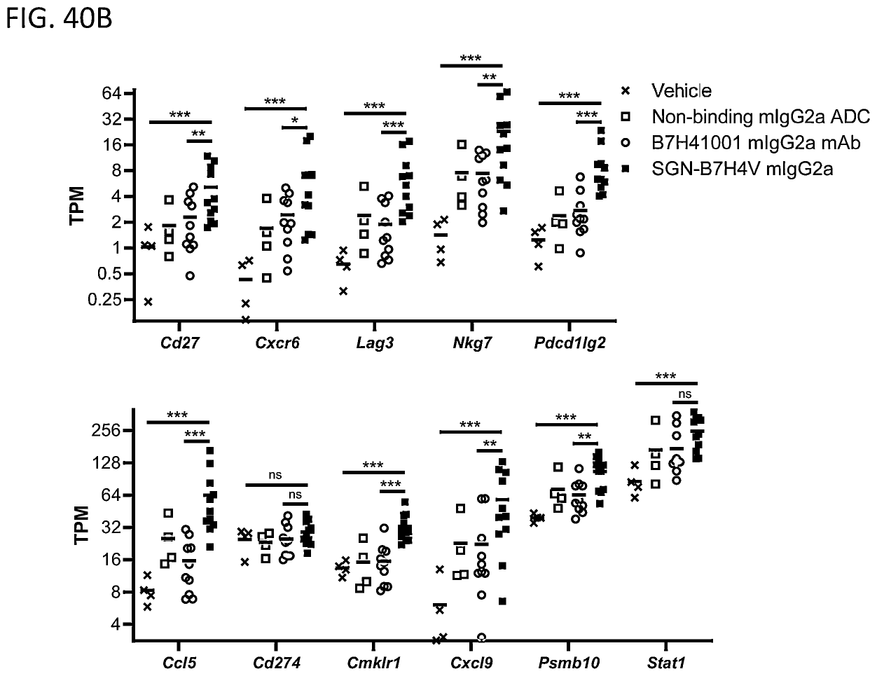

Response to anti-PD(L)1 agents in the clinic has been associated with expression of PD-L1 and/or expression of a “T cell-inflamed” gene signature (Ayers et al). Quantification of stained tumor sections also revealed an increase in the proportion of PD-L1+ cells (FIG. 40A). Moreover, RNAseq analysis revealed an increase in multiple murine genes associated with a “T cell-inflamed” gene signature (Ayers et al, FIG. 40B). Altogether, this suggests SGN-B7H4V may elicit immunomodulatory changes to the tumor microenvironment that may promote responsiveness to anti-PD(L)1 agents.

Additional Immunomodulatory Changes in Murine B7-H4-Expressing Renca Tumors Following Treatment with SGN-B7H4V mIgG2a

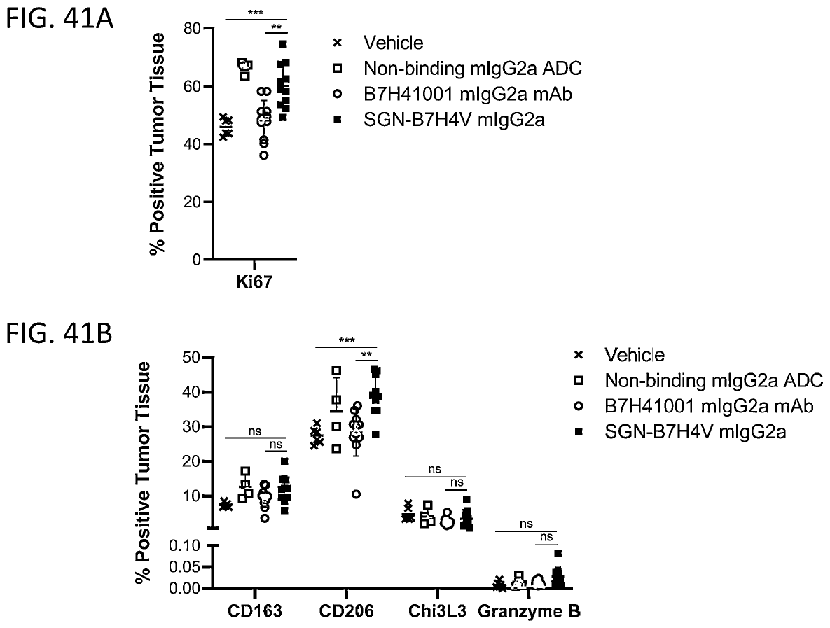

Tumor sections were also stained by immunohistochemistry for the cell cycle protein Ki67, the M2-like macrophage markers CD163, CD206, and Chi3L3, and the protease Granzyme B (which is found in granules of cytotoxic lymphocytes). A significant increase in the percentage of Ki67+ cells (FIG. 41A) and CD206+ cells (FIG. 41B) was observed in SGN-B7H4V mIgG2a-treated murine B7-H4-expressing Renca tumors. There was also a trend, though not statistically significant, towards an increase in CD163+ and Granzyme B+ cells in SGN-B7H4V mIgG2a-treated murine B7-H4-expressing Renca tumors. The increase in Ki67+ cells in the tumor following treatment with B7H4V mIgG2a is consistent with MMAE-induced G2/M tumor cell cycle arrest and/or infiltration of proliferating immune cells.

Multiple additional genes of interest were also evaluated by RNAseq and found to be altered in murine B7-H4-expressing Renca tumors following treatment with SGN-B7H4V mIgG2a (Table 23). For example, a decrease in Vtcn1 (which encodes B7-H4) transcripts was observed following treatment with SGN-B7H4V mIgG2a, but not the unconjugated B7H41001 mAb. Additionally, transcripts encoding multiple additional cytokines (e.g. CCL20, IFNy) and type I IFN response genes (TREX1, RSAD2) were elevated following treatment with SGN-B7H4V mIgG2a. Finally, gene ontology (GO) term analysis revealed upregulation of multiple immune-related gene categories following treatment with SGN-B7H4V mIgG2a compared to the unconjugated B7H41001 mIgG2a mAb (Table 24). Altogether, this suggests that treatment with SGN-B7H4V mIgG2a induces robust immunomodulatory changes to tumors. SGN-B7H4V mIgG2a treatment may both remove tumor cells that express the inhibitory ligand B7-H4 from the tumor microenvironment (TME) and increase expression of cytokine and type I IFN response genes to promote innate and adaptive immune cell activation and recruitment to tumors.

TABLE 23
RNAseq analysis of expression of additional genes of interest
by SGN-B7H4V mIgG2a-treated mB7-H4-expressing Renca tumors

| | Gene | Adjusted | Fold |
|---|---|---|---|
| Comparison | Symbol | p-value | Change |
| Non-binding mIgG2a ADC vs. vehicle | Vtcn1 | 0.846 | 0.974 |
| B7H41001 mIgG2a mAb vs. vehicle | | 0.884 | 0.978 |
| SGN-B7H4V mIgG2a vs. vehicle | | 0.0578 | 0.862 |
| SGN-B7H4V mIgG2avs. B7H41001 | | 0.0023 | 0.881 |
| mIgG2a mAb | | | |
| Non-binding mIgG2a ADC vs. vehicle | B2m | 0.000276 | 1.31 |
| B7H41001 mIgG2a mAb vs. vehicle | | 0.541 | 1.14 |
| SGN-B7H4V mIgG2a vs. vehicle | | 0.118 | 1.25 |
| SGN-B7H4V mIgG2a vs. B7H41001 | | 0.417 | 1.09 |
| mIgG2a mAb | | | |
| Non-binding mIgG2a ADC vs. vehicle | H2-D1 | 0.014 | 1.28 |
| B7H41001 mIgG2a mAb vs. vehicle | | 0.193 | 1.26 |
| SGN-B7H4V mIgG2a vs. vehicle | | 0.0633 | 1.27 |
| SGN-B7H4V mIgG2a vs. B7H41001 | | 0.941 | 1.01 |
| mIgG2a mAb | | | |
| Non-binding mIgG2a ADC vs. vehicle | H2-K1 | 0.0179 | 1.27 |
| B7H41001 mIgG2a mAb vs. vehicle | | 0.322 | 1.27 |
| SGN-B7H4V mIgG2a vs. vehicle | | 0.134 | 1.25 |
| SGN-B7H4V mIgG2a vs. B7H41001 | | 0.914 | 0.983 |
| mIgG2a mAb | | | |
| Non-binding mIgG2a ADC vs. vehicle | Mr1 | 0.0478 | 0.816 |
| B7H41001 mIgG2a mAb vs. vehicle | | 0.579 | 0.862 |
| SGN-B7H4V mIgG2a vs. vehicle | | 0.00402 | 0.783 |
| SGN-B7H4V mIgG2a vs. B7H41001 | | 0.26 | 0.908 |
| mIgG2a mAb | | | |
| Non-binding mIgG2a ADC vs. vehicle | Tap1 | 0.0313 | 1.47 |
| B7H41001 mIgG2a mAb vs. vehicle | | 0.221 | 1.48 |
| SGN-B7H4V mIgG2a vs. vehicle | | 0.0124 | 1.63 |
| SGN-B7H4V mIgG2a vs. B7H41001 | | 0.617 | 1.10 |
| mIgG2a mAb | | | |
| Non-binding mIgG2a ADC vs. vehicle | Tap2 | 0.0244 | 1.37 |
| B7H41001 mIgG2a mAb vs. vehicle | | 0.412 | 1.30 |
| SGN-B7H4V mIgG2a vs. vehicle | | 0.00209 | 1.53 |
| SGN-B7H4V mIgG2a vs. B7H41001 | | 0.214 | 1.18 |
| mIgG2a mAb | | | |
| Non-binding mIgG2a ADC vs. vehicle | H2-Aa | 0.00564 | 2.75 |
| B7H41001 mIgG2a mAb vs. vehicle | | 0.419 | 2.01 |
| SGN-B7H4V mIgG2a vs. vehicle | | 0.0000492 | 6.50 |
| SGN-B7H4V mIgG2a vs. B7H41001 | | 0.000345 | 3.22 |
| mIgG2a mAb | | | |
| Non-binding mIgG2a ADC vs. vehicle | H2-Eb1 | 0.048 | 2.70 |
| B7H41001 mIgG2a mAb vs. vehicle | | 0.408 | 2.23 |
| SGN-B7H4V mIgG2a vs. vehicle | | 0.0000286 | 7.25 |
| SGN-B7H4V mIgG2a vs. B7H41001 | | 0.000382 | 3.25 |
| mIgG2a mAb | | | |
| Non-binding mIgG2a ADC vs. vehicle | Cd80 | 0.346 | 0.613 |
| B7H41001 mIgG2a mAb vs. vehicle | | 0.665 | 0.662 |
| SGN-B7H4V mIgG2a vs. vehicle | | 0.745 | 1.14 |
| SGN-B7H4V mIgG2a vs. B7H41001 | | 0.00769 | 1.71 |
| mIgG2a mAb | | | |
| Non-binding mIgG2a ADC vs. vehicle | Cd86 | 0.415 | 1.17 |
| B7H41001 mIgG2a mAb vs. vehicle | | 0.882 | 1.06 |
| SGN-B7H4V mIgG2a vs. vehicle | | 8.24e−7 | 2.24 |
| SGN-B7H4V mIgG2a vs. B7H41001 | | 1.34e−10 | 2.11 |
| mIgG2a mAb | | | |
| Non-binding mIgG2a ADC vs. vehicle | Icosl | 0.78 | 1.08 |
| B7H41001 mIgG2a mAb vs. vehicle | | 0.822 | 0.868 |
| SGN-B7H4V mIgG2a vs. vehicle | | 0.00961 | 1.63 |
| SGN-B7H4V mIgG2a vs. B7H41001 | | 0.0000641 | 1.88 |
| mIgG2a mAb | | | |
| Non-binding mIgG2a ADC vs. vehicle | Tnfsf4 | 0.619 | 1.79 |
| B7H41001 mIgG2a mAb vs. vehicle | | | 1.39 |
| SGN-B7H4V mIgG2a vs. vehicle | | 0.0118 | 3.35 |
| SGN-B7H4V mIgG2a vs. B7H41001 | | 0.00828 | 2.37 |
| mIgG2a mAb | | | |
| Non-binding mIgG2a ADC vs. vehicle | Ccl20 | | 2.12 |
| B7H41001 mIgG2a mAb vs. vehicle | | | 0.402 |
| SGN-B7H4V mIgG2a vs. vehicle | | 0.0834 | 4.32 |
| SGN-B7H4V mIgG2a vs. B7H41001 | | 0.000243 | 10.8 |
| mIgG2a mAb | | | |
| Non-binding mIgG2a ADC vs. vehicle | Cxcl15 | 1.43e−15 | 215 |
| B7H41001 mIgG2a mAb vs. vehicle | | 0.419 | 63.9 |
| SGN-B7H4V mIgG2a vs. vehicle | | 0.125 | 35 |
| SGN-B7H4V mIgG2a vs. B7H41001 | | 0.765 | 0.546 |
| mIgG2a mAb | | | |
| Non-binding mIgG2a ADC vs. vehicle | Cxcl9 | 0.0095 | 4.25 |
| B7H41001 mIgG2a mAb vs. vehicle | | 0.198 | 3.47 |
| SGN-B7H4V mIgG2a vs. vehicle | | 0.00000644 | 9.84 |
| SGN-B7H4V mIgG2a vs. B7H41001 | | 0.00684 | 2.84 |
| mIgG2a mAb | | | |
| Non-binding mIgG2a ADC vs. vehicle | Batf3 | 0.0402 | 2.55 |
| B7H41001 mIgG2a mAb vs. vehicle | | 0.612 | 1.50 |
| SGN-B7H4V mIgG2a vs. vehicle | | 0.0000252 | 4.45 |
| SGN-B7H4V mIgG2a vs. B7H41001 | | 0.0000114 | 2.96 |
| mIgG2a mAb | | | |
| Non-binding mIgG2a ADC vs. vehicle | Itgae | 0.998 | 1.00 |
| B7H41001 mIgG2a mAb vs. vehicle | | 0.663 | 1.43 |
| SGN-B7H4V mIgG2a vs. vehicle | | 0.168 | 1.52 |
| SGN-B7H4V mIgG2a vs. B7H41001 | | 0.856 | 1.06 |
| mIgG2a mAb | | | |
| Non-binding mIgG2a ADC vs. vehicle | Itgax | 0.0301 | 1.47 |
| B7H41001 mIgG2a mAb vs. vehicle | | 0.539 | 1.47 |
| SGN-B7H4V mIgG2a vs. vehicle | | 2.95e−24 | 3.54 |
| SGN-B7H4V mIgG2a vs. B7H41001 | | 2.78e 8 | 2.41 |
| mIgG2a mAb | | | |
| Non-binding mIgG2a ADC vs. vehicle | Gzmb | 0.0884 | 3.29 |
| B7H41001 mIgG2a mAb vs. vehicle | | 0.508 | 1.95 |
| SGN-B7H4V mIgG2a vs. vehicle | | 0.00294 | 5.46 |
| SGN-B7H4V mIgG2a vs. B7H41001 | | 0.00851 | 2.81 |
| mIgG2a mAb | | | |
| Non-binding mIgG2a ADC vs. vehicle | Havcr2 | 0.000561 | 0.548 |
| B7H41001 mIgG2a mAb vs. vehicle | | 0.394 | 0.569 |
| SGN-B7H4V mIgG2a vs. vehicle | | 0.468 | 0.805 |
| SGN-B7H4V mIgG2a vs. B7H41001 | | 0.158 | 1.41 |
| mIgG2a mAb | | | |
| Non-binding mIgG2a ADC vs. vehicle | Ifng | 0.0555 | 4.32 |
| B7H41001 mIgG2a mAb vs. vehicle | | 0.484 | 3.38 |
| SGN-B7H4V mIgG2a vs. vehicle | | 0.000741 | 10.8 |
| SGN-B7H4V mIgG2a vs. B7H41001 | | 0.0258 | 3.20 |
| mIgG2a mAb | | | |
| Non-binding mIgG2a ADC vs. vehicle | Lag3 | 0.00751 | 3.78 |
| B7H41001 mIgG2a mAb vs. vehicle | | 0.167 | 2.94 |
| SGN-B7H4V mIgG2a vs. vehicle | | 3.80e−8 | 11.1 |
| SGN-B7H4V mIgG2a vs. B7H41001 | | 0.000329 | 3.78 |
| mIgG2a mAb | | | |
| Non-binding mIgG2a ADC vs. vehicle | Pdcd1 | 0.102 | 2.04 |
| B7H41001 mIgG2a mAb vs. vehicle | | 0.292 | 2.52 |
| SGN-B7H4V mIgG2a vs. vehicle | | 0.0000729 | 7.48 |
| SGN-B7H4V mIgG2a vs. B7H41001 | | 0.00754 | 2.96 |
| mIgG2a mAb | | | |
| Non-binding mIgG2a ADC vs. vehicle | Tox | 0.222 | 2.42 |
| B7H41001 mIgG2a mAb vs. vehicle | | 0.337 | 2.90 |
| SGN-B7H4V mIgG2a vs. vehicle | | 0.00000555 | 9.11 |
| SGN-B7H4V mIgG2a vs. B7H41001 | | 0.00217 | 3.14 |
| mIgG2a mAb | | | |
| Non-binding mIgG2a ADC vs. vehicle | Cd274 | 0.817 | 0.931 |
| B7H41001 mIgG2a mAb vs. vehicle | | 0.992 | 0.991 |
| SGN-B7H4V mIgG2a vs. vehicle | | 0.523 | 1.16 |
| SGN-B7H4V mIgG2a vs. B7H41001 | | 0.382 | 1.17 |
| mIgG2a mAb | | | |
| Non-binding mIgG2a ADC vs. vehicle | Cd47 | 0.116 | 0.74 |
| B7H41001 mIgG2a mAb vs. vehicle | | 0.662 | 0.851 |
| SGN-B7H4V mIgG2a vs. vehicle | | 0.0359 | 0.759 |
| SGN-B7H4V mIgG2a vs. B7H41001 | | 0.156 | 0.891 |
| mIgG2a mAb | | | |
| Non-binding mIgG2a ADC vs. vehicle | Ctla4 | 0.513 | 1.50 |
| B7H41001 mIgG2a mAb vs. vehicle | | 0.902 | 1.19 |
| SGN-B7H4V mIgG2a vs. vehicle | | 0.0295 | 2.99 |
| SGN-B7H4V mIgG2a vs. B7H41001 | | 0.0167 | 2.49 |
| mIgG2a mAb | | | |
| Non-binding mIgG2a ADC vs. vehicle | Pdcd1 | 0.102 | 2.04 |
| B7H41001 mIgG2a mAb vs. vehicle | | 0.292 | 2.52 |
| SGN-B7H4V mIgG2a vs. vehicle | | 0.0000729 | 7.48 |
| SGN-B7H4V mIgG2a vs. B7H41001 | | 0.00754 | 2.96 |
| mIgG2a mAb | | | |
| Non-binding mIgG2a ADC vs. vehicle | Sirpa | 0.569 | 1.06 |
| B7H41001 mIgG2a mAb vs. vehicle | | 0.235 | 1.15 |
| SGN-B7H4V mIgG2a vs. vehicle | | 0.112 | 1.15 |
| SGN-B7H4V mIgG2a vs. B7H41001 | | 0.977 | 0.998 |
| mIgG2a mAb | | | |
| Non-binding mIgG2a ADC vs. vehicle | Tigit | 0.0858 | 2.62 |
| B7H41001 mIgG2a mAb vs. vehicle | | 0.538 | 1.95 |
| SGN-B7H4V mIgG2a vs. vehicle | | 0.00000556 | 7.71 |
| SGN-B7H4V mIgG2a vs. B7H41001 | | 0.0000543 | 3.95 |
| mIgG2a mAb | | | |
| Non-binding mIgG2a ADC vs. vehicle | Arg1 | 0.336 | 0.443 |
| B7H41001 mIgG2a mAb vs. vehicle | | 0.679 | 0.535 |
| SGN-B7H4V mIgG2a vs. vehicle | | 0.285 | 0.472 |
| SGN-B7H4V mIgG2a vs. B7H41001 | | 0.823 | 0.881 |
| mIgG2a mAb | | | |
| Non-binding mIgG2a ADC vs. vehicle | Cd163 | 0.784 | 0.743 |
| B7H41001 mIgG2a mAb vs. vehicle | | 0.865 | 1.23 |
| SGN-B7H4V mIgG2a vs. vehicle | | 0.652 | 1.27 |
| SGN-B7H4V mIgG2a vs. B7H41001 | | 0.931 | 1.02 |
| mIgG2a mAb | | | |
| Non-binding mIgG2a ADC vs. vehicle | Cd68 | 0.00636 | 1.27 |
| B7H41001 mIgG2a mAb vs. vehicle | | 0.949 | 1.03 |
| SGN-B7H4V mIgG2a vs. vehicle | | 0.0275 | 1.32 |
| SGN-B7H4V mIgG2a vs. B7H41001 | | 0.0127 | 1.28 |
| mIgG2a mAb | | | |
| Non-binding mIgG2a ADC vs. vehicle | Chil1 | 0.0614 | 3.14 |
| B7H41001 mIgG2a mAb vs. vehicle | | 0.837 | 1.40 |
| SGN-B7H4V mIgG2a vs. vehicle | | 0.834 | 1.15 |
| SGN-B7H4V mIgG2a vs. B7H41001 | | 0.679 | 0.822 |
| mIgG2a mAb | | | |
| Non-binding mIgG2a ADC vs. vehicle | Mertk | 0.00774 | 0.63 |
| B7H41001 mIgG2a mAb vs. vehicle | | 0.461 | 0.703 |
| SGN-B7H4V mIgG2a vs. vehicle | | 0.154 | 1.46 |
| SGN-B7H4V mIgG2a vs. B7H41001 | | 0.0000147 | 2.08 |
| mIgG2a mAb | | | |
| Non-binding mIgG2a ADC vs. vehicle | Mrc1 | 0.401 | 0.806 |
| B7H41001 mIgG2a mAb vs. vehicle | | 0.756 | 1.14 |
| SGN-B7H4V mIgG2a vs. vehicle | | 0.137 | 1.26 |
| SGN-B7H4V mIgG2a vs. B7H41001 | | 0.464 | 1.10 |
| mIgG2a mAb | | | |
| Non-binding mIgG2a ADC vs. vehicle | Nos2 | 0.829 | 0.792 |
| B7H41001 mIgG2a mAb vs. vehicle | | 0.945 | 1.12 |
| SGN-B7H4V mIgG2a vs. vehicle | | 0.212 | 0.509 |
| SGN-B7H4V mIgG2a vs. B7H41001 | | 0.0041 | 0.455 |
| mIgG2a mAb | | | |
| Non-binding mIgG2a ADC vs. vehicle | Cd14 | 0.693 | 0.799 |
| B7H41001 mIgG2a mAb vs. vehicle | | 0.573 | 0.723 |
| SGN-B7H4V mIgG2a vs. vehicle | | 0.245 | 0.767 |
| SGN-B7H4V mIgG2a vs. B7H41001 | | 0.654 | 1.06 |
| mIgG2a mAb | | | |
| Non-binding mIgG2a ADC vs. vehicle | Itgam | 0.265 | 0.66 |
| B7H41001 mIgG2a mAb vs. vehicle | | 0.653 | 0.755 |
| SGN-B7H4V mIgG2a vs. vehicle | | 0.435 | 0.843 |
| SGN-B7H4V mIgG2a vs. B7H41001 | | 0.546 | 1.12 |
| mIgG2a mAb | | | |
| Non-binding mIgG2a ADC vs. vehicle | Ly6c1 | 0.00141 | 1.59 |
| B7H41001 mIgG2a mAb vs. vehicle | | 0.0875 | 1.74 |
| SGN-B7H4V mIgG2a vs. vehicle | | 0.352 | 1.31 |
| SGN-B7H4V mIgG2a vs. B7H41001 | | 0.177 | 0.753 |
| mIgG2a mAb | | | |
| Non-binding mIgG2a ADC vs. vehicle | Ly6g | 0.769 | 0.52 |
| B7H41001 mIgG2a mAb vs. vehicle | | | 0.682 |
| SGN-B7H4V mIgG2a vs. vehicle | | 0.585 | 0.506 |
| SGN-B7H4V mIgG2a vs. B7H41001 | | 0.793 | 0.738 |
| mIgG2a mAb | | | |
| Non-binding mIgG2a ADC vs. vehicle | Cd19 | 0.0826 | 14.6 |
| B7H41001 mIgG2a mAb vs. vehicle | | | 4.85 |
| SGN-B7H4V mIgG2a vs. vehicle | | 0.113 | 11.8 |
| SGN-B7H4V mIgG2a vs. B7H41001 | | 0.327 | 2.43 |
| mIgG2a mAb | | | |
| Non-binding mIgG2a ADC vs. vehicle | Cd34 | 0.745 | 0.786 |
| B7H41001 mIgG2a mAb vs. vehicle | | 0.824 | 0.829 |
| SGN-B7H4V mIgG2a vs. vehicle | | 0.813 | 1.09 |
| SGN-B7H4V mIgG2a vs. B7H41001 | | 0.145 | 1.32 |
| mIgG2a mAb | | | |
| Non-binding mIgG2a ADC vs. vehicle | Cd40 | 0.729 | 0.911 |
| B7H41001 mIgG2a mAb vs. vehicle | | 0.664 | 0.839 |
| SGN-B7H4V mIgG2a vs. vehicle | | 0.44 | 1.13 |
| SGN-B7H4V mIgG2a vs. B7H41001 | | 0.000644 | 1.35 |
| mIgG2a mAb | | | |
| Non-binding mIgG2a ADC vs. vehicle | Cd40lg | | 13 |
| B7H41001 mIgG2a mAb vs. vehicle | | | 6.98 |
| SGN-B7H4V mIgG2a vs. vehicle | | 0.0845 | 10.5 |
| SGN-B7H4V mIgG2a vs. B7H41001 | | 0.7 | 1.46 |
| mIgG2a mAb | | | |
| Non-binding mIgG2a ADC vs. vehicle | Pecam1 | 0.726 | 0.822 |
| B7H41001 mIgG2a mAb vs. vehicle | | 0.715 | 0.781 |
| SGN-B7H4V mIgG2a vs. vehicle | | 0.97 | 0.986 |
| SGN-B7H4V mIgG2a vs. B7H41001 | | 0.233 | 1.26 |
| mIgG2a mAb | | | |
| Non-binding mIgG2a ADC vs. vehicle | Ptprc | 0.837 | 1.06 |
| B7H41001 mIgG2a mAb vs. vehicle | | 0.952 | 1.04 |
| SGN-B7H4V mIgG2a vs. vehicle | | 0.000248 | 1.92 |
| SGN-B7H4V mIgG2a vs. B7H41001 | | 0.0000227 | 1.85 |
| mIgG2a mAb | | | |
| Non-binding mIgG2a ADC vs. vehicle | Slfn8 | 0.919 | 0.976 |
| B7H41001 mIgG2a mAb vs. vehicle | | 0.65 | 1.18 |
| SGN-B7H4V mIgG2a vs. vehicle | | 0.516 | 1.09 |
| SGN-B7H4V mIgG2a vs. B7H41001 | | 0.471 | 0.925 |
| mIgG2a mAb | | | |
| Non-binding mIgG2a ADC vs. vehicle | Slfn9 | 0.457 | 0.857 |
| B7H41001 mIgG2a mAb vs. vehicle | | 0.922 | 1.05 |
| SGN-B7H4V mIgG2a vs. vehicle | | 0.54 | 1.11 |
| SGN-B7H4V mIgG2a vs. B7H41001 | | 0.671 | 1.06 |
| mIgG2a mAb | | | |
| Non-binding mIgG2a ADC vs. vehicle | Abcb1a | 0.284 | 0.657 |
| B7H41001 mIgG2a mAb vs. vehicle | | 0.461 | 0.644 |
| SGN-B7H4V mIgG2a vs. vehicle | | 0.74 | 0.896 |
| SGN-B7H4V mIgG2a vs. B7H41001 | | 0.0354 | 1.39 |
| mIgG2a mAb | | | |
| Non-binding mIgG2a ADC vs. vehicle | Abcb1b | 0.57 | 1.22 |
| B7H41001 mIgG2a mAb vs. vehicle | | 0.559 | 1.30 |
| SGN-B7H4V mIgG2a vs. vehicle | | 0.00383 | 1.72 |
| SGN-B7H4V mIgG2a vs. B7H41001 | | 0.0279 | 1.32 |
| mIgG2a mAb | | | |
| Non-binding mIgG2a ADC vs. vehicle | Mki67 | 0.844 | 1.05 |
| B7H41001 mIgG2a mAb vs. vehicle | | 0.77 | 1.12 |
| SGN-B7H4V mIgG2a vs. vehicle | | 0.308 | 1.17 |
| SGN-B7H4V mIgG2a vs. B7H41001 | | 0.725 | 1.04 |
| mIgG2a mAb | | | |
| Non-binding mIgG2a ADC vs. vehicle | Cd3e | 0.00839 | 3.35 |
| B7H41001 mIgG2a mAb vs. vehicle | | 0.143 | 3.16 |
| SGN-B7H4V mIgG2a vs. vehicle | | 0.00000712 | 8.87 |
| SGN-B7H4V mIgG2a vs. B7H41001 | | 0.00529 | 2.80 |
| mIgG2a mAb | | | |
| Non-binding mIgG2a ADC vs. vehicle | Cd4 | 0.0476 | 2.79 |
| B7H41001 mIgG2a mAb vs. vehicle | | 0.501 | 1.87 |
| SGN-B7H4V mIgG2a vs. vehicle | | 5.10e−10 | 13.4 |
| SGN-B7H4V mIgG2a vs. B7H41001 | | 3.10e−11 | 7.17 |
| mIgG2a mAb | | | |
| Non-binding mIgG2a ADC vs. vehicle | Cd8a | 0.000368 | 5.29 |
| B7H41001 mIgG2a mAb vs. vehicle | | 0.00184 | 6.02 |
| SGN-B7H4V mIgG2a vs. vehicle | | 2.17e−8 | 17.9 |
| SGN-B7H4V mIgG2a vs. B7H41001 | | 0.00752 | 2.97 |
| mIgG2a mAb | | | |
| Non-binding mIgG2a ADC vs. vehicle | Foxp3 | 0.121 | 1.93 |
| B7H41001 mIgG2a mAb vs. vehicle | | 0.711 | 1.48 |
| SGN-B7H4V mIgG2a vs. vehicle | | 0.000442 | 3.88 |
| SGN-B7H4V mIgG2a vs. B7H41001 | | 0.00137 | 2.62 |
| mIgG2a mAb | | | |
| Non-binding mIgG2a ADC vs. vehicle | Cd27 | 0.196 | 2.19 |
| B7H41001 mIgG2a mAb vs. vehicle | | 0.394 | 2.41 |
| SGN-B7H4V mIgG2a vs. vehicle | | 0.0000759 | 6.87 |
| SGN-B7H4V mIgG2a vs. B7H41001 | | 0.0043 | 2.84 |
| mIgG2a mAb | | | |
| Non-binding mIgG2a ADC vs. vehicle | Cd28 | 0.166 | 2.18 |
| B7H41001 mIgG2a mAb vs. vehicle | | 0.508 | 2.33 |
| SGN-B7H4V mIgG2a vs. vehicle | | 0.000161 | 6.34 |
| SGN-B7H4V mIgG2a vs. B7H41001 | | 0.00963 | 2.72 |
| mIgG2a mAb | | | |
| Non-binding mIgG2a ADC vs. vehicle | Icos | 0.476 | 1.54 |
| B7H41001 mIgG2a mAb vs. vehicle | | 0.521 | 1.81 |
| SGN-B7H4V mIgG2a vs. vehicle | | 0.000692 | 4.04 |
| SGN-B7H4V mIgG2a vs. B7H41001 | | 0.00587 | 2.23 |
| mIgG2a mAb | | | |
| Non-binding mIgG2a ADC vs. vehicle | Cxcl10 | 0.00385 | 2.94 |
| B7H41001 mIgG2a mAb vs. vehicle | | 0.1 | 1.83 |
| SGN-B7H4V mIgG2a vs. vehicle | | 1.85e−9 | 4.85 |
| SGN-B7H4V mIgG2a vs. B7H41001 | | 4.20e−7 | 2.66 |
| mIgG2a mAb | | | |
| Non-binding mIgG2a ADC vs. vehicle | Ifit1 | 0.28 | 1.47 |
| B7H41001 mIgG2a mAb vs. vehicle | | 0.781 | 1.20 |
| SGN-B7H4V mIgG2a vs. vehicle | | 0.00834 | 1.80 |
| SGN-B7H4V mIgG2a vs. B7H41001 | | 0.00955 | 1.50 |
| mIgG2a mAb | | | |
| Non-binding mIgG2a ADC vs. vehicle | Ifit2 | 0.314 | 1.32 |
| B7H41001 mIgG2a mAb vs. vehicle | | 0.774 | 1.13 |
| SGN-B7H4V mIgG2a vs. vehicle | | 0.00126 | 1.64 |
| SGN-B7H4V mIgG2a vs. B7H41001 | | 0.000425 | 1.45 |
| mIgG2a mAb | | | |
| Non-binding mIgG2a ADC vs. vehicle | Ifit3 | 0.0255 | 2.19 |
| B7H41001 mIgG2a mAb vs. vehicle | | 0.237 | 1.58 |
| SGN-B7H4V mIgG2a vs. vehicle | | 0.0000277 | 2.52 |
| SGN-B7H4V mIgG2a vs. B7H41001 | | 0.00301 | 1.60 |
| mIgG2a mAb | | | |
| Non-binding mIgG2a ADC vs. vehicle | Isg15 | 0.332 | 1.18 |
| B7H41001 mIgG2a mAb vs. vehicle | | 0.811 | 1.09 |
| SGN-B7H4V mIgG2a vs. vehicle | | 0.734 | 1.06 |
| SGN-B7H4V mIgG2a vs. B7H41001 | | 0.81 | 0.968 |
| mIgG2a mAb | | | |
| Non-binding mIgG2a ADC vs. vehicle | Mx1 | 0.0518 | 2.24 |
| B7H41001 mIgG2a mAb vs. vehicle | | 0.809 | 1.14 |
| SGN-B7H4V mIgG2a vs. vehicle | | 2.21e−10 | 3.36 |
| SGN-B7H4V mIgG2a vs. B7H41001 | | 4.98e−18 | 2.96 |
| mIgG2a mAb | | | |
| Non-binding mIgG2a ADC vs. vehicle | Oas1a | 0.0666 | 1.31 |
| B7H41001 mIgG2a mAb vs. vehicle | | 0.391 | 1.19 |
| SGN-B7H4V mIgG2a vs. vehicle | | 0.0101 | 1.36 |
| SGN-B7H4V mIgG2a vs. B7H41001 | | 0.157 | 1.14 |
| mIgG2a mAb | | | |
| Non-binding mIgG2a ADC vs. vehicle | Oas2 | 0.87 | 1.05 |
| B7H41001 mIgG2a mAb vs. vehicle | | 0.848 | 1.09 |
| SGN-B7H4V mIgG2a vs. vehicle | | 0.335 | 1.17 |
| SGN-B7H4V mIgG2a vs. B7H41001 | | 0.663 | 1.07 |
| mIgG2a mAb | | | |
| Non-binding mIgG2a ADC vs. vehicle | Rsad2 | 0.697 | 1.30 |
| B7H41001 mIgG2a mAb vs. vehicle | | 0.911 | 1.08 |
| SGN-B7H4V mIgG2a vs. vehicle | | 0.0112 | 1.80 |
| SGN-B7H4V mIgG2a vs. B7H41001 | | 0.00142 | 1.66 |
| mIgG2a mAb | | | |
| Non-binding mIgG2a ADC vs. vehicle | Trex1 | 0.0203 | 1.79 |
| B7H41001 mIgG2a mAb vs. vehicle | | 0.553 | 1.33 |
| SGN-B7H4V mIgG2a vs. vehicle | | 0.000134 | 2.25 |
| SGN-B7H4V mIgG2a vs. B7H41001 | | 0.00161 | 1.69 |
| mIgG2a mAb | | | |

TABLE 24
Top 50 significantly changed GO terms in SGN-B7H4V mIgG2a vs.
B7H41001 mIgG2a mAb-treated mB7-H4-expressing Renca tumors

| Comparison | Direction | ONTOLOGY | Description | p. adjust |
|---|---|---|---|---|
| SGN-B7H4V mIgG2a vs. | Up | BP | adaptive immune response | 3.46E−26 |
| B7H41001 mIgG2a mAb | | | | |
| SGN-B7H4V mIgG2a vs. | Up | BP | mononuclear cell differentiation | 1.13E−22 |
| B7H41001 mIgG2a mAb | | | | |
| SGN-B7H4V mIgG2a vs. | Up | BP | regulation of leukocyte cell-cell | 1.71E−22 |
| B7H41001 mIgG2a mAb | | | adhesion | |
| SGN-B7H4V mIgG2a vs. | Up | BP | regulation of T cell activation | 1.69E−21 |
| B7H41001 mIgG2a mAb | | | | |
| SGN-B7H4V mIgG2a vs. | Up | BP | mononuclear cell proliferation | 1.69E−21 |
| B7H41001 mIgG2a mAb | | | | |
| SGN-B7H4V mIgG2a vs. | Up | BP | positive regulation of cytokine | 3.34E−21 |
| B7H41001 mIgG2a mAb | | | production | |
| SGN-B7H4V mIgG2a vs. | Up | BP | lymphocyte proliferation | 3.88E−21 |
| B7H41001 mIgG2a mAb | | | | |
| SGN-B7H4V mIgG2a vs. | Up | BP | leukocyte proliferation | 1.24E−20 |
| B7H41001 mIgG2a mAb | | | | |
| SGN-B7H4V mIgG2a vs. | Up | BP | regulation of cell-cell adhesion | 4.21E−20 |
| B7H41001 mIgG2a mAb | | | | |
| SGN-B7H4V mIgG2a vs. | Up | BP | leukocyte cell-cell adhesion | 7.12E−20 |
| B7H41001 mIgG2a mAb | | | | |
| SGN-B7H4V mIgG2a vs. | Up | BP | negative regulation of immune | 1.00E−19 |
| B7H41001 mIgG2a mAb | | | system process | |
| SGN-B7H4V mIgG2a vs. | Up | BP | alpha-beta T cell activation | 2.11E−19 |
| B7H41001 mIgG2a mAb | | | | |
| SGN-B7H4V mIgG2a vs. | Up | BP | lymphocyte differentiation | 3.11E−19 |
| B7H41001 mIgG2a mAb | | | | |
| SGN-B7H4V mIgG2a vs. | Up | BP | regulation of immune effector | 6.27E−19 |
| B7H41001 mIgG2a mAb | | | process | |
| SGN-B7H4V mIgG2a vs. | Up | BP | regulation of response to biotic | 9.21E−19 |
| B7H41001 mIgG2a mAb | | | stimulus | |
| SGN-B7H4V mIgG2a vs. | Up | BP | regulation of mononuclear cell | 1.76E−18 |
| B7H41001 mIgG2a mAb | | | proliferation | |
| SGN-B7H4V mIgG2a vs. | Up | BP | positive regulation of leukocyte | 1.76E−18 |
| B7H41001 mIgG2a mAb | | | cell-cell adhesion | |
| SGN-B7H4V mIgG2a vs. | Up | BP | regulation of lymphocyte | 5.15E−18 |
| B7H41001 mIgG2a mAb | | | proliferation | |
| SGN-B7H4V mIgG2a vs. | Up | BP | regulation of innate immune | 1.45E−17 |
| B7H41001 mIgG2a mAb | | | response | |
| SGN-B7H4V mIgG2a vs. | Up | BP | positive regulation of response | 1.86E−17 |
| B7H41001 mIgG2a mAb | | | to external stimulus | |
| SGN-B7H4V mIgG2a vs. | Up | BP | regulation of leukocyte | 5.55E−17 |
| B7H41001 mIgG2a mAb | | | proliferation | |
| SGN-B7H4V mIgG2a vs. | Up | BP | T cell proliferation | 7.18E−17 |
| B7H41001 mIgG2a mAb | | | | |
| SGN-B7H4V mIgG2a vs. | Up | BP | leukocyte mediated immunity | 7.23E−17 |
| B7H41001 mIgG2a mAb | | | | |
| SGN-B7H4V mIgG2a vs. | Up | BP | positive regulation of T cell | 1.00E−16 |
| B7H41001 mIgG2a mAb | | | activation | |
| SGN-B7H4V mIgG2a vs. | Up | BP | T cell differentiation | 1.71E−16 |
| B7H41001 mIgG2a mAb | | | | |
| SGN-B7H4V mIgG2a vs. | Up | BP | positive regulation of cell-cell | 1.52E−15 |
| B7H41001 mIgG2a mAb | | | adhesion | |
| SGN-B7H4V mIgG2a vs. | Up | BP | positive regulation of cell | 1.59E−15 |
| B7H41001 mIgG2a mAb | | | activation | |
| SGN-B7H4V mIgG2a vs. | Up | BP | immune response-regulating | 3.90E−15 |
| B7H41001 mIgG2a mAb | | | signaling pathway | |
| SGN-B7H4V mIgG2a vs. | Up | BP | positive regulation of defense | 5.75E−15 |
| B7H41001 mIgG2a mAb | | | response | |
| SGN-B7H4V mIgG2a vs. | Up | BP | lymphocyte mediated immunity | 6.38E−15 |
| B7H41001 mIgG2a mAb | | | | |
| SGN-B7H4V mIgG2a vs. | Up | BP | regulation of leukocyte mediated | 6.38E−15 |
| B7H41001 mIgG2a mAb | | | immunity | |
| SGN-B7H4V mIgG2a vs. | Up | BP | positive regulation of leukocyte | 6.46E−15 |
| B7H41001 mIgG2a mAb | | | activation | |
| SGN-B7H4V mIgG2a vs. | Up | BP | positive regulation of immune | 8.09E−15 |
| B7H41001 mIgG2a mAb | | | effector process | |
| SGN-B7H4V mIgG2a vs. | Up | BP | adaptive immune response based | 1.93E−14 |
| B7H41001 mIgG2a mAb | | | on somatic recombination of | |
| | | | immune receptors built from | |
| | | | immunoglobulin superfamily | |
| | | | domains | |
| SGN-B7H4V mIgG2a vs. | Up | BP | regulation of adaptive immune | 2.12E−14 |
| B7H41001 mIgG2a mAb | | | response | |
| SGN-B7H4V mIgG2a vs. | Up | BP | regulation of T cell proliferation | 2.12E−14 |
| B7H41001 mIgG2a mAb | | | | |
| SGN-B7H4V mIgG2a vs. | Up | BP | negative regulation of cell | 1.07E−13 |
| B7H41001 mIgG2a mAb | | | activation | |
| SGN-B7H4V mIgG2a vs. | Up | BP | regulation of lymphocyte mediated | 1.75E−13 |
| B7H41001 mIgG2a mAb | | | immunity | |
| SGN-B7H4V mIgG2a vs. | Up | BP | regulation of tumor necrosis factor | 3.78E−13 |
| B7H41001 mIgG2a mAb | | | superfamily cytokine production | |
| SGN-B7H4V mIgG2a vs. | Up | BP | regulation of tumor necrosis factor | 4.54E−13 |
| B7H41001 mIgG2a mAb | | | production | |
| SGN-B7H4V mIgG2a vs. | Up | BP | negative regulation of cytokine | 4.54E−13 |
| B7H41001 mIgG2a mAb | | | production | |
| SGN-B7H4V mIgG2a vs. | Up | BP | positive regulation of lymphocyte | 4.72E−13 |
| B7H41001 mIgG2a mAb | | | activation | |
| SGN-B7H4V mIgG2a vs. | Up | BP | positive regulation of response to | 7.52E−13 |
| B7H41001 mIgG2a mAb | | | biotic stimulus | |
| SGN-B7H4V mIgG2a vs. | Up | BP | regulation of interleukin-6 | 1.11E−12 |
| B7H41001 mIgG2a mAb | | | production | |
| SGN-B7H4V mIgG2a vs. | Up | BP | negative regulation of leukocyte | 1.15E−12 |
| B7H41001 mIgG2a mAb | | | activation | |
| SGN-B7H4V mIgG2a vs. | Up | BP | regulation of leukocyte | 1.45E−12 |
| B7H41001 mIgG2a mAb | | | differentiation | |
| SGN-B7H4V mIgG2a vs. | Up | BP | myeloid leukocyte activation | 1.67E−12 |
| B7H41001 mIgG2a mAb | | | | |
| SGN-B7H4V mIgG2a vs. | Up | BP | tumor necrosis factor superfamily | 1.72E−12 |
| B7H41001 mIgG2a mAb | | | cytokine production | |
| SGN-B7H4V mIgG2a vs. | Up | BP | tumor necrosis factor production | 2.13E−12 |
| B7H41001 mIgG2a mAb | | | | |
| SGN-B7H4V mIgG2a vs. | Up | BP | interferon-gamma production | 3.56E−12 |
| B7H41001 mIgG2a mAb | | | | |

### Example 19: Combination of SGN-B7H4V mIgG2a with an Anti-PD-1 Agent Demonstrates Enhanced Anti-Tumor Activity Compared to Either Agent Alone

The activity of SGN-B7H4V mIgG2a was then evaluated in combination with an anti-PD-1 agent in murine B7-H4-expressing Renca tumor-bearing immunocompetent mice. Tumor-bearing mice were treated with 3 weekly doses of a sub-therapeutic dose of SGN-B7H4V mIgG2a (1 mg/kg) and unconjugated anti-PD-1 antibody (0.3 mg/kg) alone or in combination when tumor volumes reached 100 mm³.

SGN-B7H4V mIgG2a in Combination with an Anti-PD-1 mAb Elicited Enhanced Antitumor Activity

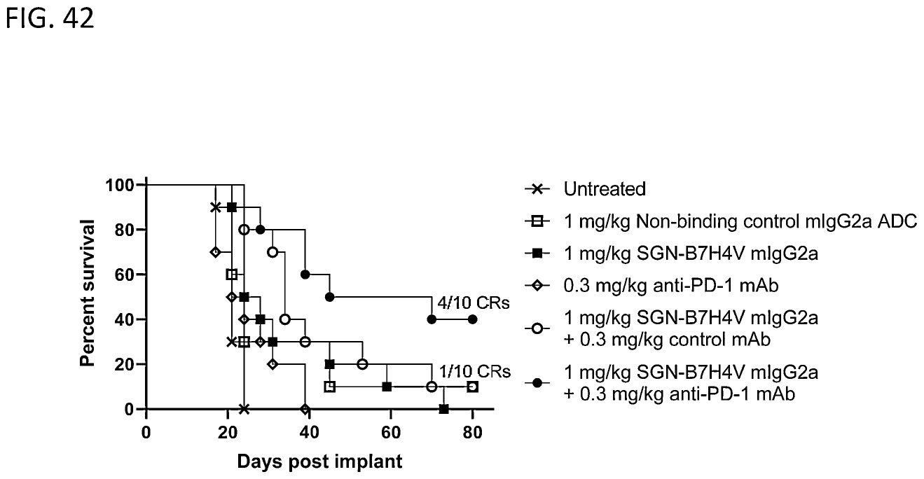

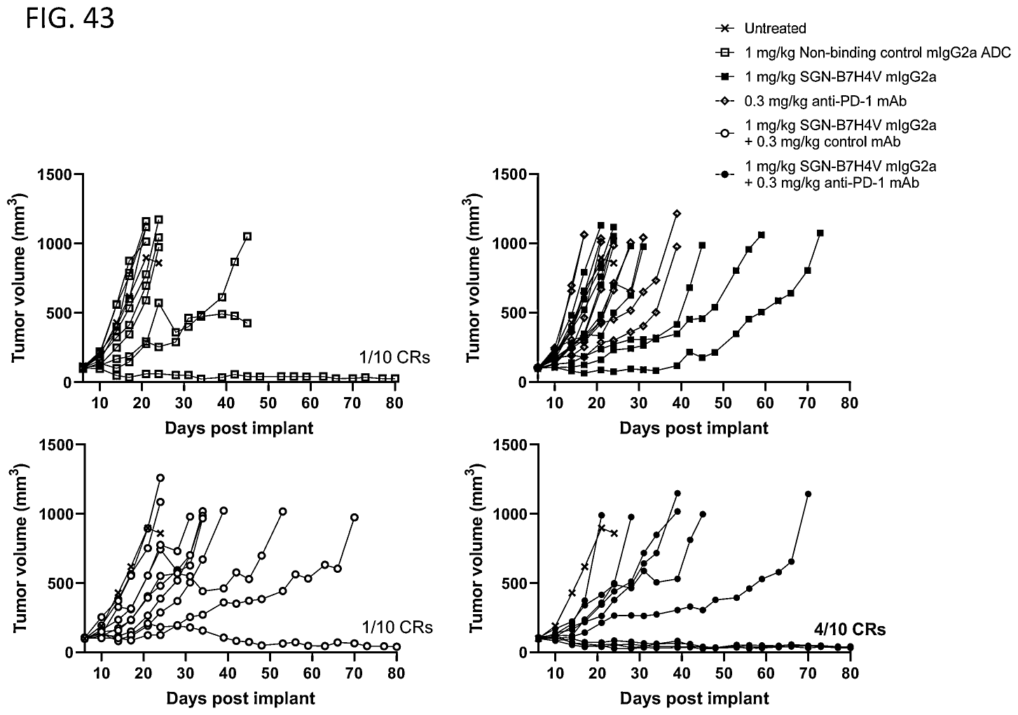

Treatment SGN-B7H4V mIgG2a in combination with the anti-PD-1 mAb led to enhanced survival and anti-tumor activity (with 4/10 complete responses observed), as compared to either treatment alone or SGN-B7H4V mIgG2a in combination with a rat isotype control mAb (FIGS. 42 and 43).

SGN-B7H4V mIgG2a in Combination with an Anti-PD-1 mAb Elicits Robust Immune Memory

Next, the ability of SGN-B7H4V mIgG2a in combination with an anti-PD-1 agent to elicit durable immune memory was evaluated in tumor rechallenge studies. Mice from Example 17 (i.e., treated with SGN-B7H4V mIgG2a, See FIGS. 36A, B) and from above (i.e., treated with SGN-B7H4V mIgG2a+anti-PD-1 mAb, See FIGS. 42 and 43) which achieved durable tumor regression were re-challenged with the parental (i.e., non-B7-H4-expressing) Renca tumor cells. All four mice previously treated with SGN-B7H4V mIgG2a in combination with an anti-PD-1 mAb were protected from tumor rechallenge (Table 25), suggesting that the combination of SGN-B7H4V in combination with an anti-PD-1 agents is synergistic and elicits durable immune memory.

TABLE 25
SGN-B7H4V mIgG2a in combination with an anti-
PD-1 mAb elicits robust immune memory.

| | Tumor cells | % Protection |
|---|---|---|
| Mice with complete responses | used for | from tumor |
| being rechallenged | rechallenge | rechallenge |
| 3 mg/kg SGN-B7H4V mIgG2a | Parental Renca | 40% (2/5 mice) |
| (FIGS. 36A, B) | | |
| 1 mg/kg SGN-B7H4V mIgG2a + | Parental Renca | 100% (4/4 mice) |
| 0.3 mg/kg anti-PD-1 mAb | | |
| (FIGS. 42, 43) | | |

1. A B7-H4 antibody-drug conjugate (B7-H4-ADC), wherein the B7-H4-ADC comprises an anti-B7-H4 antibody conjugated to a vcMMAE (valine-citruline-monomethyl auristatin E), wherein the anti-B7-H4 antibody comprises heavy chain variable region (VH)-complementarity determining region (CDR) 1, VH-CDR2, VH-CDR3 and light chain variable region (VL)-CDR1, VL-CDR2, and VL-CDR3 sequences of SEQ ID NOs: 5-10, respectively; wherein the vcMMAE comprises the structure: 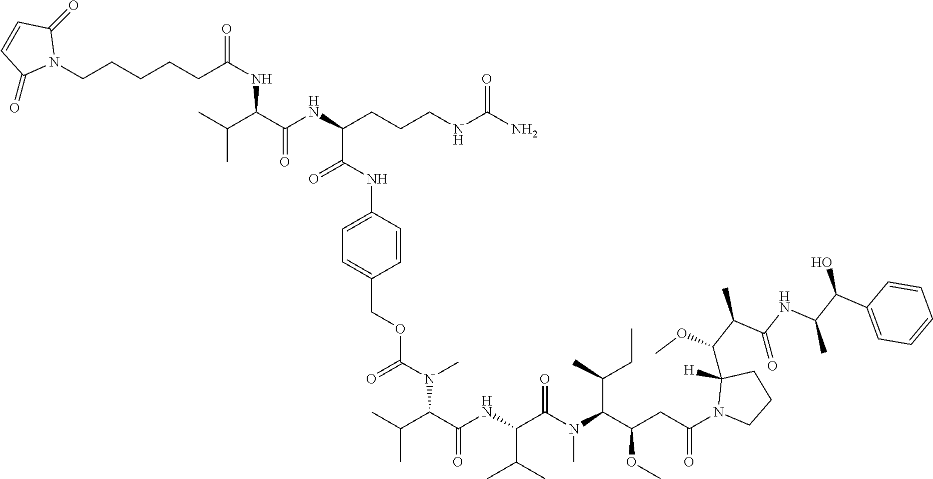 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
or a pharmaceutically acceptable salt thereof.

2. The B7-H4-ADC of claim 1, wherein the anti-B7-H4 antibody comprises a heavy chain variable region (HCVR) having at least 95% identity to SEQ ID NO: 11, and a light chain variable region (LCVR) having at least 95% identity to SEQ ID NO: 12.

3. A B7-H4 antibody-drug conjugate (B7-H4-ADC), wherein the B7-H4-ADC comprises an anti-B7-H4 antibody conjugated to a vcMMAE (valine-citruline-monomethyl auristatin E), wherein the anti-B7-H4 antibody comprises a heavy chain variable region (HCVR) having at least 95% identity to SEQ ID NO: 11, and a light chain variable region (LCVR) having at least 95% identity to SEQ ID NO: 12,
wherein the vcMMAE comprises the structure: 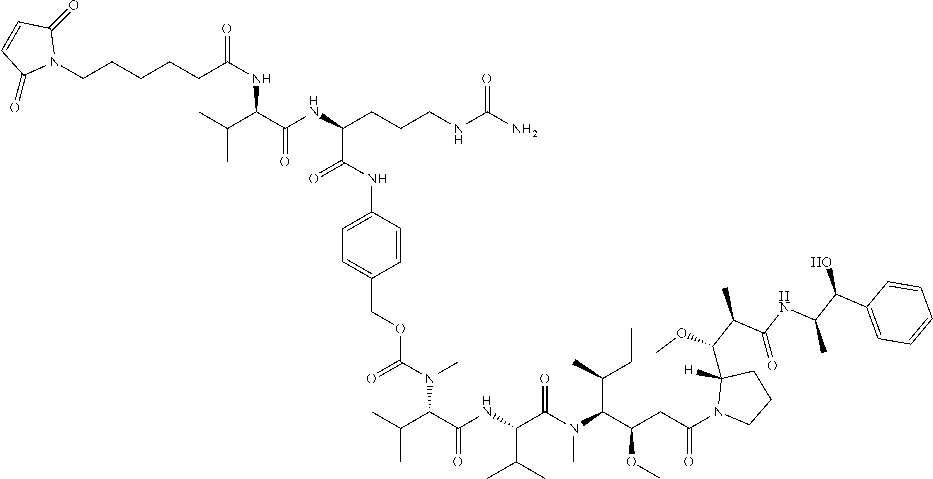 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
or a pharmaceutically acceptable salt thereof.

4. The B7-H4-ADC of claim 3, wherein the heavy chain variable region of the anti-B7-H4 antibody comprises the three complementarity determining regions (CDRs) of any one of SEQ ID NO: 11, and the light chain variable region of the antibody or antigen-binding fragment thereof comprises the three CDRs of SEQ ID NO: 12.

5. The B7-H4-ADC of claim 1, wherein the heavy chain variable region has at least 98% identity to SEQ ID NO:11 and the light chain variable region has at least 98% identity to SEQ ID NO:12.

6. (canceled)

7. The B7-H4-ADC of claim 1, wherein the heavy chain variable region comprises the sequence of SEQ ID NO: 11 and the light chain variable region comprises the sequence of SEQ ID NO: 12.

8. The B7-H4-ADC of claim 1, wherein the B7-H4-ADC comprises the structure:

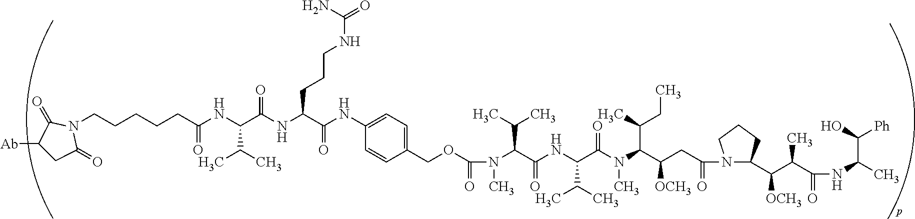

9. The B7-H4-ADC of claim 1, wherein the B7-H4-ADC comprises the structure:
(a)

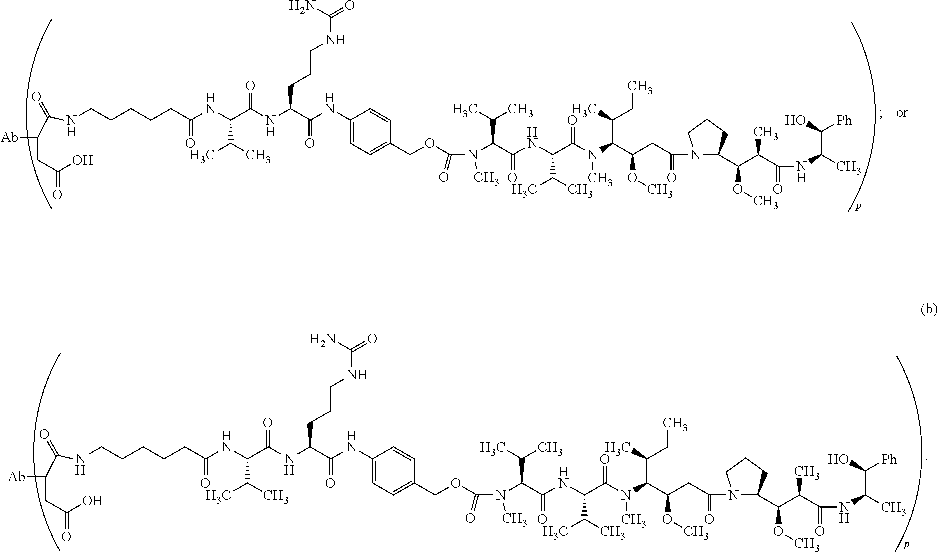

10. The B7-H4-ADC of claim 1, wherein a vcMMAE to antibody ratio is from about 1 to about 8.

11. The B7-H4-ADC of claim 1, wherein the vcMMAE to antibody ratio is about 4.

12. The B7-H4-ADC of claim 1, wherein the anti-B7-H4 antibody is a fully human antibody.

13. The B7-H4-ADC of claim 1, wherein the anti-B7-H4 antibody is a IgG1 monoclonal antibody.

14. The B7-H4-ADC of claim 1, wherein the B7-H4-ADC is within a heterogeneous population of B7-H4-ADCs, wherein the anti-B7-H4 antibodies comprised within the heterogeneous population of B7-H4-ADCs exhibit variable post-translational modifications.

15. The B7-H4-ADC of claim 14, wherein within at least 50%, of the anti-B7-H4 antibodies comprised within the heterogeneous population of B7-H4-ADCs:
(i) the C-terminal lysine residues are removed from both heavy chains; and/or
(ii) the N-terminal glutamine of each heavy chain cyclized to pyroglutamic acid; and/or
(iii) the consensus glycosylation site at Asn300 of each heavy chain occupied predominantly with biantennary, core fucosylated glycans without terminal galactose residues.

16. A method of treating a subject having or at risk of having a B7-H4-associated cancer, comprising:
administering to the subject a therapeutically effective dose of a B7-H4 antibody-drug conjugate (B7-H4-ADC),
wherein the B7-H4-ADC comprises an anti-B7-H4 antibody conjugated to a vcMMAE (valine-citruline-monomethyl auristatin E), wherein the anti-B7-H4 antibody comprises heavy chain variable region (VH)-complementarity determining region (CDR) 1, VH-CDR2, VH-CDR3 and light chain variable region (VL)-CDR1, VL-CDR2, and VL-CDR3 sequences of SEQ ID NOs: 5-10, respectively;
wherein the vcMMAE comprises the structure: 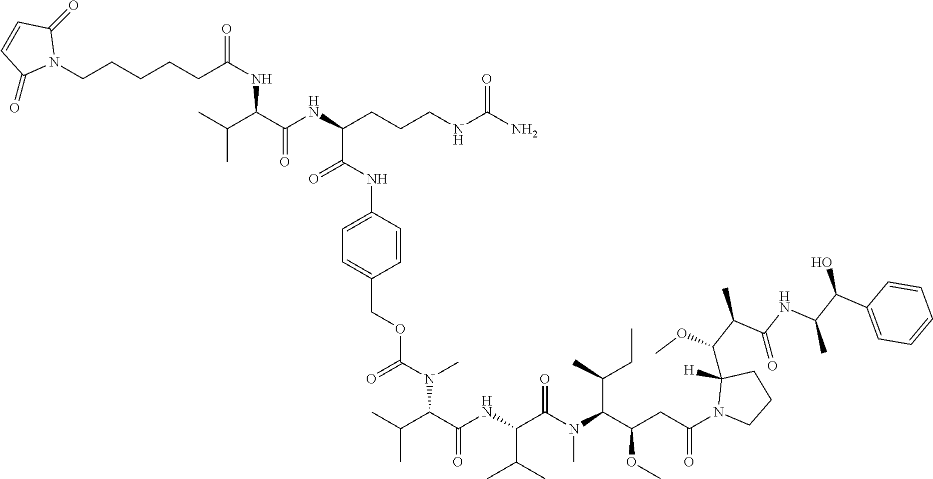 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
or a pharmaceutically acceptable salt thereof.

17. The method of claim 16, wherein the anti-B7-H4 antibody comprises a heavy chain variable region (HCVR) having at least 95% identity to SEQ ID NO: 11, and a light chain variable region (LCVR) having at least 95% identity to SEQ ID NO: 12.

18. A method of treating a subject having or at risk of having a B7-H4-associated cancer, comprising:
administering to the subject a therapeutically effective dose of a B7-H4 antibody-drug conjugate (B7-H4-ADC),
wherein the B7-H4-ADC comprises an anti-B7-H4 antibody conjugated to a vcMMAE (valine-citruline-monomethyl auristatin E), wherein the anti-B7-H4 antibody comprises a heavy chain variable region (HCVR) having at least 95% identity to SEQ ID NOs: 11, and a light chain variable region (LCVR) having at least 95% identity to SEQ ID NO: 12, wherein the vcMMAE has the structure: 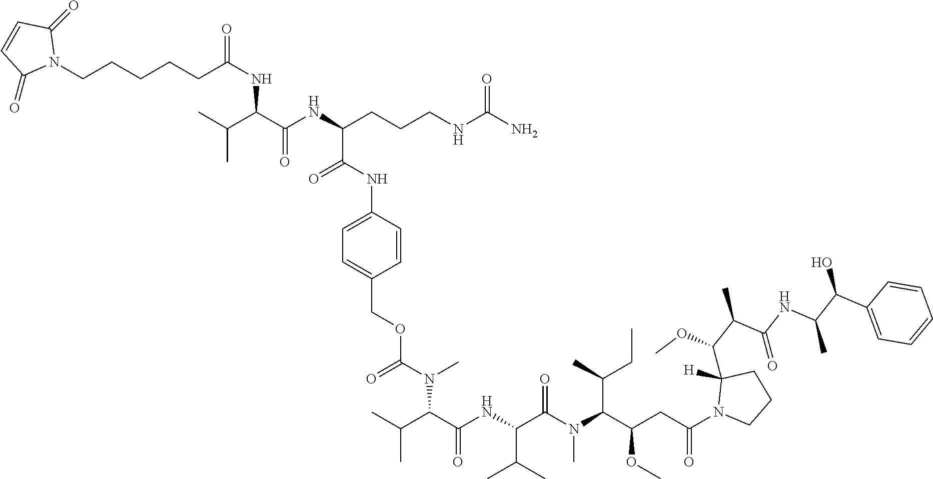 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](NC(=O)CCCCCN2C(=O)C=CC2=O)C(C)C)cc1)C(C)C]
or a pharmaceutically acceptable salt thereof.

19-33. (canceled)

34. The method of claim 16, wherein the cancer is an advanced stage cancer.

35. The method of claim 16, wherein the cancer is selected from the group consisting of breast cancer, ovarian cancer, lung cancer, cholangiocarcinoma and endometrial cancer.

36-61. (canceled)

62. A kit comprising:
(a) a B7-H4-ADC, or an antibody or antigen-binding fragment thereof that binds B7-H4; and
(b) instructions for using the B7-H4-ADC.

63. (canceled)
